(19) United States
(12) Patent Application Publication
(10) Pub. No.: US 2024/0269203 A1
(43) Pub. Date: Aug. 15, 2024

(54) ONCOLYTIC VIRUS AND USES THEREOF

(71) Applicant: Janssen Biotech, Inc., Horsham, PA (US)

(72) Inventors: David Baillat, Washington, DC (US); Charles Drake, New Hope, PA (US); Steven R. Fuhrmann, Germantown, MD (US); Paul Harvilla, Blue Bell, PA (US); Sylvie Laquerre, Chesterbrook, PA (US); Matthew Mulvey, Gaithersburg, MD (US); Manuel Sepulveda, West Windsor, NJ (US); Raghavendra Shamanna, Exton, PA (US); Ninkka Tamot, Colmar, PA (US); Kathryne Taylor, Rockville, MD (US); Douglas Yamada, Ardmore, PA (US)

(73) Assignee: Janssen Biotech, Inc., Horsham, PA (US)

(21) Appl. No.: 18/545,113

(22) Filed: Dec. 19, 2023

Related U.S. Application Data
(60) Provisional application No. 63/433,781, filed on Dec. 20, 2022.

(51) Int. Cl. A61K 35/763; A61P 35/00; C07K 14/475; C07K 14/54; C07K 16/28; C12N 15/86

(52) U.S. Cl. CPC A61K 35/763; A61P 35/00; C07K 14/475; C07K 14/5434; C07K 16/2818; C07K 16/2878; C12N 15/86; C07K 2317/75; C12N 2710/16622; C12N 2710/16632; C12N 2710/16643; C12N 2710/16671

(57) ABSTRACT

Provided herein are oncolytic viruses including payload genes, encompassing genes that encode IL-12, FLT3L, CD40 agonists, and/or CTLA-4 antibodies. Also provided are expression cassettes, pharmaceutical compositions, and methods of treatment employing these viruses. Furthermore, expression cassettes and CD40 agonist molecules are also provided in this disclosure.

### CROSS-REFERENCE TO RELATED APPLICATIONS

This application claims priority to U.S. Provisional Application Ser. No. 63/433,781, filed 20 Dec. 2022, the entire contents of which is incorporated herein by reference.

### REFERENCE TO SEQUENCE LISTING SUBMITTED ELECTRONICALLY

This application contains a sequence listing, which is submitted electronically via EFS-Web as an XML formatted sequence listing with a file name “JBI6709USNP1 Seqlist.xml” creation date of 18 Dec. 2023 and having a size of 209 KB. The sequence listing submitted via EFS-Web is part of the specification and is herein incorporated by reference in its entirety.

### FIELD OF THE INVENTION

The present invention relates to oncolytic viruses, including those derived from herpes simplex virus 1 (HSV-1), expression cassettes, and proteins expressed from said oncolytic viruses and/or expression cassettes.

### BACKGROUND

Oncolytic virus therapy is a form of immunotherapy that exploits the cytotoxic and/or vector ability of viruses to selectively target and destroy tumor cells. Oncolytic virus therapy can also work to stimulate immune responses against a target tumor or tumors distal to a target tumor. Safety issues limited the use of live, infectious viruses in cancer patients, but the development of robust genetic engineering has allowed the field to mature by the development of improved viruses. Kelly and Russell, “History of oncolytic viruses: genesis to genetic engineering,” Mol. Ther. 2007 April; 15(4):651-9. Oncolytic viruses may be engineered to have enhanced selectively for tumor cells (for example, by enhanced cytotoxicity in cancer cells and/or reduced cytotoxicity in normal cells) and to express therapeutic payloads, such as immunostimulatory proteins.

Herpes simplex viruses (HSV) are candidates for additional oncolytic virus development. HSV has a broad host cell range in humans, a short replication cycle, a large genome which is amenable to multiple payload genes, and effective antiviral options to control infection. Sanchala et al., “Oncolytic Herpes Simplex Viral Therapy: A Strike toward Selective Targeting of Cancer Cells,” Front. Pharmacol. 2017; 8:270.

Next generation oncolytic viruses, including those derived from HSV, that exhibit increased safety and increased efficacy are needed in the art.

All references cited herein, including patent applications, patent publications, and scientific literature, are herein incorporated by reference in their entirety, as if each individual reference were specifically and individually indicated to be incorporated by reference

### SUMMARY OF THE INVENTION

Provided herein are oncolytic viruses comprising one or more expression cassettes comprising a polynucleotide encoding IL-12, a polynucleotide encoding a CD40 agonist, a polynucleotide encoding a CTLA-4 binding protein and/or a polynucleotide encoding a FLT3 ligand. Also provided here are expression cassettes comprising such polynucleotides. In some embodiments, provided herein are methods of treating cancer in an individual comprising administering the oncolytic viruses provided herein to the individual. In some embodiments, the oncolytic viruses provided herein trigger an abscopal response to a distant tumor and/or cause an immunological memory of a tumor.

### BRIEF DESCRIPTION OF THE DRAWINGS

The present application can be understood by reference to the following description taken in conjunction with the accompanying figures.

FIG. 1A shows the design of a viral genome mimicking a previously-reported genetically engineered oncolytic herpesvirus, with only one copy of IE-US11 (left panel), and of an engineered innate Stealth virus with both endogenous L-US11 and IE-US11 expression (right panel). IR_{L}, internal repeat long; IR_{S}, internal repeat short; TR_{L}, terminal repeat long; TR_{S}, terminal repeat short; U_{L}, unique long; U_{S}, unique short; β-Gluc, β-glucuronidase. S shown in a hexagon represents a stop codon. Co-US11 represents codon-optimized US11 as an IE gene. A star represents a promoter or transcription start site. An arrow originating from a star represents an RNA transcript.

FIG. 1B shows the results of a Western blot experiment that measured the levels of US11 and phosphorylated (p-)eIF2α in A549 human lung cancer cells infected (MOI=5) with wild type (WT) HSV-1, a Δγ34.5 HSV-1, a virus mimicking a previously-reported genetically engineered oncolytic herpesvirus, and an engineered innate Stealth virus that expresses both immediate early (IE) and late (L) US11 (see, FIG. 1A and Example 1 herein). ICP27 viral protein and actin were used for viral infection and loading controls, respectively. MOI, multiplicity of infection. 6 h=6 hours post infection; 12 h=12 hours post infection; 18 h=18 hours post infection.

FIG. 2A shows the design of a viral genome expressing a model antigen SIINFEKL (SEQ ID NO: 319) (SIINFEKL (SEQ ID NO: 319)) and UL49.5 (left panel: “41T_2B”), and of a viral genome that is genetically identical to 41T_2B, except for the presence of a stop codon (shown as a hexagon) inserted within UL49.5 to prevent its expression (right panel: “41T_4B”). iRFP, near-infrared fluorescent protein. Figure discloses “SIINFEKL” as SEQ ID NO: 319.

FIG. 2B provides the results of two independent flow cytometry experiments (Run 1 and Run 2 indicated by circles and triangles, respectively) using MB49 cells infected with the indicated viruses (MOI=10) and stained for H2Kb (MHC) and SIINFEKL-H2Kb (MHC-SIINFEKL (“SIINFEKL” disclosed as SEQ ID NO: 319)). The top panel shows the percent of live, 41T_2B- or 41T_4B-infected (iRFP) cells expressing MHC. The bottom panel shows the percent of live, 41T_2B- or 41T_4B-infected cells presenting the SIINFEKL (SEQ ID NO: 319) antigen in context of MHC.

FIG. 3 is a diagram depicting the rationale for the selection of four immunomodulatory payloads for HSV-1 OV development, as described in Example 2 herein. APC, antigen-presenting cell; CTLA-4, cytotoxic T lymphocyte-associated protein 4; CD, cluster of differentiation; DC, dendritic cell; FLT3L, fms like tyrosine kinase 3 ligand; IL, interleukin; Th, T helper; TME, tumor microenvironment.

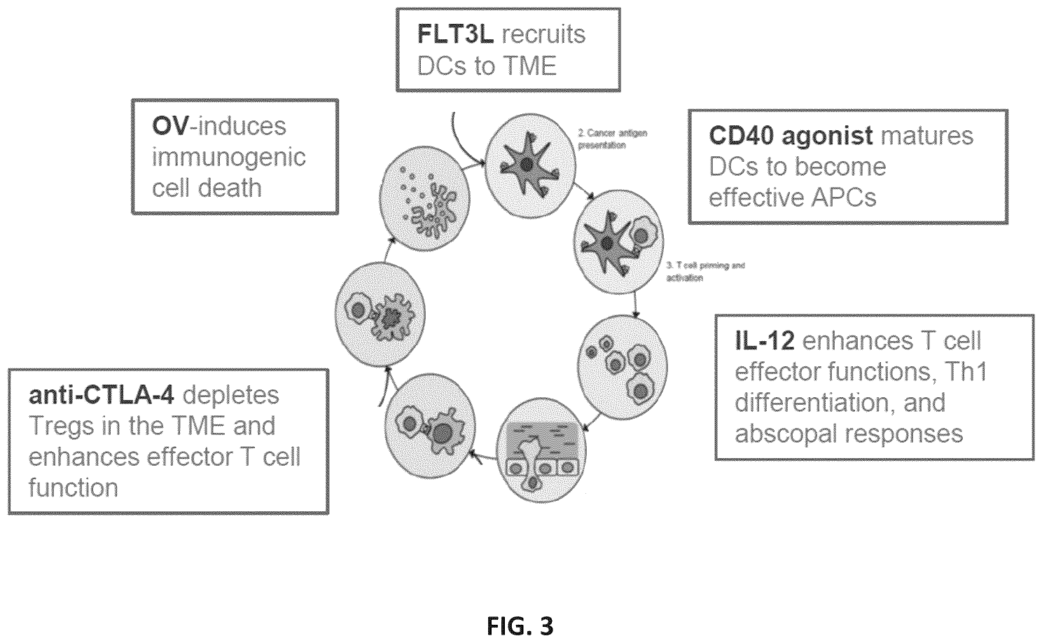

FIG. 4 shows that bivalency of anti-CTLA-4 molecules is required to block CTLA-4 interaction with CD80:CD86. The indicated anti-CTLA-4 molecules (i.e., anti-CTLA-4 mAb, Fab′2, Fab, scFv, scFv-G1m1, and scFv-G1m(17)) were tested using a reporter assay for their ability to block CTLA-4 interaction with CD80:CD86. Reporter luminescence (RLU) indicates the ability to block CTLA-4 interaction with CD80:CD86. Fab, fragment antigen-binding; mAb, monoclonal antibody; RLU, relative light unit; scFv, single-chain variable fragment; anti-CTLA-4 mAb is anti-CTLA-4 monoclonal antibody.

FIG. 5A-5B show that an anti-CTLA-4 payload for use in an HSV-1 OV should have an active Fc region (capable of binding Fc-gamma receptors) for in vivo activity. FIG. 5A shows the effect of the indicated anti-CTLA-4 molecules on tumor growth (assessed as mean tumor volume, mm³±standard error of the mean (SEM)) on the indicated days post tumor implant. The anti-CTLA-4 molecules were administered intratumorally (IT) biweekly for a total of 4 treatments (b.i.w×4). FIG. 5B shows the effects of the indicated anti-CTLA-4 molecules on Treg depletion (assessed by the percent of CD4+ T cells expressing FoxP3 and CD25 using flow cytometry) or T cell activation (assessed by the percent of CD8+ T cells expressing IFNγ and TNFα using flow cytometry following restimulation with tumor antigen peptides) in tumors from the mice treated similar to those in the experiment shown in FIG. 5A. The anti-CTLA-4 molecules shown in FIGS. 5A-5B are a positive (+) control anti-CTLA4 mAb, an scFv containing anti-CTLA4 mAb variable regions fused to and IgG1-Fc, and an Fc-less construct composed of two scFvs containing anti-CTLA mAb variable regions attached by a linker (i.e., Ipi-Fab tandem-scFv).

FIG. 6A-6B provide diagrams of the anti-CTLA4 and CD40 agonist payloads selected for inclusion in an HSV-1 OV. FIG. 6A shows the design of the selected anti-CTLA-4 antagonist payload, termed hαCTLA-4. The anti-CTLA-4 antagonist is an anti-CTLA-4 single-chain variable fragment (scFv) fused to the N-terminus of the heavy chain of human IgG1_G1m(17). FIG. 6B shows the design of the selected CD40 agonist payload, termed hCD40ag. The CD40 agonist is a trimerized, C-terminal fusion protein containing trimeric bundles of human CD40L ectodomains that bind to the CD40 receptor protein. hCD40ag has the 27 amino acid trimerization motif from the fibritin protein of T4 phage fused with a glycine/serine linker to the N-terminal CD40L ectodomain trimer bundles. The CD40L ectodomain trimer bundles consist of individual CD40L peptide fused together by glycine/serine linkers from the C-terminus of one peptide to the N-terminus of the subsequent peptide to form single polypeptide trimeric bundles. FIGS. 6A and 6B disclose SEQ ID NOS 23, 22, and 320, respectively, in order of appearance.

FIG. 6C-6D provide diagrams of the FLT3L and IL 12t payloads selected for inclusion in an HSV-1 OV shows a crystal structure of the selected FLT3L payload, termed hFLT3L, which is a full-length human FLT3L molecule. The molecule features a native signal peptide to allow for efficient secretion to the plasma membrane where it is anchored by a transmembrane helix. FLT3L forms a functional dimer and can be processed into a soluble version. FIG. 6D shows a crystal structure of the selected IL-12 payload, termed human single chain IL-12 (hscIL-12). The hscIL-12 payload is a human IL-12 fusion protein containing the human p40 subunit, also known as IL-12 subunit beta (Uniprot: P29460), from IL-12 fused with a glycine/serine linker to the N-terminus of human p35, also known as IL-12 subunit alpha (Uniprot: P29459). A 6-mer polyhistidine sequence (6×-His Tag (SEQ ID NO: 320)) C-terminal of the p35 subunit was added to aid in purification of recombinant protein by Ni-NTA resin. FIG. 6C and CD disclose SEQ ID NOS 3 and 320, respectively, in order of appearance.

FIG. 7A-7B show that an Fc-less CD40 agonist termed hCD40ag is bioactive in vitro. FIG. 7A shows the effect of CD40 agonists on CD40 pathway activity using a reporter cell line that emits signal following activation of the CD40 pathway. The CD40 agonists tested were hCD40ag (an Fc-less trimer of CD40L trimer bundles), and hCD40ag2 (includes bivalent CD40L trimer bundles connected to an Fc). Two positive controls (Positive Controls 1 & 2) were tested, as well as a media-only negative control. Activation of the CD40 pathway is represented as optical density (OD) at 650 nm (y-axis) using the CD40 agonists at the concentrations indicated on the x-axis (ng/mL). FIG. 7B shows the effects of the indicated CD40 agonists on dendritic cell (DC) activation, assessed by CD86 expression using flow cytometry. The CD40 agonists tested included hCD40ag, as well as several controls: Positive Controls 1 & 2, wild type CD40 agonist (soluble monomer), and an IgG1 isotype control antibody. Each CD40 agonist or control was used at the concentration indicated on the x-axis. gMFI, geometric mean fluorescence intensity.

FIG. 8A-8B show the anti-tumor efficacy of several CD40 agonists in vivo using MC38-5AG tumors in human CD40 knock-in mice. The CD40 agonists indicated in FIG. 8A were injected IT on Days 1, 4, and 7 to mimic expression from an OV, and tumor volume (mm³) was measured on the indicated days after treatment. The tested agonists were hCD40ag (i.e., an Fc-less trimer of CD40L trimer bundles), hCD40ag2 (i.e., bivalent CD40L trimer bundles connected to an Fc), and WT CD40 agonist (CD40L) monomer. An isotype control antibody (IgG1 mAb) was also tested. The confidence interval (CI) values are the lower and upper limits of the 95% CI of the tumor growth inhibition (TGI) value, and the p-value compared to isotype control were calculated for each construct tested by InVivoLDA Version 4.8. FIG. 8B provides a comparison of average tumor volumes as measured in mice treated with hCD40ag or isotype control antibody (IgG1 mAb). The arrows in FIG. 8B indicate the days on which the mice were treated with hCD40ag or isotype control antibody.

FIG. 9A-9B show the functionality and bioactivity of the selected FLT3L payload, termed hFLT3L. FIG. 9A shows the levels of soluble FLT3L in the supernatant of the indicated human tumor cell lines (A375, A549, H1299, HT29, and 22Rv1) infected with three viruses expressing hFLT3L. Each bar within the groups of three bars for each tumor cell line on the x-axis corresponds to a different HSV-1 virus that expresses hFLT3L. FIG. 9B shows the effect of human WT FLT3L protein on DC differentiation. Differentiation of DCs (MHCII CD11c) from mouse bone marrow was assessed by flow cytometry upon treatment with recombinant human FLT3L (rhFLT3L), or the supernatant of Vero cells infected (MOI=1) with either an HSV-1 virus expressing hFLT3L or a negative control virus with similar architecture, but which did not express hFLT3L. The amount of hFLT3L in the supernatant was determined by enzyme-linked immunosorbent assay (ELISA). The activity of rhFLT3L was assessed alone or spiked in the supernatant from cells infected with the negative control virus.

FIG. 10A shows the results of an enzyme-linked immunosorbent assay (ELISA) that was used to measure IFNγ (pg/mL) produced by human peripheral blood mononuclear cells (PBMCs) treated with recombinant hIL-12 (2 subunits) or the selected hscIL-12 payload at the concentrations indicated on the x-axis. The bars corresponding to the selected hscIL-12 payload are marked with an arrow; the bars corresponding to recombinant hIL-12 (Commercial rhIL-12) are not marked.

FIG. 10B shows the results of an ELISA that was used to measure IFNγ (pg/mL) produced by human PBMCs treated with suboptimal PHA/PMA stimulation for T cell activation and then recombinant hIL-12 (rhIL-12) or the supernatant of Vero cells infected (MOI=1) with a virus expressing the hscIL-12 payload, or a negative control virus not expressing the hscIL-12 payload. The amount of hIL-12 in the supernatant was determined by ELISA, as indicated on the x-axis. The activity of rhIL-12 was assessed alone or spiked in the supernatant from cells infected with the negative control virus. The bars corresponding to rhIL-12 are marked with “R”; the bars corresponding to the virus expressing the hscIL-12 payload are marked with an arrow; the bars corresponding to rhIL-12 spiked in the supernatant from cells infected with the negative control virus are marked with “R/−”; and the bars corresponding to the negative control virus are marked with “−”.

FIG. 11 shows that the anti-CTLA-4 antagonist (hαCTLA-4), CD40 agonist (hCD40ag), and IL-12 (hscIL-12) payloads have a synergistic effect on T cell activation. Human PBMCs were incubated with a fixed concentration of T-cell-activating super antigen in the presence of increasing concentrations of hαCTLA-4) alone, or the double combination of hαCTLA-4 and hscIL-12 (FIG. 11, left). In addition, PBMCs were exposed to a fixed concentration of hCD40ag to activate APCs, then combined with increasing concentrations of hαCTLA-4 or the combination of hαCTLA-4 and hscIL-12 (FIG. 11, right). T-cell activation was measured by IL2 secretion into the supernatant, which was harvested on Day 5.

FIG. 12A is a diagram showing the architecture of cassettes with eight different promoter combinations and orientations. Each of the eight promoter combinations and orientations includes promoters selected from mCMV, MMLV, AoHV1, and Pbidir3 (described in WO 2016/166088).

FIG. 12B shows the 10 sets of polyadenylation signal pairs (pA1 and pA2), leading to 80 reporter cassettes. A reporter system was used to screen the 80 designed cassettes. The payloads were replaced by two fluorescent proteins (i.e., DsRed and TagBFP2) and a plasma membrane marker easily detectable by flow cytometry (i.e., mThy1.1). The positions of the reporter genes (i.e., mThy1.1, DsRed, and TagBFP2) were fixed, as was the 3′ most polyadenylation signal (US9-10 pA).

FIG. 13A is a schematic of payload expression Cassettes 17E, 37E and 75E.

FIG. 13B provides the expression levels of the anti-CTLA-4 antagonist(hαCTLA-4), CD40 agonist (hCD40ag), and IL-12 (hscIL-12) payloads for each of Cassettes 17E, 37E and 75E in supernatants of two different cell lines (HEK293T and H1299) collected after transient transfection after 24 hours (H1299) and 48 hours (HEK293T) (mean±SD, n=2 technical replicates). Payload expression levels were assessed by a blockade assay, secreted alkaline phosphatase assay, and an ELISA assay. The pUC57 plasmid vector backbone (without the expression cassettes) was used as a negative control.

FIG. 14 shows the expression levels of the hFLT3L, anti-CTLA-4 antagonist (hαCTLA-4), CD40 agonist (hCD40ag), and IL-12 (hscIL-12) payloads from the indicated OVs upon infection of five different tumor cell lines. The OVs tested were JP-OV-1 (containing the 17E cassette), JP-OV-2 (containing the 37E cassette), and a parental OV which expresses hFLT3L. Payload expression was quantified by ELISA (Mean±SD, n=3 technical replicates) from the culture medium of five cancer cell lines infected at MOI=1 after 24 hour infection. The parental virus (expressing hFLT3L) was used as positive control for hFLT3L and as negative control for the three other payloads. To account for differences of total cell count at same culture cell density, concentrations of the payloads were expressed per 106 cells (at time of infection). * indicates concentrations below the level of detection of the assay. Bars corresponding to the parental OV are marked with a “P”; bars corresponding to JP-OV-1 are marked with “1”; and bars corresponding to JP-OV-2 are marked with “2”.

FIG. 15A-15B are diagrams showing the multi-step construction of oncolytic virus JP-OV-2. FIG. 15A is a diagram of the genome structure of the ΔXN1 parental virus used to generate JP-OV-2. β-Gluc represents replacement of neurovirulence gene γ34.5 with β-glucuronidase in the γ34.5 locus. US12, along with its intron sequence (shown as latch), were deleted from the US10-12 locus, represented in light grey color. The virus expresses US11 as an immediate-early gene using the US12 promoter. FIG. 15B is a diagram of the genome structure of the ΔXN1-GFP virus. The virus expresses eGFPS from the γ34.5 locus using a CMV promoter and US11 from the US10-12 locus using the US12 promoter.

FIG. 15C-15D are additional diagrams showing the multi-step construction of oncolytic virus JP-OV-2. FIF 15C is a diagram showing the genome structure of the Step 1 virus. The virus was engineered to express the hFLT3L and UL49.5 transgenes using a CMV promoter from γ34.5 locus. US11 is expressed using the immediate early US12 promoter. FIG. 15D is a diagram of the genome structure of the Step 2 virus. The US10-12 locus was engineered to express eGFP. The US12 intron (shown as dark colored latch) was added back to US10-12 locus. US11 is expressed using the US12 promoter.

FIG. 15E is a diagram of the genome structure of Step 3 virus, which is the final virus construct in JP-OV-2. The virus was engineered to express the anti-CTLA-4 antagonist (hαCTLA-4), CD40 agonist (hCD40ag), and IL-12 (hscIL-12) payloads from the US10-12 locus, and hFLT3L and UL49.5 from the γ34.5 locus. The virus expresses codon-optimized US11 (hCoUS11) using the US12 immediate early promoter and endogenous US11 using late US11 promoter. “S” shown in the hexagon represents a stop codon between hCoUS11 and US12, which inhibits US12 expression. US12 in grey indicates that the gene does not express. IR_{L}, internal repeat long; IR_{S}, internal repeat short; TR_{L}, terminal repeat long; TR_{S}, terminal repeat short; U_{L}, unique long; U_{S}, unique short.

FIG. 16A shows immunoblots for ICP27, US11, UL49.5, and actin expression from Vero cell lysates infected with the indicated viruses. ICP27 and actin were used as controls.

FIG. 16B provides a quantification of the immunoblots shown in FIG. 16A. US11 and UL49.5 protein levels were quantitated and normalized to ICP27 and actin. The relative fold expression of UL49.5 and US11 from Step 3 clones 1˜4 was compared either to Δγ34.5 or to Step 2 viruses.

FIG. 17 shows the expression of the hFLT3L, anti-CTLA-4 antagonist (hαCTLA-4), CD40 agonist (hCD40ag), and IL-12 (hscIL-12) payload proteins from Vero cells infected at MOI=1 with the indicated viruses. Expression of the payload proteins was analyzed by ELISA using the cell culture supernatants. The Δγ34.5 virus served as negative control virus for hFLT3L and hscIL-12 expression, and the Step 2 virus served as parental control virus not encoding the hαCTLA-4, hCD40ag, and hscIL-12 payloads (i.e., within the 37E cassette). PY23_40A_P2_1_1 served as positive control for hscIL-12 and hαCTLA-4 expression. PY16_26C_1_1, an OV HSV-1 virus, served as positive control for hCD40ag expression.

FIG. 18 shows the cytotoxicity of Step 3 virus clones 1-4, assessed by cell viability of virus-infected cells. HT29 cells (top panel), stringent for virus replication, or Vero cells (bottom panel), highly permissive and used and used to confirm that similar amounts of each virus were used in the experiment, were infected (MOI=0.01) by the indicated viruses. Virus-induced cytopathic effect was monitored using xCelligence system at 6 hour intervals for 120 hours post infection. Cytotoxicity and percent viability of cells in the presence of virus was calculated based on cell index of mock-infected cells. The results shown represent the mean of six replicate values.

FIG. 19 shows the genetic stability of JP-OV-2 upon multiple cell culture replication cycles, assessed by key protein expression levels. Vero cells were infected (MOI=5) by JP-OV-2 collected following replication Cycle 6, 7, and 8. Expression of ICP27, US11, UL49.5, and the hCD40ag payload was detected by Western blot in total cell extracts and culture supernatants (hCD40ag only) at 6 hours post infection. Δγ34.5 virus was used as a negative control, and an early-passage JP-OV-2 was also used as a control (shown as “JP-OV-2”).

FIG. 20 shows the genetic stability of JP-OV-2 upon multiple cell culture replication cycles, assessed by immunomodulatory payload protein expression level. The expression of hFLT3L, CD40 agonist (hCD40ag), anti-human CTLA-4 antagonist (hαCTLA-4), and single-chain human IL-12 (hscIL-12) payload proteins was determined by ELISA (mean±SD; * indicates concentrations below the level of detection of the assay) in the culture supernatant of Vero cells infected (MOI=1, 24 hours post infection) with JP-OV-2 collected following replication Cycles 6, 7, and 8. Δγ34.5 virus was used as a negative control, and an early-passage JP-OV-2 was also used is as a control (shown as “JP-OV-2”).

FIG. 21 shows the functional stability of JP-OV-2 over multiple passages (cycles) on Vero cells, as determined by virus-induced cancer cell killing. HT29 cells (top two panels), stringent for OV replication, or Vero cells (bottom two panels), highly permissive for OV replication and used to confirm that similar amounts of each virus were used in the experiment, were infected with JP-OV-2 collected after multiple replication cycles (Cycles 6, 7, and 8; MOI=0.01), and compared to an early-passage JP-OV-2 (shown as “JP-OV-2”) for virus-induced cell death as monitored by xCelligence (% viability, y-axis).

FIG. 22A-22B show that JP-OV-2 is sensitive to acyclovir treatment. FIG. 22A shows an acyclovir dose-response experiment using wild-type HSV-1 (WT-HSV), a previously-reported genetically engineered oncolytic herpesvirus mimic, and JP-OV-2 viruses. Viral replication in Vero cells was assessed based on virus-induced cell death, as measured by xCelligence. FIG. 22B provides a comparison of virus replication in the absence of acyclovir in Vero cells, assessed by xCelligence, to confirm that similar amounts of each virus were used in the experiment shown in FIG. 22A.

FIGS. 23A-23B show that JP-OV-2 had an intermediate sensitivity to IFNβ. FIG. 23A shows a dose-response experiment of IFNβ-mediated inhibition of cell death induced by wild-type HSV-1 (WT-HSV), a virus only deleted for γ34.5 (Δγ34.5), a previously-reported genetically engineered oncolytic herpesvirus mimic, and JP-OV-2 (MOI=0.01) on normal human fibroblasts (HFFs). Virus-induced cell death was measured by xCelligence. FIG. 23B provides a comparison of virus replication on Vero cells in the absence of IFNβ, as measured by xCelligence, to confirm that similar amounts of each virus were used in the experiment shown in FIG. 23A.

FIG. 24 shows that JP-OV-2 has tumor growth inhibition (TGI) activity in vivo. Mice with H1299 human xenograft tumors were treated intratumorally (IT) with vehicle control or JP-OV-2 at 5×10⁴ or 5×106 PFU/injection once every 3 days for 3 doses (q3dx3). Tumor volume measurements were captured twice weekly for the duration of the study. TGI was calculated at Day 45 (D45) when 70% of animals remained in all groups. Dosing is indicated by black bar on the graph in top panel (below the x-axis). P-values (bottom panel) were calculated by Linear-Mixed Effects (LME) analysis.

FIG. 25 shows that hFLT3L expressed from JP-OV-2 has bioactivity, assessed using a DC differentiation assay. Differentiation of DCs (MHCII⁺CD11c⁺) from mouse bone marrow was assessed by flow cytometry upon treatment with recombinant hFLT3L (rhFLT3L), or the supernatant of Vero cells infected (MOI=1) with JP-OV-2, or a negative control virus with similar architecture but lacking hFLT3L. The amount of hFLT3L in the supernatant was determined by ELISA. The activity of rhFLT3L was assessed alone or spiked in the supernatant from cells infected with the negative control virus.

FIG. 26 shows that the hscIL-12 payload expressed from JP-OV-2 has bioactivity, assessed based on production of IFNγ by human PBMCs. IFNγ produced by PBMCs was assessed by ELISA upon treatment with suboptimal PHA/PMA stimulation for T cell activation and then recombinant hIL-12 (rhIL-12) or the supernatant of Vero cells infected (MOI=1) with JP-OV-2, or negative control Step 2 virus (lacking hIL-12). The amount of hIL-12 in the supernatant was determined by ELISA. The activity of rhIL-12 was assessed alone or spiked in the supernatant from cells infected with the negative control virus (rhIL-12+Step 2 Virus).

FIGS. 27A-27B provide diagrams of three payloads selected for inclusion in mJP-OV-2, a mouse surrogate HSV-1 OV as described in Example 6 herein. FIG. 27A is a diagram of the mouse surrogate CD40 agonist payload (termed “mCD40ag”). mCD40ag is a trimerized, C-terminal fusion protein consisting of trimeric bundles of mouse CD40L ectodomains. The molecule features the 27-amino-acid trimerization motif from the fibritin protein of T4 phage fused with a glycine/serine linker to the N-terminus CD40L ectodomain bundles. The CD40L ectodomain trimer bundles consist of individual CD40L protomers fused together by glycine/serine linkers from the C-terminus of one protomer to the N-terminus of the subsequent protomer to form single polypeptide trimeric bundles. A 6-mer polyhistidine (6×-His Tag (SEQ ID NO: 320)) sequence was added to the C-terminus of the final CD40L protomer to aid in purification of recombinant protein by Ni-NTA resin, however this feature is not present in the final virus construct. Figure discloses SEQ ID NOS 27, 22, and 320, respectively, in order of appearance. FIG. 27B is a crystal structure of the mouse surrogate IL-12 payload (mscIL-12). The mouse surrogate payload is a mouse IL-12 fusion protein containing the mouse p40 subunit from IL-12 fused with a glycine/serine linker to the N-terminus of mouse p35. A 6-mer polyhistidine sequence (6×-His Tag (SEQ ID NO: 320)), C-terminal to the p35 subunit was added to aid in purification of recombinant protein by Ni-NTA resin. However, this purification tag is not present in the final virus construct. FIGS. 27A and 27B disclose SEQ ID NOS 3 and 320, respectively, in order of appearance.

FIG. 27C is a diagram of the design of the mouse surrogate anti-CTLA-4 antagonist payload, termed mαCTLA-4, which is an antibody that binds to mouse CTLA-4 and comprises an anti-CTLA-4 VHH fused to the heavy chain of mouse IgG2a Fc.

FIG. 28 shows that the mouse surrogate CD40 agonist payload (mCD40ag) had comparable bioactivity to the human CD40 agonist payload (hCD40ag). The bioactivities of purified hCD40ag and mCD40ag were assessed using a HEK-Blue reporter assay, as described in Example 6 herein. CD40 agonist activity results in the production of a colorimetric readout, assessed by optical density at 655 nm (OD655). Recombinant WT mouse CD40L (rmCD40L) and recombinant WT human CD40L (rhCD40L) were included as controls.

FIG. 29 shows that the human IL-12 payload (hscIL-12) and the mouse surrogate IL-12 payload (mscIL-12) had comparable bioactivity. The bioactivity of purified hscIL-12 and mscIL-12 payloads was assessed using an hIL-12 receptor HEK-Blue reporter assay, as described in Example 6 herein. IL-12 activity results in the production of a colorimetric readout, assessed by optical density at 655 nm (OD655). Recombinant WT mouse IL-12 (rmIL-12) and recombinant WT human IL-12 (rhIL-12) were included as controls.

FIG. 30 shows that mαCTLA-4, the mouse surrogate for the human anti-CTLA-4 antagonist payload is efficacious in vivo, assessed in an MC38-5AG syngeneic tumor model. MC38-5AG is the MC38 line that was confirmed to contain neoantigen mutations as described in Yadav et al, Nature 515:572-76 (2014) WT mice were implanted with single MC38-5AG tumors and treated IT once every 3 days for 4 doses (q3dx4) with purified mαCTLA-4 payload. Tumor volume (mm³; mean±SEM) was monitored on the days post tumor implant indicated on the x-axis. ***p<0.001.

FIG. 31 is a diagram of the genome structure and transgene cassettes of the mouse surrogate virus, mJP-OV-2. The virus was engineered to express the anti-CTLA-4 antagonist (mαCTLA-4), CD40 agonist (mCD40ag), and IL-12 (mscIL-12) payloads from the US10-12 locus, and hFLT3L and UL49.5 from the γ34.5 locus. The virus expresses codon-optimized US11 (hCoUS11) using the US12 immediate early promoter and endogenous US11 using late US11 promoter. “S” shown in the hexagon represents a stop codon between hCoUS11 and US12, which inhibits US12 expression. US12 in grey indicates that the gene does not express. IR_{L}, internal repeat long; IR_{S}, internal repeat short; TR_{L}, terminal repeat long; TR_{S}, terminal repeat short; U_{L}, unique long; U_{S}, unique short.

FIG. 32A provides immunoblots showing ICP27, US11, UL49.5, and β-actin levels in whole-cell extracts from Vero cells infected with the indicated viruses (MOI=5, 6 hours post infection).

FIG. 32B provides immunoblots showing mCD40ag payload and actin levels in whole-cell extracts (top panel) and culture supernatants (bottom panel, mCD40ag only) from Vero cells infected with the indicated viruses (MOI=5, 6 hours post infection). In FIGS. 32A-32B, Δγ34.5 was used as a negative control for immediate-early expression of US11, ΔXN1 virus was used as a negative control for UL49.5 expression, Step 2 virus was the parental virus and used as a negative control for the mCD40ag payload expression, and a virus containing a cassette expressing mCD40L and was used as a positive control for the mCD40ag payload. The viral protein ICP27 was used as an infection control and β-actin was used as a protein loading control for the whole cell extracts.

FIG. 33 shows the expression levels of the hFLT3L, mscIL-12, mαCTLA-4, and mCD40ag mouse surrogate payload proteins in cells infected with the indicated viruses. Vero cells were infected (MOI=1) for 24 hours with control Δγ34.5, precursor Step 2 virus, and mouse Step 3 clones 1-4. Supernatants of infected cells were analyzed by ELISA. Δγ34.5 virus was used as a negative control for hFLT3L expression (top left panel), and Step 2 virus was used as a positive control for hFLT3L and a negative control for the other payloads. The expression levels of the four payload proteins are provided as the mean±standard deviation (SD).

FIG. 34 shows the cytotoxicity of the mouse surrogate virus, mJP-OV-2, assessed based on cell viability of virus-infected cells. HT29 cells (top panel), stringent for virus replication, or Vero cells (bottom panel), highly permissive and used and used to confirm that similar amounts of each virus were used in the experiment, were infected (MOI=0.01) by the indicated viruses. Virus-induced cell death was monitored by xCelligence. Unarmed virus ΔXN1 and attenuated virus Δγ34.5 were used as positive and negative controls, respectively.

FIG. 35 shows that the mCD40ag mouse surrogate payload protein is active when expressed from the mouse surrogate virus, mJP-OV-2. The bioactivity of mCD40ag in supernatant media from Vero cells infected (MOI=1) with mJP-OV-2 was compared to that of recombinant WT mouse CD40L (rmCD40L) or purified mCD40ag protein diluted in either media or supernatant from cells infected with the negative control Step 2 virus. CD40 agonist activity was assessed using an hCD40 receptor HEK-Blue reporter assay. CD40 agonist activity results in the production of a colorimetric readout, assessed by optical density at 655 nm (OD655).

FIG. 36 shows that the mscIL-12 mouse surrogate payload protein is active when expressed from the mouse surrogate virus, mJP-OV-2. The bioactivity of mscIL-12 in supernatant media from Vero cells infected (MOI=1) with the mouse surrogate virus mJP-OV-2 was compared to that of recombinant WT IL-12 (rmIL-12), or purified mscIL-12 protein diluted in either media or supernatant from cells infected with the negative control Step 2 virus. Bioactivity was assessed using an hIL-12 receptor HEK-Blue reporter assay. IL-12 activity results in the production of a colorimetric readout, assessed by optical density at 655 nm (OD655).

FIG. 37A shows the efficacy of mouse payload proteins on treated tumors in vivo. One of two tumors in an MC38-5AG bilateral syngeneic mouse tumor model was treated intratumorally (IT) with 5×106 PFU/injection of Step 2 virus (expressing the hFLT3L payload), alone or combined with a commercial anti-mouse CD40 agonist antibody (FGK4.5, 5 μg/injection). The tumors were treated once every 3 days for 3 doses (q3dx3). Treated or untreated (contralateral) tumor volumes were monitored over time (mm³; shown as mean±SEM). The difference in tumor growth inhibition (ATGI) compared to vehicle control is indicated for the treated tumor at Day 31 post tumor implant. *p<0.05, ***p<0.001. Arrows indicate dosing days.

FIG. 37B shows the efficacy of mouse payload proteins on treated tumors in vivo. One of two tumors in an MC38-5AG bilateral syngeneic mouse tumor model was treated intratumorally (IT) with 5×10⁶ PFU/injection of Step 2 virus (expressing the hFLT3L payload), alone or combined with purified mouse surrogate mscIL-12 protein (0.5 ng/injection). The tumors were treated once every 3 days for 3 doses (q3dx3). Treated or untreated (contralateral) tumor volumes were monitored over time (mm³; shown as mean±SEM). The difference in tumor growth inhibition (ΔTG1) compared to vehicle control is indicated for the treated tumor at Day 27 post tumor implant. *p<0.05, ***p<0.001. Arrows indicate dosing days.

FIG. 37C shows the efficacy of mouse payload proteins on treated tumors in vivo. One of two tumors in an MC38-5AG bilateral syngeneic mouse tumor model was treated intratumorally (IT) with 5×10⁶ PFU/injection of Step 2 virus (expressing the hFLT3L payload), alone or combined with a commercial anti-mouse CTLA-4 antibody (9H10, 20 μg/injection; F. The tumors were treated once every 3 days for 3 doses (q3dx3). Treated or untreated (contralateral) tumor volumes were monitored over time (mm³; shown as mean±SEM). The difference in tumor growth inhibition (ΔTGI) compared to vehicle control is indicated for the treated tumor at Day 27 post tumor implant. *p<0.05, ***p<0.001. Arrows indicate dosing days.

FIG. 38A shows the synergistic efficacy of mouse immunomodulatory payload proteins on treated tumors and abscopal tumors in vivo. One of two tumors in an MC38-5AG bilateral syngeneic tumor model mice was treated with 5×106 PFU/injection of Step 2 virus (expressing the hFLT3L payload) alone or combined with purified mscIL-12 (50 ng/injection), mCD40ag (13 μg/injection), and mαCTLA-4 (10.8 μg/injection) mouse surrogate payloads. The tumors were treated once every other day for 6 doses (q2dx6, indicated by the arrows in FIG. 38A). FIG. 38A shows the tumor volumes (mm³; mean±SEM; ***p<0.001) at the indicated times post-tumor implant for treated and contralateral tumors.

FIG. 38B shows a survival analysis for the mice used in the experiment shown in FIG. 38A. The mice were followed for 2× the median survival of the vehicle control.

FIG. 39A shows the synergistic efficacy of mouse immunomodulatory payload proteins on treated tumors and/or abscopal tumors in vivo using multiple doses of payload proteins. MC38-5AG tumor cells were implanted bilaterally on each flank of WT mice and treated IT in one tumor once every other day for 6 doses (q2dx6; indicated by arrows) with Step 2 virus (expressing hFLT3L) alone or combined with the remaining mouse surrogate payloads (i.e., the mαCTLA-4, mCD40ag, and mscIL-12 payloads). The amount of virus and mscIL-12 were kept constant (5×106 PFU and 50 ng, respectively). A dose-response was performed for the remaining two payloads, which was dosed at a High dose (13 μg mCD40ag and 10.8 μg mαCTLA-4). Tumor volumes (mm³) for the treated and untreated (contralateral) tumors were monitored over time, and are shown as mean±SEM. ***p<0.001.

FIG. 39B shows the synergistic efficacy of mouse immunomodulatory payload proteins on treated tumors and/or abscopal tumors in vivo using multiple doses of payload proteins. MC38-5AG tumor cells were implanted bilaterally on each flank of WT mice and treated IT in one tumor once every other day for 6 doses (q2dx6; indicated by arrows) with Step 2 virus (expressing hFLT3L) alone or combined with the remaining mouse surrogate payloads (i.e., the mαCTLA-4, mCD40ag, and mscIL-12 payloads). The amount of virus and mscIL-12 were kept constant (5×10⁶ PFU and 50 ng, respectively). A dose-response was performed for the remaining two payloads, which was dosed at a Medium dose (1.3 μg mCD40ag and 1.1 μg mαCTLA-4). Tumor volumes (mm³) for the treated and untreated (contralateral) tumors were monitored over time, and are shown as mean±SEM. ***p<0.001.

FIG. 39C shows the synergistic efficacy of mouse immunomodulatory payload proteins on treated tumors and/or abscopal tumors in vivo using multiple doses of payload proteins. MC38-5AG tumor cells were implanted bilaterally on each flank of WT mice and treated IT in one tumor once every other day for 6 doses (q2dx6; indicated by arrows) with Step 2 virus (expressing hFLT3L) alone or combined with the remaining mouse surrogate payloads (i.e., the mαCTLA-4, mCD40ag, and mscIL-12 payloads). The amount of virus and mscIL-12 were kept constant (5×106 PFU and 50 ng, respectively). A dose-response was performed for the remaining two payloads, which was dosed at a Low dose (0.13 μg mCD40ag and 0.11 μg mαCTLA-4). Tumor volumes (mm³) for the treated and untreated (contralateral) tumors were monitored over time, and are shown as mean±SEM. ***p<0.001.

FIG. 40A shows the survival of mice administered the High payload dose (i.e., 13 μg mCD40ag and 10.8 μg mαCTLA-4). *p<0.05, ***p<0.001.

FIG. 40B shows the survival of mice administered the Medium payload dose (1.3 μg mCD40ag and 1.1 μg mαCTLA-4). *p<0.05, ***p<0.001.

FIG. 40C shows the survival of mice administered the Low payload dose (0.13 μg mCD40ag and 0.11 μg mαCTLA-4). *p<0.05, ***p<0.001.

FIG. 41 shows that the combination of all mouse surrogate payloads resulted in a durable and specific anti-tumor response in mice that was protective of re-challenge with the same tumor type. Naïve mice, or mice previously cured of bilateral MC38-5AG tumors by treatment with Step 2 virus combined with all remaining mouse surrogate payloads (i.e., the mαCTLA-4, mCD40ag, and mscIL-12 payloads), were challenged or re-challenged with either MC38-5AG or AE17 single tumors. Tumor volume was monitored over time, and mice were sacrificed when they reached their endpoint tumor burden. Survival percent over time is shown on the y-axis on the days post-tumor implant indicated on the x-axis.

FIG. 42A shows the anti-tumor efficacy of the mouse surrogate virus, mJP-OV-2, on both treated and abscopal tumors in vivo. Mice bearing bilateral syngeneic MC38-5AG tumors were treated with mJP-OV-2 using the dosing regimen: once every 3 days for 3 doses (q3dx3). mJP-OV-2 efficacy was compared to that of a similar virus with no immune payloads, referred to as unarmed backbone virus. The vehicle group was treated q2dx6. Tumor volumes (mm³) for treated and untreated (contralateral) tumors were monitored over time and are shown as mean±SEM on the days post-tumor implant indicated on the x-axis. The arrows indicate dosing with the corresponding virus or vehicle control. *p<0.05, ***p<0.001.

FIG. 42B shows the anti-tumor efficacy of the mouse surrogate virus, mJP-OV-2, on both treated and abscopal tumors in vivo. Mice bearing bilateral syngeneic MC38-5AG tumors were treated with mJP-OV-2 using the dosing regimen: once every other day for 6 doses (q2dx6). mJP-OV-2 efficacy was compared to that of a similar virus with no immune payloads, referred to as unarmed backbone virus, as well as to a previously-reported genetically engineered oncolytic herpesvirus mimic. The vehicle group was treated q2dx6. Tumor volumes (mm³) for treated and untreated (contralateral) tumors were monitored over time and are shown as mean±SEM on the days post-tumor implant indicated on the x-axis. The arrows indicate dosing with the corresponding virus or vehicle control. *p<0.05, ***p<0.001.

FIG. 43A-43B show survival of the mice used in the experiments shown in FIGS. 42A-42B. FIG. 43A shows the survival of mice treated once every 3 days for 3 doses (q3dx3) with the indicated viruses, and FIG. 43B shows the survival of mice treated once every other day for 6 doses (q2dx6) with the indicated viruses. In FIG. 43A-43B, the vehicle group was treated q2dx6. Survival was monitored over time. **p<0.01, ***p<0.001.

FIG. 44A shows expression levels of the hFLT3L, mαCTLA-4, mCD40ag, and mscIL-12 payloads in MC38-5AG mouse tumors implanted bilaterally and treated IT (5×106 PFU/injection in 1 tumor) with mJP-OV-2. Four treated tumors were harvested at the indicated times post injection (x-axis), homogenized, and payload expression was quantitated by ELISA. Expression levels are shown as mean±SEM. The dotted lines were added to visualize payload expression levels over time.

FIG. 44B shows expression levels of the hFLT3L, hαCTLA-4, hCD40ag, and hscIL-12 payloads in H1299 human tumors implanted as single tumors in Nude mice and treated IT with JP-OV-2 (5×106 PFU/injection). Five treated tumors were harvested at the indicated times post injection (x-axis), homogenized, and payload expression was quantitated by ELISA. Expression levels are shown as mean±SEM. The dotted lines were added to visualize payload expression levels over time.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X”.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

As used herein, the terms “including,” “containing,” and “comprising” are used in their open, non-limiting sense.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International d′Unités (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “antibody” herein is used in the broadest sense and encompasses various antibody structures (immunoglobulin molecules, fragments of an immunoglobulin molecule, or a derivative of either thereof, which has the ability to specifically bind to an antigen under typical physiological conditions), including but not limited to monoclonal antibodies, 4-chain antibodies (such as IgG antibodies), heavy chain antibodies, and antibody fragments thereof so long as they exhibit the desired antigen-binding activity. The term “4-chain antibody” is used herein to refer to an antibody or antigen-binding fragment having two heavy chains and two light chains. The term “heavy chain antibody,” also known as “heavy chain-only antibody” or “HCAb” refers to a functional antibody, which comprises two heavy chains, but lacks two light chains usually found in 4-chain antibodies. Camelid animals (such as camels, llamas, or alpacas) are known to produce HCAbs.

An “isolated antibody” refers to an antibody that is substantially free of other antibodies having different antigenic specificities. An isolated antibody that binds specifically to an antigen can, however, have cross-reactivity to other antigens, such as homologous antigens from other species. Moreover, an isolated antibody can be substantially free of other cellular material and/or chemicals.

“Antibody fragments” comprise a portion of an antibody, preferably the antigen binding or variable region of the antibody. Examples of antibody fragments include VHHs, single-domain antibodies, Fab, Fab′, F(ab′)2, and Fv fragments; diabodies; linear antibodies (see U.S. Pat. No. 5,641,870, Example 2; Zapata et al., Protein Eng. 8(10):1057-1062 (1995)); single-chain antibody molecules; and multispecific antibodies formed from antibody fragments. The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

As used herein, the terms “binding”, “binds” or “specifically binds” in the context of the binding of an antibody to a pre-determined antigen typically is a binding with an affinity corresponding to a K_{D} of about 10⁶ M or less, e.g. 10⁷ M or less, such as about 10⁸ M or less, such as about 10⁹ M or less, about 10¹⁰ M or less, or about 10¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a Kp that is at least ten-fold lower, such as at least 100-fold lower, for instance at least 1,000-fold lower, such as at least 10,000-fold lower, for instance at least 100,000-fold lower than its Ko of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

A “CDR” refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32, 1-75. CDR region sequences also have been defined structurally by Chothia as those residues that are not part of the conserved β-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods. 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, 4-chain antibodies and antigen-binding antibody fragments thereof comprise six HVRs: three in the VH (H1, H2, H3), and three in the VL (L1, L2, L3). Generally, heavy-chain antibodies comprise three HVRs (HVR1, HVR2, HVR3).

A number of HVR delineations are in use and are encompassed herein. Exemplary HVRs for 4-chain antibodies and antigen-binding antibody fragments thereof herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

The amino acid residues of a single-domain antibody (such as VHH) can be numbered according to the general numbering for VHI domains given by Kabat et al. (“Sequence of proteins of immunological interest”, US Public Health Services, NIH Bethesda, Md., Publication No. 91), as applied to VHH domains from Camelids in the article of Riechmann and Muyldermans, J. Immunol. Methods 2000 Jun. 23; 240 (1-2): 185-195. According to this numbering, FR1 of a VHH comprises the amino acid residues at positions 1-30, CDR1 of a VHH comprises the amino acid residues at positions 31-35, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. In this respect, it should be noted that—as is well known in the art for Vn domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering).

Unless otherwise indicated, CDR residues and other residues in the variable domain (e.g., framework, “FR,” residues) are numbered herein according to Kabat et al.

The term “cassette”, “expression cassette,” or “gene cassette” refers to a sequence of DNA carrying, and capable of directing the expression of, one or more genes of interest between one or more sets of restriction sites. It can be transferred from one DNA sequence (usually a vector) to another by “cutting” the fragment out using restriction enzymes and “pasting” it back into the new context (such as a viral genome). Typically, the DNA fragment (nucleic acid sequence) is operatively associated with expression control sequence elements which provide for the proper transcription and translation of the target nucleic acid sequence(s) (genes). Such sequence elements may include a promoter and a polyadenylation signal.

A sequence “encoding” an expression product, such as a polypeptide, is a minimum nucleotide sequence that, when expressed, results in the production of that polypeptide.

The term “exogenous” refers to a combination of elements not naturally occurring. For example, an “exogenous gene” refers to a gene to be introduced to the genome of a virus, wherein that gene is not normally found in the genome of the virus or is a homolog of a gene expressed in the virus from a different species (e.g., the bovine herpes virus UL49.5 gene, which encodes for a TAP inhibitor, is exogenous when inserted into a viral genome that does not natively encode UL49.5).

As used herein, the term “herpes simplex virus” or “HSV” refers to members of the Herpesviridae family. Herpes simplex virus 1 and 2 (HSV-1 and HSV-2), also known by their taxonomical names Human alphaherpesvirus I and Human alphaherpesvirus 2, are two members of the human Herpesviridae family, a set of viruses that produce viral infections in the majority of humans.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

A coding sequence is “under the control of” or “operatively associated with” a promoter in a virus or cell when RNA polymerase transcribes the coding sequence into RNA, particularly mRNA, which is then spliced (if it contains introns) and translated into the polypeptide encoded by the coding sequence.

The term “specific binding” or “specifically binds” or is “specific for” a particular polypeptide or an epitope on a particular polypeptide target as used herein can be exhibited, for example, by a molecule having a K_{D} for the target of at least about 10″ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In some embodiments, the term “specific binding” refers to binding where a molecule binds a particular polypeptide or epitope on a particular polypeptide without substantially binding to any other polypeptide or polypeptide epitope. K_{D} can be determined by methods known in the art, such as ELISA, surface plasmon resonance (SPR), fluorescence activated cell sorting (FACS) analysis, or radioimmunoprecipitation (RIA). Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

An “individual” or “subject” is a mammal. Mammals include, but are not limited to, domesticated animals (e.g., cows, sheep, cats, dogs, and horses), primates (e.g., humans and non-human primates such as rhesus and cynomolgus monkeys), rabbits, and rodents (e.g., mice and rats). In some embodiments, the individual or subject is a human.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. As used herein “cancer” refers to solid tumors including sarcomas, carcinomas, and lymphomas.

“Treatment” or “therapy” of a subject refers to any type of intervention or process performed on, or the administration of an active agent to, the subject with the objective of curing, reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The phrase “pharmaceutically acceptable” indicates that the substance or composition must be compatible chemically and/or toxicologically, with the other ingredients comprising a formulation, and/or the mammal being treated therewith.

As described herein, any concentration range, percentage range, ratio range, or integer range is to be understood to include the value of any integer within the recited range and, when appropriate, fractions thereof (such as one tenth and one hundredth of an integer), unless otherwise indicated. Description of endpoints includes ranges between all endpoints disclosed. For example description of 1, 2, or 3 includes the ranges 1-2, 2-3 and 1-3.

### II. Oncolytic Viruses

Immunotherapy of cancer with oncolytic viruses is an emerging and maturing treatment modality which uses replication-competent viruses that selectively infect and damage tumor cells and may also, preferably, induce an immunological response which can control both the target tumor and distal tumors. Each species of oncolytic virus has a different cellular tropism, which helps determine which tissues are preferentially infected. Engineering of the virus can expand, restrict, or modulate this host range. A variety of species of virus have been investigated for use in oncolytic therapies, including those derived from HSV, vaccinia, and reovirus.

Thus, the present application provides oncolytic viruses that are effective for treating cancer. Non-limiting examples of oncolytic viruses include those derived from a herpes simplex virus, a vaccinia virus, an adenovirus, a reovirus, or a vesicular stomatitis virus. Preferentially, the oncolytic virus (such as an oncolytic HSV) preferentially triggers an immune response that results in killing of tumor cells. As used herein, the virus “preferentially kills” tumor cells when certain infectious doses of the virus are more likely to kill tumor cells than neighboring healthy cells (such as at least two times more likely to kill tumor cells than neighboring healthy cells at a given dose). Preferentially, the oncolytic virus expresses one or more payload proteins described below. Preferentially, the oncolytic virus induces an immune response to the tumor, which, in some embodiments, causes tumor cells at sites distal to the site of infection to be killed. Preferentially, the oncolytic virus is capable of evading an individual's immune system after administration to the individual. As used herein, evading the individual's immune system means that the oncolytic virus is able to preferentially replicate in tumor cells. In some embodiments, the oncolytic viruses provided herein are more sensitive to an innate antiviral response than a wild-type virus, enabling preferential replication in tumor cells. In some embodiments, the oncolytic viruses provided herein have an intermediate resistance to interferon.

### II-A. Oncolytic Herpes Simplex Virus

Herpes simplex virus (HSV) replicates in a variety of cell types including epithelial cells and fibroblasts. Two members of the human Herpesviridae family are HSV-1 and HSV-2. Native HSV establishes a life-long latent infection in neuronal cell bodies within the sensory ganglia of infected individuals. During the productive stage, HSV genes fall into three broad classes based on their temporal order of expression: immediate-early (IE), early (E), and late (L). Late genes may further be divided into two subclasses: leaky-late genes, which are expressed at low levels early after infection and upregulated later in infection, and true late genes, which are expressed exclusively after, and dependent upon, DNA replication.

Wild-type, mature HSV comprises a linear double stranded DNA genome of about 152 kb encoding at least 74 genes encased in an icosapentahedral capsid composed of 162 capsomers from six different viral proteins, 20-23 distinct viral tegument proteins, and an envelope comprising different glycoproteins. During adsorption to a host cell, the glycoproteins interact with surface receptors (and also with each other) to promote fusion of the viral envelope with the host membrane, allowing the virus to enter the cell.

In one aspect, the present disclosure pertains to oncolytic herpes simplex virus (HSV). In some embodiments, the oncolytic HSV is derived from HSV-1. In some embodiments, the oncolytic HSV comprises one or more expression cassettes described herein. In some embodiments, the oncolytic HSV expresses one or more payload proteins described herein. In some embodiments, the oncolytic HSV lacks one or more native HSV genes. In some embodiments, the oncolytic HSV lacks one or both copies of γ34.5. In some embodiments, the oncolytic HSV does not express one or more native HSV proteins, such as US12. In some embodiments, the oncolytic HSV expresses one or more additional copies of a native HSV protein, such as US11. In some embodiments, the oncolytic HSV expresses a native HSV protein in a different temporal order, such as expressing immediate-early US11. The oncolytic HSV may be a component of a pharmaceutical composition described herein. The oncolytic HSV, or a pharmaceutical composition comprising the oncolytic HSV, may be administered to individual according to the methods described herein (such as the methods of treatment described herein). In some embodiments, the oncolytic HSV preferentially triggers an immune response that results in killing of tumor cells compared to the wild-type HSV from which it is derived. In some embodiments, the oncolytic HSV is capable of triggering an immune response that triggers killing tumor cells at one or more sites distal to a target site.

### III. Viral Payloads

In some embodiments, the oncolytic virus (such as an oncolytic HSV), or the gene cassette otherwise described herein, comprises one or more genes encoding one or more payload molecules. The payload molecules are generally intended to enhance the therapeutic efficacy of the oncolytic virus (such as an oncolytic HSV). For example, a payload molecule may promote an immune response (e.g., against the tumor target) or may enhance the cytotoxicity of the oncolytic virus.

### III-A. IL-12

In some embodiments, an oncolytic virus (such as an oncolytic HSV), or an expression cassette otherwise described herein, comprises a polynucleotide encoding interleukin 12 (IL-12).

IL-12 is a heterodimeric protein comprising two subunits: p35 and p40. The native p35 subunit is linked to the p40 subunit by a disulfide bond. The human and mouse p40 subunits are 70% identical, while the p35 subunits share 60% amino acid sequence homology. The p35 and p40 subunits may function in receptor binding and signal transduction, respectively (Zou, J. J., et al. (1995). Structure-function analysis of the p35 subunit of mouse interleukin 12. The Journal of biological chemistry, 270(11), 5864-5871). IL-12 is normally secreted by antigen-presenting cells, such as macrophages and dendritic cells. Biologically active IL-12 (comprising both subunits in a heterodimer) functions to differentiate naïve T cells into Th1 cells, promote cytotoxic activity of NK cells and T cells, and block angiogenesis.

In some embodiments, the oncolytic virus (such as an oncolytic HSV), or the expression cassette otherwise described herein, comprises a polynucleotide encoding the p35 subunit of IL-12 and/or a polynucleotide encoding the p40 subunit of IL-12. In some embodiments, the p35 subunit and/or p40 subunit of IL-12 is human. In some embodiments, the p35 subunit and/or p40 subunit of IL-12 is murine. In some embodiments, the oncolytic virus (such as an oncolytic HSV), or the expression cassette otherwise described herein, comprises a polynucleotide encoding an IL-12 heterodimer comprising a p35 subunit and a p40 subunit. In some embodiments, the IL-12 heterodimer comprises a polypeptide comprising a p35 subunit of IL-12 and a p40 subunit of IL-12 connected by a peptide linker.

In some embodiments, the oncolytic virus (such as an oncolytic HSV), or the expression cassette otherwise described herein, comprises a polynucleotide encoding a human p35 subunit of IL-12 and/or a polynucleotide encoding a human p40 subunit of IL-12. In some embodiments, the human p35 subunit comprises the amino acid sequence of SEQ ID NO: 1, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:1. In some embodiments, the human p40 subunit comprises the amino acid sequence of SEQ ID NO:2, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:2. In some embodiments, the human p40 subunit comprises the amino acid sequence of SEQ ID NO:9, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:9. In some embodiments, the oncolytic virus (such as an oncolytic HSV), or the expression cassette otherwise described herein, comprises a polynucleotide encoding an IL-12 heterodimer comprising a human p35 subunit and a human p40 subunit. In some embodiments, the IL-12 heterodimer comprises a polypeptide comprising a human p35 subunit of IL-12 and a human p40 subunit of IL-12 connected by a peptide linker. In some embodiments, the peptide linker comprises an amino acid sequence comprising glycine and serine residues. In some embodiments, the peptide linker comprises the amino acid sequence of SEQ ID NO:3, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:3. In some embodiments, the peptide linker comprises the amino acid sequence of SEQ ID NO:7, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:7. In some embodiments, the IL-12 heterodimer comprises the amino acid sequence of SEQ ID NO:4, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:4. In some embodiments, the IL-12 heterodimer comprises the amino acid sequence of SEQ ID NO:10, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:10.

In some embodiments, the oncolytic virus (such as an oncolytic HSV), or the expression cassette otherwise described herein, comprises a polynucleotide encoding a murine p35 subunit of IL-12 and/or a polynucleotide encoding a murine p40 subunit of IL-12. In some embodiments, the murine p35 subunit comprises the amino acid sequence of SEQ ID NO:5, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:5. In some embodiments, the murine p40 subunit comprises the amino acid sequence of SEQ ID NO:6, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:6. In some embodiments, the murine p40 subunit comprises the amino acid sequence of SEQ ID NO:11, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:11. In some embodiments, the oncolytic virus (such as an oncolytic HSV), or the expression cassette otherwise described herein, comprises a polynucleotide encoding an IL-12 heterodimer comprising a murine p35 subunit and a murine p40 subunit. In some embodiments, the IL-12 heterodimer comprises a polypeptide comprising a murine p35 subunit of IL-12 and a murine p40 subunit of IL-12 connected by a peptide linker. In some embodiments, the peptide linker comprises an amino acid sequence comprising glycine and serine residues. In some embodiments, the peptide linker comprises the amino acid sequence of SEQ ID NO:3, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:3. In some embodiments, the peptide linker comprises the amino acid sequence of SEQ ID NO:7, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:7. In some embodiments, the IL-12 heterodimer comprises the amino acid sequence of SEQ ID NO:8, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:8. In some embodiments, the IL-12 heterodimer comprises the amino acid sequence of SEQ ID NO:12, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO: 12.

### III-B. CD40 Agonist

Cluster of differentiation 40 (CD40) is a costimulatory polypeptide expressed on numerous cell types, from antigen presenting cells (APCs) to epithelial cells. It is additionally present on various cancer cells. CD40 agonist, also known as cluster of differentiation 154 (CD154), comprises 261 amino acids and is a type II membrane glycopolypeptide that is expressed on the surface of activated T cells. Native CD40 agonist promotes B cell maturation. It is additionally essential for immunoglobulin class switching, as lack of CD40 agonist is associated with hyper IgM syndrome. CD40 agonist exists as a membrane-bound form, in which the extracellular domain forms a homotrimer, and a proteolytically-cleaved, soluble form, which has been shown to be biologically active.

In some embodiments, provided herein is an oncolytic virus comprising a polynucleotide encoding a CD40 agonist. In some embodiments, an oncolytic virus (such as an oncolytic HSV), or an expression cassette otherwise described herein, comprises a polynucleotide encoding CD40 agonist. In some embodiments, the CD40 agonist is a CD40 ligand. In some embodiments, the CD40 agonist comprises a CD40 ligand ectodomain. In some embodiments, the CD40 agonist is a trimer of three single-chain trimeric CD40 ligand ectodomains. In some embodiments, each of the three single-chain trimeric CD40 ligand ectodomains is fused to a trimerization motif, e.g., to direct formation of the trimer of three single-chain trimeric CD40 ligand ectodomains. In some embodiments, each of the three single-chain trimeric CD40 ligand ectodomains is fused to an Fc region, e.g., to direct formation of the trimer of three single-chain trimeric CD40 ligand ectodomains. In some embodiments, said Fc region is an IgG Fc region e.g., a human IgG1, IgG2, IgG3 or IgG4 Fc region. In some embodiments, said Fc region comprises one or more amino acid substitutions, insertions, or deletions that disfavor binding of said Fc region to another Fc region, such as an IgG Fc region, e.g., a human IgG1, IgG2, IgG3 or IgG4 Fc region. In some embodiments, said Fc region comprises a substitution of the IgG interaction domain with an IgA interaction domain. In some embodiments, each of the three single-chain trimeric CD40 ligand ectodomains is bivalent. In some embodiments, the CD40 agonist is an agonist antibody.

In some embodiments, the CD40 agonist comprises a human CD40 ligand ectodomain. In some embodiments, the human CD40 ligand ectodomain comprises the amino acid sequence set forth in SEQ ID NO:20, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:20. In some embodiments, the CD40 agonist is a trimer of three single-chain trimeric human CD40 ligand ectodomains. In some embodiments, the single-chain trimeric human CD40 ligand ectodomains comprise a polypeptide comprising three human CD40 ligand ectodomains connected by peptide linkers. In some embodiments, the single-chain trimeric human CD40 ligand ectodomain polypeptide comprises a first human CD40 ligand ectodomain connected by a peptide linker to a second human CD40 ligand ectodomain which is connected by a peptide linker to a third human CD40 ligand ectodomain. In some embodiments, the peptide linker comprises glycine and serine residues. In some embodiments, the peptide linker comprises the amino acid sequence set forth in SEQ ID NO:22, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:22. In some embodiments, the CD40 agonist comprises a trimerization motif operably linked to each of the three single-chain trimeric CD40 ligand ectodomains. In some embodiments, the trimerization motif is a T4 fibritin trimerization motif. In some embodiments, the T4 fibritin trimerization motif comprises the amino acid sequence set forth in SEQ ID NO:21, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:21. In some embodiments, the trimerization motif is linked to each of the three single-chain trimeric CD40 ligand ectodomains by a peptide linker. In some embodiments, the peptide linker connecting the trimerization motif to each of the three single-chain trimeric CD40 ligand ectodomains comprises an amino acid sequence comprising of glycine and/or serine residues. In some embodiments, the peptide linker comprises the amino acid sequence of SEQ ID NO:23, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:23. In some embodiments, the peptide linker comprises the amino acid sequence of SEQ ID NO:27, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:27. In some embodiments, the CD40 agonist further comprises a signal peptide sequence operably linked to each of the three single-chain trimeric CD40 ligand ectodomains. In some embodiments, the signal peptide sequence comprises the amino acid sequence of SEQ ID NO:24. In some embodiments, the CD40 agonist comprises a polypeptide comprising the amino acid sequence of SEQ ID NO:25, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:25. In some embodiments, the CD40 agonist comprises a polypeptide comprising the amino acid sequence of SEQ ID NO:30, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:30. In some embodiments, the CD40 agonist forms a trimer comprising three polypeptides comprising the amino acid sequence of SEQ ID NO:25, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:25. In some embodiments, the CD40 agonist forms a trimer comprising three polypeptides comprising the amino acid sequence of SEQ ID NO:30, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:30.

In some embodiments, the CD40 agonist comprises a murine CD40 ligand ectodomain. In some embodiments, the murine CD40 ligand ectodomain comprises the amino acid sequence set forth in SEQ ID NO:26, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:26. In some embodiments, the CD40 agonist is a trimer of three single-chain trimeric murine CD40 ligand ectodomains. In some embodiments, the single-chain trimeric murine CD40 ligand ectodomains comprise a polypeptide comprising three murine CD40 ligand ectodomains connected by peptide linkers. In some embodiments, the single-chain trimeric murine CD40 ligand ectodomain polypeptide comprises a first murine CD40 ligand ectodomain connected by a peptide linker to a second murine CD40 ligand ectodomain which is connected by a peptide linker to a third murine CD40 ligand ectodomain. In some embodiments, the peptide linker comprises glycine and serine residues. In some embodiments, the peptide linker comprises the amino acid sequence set forth in SEQ ID NO:22, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:22. In some embodiments, the CD40 agonist comprises a trimerization motif operably linked to each of the three single-chain trimeric CD40 ligand ectodomains. In some embodiments, the trimerization motif is a T4 fibritin trimerization motif. In some embodiments, the T4 fibritin trimerization motif comprises the amino acid sequence set forth in SEQ ID NO:21, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:21. In some embodiments, the trimerization motif is linked to each of the three single-chain trimeric CD40 ligand ectodomains by a peptide linker. In some embodiments, the peptide linker connecting the trimerization motif to each of the three single-chain trimeric CD40 ligand ectodomains comprises an amino acid sequence comprising of leucine, glycine, and/or serine residues. In some embodiments, the peptide linker comprises the amino acid sequence of SEQ ID NO:23, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:23. In some embodiments, the peptide linker comprises the amino acid sequence of SEQ ID NO:27, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:27. In some embodiments, the CD40 agonist further comprises a signal peptide sequence operably linked to each of the three single-chain trimeric CD40 ligand ectodomains. In some embodiments, the signal peptide sequence comprises the amino acid sequence of SEQ ID NO:24. In some embodiments, the CD40 agonist comprises a polypeptide comprising the amino acid sequence of SEQ ID NO:28, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:28. In some embodiments, the CD40 agonist comprises a polypeptide comprising the amino acid sequence of SEQ ID NO:29, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:29. In some embodiments, the CD40 agonist forms a trimer comprising three polypeptides comprising the amino acid sequence of SEQ ID NO:28, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:28. In some embodiments, the CD40 agonist forms a trimer comprising three polypeptides comprising the amino acid sequence of SEQ ID NO:29, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:29.

### III-C. CTLA-4 Binding Protein

Cytotoxic T-lymphocyte-associated protein 4 (CTLA-4 or CTLA-4), also known as cluster of differentiation 152 (CD152), is a polypeptide receptor that functions as an immune checkpoint and downregulates immune responses. The polypeptide contains an extracellular V-like domain, a transmembrane domain, and a cytoplasmic tail. Alternate isoforms have been characterized. CTLA-4 is constitutively expressed in regulatory T cells, but is only upregulated in conventional T cells after activation, and contributes to the inhibitory function of regulatory T cells. CTLA-4 binds to CD80 and CD86, also known as B7-1 and B7-2 respectively, on APCs in order to induce its inhibitory function to T cells.

In some embodiments, an oncolytic virus (such as an oncolytic HSV), or an expression cassette otherwise described herein, comprises a polynucleotide encoding a CTLA-4 binding protein. In some embodiments, the CTLA-4 binding protein is a CTLA-4 antagonist. For example, in some instances, the CTLA-4 binding protein inhibits the interaction between CTLA-4 and one or more CTLA-4 ligands, such as CD80 and/or CD86. In some embodiments, the CTLA-4 binding protein specifically binds to human CTLA-4, murine CTLA-4, or both human and murine CTLA-4.

In some embodiments, the CTLA-4 binding protein is an anti-CTLA-4 antibody or antigen binding fragment thereof. In some embodiments, the anti-CTLA-4 antibody or antigen binding fragment thereof specifically binds to human CTLA-4, murine CTLA-4, or both human and murine CTLA-4. In some embodiments, the anti-CTLA-4 antibody or antigen binding fragment is bivalent. In some embodiments, the anti-CTLA-4 antibody or antigen binding fragment comprises an Fc region, such as an active Fc region. In some embodiments, the anti-CTLA-4 antibody or antigen binding fragment comprises an IgG1, IgG2, IgG3, or IgG4 constant domain, e.g., a human or mouse IgG1, IgG2, IgG3, or IgG4 constant domain. In some embodiments, the anti-CTLA-4 antibody or antigen binding fragment thereof comprises a single-chain variable fragment (scFv). In some embodiments, the anti-CTLA-4 scFv is fused to the N-terminus of an IgG1, IgG2, IgG3, or IgG4 constant domain, e.g., a human or mouse IgG1, IgG2, IgG3, or IgG4 constant domain. In some embodiments, the anti-CTLA-4 antibody or antigen binding fragment thereof comprises an anti-CTLA-4 VHH, e.g., a camelid antibody comprising an anti-CTLA-4 VHH. In some embodiments, the anti-CTLA-4 VHH is fused to the heavy chain of an IgG1, IgG2, IgG3, or IgG4 Fc, e.g., a human or mouse IgG1, IgG2, IgG3, or IgG4 Fc.

In some embodiments, the anti-CTLA-4 antibody or antigen binding fragment thereof, such as the anti-CTLA-4 scFv, specifically binds to human CTLA-4. In some embodiments, the anti-CTLA-4 scFv is fused to the N-terminus of a IgG1 constant domain, e.g., a human IgG1 constant domain. In some embodiments, the human IgG1 is a variant human IgG1 comprising a C220S substitution, wherein the numbering of the residues is according to EU numbering. In some embodiments, the human IgG1 is a G1m(17) IgG1. In some embodiments, the anti-CTLA-4 antibody causes depletion of regulatory T (Treg) cells.

In some embodiments, the anti-CTLA-4 antibody or antigen binding fragment thereof (e.g., the anti-CTLA-4 scFv) comprises a variable heavy chain (VH) and a variable light chain (VL), wherein the VH comprises one or more of: (a) a CDR-H1 comprising the amino acid sequence set forth in SEQ ID NO:40; (b) a CDR-H2 comprising the amino acid sequence set forth in SEQ ID NO:41; and (c) a CDR-H3 comprising the amino acid sequence set forth in SEQ ID NO:42; and/or wherein the VL comprises one or more of: (a) a CDR-L1 comprising the amino acid sequence set forth in SEQ ID NO:43; (b) a CDR-L2 comprising the amino acid sequence set forth in SEQ ID NO:44; and (c) a CDR-L3 comprising the amino acid sequence set forth in SEQ ID NO:45. In some embodiments, the anti-CTLA-4 antibody or antigen binding fragment thereof (e.g., the anti-CTLA-4 scFv) comprises a VH and a VL, wherein the VH comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:42; and wherein the VL comprises a CDR-L1 comprising the amino acid sequence set forth in SEQ ID NO:43, a CDR-L2 comprising the amino acid sequence set forth in SEQ ID NO:44, and a CDR-L3 comprising the amino acid sequence set forth in SEQ ID NO:45.

In some embodiments, the anti-CTLA-4 antibody or antigen binding fragment thereof (e.g., the anti-CTLA-4 scFv) comprises a VH comprising the amino acid sequence set forth in SEQ ID NO:46, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:46; and/or a VL comprising the amino acid sequence set forth in SEQ ID NO:47, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:47. In some embodiments, the variable heavy chain and variable light chain are connected via a linker sequence. In some embodiments, the linker sequence comprises an amino acid sequence set forth in SEQ ID NO:61.

In some embodiments, the anti-CTLA-4 antibody or antigen binding fragment thereof (e.g., the anti-CTLA-4 scFv) comprises a VH comprising an amino acid sequence with at least 90%, at least 91%, at least 92%, at least 93%, at least 94%, at least 95%, at least 96%, at least 97%, at least 98%, at least 99%, or 100% homology to a VH amino acid sequence of SEQ ID NO:46, wherein the VH comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:42. In some embodiments, the anti-CTLA-4 antibody or antigen binding fragment thereof (e.g., the anti-CTLA-4 scFv) comprises a VL comprising an amino acid sequence with at least 90%, at least 91%, at least 92%, at least 93%, at least 94%, at least 95%, at least 96%, at least 97%, at least 98%, at least 99%, or 100% homology to a VL amino acid sequence of SEQ ID NO:47, wherein the VL comprises a CDR-L1 comprising the amino acid sequence set forth in SEQ ID NO:43, a CDR-L2 comprising the amino acid sequence set forth in SEQ ID NO:44, and a CDR-L3 comprising the amino acid sequence set forth in SEQ ID NO:45. In some embodiments, the anti-CTLA-4 antibody or antigen binding fragment thereof (e.g., the anti-CTLA-4 scFv) comprises a VH sequence having at least 90%, at least 91%, at least 92%, at least 93%, at least 94%, at least 95%, at least 96%, at least 97%, at least 98%, at least 99%, or 100% homology to a VH amino acid sequence of SEQ ID NO:46 and contains substitutions (e.g., conservative substitutions, insertions, or deletions relative to the reference sequence), but the anti-CTLA-4 antibody or antigen binding fragment thereof comprising that sequence retains the ability to bind to CTLA-4. In certain embodiments, a total of 1 to 10 amino acids have been substituted, inserted, and/or deleted in the VH amino acid sequence of SEQ ID NO:46. In certain embodiments, a total of 1 to 5 amino acids have been substituted, inserted and/or deleted in the VH amino acid sequence of SEQ ID NO:46. In certain embodiments, substitutions, insertions, or deletions occur in regions outside the CDRs (i.e., in the FR regions). In some embodiments, the substitutions, insertions, or deletions occur in the FR regions. Optionally, the anti-CTLA-4 antibody or antigen binding fragment thereof (e.g., the anti-CTLA-4 scFv) comprises the VH sequence of SEQ ID NO:46, including post-translational modifications of that sequence. In a particular embodiment, the VH comprises one, two or three CDRs selected from a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:42. In some embodiments, the anti-CTLA-4 antibody or antigen binding fragment thereof (e.g., the anti-CTLA-4 scFv) comprises a VL sequence having at least 90%, at least 91%, at least 92%, at least 93%, at least 94%, at least 95%, at least 96%, at least 97%, at least 98%, at least 99%, or 100% homology to a VL amino acid sequence of SEQ ID NO:47 and contains substitutions (e.g., conservative substitutions, insertions, or deletions relative to the reference sequence), but the anti-CTLA-4 antibody or antigen binding fragment thereof comprising that sequence retains the ability to bind to CLTA-4. In certain embodiments, a total of 1 to 10 amino acids have been substituted, inserted, and/or deleted in the VL amino acid sequence of SEQ ID NO:47. In certain embodiments, a total of 1 to 5 amino acids have been substituted, inserted and/or deleted in the VL amino acid sequence of SEQ ID NO:47. In certain embodiments, substitutions, insertions, or deletions occur in regions outside the CDRs (i.e., in the FR regions). In some embodiments, the substitutions, insertions, or deletions occur in the FR regions. Optionally, the anti-CTLA-4 antibody or antigen binding fragment thereof (e.g., the anti-CTLA-4 scFv) comprises the VL sequence of SEQ ID NO:47, including post-translational modifications of that sequence. In a particular embodiment, the VL comprises one, two or three CDRs selected from: a CDR-L1 comprising the amino acid sequence set forth in SEQ ID NO:43, a CDR-L2 comprising the amino acid sequence set forth in SEQ ID NO:44, and a CDR-L3 comprising the amino acid sequence set forth in SEQ ID NO:45. In some embodiments, the anti-CTLA-4 antibody or antigen binding fragment thereof (e.g., the anti-CTLA-4 scFv) comprises a VH comprising the amino acid sequence set forth in SEQ ID NO:46, and a VL comprising the amino acid sequence set forth in SEQ ID NO:47.

In some embodiments, the anti-CTLA-4 antibody or antigen binding fragment thereof (e.g., the anti-CTLA-4 scFv) comprises an IgG1 constant domain comprising the amino acid sequence set forth in SEQ ID NO:48, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:48. In some embodiments, the heavy chain of the CTLA-4 antibody comprises the amino acid sequence set forth in SEQ ID NO:48, with or without the C terminal lysine.

In some embodiments, the anti-CTLA-4 antibody or antigen binding fragment thereof (e.g., the anti-CTLA-4 scFv) comprises the amino acid sequence set forth in SEQ ID NO:60, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:60.

In some embodiments, the anti-CTLA-4 antibody or antigen binding fragment thereof (e.g., the anti-CTLA-4 scFv) comprises a signal peptide sequence comprising the amino acid sequence set forth in SEQ ID NO:49. In some embodiments, the anti-CTLA-4 antibody or antigen binding fragment thereof (e.g., the anti-CTLA-4 scFv) comprises the amino acid sequence set forth in SEQ ID NO:50, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:50.

In some embodiments, the anti-CTLA-4 antibody or antigen binding fragment thereof, such as the anti-CTLA-4 VHH, specifically binds to murine CTLA-4. In some embodiments, the anti-CTLA-4 VHH is fused to the heavy chain of a murine IgG2a Fc.

In some embodiments, the anti-CTLA-4 antibody or antigen binding fragment thereof (e.g., the anti-CTLA-4 VHH) comprises a variable heavy chain (VH), wherein the VH comprises one or more of: (a) a CDR-H1 comprising the amino acid sequence set forth in SEQ ID NO:51; (b) a CDR-H2 comprising the amino acid sequence set forth in SEQ ID NO:52; and (c) a CDR-H3 comprising the amino acid sequence set forth in SEQ ID NO:53. In some embodiments, the anti-CTLA-4 antibody or antigen binding fragment thereof (e.g., the anti-CTLA-4 VHH) comprises a VH, wherein the VH comprises a CDR-H1 comprising the amino acid sequence set forth in SEQ ID NO:51, a CDR-H2 comprising the amino acid sequence set forth in SEQ ID NO:52, and a CDR-H3 comprising the amino acid sequence set forth in SEQ ID NO:53. In some embodiments, the anti-CTLA-4 antibody or antigen binding fragment thereof (e.g., the anti-CTLA-4 VHH) comprises a VH comprising the amino acid sequence set forth in SEQ ID NO:54, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:54. In some embodiments, the anti-CTLA-4 antibody or antigen binding fragment thereof (e.g., the anti-CTLA-4 VHH) comprises a VH comprising an amino acid sequence with at least 90%, at least 91%, at least 92%, at least 93%, at least 94%, at least 95%, at least 96%, at least 97%, at least 98%, at least 99%, or 100% homology to a VH amino acid sequence of SEQ ID NO:54, wherein the VH comprises a CDR-H1 comprising the amino acid sequence set forth in SEQ ID NO:51, a CDR-H2 comprising the amino acid sequence set forth in SEQ ID NO:52, and a CDR-H3 comprising the amino acid sequence set forth in SEQ ID NO:53. In some embodiments, the anti-CTLA-4 antibody or antigen binding fragment thereof (e.g., the anti-CTLA-4 VHH) comprises a VH sequence having at least 90%, at least 91%, at least 92%, at least 93%, at least 94%, at least 95%, at least 96%, at least 97%, at least 98%, at least 99%, or 100% homology to a VH amino acid sequence of SEQ ID NO:54 and contains substitutions (e.g., conservative substitutions, insertions, or deletions relative to the reference sequence), but the anti-CTLA-4 antibody or antigen binding fragment thereof comprising that sequence retains the ability to bind to CTLA-4. In certain embodiments, a total of 1 to 10 amino acids have been substituted, inserted, and/or deleted in the VH amino acid sequence of SEQ ID NO:54. In certain embodiments, a total of 1 to 5 amino acids have been substituted, inserted and/or deleted in the VH amino acid sequence of SEQ ID NO:54. In certain embodiments, substitutions, insertions, or deletions occur in regions outside the CDRs (i.e., in the FR regions). In some embodiments, the substitutions, insertions, or deletions occur in the FR regions. Optionally, the anti-CTLA-4 antibody or antigen binding fragment thereof (e.g., the anti-CTLA-4 VHH) comprises the VH sequence of SEQ ID NO:54, including post-translational modifications of that sequence. In a particular embodiment, the VH comprises one, two or three CDRs selected from a CDR-H1 comprising the amino acid sequence set forth in SEQ ID NO:51, a CDR-H2 comprising the amino acid sequence set forth in SEQ ID NO:52, and a CDR-H3 comprising the amino acid sequence set forth in SEQ ID NO:53. In some embodiments, the anti-CTLA-4 antibody or antigen binding fragment thereof (e.g., the anti-CTLA-4 VHH) comprises a VH comprising the amino acid sequence set forth in SEQ ID NO:54.

In some embodiments, the anti-CTLA-4 antibody or antigen binding fragment thereof (e.g., the anti-CTLA-4 VHH) comprises the amino acid sequence set forth in SEQ ID NO:58, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:58. In some embodiments, the anti-CTLA-4 antibody or antigen binding fragment thereof (e.g., the anti-CTLA-4 VHH) comprises the amino acid sequence set forth in SEQ ID NO:59, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:59.

In some embodiments, the anti-CTLA-4 antibody or antigen binding fragment thereof (e.g., the anti-CTLA-4 VHH) comprises a signal peptide sequence comprising the amino acid sequence set forth in SEQ ID NO:55. In some embodiments, the anti-CTLA-4 antibody or antigen binding fragment thereof (e.g., the anti-CTLA-4 VHH) comprises the amino acid sequence set forth in SEQ ID NO:56, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:56. In some embodiments, the anti-CTLA-4 antibody or antigen binding fragment thereof (e.g., the anti-CTLA-4 VHH) comprises the amino acid sequence set forth in SEQ ID NO:57, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:57.

### III-D. FLT3 Ligand

In some embodiments, an oncolytic virus (such as an oncolytic HSV), or an expression cassette otherwise described herein, comprises a polynucleotide encoding a fms-like tyrosine kinase 3 (FLT3) ligand (FLT3L). FLT3L is a growth and differentiation factor that enhances and expands dendritic cells (DCs) as well as recruits DCs to the tumor microenvironment. Intratumoral DCs (IT DCs) have been identified as key mediators of antitumor T cell responses by processing tumor antigens and restimulating effector T cells in tertiary lymphoid structures or priming naïve T cells in the draining lymph nodes (Broz et al., Dissecting the tumor myeloid compartment reveals rare activating antigen-presenting cells critical for T cell immunity. Cancer Cell. 2014; 26(5):638-652. doi:10.1016/j.ccell.2014.09.007; and Cueto and Sancho, The Flt3L/Flt3 axis in dendritic cell biology and cancer immunotherapy. Cancers (Basel). 2021; 13(7): 1525. Published 2021 Mar. 26. doi: 10.3390/cancers13071525). Multiple clinical trials are investigating the use of systemic FLT3L to boost antitumor T cell responses.

FLT3L functions natively as a cytokine and growth factor. It binds FLT3 (CD135). The human FLT3L polynucleotide encodes a 235-amino acid type I transmembrane protein. Human FLT3L comprises an N-terminal 26-residue signal peptide, a 156-residue extracellular domain, a 23-residue transmembrane domain, and a 30-residue cytoplasmic domain. FLT3L can be released from the cell membrane by proteolytic cleavage. Soluble FLT3L natively forms a noncovalent dimer through interaction of six cysteine residues.

In some embodiments, the oncolytic virus (such as an oncolytic HSV), or the expression cassette otherwise described herein, comprises a polynucleotide encoding a human FLT3L. In some embodiments, the human FLT3L comprises the amino acid sequence of SEQ ID NO:72, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:72.

In some embodiments, the human FLT3L comprises a signal peptide directing secretion to the plasma membrane. In some embodiments, the signal peptide comprises the amino acid sequence of SEQ ID NO:70. In some embodiments, the human FLT3L comprises the amino acid sequence of SEQ ID NO:71, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:71.

In some embodiments, the FLT3L, e.g., the human FLT3L, is a homodimer. In some embodiments, the human FLT3L is proteolytically processed into soluble FLT3L. In some embodiments, the soluble FLT3L forms a homodimer.

### III-E. Other Payload Molecules

In some embodiments, an oncolytic virus (such as an oncolytic HSV), or an expression cassette otherwise described herein, comprises one or more polynucleotides encoding a US11 protein, such as a US11 protein from an HSV, e.g., an HSV-1 or HSV-2.

The protein kinase R (PKR) pathway is a component of the host cellular innate anti-viral response. PKR becomes activated in response to binding double-stranded RNA (dsRNA), a byproduct of viral replication, leading to phosphorylation and inactivation of eukaryotic translation initiation Factor 2 Subunit 1 (eIF2α), a translation initiation factor. Phosphorylated eIF2α prevents translation initiation, a cellular defense mechanism aimed at blocking the production of viral proteins. The US11 protein is believed to bind and sequester dsRNA, preventing the activation of the PKR pathway in host cells, and enabling enhanced viral replication.

In some embodiments, the US11 protein comprises the amino acid sequence of SEQ ID NO:80, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:80.

In some embodiments, the oncolytic virus (such as an oncolytic HSV), or the expression cassette otherwise described herein, comprises a polynucleotide encoding a US11 protein, wherein the polynucleotide comprises a native US11 gene nucleotide sequence, e.g., from an HSV, such as an HSV-1 or an HSV-2. In some embodiments, the native US11 gene is a native US11 late gene, wherein the US11 protein is expressed in the late stage of viral replication. In some embodiments, the native US11 late gene is under the control of the endogenous US11 promoter, e.g., from an HSV, such as an HSV-1 or an HSV-2.

In some embodiments, the oncolytic virus (such as an oncolytic HSV), or the expression cassette otherwise described herein, comprises a polynucleotide comprising a variant US11 gene. In some embodiments, the variant US11 gene is codon optimized for expression of the US11 protein in human cells. In some embodiments, the variant US11 gene encodes a wild type US11 protein, e.g., from an HSV, such as an HSV-1 or an HSV-2. In some embodiments, the variant US11 gene comprises the nucleotide sequence of SEQ ID NO:204, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:204. In some embodiments, the variant US11 gene is operably linked to a promoter. In some embodiments, the promoter directs immediate early expression of the US11 protein during viral replication. In some embodiments, the promoter is an endogenous US12 promoter from an HSV, such as HSV-1 or HSV-2, or a portion thereof.

In some embodiments, the oncolytic virus (such as an oncolytic HSV), or the expression cassette otherwise described herein, comprises both a polynucleotide encoding a US11 protein and comprising a native US11 gene nucleotide sequence, e.g., as described above; and a polynucleotide comprising a variant US11 gene, e.g., as described above.

In some embodiments, an oncolytic virus (such as an oncolytic HSV), or an expression cassette otherwise described herein, comprises a polynucleotide encoding a transporter associated with antigen processing (TAP) inhibitor, such as a viral TAP inhibitor. In general, viral TAP inhibitors prevent TAP from transporting peptides into the lumen of the endoplasmic reticulum, thus impairing peptide loading onto major histocompatibility complex (MHC) Class I molecules for display at the cell surface (Verweij et al. Viral inhibition of the transporter associated with antigen processing (TAP): A striking example of functional convergent evolution. PLOS Pathog. 2015; 11(4):e1004743). Although TAP inhibition disrupts the transport of newly-expressed MHC molecules to the cell surface, this does not block pre-existing antigen display. Thus, TAP inhibition by a TAP inhibitor can prevent the display of viral antigens on the cell surface, preventing premature clearance of infected cells and enabling virus persistence throughout multiple rounds of virus replication.

In some embodiments, the TAP inhibitor is derived from herpes virus 1 or herpes virus 2. In some embodiments, the TAP inhibitor is derived from bovine herpes virus 1. In some embodiments, the TAP inhibitor is any of UL49.5, US6, or ICP47. In some embodiments, the TAP inhibitor is UL49.5. In some embodiments, the TAP inhibitor comprises the amino acid sequence of SEQ ID NO:83, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:83. In some embodiments, the TAP inhibitor further comprises a signal peptide sequence. In some embodiments, the signal peptide sequence comprises the amino acid sequence of SEQ ID NO:81. In some embodiments, the TAP inhibitor comprises the amino acid sequence of SEQ ID NO:82, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:82. In some embodiments, the TAP inhibitor is expressed during the immediate early phase of viral replication, i.e., it is expressed as an immediate early gene. In some embodiments, the polynucleotide encoding the TAP inhibitor is expressed under the control of an immediate early promoter, such as a CMV promoter, e.g., an hCMV promoter.

### IV. Expression Cassettes

Provided herein are one or more expression cassettes comprising a polynucleotide encoding IL-12, a polynucleotide encoding a CD40 agonist, a polynucleotide encoding a CTLA-4 binding protein, a polynucleotide encoding an FLT3 ligand (FLT3L), or any combination thereof.

### IV-A. Expression Cassettes Encoding IL-12, a CD40 Agonist, and/or a CTLA-4 Binding Protein

Provided herein are expression cassettes comprising a polynucleotide encoding IL-12, a polynucleotide encoding a CD40 agonist, and/or a polynucleotide encoding a CTLA-4 binding protein.

In some embodiments, the expression cassettes of the disclosure comprise a promoter operably linked to each of the polynucleotide encoding IL-12, the polynucleotide encoding the CD40 agonist, and/or the polynucleotide encoding the CTLA-4 binding protein. Any suitable promoter may be used in the cassettes of the disclosure, so long as the promoter drives expression of the associated polynucleotide. Exemplary and non-limiting promoters that may be used include the human cytomegalovirus (hCMV) promoter, the murine cytomegalovirus (mCMV) promoter, the Aotine betaherpesvirus 1 (AoHV 1) promoter, the CAG promoter, a CMV hybrid promoter, the EFla promoter, the MMLV 5′ long terminal repeat (LTR) from the Moloney murine leukemia virus promoter (i.e., the MMLV promoter), the Pbidir3 promoter, and a native HSV promoter sequence, such as the HSV-1 or HSV-2 US12 promoter, or the HSV-1 or HSV-2 US11 promoter.

In some embodiments, the expression cassettes of the disclosure comprise a polyadenylation signal operably linked to each of the polynucleotide encoding IL-12, the polynucleotide encoding the CD40 agonist, and/or the polynucleotide encoding the CTLA-4 binding protein. Any suitable polyadenylation signal may be used in the cassettes of the disclosure. Exemplary and non-limiting polyadenylation signals (polyA or pA) that may be used include the simian vacuolating virus 40 polyA (SV40 pA), the human beta globin poly A (hBGpA), the human growth hormone polyA(hGH polyA), the rabbit beta globin polyA (rBGpA), a bovine growth hormone polyadenylation (BGHpA), a polyA derived from the human GAPDH gene, and a native HSV polyA sequence, such as the US10-12 polyA or the US9-10 poly A from HSV-1 or HSV-2.

In some embodiments, the expression cassettes of the disclosure may comprise any suitable promoter and/or polyadenylation signal known in the art or described herein operably linked to any of the polynucleotide encoding IL-12, the polynucleotide encoding the CD40 agonist, and/or the polynucleotide encoding the CTLA-4 binding protein.

In some embodiments, the expression cassettes of the disclosure comprise an RNA Polymerase II transcriptional pause signal positioned after each of the polynucleotide encoding IL-12, the polynucleotide encoding the CD40 agonist, and/or the polynucleotide encoding the CTLA-4 binding protein. Any suitable RNA Polymerase II transcriptional pause signal may be used in the cassettes of the disclosure. Exemplary and non-limiting RNA polymerase II transcriptional pause signals include the human complement C2 protein terminator (C2) and the human Gastrin terminator (hGT).

In some embodiments, an expression cassette of the disclosure comprises, in order, the polynucleotide encoding the CTLA-4 binding protein, the polynucleotide encoding the CD40 agonist, and the polynucleotide encoding the IL-12. In some embodiments, the polynucleotide encoding the CTLA-4 binding protein and the polynucleotide encoding the IL-12 are in the same orientation in the expression cassette, and the polynucleotide encoding the CD40 agonist is in the reverse orientation relative to the polynucleotide encoding the CTLA-4 binding protein and the polynucleotide encoding the IL-12.

In some embodiments, the polynucleotide encoding the CTLA-4 binding protein is operably linked to a promoter, such as any suitable promoter known in the art or described herein. In some embodiments, the promoter is an mCMV promoter. In some embodiments, the mCMV promoter comprises the nucleotide sequence of SEQ ID NO:210, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:210. In some embodiments, the expression cassette comprises a polyadenylation signal operably linked to the polynucleotide encoding the CTLA-4 binding protein, such as any suitable polyadenylation signal known in the art or described herein. In some embodiments, the polyadenylation signal is a polyA derived from the human GAPDH gene. In some embodiments, the polyadenylation signal is a GAPDH_SPA polyadenylation signal comprising the nucleotide sequence of SEQ ID NO:213, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:213. In some embodiments, the expression cassette further comprises a Kozak sequence positioned between the promoter and the polynucleotide encoding the CTLA-4 binding protein. In some embodiments, the Kozak sequence comprises the nucleotide sequence of SEQ ID NO:211, or a nucleotide sequence having any of 5, 4, 3, 2, or 1 base substitutions relative to the nucleotide sequence of SEQ ID NO:211. In some embodiments, the expression cassette further comprises an RNA polymerase II transcriptional pause signal positioned after the polyadenylation signal, such as any suitable RNA polymerase II transcriptional pause signal known in the art or described herein. In some embodiments, the RNA polymerase II transcriptional pause signal is a C2 RNA polymerase II transcriptional pause signal. In some embodiments, the C2 RNA polymerase II transcriptional pause signal comprises the nucleotide sequence of SEQ ID NO:214, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:214. In some embodiments, the encoded CTLA-4 binding protein is any of the CTLA-4 binding proteins described herein, e.g., in Section III-C, above. In some specific embodiments, the encoded CTLA-4 binding protein comprises the amino acid sequence set forth in SEQ ID NO:50, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:50. In some embodiments, the CTLA-4 binding protein comprises the amino acid sequence set forth in SEQ ID NO:50 with or without the C terminal lysine. In some specific embodiments, the encoded CTLA-4 binding protein comprises the amino acid sequence set forth in SEQ ID NO:60, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:60. In some embodiments the encoded CTLA-4 binding protein comprises the amino acid sequence set forth in SEQ ID NO:60, with our without the C terminal lysine. In other embodiments, the encoded CTLA-4 binding protein comprises the amino acid sequence set forth in SEQ ID NO:56, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:56. In other embodiments, the encoded CTLA-4 binding protein comprises the amino acid sequence set forth in SEQ ID NO:57, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:57. In other embodiments, the encoded CTLA-4 binding protein comprises the amino acid sequence set forth in SEQ ID NO:58, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:58. In other embodiments, the encoded CTLA-4 binding protein comprises the amino acid sequence set forth in SEQ ID NO:59, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:59. In some embodiments, the polynucleotide encoding the CTLA-4 binding protein comprises the nucleotide sequence of SEQ ID NO:212, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:212. In some embodiments, the polynucleotide encoding the CTLA-4 binding protein comprises the nucleotide sequence of SEQ ID NO:303, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:303.

In some embodiments, the polynucleotide encoding the CD40 agonist is operably linked to a promoter, such as any suitable promoter known in the art or described herein. In some embodiments, the promoter is the AOHV1 promoter. In some embodiments, the AOHV1 promoter comprises the nucleotide sequence of SEQ ID NO:219, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:219. In some embodiments, the expression cassette further comprises a polyadenylation signal operably linked to the polynucleotide encoding the CD40 agonist, such as any suitable polyadenylation signal known in the art or described herein. In some embodiments, the polyadenylation signal is a hBGpA. In some embodiments, the hBGpA comprises the nucleotide sequence of SEQ ID NO:216, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:216. In some embodiments, the expression cassette further comprises a Kozak sequence positioned between the promoter and the polynucleotide encoding the CD40 agonist. In some embodiments, the Kozak sequence comprises the nucleotide sequence of SEQ ID NO:218, or a nucleotide sequence having any of 5, 4, 3, 2, or 1 base substitutions relative to the nucleotide sequence of SEQ ID NO:218. In some embodiments, the expression cassette further comprises an RNA polymerase II transcriptional pause signal positioned after the polyadenylation signal, such as any suitable RNA polymerase II transcriptional pause signal known in the art or described herein. In some embodiments, the RNA polymerase II transcriptional pause signal is the hGT RNA polymerase II transcriptional pause signal. In some embodiments, the hGT RNA polymerase II transcriptional pause signal comprises the nucleotide sequence of SEQ ID NO:215, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:215. In some embodiments, the encoded CD40 agonist is any of the CD40 agonists described herein, e.g., in Sections III-B or V, herein. In some specific embodiments, the encoded CD40 agonist comprises a polypeptide comprising the amino acid sequence of SEQ ID NO:25, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:25. In some specific embodiments, the encoded CD40 agonist comprises a polypeptide comprising the amino acid sequence of SEQ ID NO:30, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:30. In other embodiments, the encoded CD40 agonist comprises a polypeptide comprising the amino acid sequence of SEQ ID NO:28, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:28. In other embodiments, the encoded CD40 agonist comprises a polypeptide comprising the amino acid sequence of SEQ ID NO:29, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:29. In some embodiments, the polynucleotide encoding the CD40 agonist comprises the nucleotide sequence of SEQ ID NO:217, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:217. In some embodiments, the polynucleotide encoding the CD40 agonist comprises the nucleotide sequence of SEQ ID NO:308, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:308. In some embodiments, the polynucleotide encoding the CD40 agonist is in the reverse orientation within the expression cassette relative to the polynucleotide encoding the IL-12 and the polynucleotide encoding the CTLA-4 binding protein.

In some embodiments, the polynucleotide encoding IL-12 is operably linked to a promoter, such as any suitable promoter known in the art or described herein. In some embodiments, the promoter is the MMLV promoter. In some embodiments, the MMLV promoter comprises the nucleotide sequence of SEQ ID NO:220, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:220. In some embodiments, the expression cassette further comprises a polyadenylation signal positioned after the polynucleotide encoding IL-12, such as any suitable polyadenylation signal known in the art or described herein. In some embodiments, the polyadenylation signal is the US10-12 poly A or the US9-10 poly A from HSV, such as from HSV-1 or HSV-2. In some embodiments, the US9-10 polyA comprises the nucleotide sequence of SEQ ID NO:314, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:314. In some embodiments, the US10-12 polyA comprises the nucleotide sequence of a native HSV-1 or HSV-2 US10-12 polyA, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of a native HSV-1 or HSV-2 US10-12 polyA. In some embodiments, the expression cassette further comprises a Kozak sequence positioned between the promoter and the polynucleotide encoding IL-12. In some embodiments, the Kozak sequence comprises the nucleotide sequence of SEQ ID NO:221, or a nucleotide sequence having any of 5, 4, 3, 2, or 1 base substitutions relative to the nucleotide sequence of SEQ ID NO:221. In some embodiments, the encoded IL-12 is any of the IL-12 proteins described herein, e.g., in Section III-A, above. In some specific embodiments, the encoded IL-12 comprises the amino acid sequence of SEQ ID NO:4, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:4. In some specific embodiments, the encoded IL-12 comprises the amino acid sequence of SEQ ID NO:10, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:10. In other embodiments, the encoded IL-12 comprises the amino acid sequence of SEQ ID NO:8, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:8. In other embodiments, the encoded IL-12 comprises the amino acid sequence of SEQ ID NO: 12, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO: 12. In some embodiments, the polynucleotide encoding IL-12 comprises the nucleotide sequence of SEQ ID NO:222, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:222. In some embodiments, the polynucleotide encoding IL-12 comprises the nucleotide sequence of SEQ ID NO:313, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:313.

In some embodiments, an expression cassette of the disclosure further comprises a polynucleotide encoding a US10 protein and/or a polynucleotide encoding a US11 protein; or a polynucleotide encoding a US11 protein and a US10 protein.

In some embodiments, an expression cassette of the disclosure comprises a polynucleotide encoding a US10 protein and/or a polynucleotide encoding a US11 protein. In some embodiments, the polynucleotide encoding the US11 protein is operably linked to a promoter, such as any suitable promoter known in the art or described herein. In some embodiments, the promoter is an endogenous US11 promoter from an HSV, such as HSV-1 or HSV-2. In some embodiments, the endogenous US11 promoter directs late expression of the US11 protein during viral replication. In some embodiments, the US11 promoter comprises the nucleotide sequence of SEQ ID NO:207, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:207. In some embodiments, the polynucleotide encoding the US10 protein is operably linked to a promoter, such as any suitable promoter known in the art or described herein. In some embodiments, the promoter is an endogenous US10 promoter. In some embodiments, the expression cassette comprises a polyadenylation signal operably linked to the polynucleotide encoding the US10 protein, such as any suitable polyadenylation signal known in the art or described herein. In some embodiments, the polyadenylation signal is a hGHpolyA. In some embodiments, the hGHpolyA comprises the nucleotide sequence of SEQ ID NO:209, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:209. In some embodiments, the encoded US11 protein is an HSV US11 protein, such as an HSV-1 or HSV-2 US11 protein. In some embodiments, the encoded US11 protein comprises the amino acid sequence set forth in SEQ ID NO:80, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:80. In some embodiments, the encoded US10 protein is an HSV US10 protein, such as an HSV-1 or HSV-2 US10 protein. In some embodiments, the encoded US10 protein comprises the amino acid sequence set forth in SEQ ID NO:90, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:90. In some embodiments, the polynucleotide encoding the US11 protein comprises a native US11 gene. In some embodiments, the expression cassette comprises, in order, the polynucleotide encoding the US11 protein (e.g., comprising a native US11 gene) and/or the polynucleotide encoding the US10 protein, the polynucleotide encoding the CTLA-4 binding protein, the polynucleotide that encodes the CD40 agonist, and the polynucleotide encoding the IL-12. In some embodiments, the polynucleotides encoding the CTLA-4 binding protein, the IL-12, the US11 protein and/or the US10 protein are in the same orientation in the expression cassette, and the polynucleotide that encodes the CD40 agonist is in the reverse orientation relative to the polynucleotides encoding the CTLA-4 binding protein, the IL-12, the US11 protein and/or the US10 protein.

In some embodiments, an expression cassette of the disclosure comprises a polynucleotide encoding a US11 protein and a US10 protein. In some embodiments, the polynucleotide encoding the US11 protein and the US10 protein comprises a nucleic acid sequence encoding the US11 protein, and a nucleic acid sequence encoding the US10 protein. In some embodiments, at least a portion of the nucleic acid sequence encoding the US11 protein overlaps with at least a portion of the nucleic acid sequence encoding the US10 protein. In some embodiments, the nucleic acid sequence encoding the US11 protein is operably linked to a promoter, such as any suitable promoter known in the art or described herein. In some embodiments, the promoter is an endogenous US11 promoter from an HSV, such as HSV-1 or HSV-2. In some embodiments, the endogenous US11 promoter directs late expression of the US11 protein during viral replication. In some embodiments, the US11 promoter comprises the nucleotide sequence of SEQ ID NO:207, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:207. In some embodiments, the nucleic acid sequence encoding the US10 protein is operably linked to a promoter. In some embodiments, the promoter is a native US10 promoter from an HSV, such as HSV-1 or HSV-2. In some embodiments, the promoter is embedded within the nucleic acid sequence encoding the US11 protein. In some embodiments, the encoded US11 protein is an HSV US11 protein, such as an HSV-1 or HSV-2 US11 protein. In some embodiments, the encoded US11 protein comprises the amino acid sequence set forth in SEQ ID NO:80, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:80. In some embodiments, the encoded US10 protein is an HSV US10 protein, such as an HSV-1 or HSV-2 US10 protein. In some embodiments, the encoded US10 protein comprises the amino acid sequence set forth in SEQ ID NO:90, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:90. In some embodiments, the polynucleotide encoding the US10 protein and the US11 protein comprises the nucleotide sequence of SEQ ID NO:208, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:208. In some embodiments, the expression cassette comprises a polyadenylation signal operably linked to the nucleic acid sequence encoding the US10 protein, such as any suitable polyadenylation signal known in the art or described herein. In some embodiments, the polyadenylation signal is a hGHpolyA. In some embodiments, the hGHpolyA comprises the nucleotide sequence of SEQ ID NO:209, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:209. In some embodiments, the polynucleotide encoding the US11 protein and the US10 protein comprises a native US11 gene. In some embodiments, the expression cassette comprises, in order, the polynucleotide encoding the US11 protein and the US10 protein (e.g., comprising a native US11 gene), the polynucleotide encoding the CTLA-4 binding protein, the polynucleotide that encodes the CD40 agonist, and the polynucleotide encoding the IL-12. In some embodiments, the polynucleotides encoding the CTLA-4 binding protein, the IL-12, and the US11 and US10 proteins are in the same orientation in the expression cassette, and the polynucleotide that encodes the CD40 agonist is in the reverse orientation relative to the polynucleotides encoding the CTLA-4 binding protein, the IL-12, and the US11 and US10 proteins.

In some embodiments, an expression cassette of the disclosure further comprises a polynucleotide encoding a US11 protein, wherein the polynucleotide comprises a variant US11 gene. In some embodiments, the variant US11 gene comprises a sequence that is codon optimized for expression of the US11 protein in human cells. In some embodiments, the variant US11 gene is operably linked to a promoter, such as any suitable promoter known in the art or described herein. In some embodiments, the promoter is an endogenous US12 promoter from an HSV, such as HSV-1 or HSV-2, or a portion thereof. In some embodiments, the endogenous US12 promoter, or the portion thereof, directs immediate early expression of the US11 protein during viral replication. In some embodiments, the endogenous US12 promoter comprises the nucleotide sequence of SEQ ID NO:203, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:203. In some embodiments, the encoded US11 protein comprises the amino acid sequence set forth in SEQ ID NO:80, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:80. In some embodiments, the variant US11 gene comprises the nucleotide sequence of SEQ ID NO:204, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:204. In some embodiments, the expression cassette further comprises a 5′ untranslated region (UTR) sequence positioned between the promoter and the variant US11 gene. In some embodiments, the 5′ UTR comprises the nucleotide sequence of SEQ ID NO:223, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:223. In some embodiments, the expression cassette further comprises a polynucleotide encoding a US12 protein positioned after the variant US11 gene (e.g., after a stop codon in the variant US11 gene). In some embodiments, the US12 protein is from an HSV, such as HSV-1 or HSV-2. In some embodiments, the polynucleotide encoding the US12 protein is not operably linked to a promoter. In some embodiments, the encoded US12 protein is not expressed. In some embodiments, the polynucleotide encoding the US12 protein comprises the nucleotide sequence of SEQ ID NO:206, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:206. In some embodiments, the expression cassette further comprises a spacer sequence and a UTR sequence positioned between the variant US11 gene and the polynucleotide encoding the US12 protein. In some embodiments, the spacer sequence and a UTR sequence comprise the nucleotide sequence of SEQ ID NO:205, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:205. In some embodiments, the expression cassette comprises, in order, the variant US11 gene; the polynucleotides encoding the US10 and/or US11 proteins, or the polynucleotide encoding the US10 and US11 proteins; the polynucleotide encoding the CTLA-4 binding protein; the polynucleotide that encodes the CD40 agonist; and the polynucleotide encoding the IL-12. In some embodiments, the polynucleotides encoding the CTLA-4 binding protein, the IL-12, and the US10 and/or US11 proteins are in the same orientation in the expression cassette, and the polynucleotide that encodes the CD40 agonist is in the reverse orientation relative to the polynucleotides encoding the CTLA-4 binding protein, the IL-12, and the US10 and/or US11 proteins.

In some embodiments, an expression cassette of the disclosure comprises, in order, a promoter (e.g., an HSV US12 promoter) operably linked to the polynucleotide comprising a variant US11 gene; optionally, a 5′ UTR sequence; the polynucleotide comprising the variant US11 gene; a promoter (e.g., a native HSV US11 promoter); the polynucleotide encoding the US11 protein and the US10 protein; a polyadenylation signal (e.g., a hGHpA polyA) operably linked to the polynucleotide encoding the US11 protein and the US10 protein; a promoter (e.g., a CMV promoter such as an mCMV promoter) that directs expression of the polynucleotide encoding the CTLA-4 binding protein; optionally, a Kozak sequence for expression of the polynucleotide encoding the CTLA-4 binding protein; the polynucleotide encoding the CTLA-4 binding protein; a polyadenylation signal (e.g., a GAPDH_SpA polyA) that is operably linked to the polynucleotide encoding the CTLA-4 binding protein; optionally, an RNA polymerase II pause site (e.g., a C2 pause site); an RNA polymerase II pause site (e.g., an hGT pause site); a polyadenylation signal (e.g., an hBGpA polyA) that is operably linked to the polynucleotide encoding the CD40 agonist; the polynucleotide that encodes the CD40 agonist; optionally, a Kozak sequence for expression of the polynucleotide that encodes the CD40 agonist; a promoter (e.g., an AoHV1 promoter) that controls expression of the CD40 agonist; a promoter (e.g., an MMLV promoter) that controls expression of the IL-12; optionally, a Kozak sequence for expression of the polynucleotide encoding the IL-12; the polynucleotide encoding the IL-12; and a polyadenylation signal (e.g., an HSV US10-12 polyA) that is operably linked to the polynucleotide encoding the IL-12.

In some embodiments, an expression cassette of the disclosure comprises the nucleotide sequence of SEQ ID NO:201, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:201. In some embodiments, an expression cassette of the disclosure comprises the nucleotide sequence of SEQ ID NO:202, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:202.

In some embodiments, an expression cassette of the disclosure is integrated into a genome of a virus, such as an oncolytic HSV, e.g., an oncolytic HSV-1 or oncolytic HSV-2. In some embodiments, the cassette is integrated in the US10-12 locus of an oncolytic HSV, e.g., an oncolytic HSV-1 or oncolytic HSV-2.

In some embodiments, the expression cassette comprises: (i) the polynucleotide encoding IL-12, (ii) the polynucleotide encoding the CD40 agonist, (iii) and the polynucleotide encoding the CTLA-4 binding protein, e.g., as described above. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii).

In some embodiments, the expression cassette further comprises polynucleotide(s) encoding a US10 protein and/or a US11 protein, e.g., as described above. In some such embodiments, the expression cassette comprises (i) the polynucleotide encoding IL-12, (ii) the polynucleotide encoding the CD40 agonist, (iii) the polynucleotide encoding the CTLA-4 binding protein, and (iv) the polynucleotides encoding the US10 protein and/or US11 protein. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii)-(iv). In other embodiments, the expression cassette further comprises a polynucleotide encoding a US10 protein and a US11 protein, e.g., as described above. In some such embodiments, the expression cassette comprises (i) the polynucleotide encoding IL-12, (ii) the polynucleotide encoding the CD40 agonist, (iii) the polynucleotide encoding the CTLA-4 binding protein, and (iv) the polynucleotide encoding the US10 and US11 proteins. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii)-(iv). In other embodiments, the expression cassette further comprises a polynucleotide encoding a US10 protein and a polynucleotide encoding a US11 protein, e.g., as described above. In some such embodiments, the expression cassette comprises (i) the polynucleotide encoding IL-12, (ii) the polynucleotide encoding the CD40 agonist, (iii) the polynucleotide encoding the CTLA-4 binding protein, (iv) the polynucleotide encoding the US10 protein, and (v) the polynucleotide encoding the US11 protein. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii)-(iv)-(v).

In some embodiments, the expression cassette further comprises a polynucleotide comprising a variant US11 gene, e.g., as described above. In some such embodiments, the expression cassette comprises: (i) the polynucleotide encoding IL-12, (ii) the polynucleotide encoding the CD40 agonist, (iii) the polynucleotide encoding the CTLA-4 binding protein, (iv) the polynucleotide(s) encoding the US10 protein and/or US11 protein, or the polynucleotide encoding the US10 and US11 proteins, and (v) the polynucleotide comprising the variant US11 gene. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii)-(iv)-(v). In other embodiments, the expression cassette comprises a polynucleotide comprising a variant US11 gene, e.g., as described above. In some such embodiments, the expression cassette comprises: (i) the polynucleotide encoding IL-12, (ii) the polynucleotide encoding the CD40 agonist, (iii) the polynucleotide encoding the CTLA-4 binding protein, (iv) the polynucleotide encoding the US10 protein, (v) the polynucleotide encoding the US11 protein, (vi) the polynucleotide comprising the variant US11 gene. In some embodiments, the expression cassette is in the orientation relative to the internal short repeat (IRS) region of the genome, e.g., an oncolytic HSV genome, such as an HSV-1 or HSV-2 genome, of IRS-(i)-(ii)-(iii)-(iv)-(v)-(vi).

### IV-B. Expression Cassettes Encoding FLT3L and/or TAP Inhibitor

Also provided herein are expression cassettes comprising a polynucleotide encoding FLT3L and/or a polynucleotide encoding a transporter associated with antigen processing (TAP) inhibitor.

In some embodiments, an expression cassette of the disclosure comprises a promoter operably linked to the polynucleotide encoding FLT3L and/or to the polynucleotide encoding the TAP inhibitor. Any suitable promoter may be used in the cassettes of the disclosure, so long as the promoter drives expression of the associated polynucleotide. Exemplary and non-limiting promoters that may be used include the human cytomegalovirus (hCMV) promoter, the murine cytomegalovirus (mCMV) promoter, the Aotine betaherpesvirus 1 (AoHV 1) promoter, the CAG promoter, a CMV hybrid promoter, the EFla promoter, the MMLV 5′ long terminal repeat (LTR) from the Moloney murine leukemia virus promoter (i.e., the MMLV promoter), the Pbidir3 promoter, and a native HSV promoter sequence.

In some embodiments, the expression cassette further comprises a polyadenylation signal operably linked to the polynucleotide encoding FLT3L and/or the polynucleotide encoding the TAP inhibitor. Any suitable polyadenylation signal may be used in the cassettes of the disclosure. Exemplary and non-limiting polyadenylation signals (poly A or pA) that may be used include the simian vacuolating virus 40 polyA (SV40 pA), the human beta globin polyA (hBGpA), the human growth hormone polyA (hGH polyA), the rabbit beta globin polyA (rBGpA), a bovine growth hormone polyadenylation (BGHpA), a polyA derived from the human GAPDH gene, and a native HSV polyA sequence.

In some embodiments, the expression cassettes of the disclosure may comprise any suitable promoter and/or polyadenylation signal known in the art or described herein operably linked to any of the polynucleotide encoding FLT3L and/or to the polynucleotide encoding the TAP inhibitor.

In some embodiments, the encoded FLT3L is any of the FLT3L proteins described herein, e.g., in Section III-D, above. In one specific embodiment, the encoded FLT3L comprises the amino acid sequence of SEQ ID NO:71, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:71. In one specific embodiment, the encoded FLT3L comprises the amino acid sequence of SEQ ID NO:72, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:72. In some embodiments, the polynucleotide encoding the FLT3L comprises the nucleotide sequence of SEQ ID NO: 105, or an nucleotide sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence set forth in SEQ ID NO: 105.

In some embodiments, the TAP inhibitor is derived from herpes virus 1 or herpes virus 2. In some embodiments, the TAP inhibitor is derived from bovine herpes virus 1. In some embodiments, the TAP inhibitor is any of UL49.5, US6, or ICP47. In some embodiments, the TAP inhibitor is UL49.5. In some embodiments, the encoded TAP inhibitor comprises the amino acid sequence of SEQ ID NO:83, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:83. In some embodiments, the TAP inhibitor further comprises a signal peptide sequence. In some embodiments, the signal peptide sequence comprises the amino acid sequence of SEQ ID NO:81. In some embodiments, the encoded TAP inhibitor comprises the amino acid sequence of SEQ ID NO:82, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:82. In some embodiments, the polynucleotide encoding the TAP inhibitor comprises the nucleotide sequence of SEQ ID NO: 103, or nucleotide sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence set forth in SEQ ID NO: 103.

In some embodiments, the expression cassette further comprises a polynucleotide encoding a self-cleaving peptide. Any suitable self-cleaving peptide may be used in the cassettes of the disclosure, including, but not limited to, a T2 Å, P2A, E2A, or F2 Å peptide. In some embodiments, the encoded self-cleaving peptide is a P2A peptide. In some embodiments, the encoded P2A comprises the amino acid sequence of SEQ ID NO:91, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:91. In some embodiments, the polynucleotide encoding the self-cleaving peptide comprises the nucleotide sequence of SEQ ID NO: 104, or a nucleotide sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence set forth in SEQ ID NO: 104. In some embodiments, the self-cleaving peptide is positioned between the polynucleotide encoding the FLT3L and the polynucleotide encoding the TAP inhibitor in the expression cassette.

In some embodiments, the expression cassette comprises a promoter operably linked to the polynucleotide encoding the FLT3L. In some embodiments, the promoter is the hCMV promoter. In some embodiments, the hCMV promoter comprises the nucleotide sequence of SEQ ID NO:107, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO: 107.

In some embodiments, the expression cassette further comprises a polyadenylation signal operably linked to the polynucleotide encoding the TAP inhibitor. In some embodiments, the polyadenylation sequence is a BGHpA polyadenylation signal. In some embodiments, the BGHpA polyadenylation signal comprises the nucleotide sequence of SEQ ID NO:102, or a nucleotide sequence having any of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence of SEQ ID NO:102.

In some embodiments, an expression cassette of the disclosure comprises, in order, a promoter (e.g., an hCMV promoter) operably linked to the polynucleotide encoding the FLT3L; the polynucleotide encoding the FLT3L; the polynucleotide encoding the self-cleaving peptide (e.g., a P2A peptide); the polynucleotide encoding the TAP inhibitor (e.g., a UL49.5 protein); and a polyadenylation signal (e.g., a BGHpA polyadenylation signal).

In some embodiments, an expression cassette of the disclosure comprises a polynucleotide encoding, in order, the FLT3L, the self-cleaving peptide (e.g., a P2A peptide), and the TAP inhibitor (e.g., a UL49.5 protein). In some embodiments, said polynucleotide comprises the nucleotide sequence of SEQ ID NO: 106, or a nucleotide sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence set forth in SEQ ID NO: 106. In some embodiments, the expression cassette encodes a polypeptide comprising, in order, the FLT3L, the self-cleaving peptide (e.g., a P2A peptide), and the TAP inhibitor (e.g., a UL49.5 protein). In some embodiments, said polypeptide comprises the amino acid sequence of SEQ ID NO:92, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:92. In some embodiments, said polypeptide comprises the amino acid sequence of SEQ ID NO:93, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:93. In some embodiments, the expression cassette further comprises a promoter, e.g., an hCMV promoter, that regulates expression of the polynucleotide encoding the FLT3L, the self-cleaving peptide (e.g., a P2A peptide), and the TAP inhibitor (e.g., a UL49.5 protein). In some embodiments, the expression cassette further comprises a polyadenylation signal, e.g., a BGHpA. In some embodiments, the expression cassette comprises, in order, a promoter, e.g., an hCMV promoter; a polynucleotide encoding, in order, the FLT3L, the self-cleaving peptide (e.g., a P2A peptide), and the TAP inhibitor (e.g., a UL49.5 protein); and a polyadenylation signal, e.g., a BGHpA.

In some embodiments, an expression cassette of the disclosure comprises the nucleotide sequence of SEQ ID NO: 100, or a nucleotide sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the nucleotide sequence set forth in SEQ ID NO:100.

In some embodiments, an expression cassette of the disclosure is integrated into a genome of a virus, such as an oncolytic HSV, e.g., an HSV-1 or HSV-2. In some embodiments, the cassette is integrated into one or two of the native γ34.5 loci of an oncolytic HSV, e.g., an HSV-1 or HSV-2. In some embodiments, the cassette is integrated into both of the native γ34.5 loci of an oncolytic HSV, e.g., an HSV-1 or HSV-2. In some embodiments, one or two of the native γ34.5 loci of an oncolytic HSV, e.g., an HSV-1 or HSV-2, are rendered inactive by insertion of the expression cassette. In some embodiments, both of the native γ34.5 loci of an oncolytic HSV, e.g., an HSV-1 or HSV-2, are rendered inactive by insertion of the expression cassette. In some embodiments, integration of the expression cassette into a γ34.5 locus comprises replacing all, substantially all, or a part of the native γ34.5 locus with the expression cassette. In some embodiments, the TAP inhibitor encoded by the expression cassette is expressed as an immediate-early gene during viral replication.

In some embodiments, the expression cassette comprises: (i) the polynucleotide encoding the TAP inhibitor (e.g., a UL49.5 protein), (ii) the polynucleotide encoding the self-cleaving peptide (such as a P2A peptide), and (iii) the polynucleotide encoding the FLT3L, e.g., as described above. In some embodiments, the expression cassette is integrated in (e.g., replaces in whole or in part, or otherwise incorporated in any way) the native γ34.5 locus within the long terminal repeat (TR_{L}) region of the genome, e.g. an oncolytic HSV genome, such as an HSV-1 or HSV-2 genome. In some embodiments, the expression cassette is in the orientation relative to the unique long (UL) region of the genome, e.g. an oncolytic HSV genome, such as an HSV-1 or HSV-2 genome, of (i)-(ii)-(iii)-UL.

In some embodiments, the expression cassette comprises: (i) the polynucleotide encoding the TAP inhibitor (e.g., a UL49.5 protein), (ii) the polynucleotide encoding the self-cleaving peptide (e.g., a P2A peptide), and (iii) the polynucleotide encoding the FLT3L, e.g., as described above. In some embodiments, the expression cassette is integrated in (e.g., replaces in whole or in part, or otherwise incorporated in any way) the native γ34.5 locus within the internal long repeat (IR_{L}) region of the genome, e.g. an oncolytic HSV genome, such as an HSV-1 or HSV-2 genome. In some embodiments, the expression cassette is in the orientation relative to the unique long (UL) region of the genome, e.g. an oncolytic HSV genome, such as an HSV-1 or HSV-2 genome, of UL-(iii)-(ii)-(i).

### IV-C. Oncolytic Viruses, Genomes, Vectors and Cells Comprising One or More Expression Cassettes

Also provided herein is an oncolytic virus (e.g., an oncolytic HSV, such as an oncolytic HSV-1 or oncolytic HSV-2) comprising one or more of the expression cassettes described above (e.g., in Sections IV-A and/or IV-B). In some embodiments, the oncolytic virus comprises one or more expression cassettes comprising a polynucleotide encoding IL-12, a polynucleotide encoding a CD40 agonist, and/or a polynucleotide encoding a CTLA-4 binding protein, e.g., as described above in Section IV-A. In some embodiments, the oncolytic virus comprises one or more expression cassettes comprising a polynucleotide encoding FLT3L and/or a polynucleotide encoding a transporter associated with antigen processing (TAP) inhibitor, e.g., as described above in Section IV-B. In some embodiments, the oncolytic virus comprises: (a) one or more expression cassettes comprising a polynucleotide encoding IL-12, a polynucleotide encoding a CD40 agonist, and/or a polynucleotide encoding a CTLA-4 binding protein, e.g., as described above in Section IV-A; and (b) one or more expression cassettes comprising a polynucleotide encoding FLT3L and/or a polynucleotide encoding a transporter associated with antigen processing (TAP) inhibitor, e.g., as described above in Section IV-B. In some embodiments, an oncolytic virus of the disclosure exhibits increased T cell activation relative to an oncolytic virus lacking any one, any two, or all of the polynucleotides encoding the IL-12 protein, the CD40 agonist, and the CTLA-4 binding protein. T cell activation may be assessed using any suitable method known in the art, such as using an in vitro IL-2 secretion assay, e.g., as described in Example 2, herein. In some embodiments, an oncolytic virus of the disclosure has increased abscopal efficacy relative to an oncolytic virus lacking any one, any two, or any three of the FLT3L, the IL-12, the CD40 agonist, and the CTLA-4 binding protein. Abscopal efficacy may be assessed using any suitable method known in the art, such as using an in vivo tumor or cancer animal model, e.g., as described in Example 7, herein. In some embodiments, an oncolytic virus of the disclosure is capable of evading an individual's immune system. In some embodiments, an oncolytic virus of the disclosure reduces or impairs viral antigen loading onto histocompatibility complex (MHC) Class I molecules for display at the cell surface, thereby reducing adaptive immune responses to the virus.

Also provided herein, is a modified HSV genome (e.g., an HSV-1 or HSV-2 genome) comprising one or more of the expression cassettes described above (e.g., in Sections IV-A and IV-B). In some embodiments, the modified HSV genome comprises one or more expression cassettes comprising a polynucleotide encoding IL-12, a polynucleotide encoding a CD40 agonist, and/or a polynucleotide encoding a CTLA-4 binding protein, e.g., as described above in Section IV-A. In some embodiments, the modified HSV genome comprises one or more expression cassettes comprising a polynucleotide encoding FLT3L and/or a polynucleotide encoding a transporter associated with antigen processing (TAP) inhibitor, e.g., as described above in Section IV-B. In some embodiments, the modified HSV genome comprises: (a) one or more expression cassettes comprising a polynucleotide encoding IL-12, a polynucleotide encoding a CD40 agonist, and/or a polynucleotide encoding a CTLA-4 binding protein, e.g., as described above in Section IV-A; and (b) one or more expression cassettes comprising a polynucleotide encoding FLT3L and/or a polynucleotide encoding a transporter associated with antigen processing (TAP) inhibitor, e.g., as described above in Section IV-B.

Also provided herein, is a vector comprising one or more of the expression cassettes described above (e.g., in Sections IV-A and IV-B). Suitable vectors include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR.322, pMB9, ColEl, pCRI, RP4, phage DNAs, and shuttle vectors such as pSA3 and pAT28. These and many other cloning vectors are available from commercial vendors such as BioRad, Strategene, and Invitrogen. Expression vectors generally are replicable polynucleotide constructs that contain a nucleic acid of the present disclosure. The expression vector may be replicable in the host cells either as episomes or as an integral part of the chromosomal DNA. Suitable expression vectors include but are not limited to plasmids, viral vectors, including adenoviruses, adeno-associated viruses, HSV viruses, e.g. HSV-1 or HSV-2, retroviruses, and cosmids. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually included, such as ribosome binding sites, translation initiation sites, and stop codons.

In some embodiments, cells, such as host cells, comprising one or more of the expression cassettes described above (e.g., in Sections IV-A and IV-B) are also provided. In some embodiments, the cell is an isolated cell. An isolated cell is a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the host cell is eukaryotic, e.g., a Chinese Hamster Ovary (CHO) cell, human cells such as HELA cells, HEK293 cells, etc., or lymphoid cell (e.g., YO, NSO, Sp20 cell). Host cells of the present disclosure also include, without limitation, isolated cells, in vitro cultured cells, and ex vivo cultured cells. In some embodiments, the cell is a mammalian cell.

### V. CD40 Agonist

Further provided herein is a CD40 agonist protein. In some embodiments, the CD40 agonist protein of the disclosure is an agonist of a cluster of differentiation 40 (CD40) protein. In some embodiments, the CD40 agonist comprises a CD40 ligand ectodomain. In some embodiments, the CD40 agonist is a trimer of three single-chain trimeric CD40 ligand ectodomains. In some embodiments, each of the three single-chain trimeric CD40 ligand ectodomains is fused to a trimerization motif, e.g., to direct formation of a trimer of three single-chain trimeric CD40 ligand ectodomains. In some embodiments, the CD40 agonist is a trimer of trimers, i.e., a trimer comprising three single-chain trimeric CD40 ligand ectodomains

In some embodiments, the CD40 agonist comprises a human CD40 ligand ectodomain. In some embodiments, the human CD40 ligand ectodomain comprises the amino acid sequence set forth in SEQ ID NO:20, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:20. In some embodiments, the CD40 agonist is a trimer of three single-chain trimeric human CD40 ligand ectodomains. In some embodiments, the single-chain trimeric human CD40 ligand ectodomains comprise a polypeptide comprising three human CD40 ligand ectodomains, e.g., connected by peptide linkers. In some embodiments, the single-chain trimeric human CD40 ligand ectodomain polypeptide comprises a first human CD40 ligand ectodomain connected by a peptide linker to a second human CD40 ligand ectodomain which is connected by a peptide linker to a third human CD40 ligand ectodomain. In some embodiments, the peptide linker comprises glycine and serine residues. In some embodiments, the peptide linker comprises the amino acid sequence set forth in SEQ ID NO:22, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:22. In some embodiments, the CD40 agonist comprises a trimerization motif operably linked to the three single-chain trimeric CD40 ligand ectodomains. In some embodiments, the trimerization motif is a T4 fibritin trimerization motif. In some embodiments, the T4 fibritin trimerization motif comprises the amino acid sequence set forth in SEQ ID NO:21, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:21. In some embodiments, the trimerization motif is linked to the three single-chain trimeric CD40 ligand ectodomains by a peptide linker. In some embodiments, the peptide linker connecting the trimerization motif to the three single-chain trimeric CD40 ligand ectodomains comprises an amino acid sequence comprising leucine, glycine, and/or serine residues. In some embodiments, the peptide linker comprises the amino acid sequence of SEQ ID NO:23, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:23. In some embodiments, the peptide linker comprises the amino acid sequence of SEQ ID NO:27, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:27. In some embodiments, the CD40 agonist further comprises a signal peptide sequence operably linked to the three single-chain trimeric CD40 ligand ectodomains. In some embodiments, the signal peptide sequence comprises the amino acid sequence of SEQ ID NO:24. In some embodiments, the CD40 agonist comprises a polypeptide comprising the amino acid sequence of SEQ ID NO:25, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:25. In some embodiments, the CD40 agonist comprises a polypeptide comprising the amino acid sequence of SEQ ID NO:30, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:30. In some embodiments, the CD40 agonist forms or is a trimer comprising three polypeptides comprising the amino acid sequence of SEQ ID NO:25, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:25. In some embodiments, the CD40 agonist forms or is a trimer comprising three polypeptides comprising the amino acid sequence of SEQ ID NO:30, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:30.

In some embodiments, the CD40 agonist comprises a murine CD40 ligand ectodomain. In some embodiments, the murine CD40 ligand ectodomain comprises the amino acid sequence set forth in SEQ ID NO:26, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:26. In some embodiments, the CD40 agonist is a trimer of three single-chain trimeric murine CD40 ligand ectodomains. In some embodiments, the single-chain trimeric murine CD40 ligand ectodomains comprise a polypeptide comprising three murine CD40 ligand ectodomains, e.g., connected by peptide linkers. In some embodiments, the single-chain trimeric murine CD40 ligand ectodomain polypeptide comprises a first murine CD40 ligand ectodomain connected by a peptide linker to a second murine CD40 ligand ectodomain which is connected by a peptide linker to a third murine CD40 ligand ectodomain. In some embodiments, the peptide linker comprises glycine and serine residues. In some embodiments, the peptide linker comprises the amino acid sequence set forth in SEQ ID NO:22, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:22. In some embodiments, the CD40 agonist comprises a trimerization motif operably linked to the three single-chain trimeric CD40 ligand ectodomains. In some embodiments, the trimerization motif is a T4 fibritin trimerization motif. In some embodiments, the T4 fibritin trimerization motif comprises the amino acid sequence set forth in SEQ ID NO:21, or an amino acid sequence having any of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:21. In some embodiments, the trimerization motif is linked to the three single-chain trimeric CD40 ligand ectodomains by a peptide linker. In some embodiments, the peptide linker connecting the trimerization motif to the three single-chain trimeric CD40 ligand ectodomains comprises an amino acid sequence comprising leucine, glycine, and/or serine residues. In some embodiments, the peptide linker comprises the amino acid sequence of SEQ ID NO:23, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:23. In some embodiments, the peptide linker comprises the amino acid sequence of SEQ ID NO:27, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:27. In some embodiments, the CD40 agonist further comprises a signal peptide sequence operably linked to the three single-chain trimeric CD40 ligand ectodomains. In some embodiments, the signal peptide sequence comprises the amino acid sequence of SEQ ID NO:24. In some embodiments, the CD40 agonist comprises a polypeptide comprising the amino acid sequence of SEQ ID NO:28, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:28. In some embodiments, the CD40 agonist comprises a polypeptide comprising the amino acid sequence of SEQ ID NO:29, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:29. In some embodiments, the CD40 agonist forms or is a trimer comprising three polypeptides comprising the amino acid sequence of SEQ ID NO:28, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:28. In some embodiments, the CD40 agonist forms or is a trimer comprising three polypeptides comprising the amino acid sequence of SEQ ID NO:29, or an amino acid sequence having any of about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homology to the amino acid sequence set forth in SEQ ID NO:29.

In some embodiments, the CD40 agonist provided herein is any of the CD40 agonists described in Section III-B, above.

In some embodiments, a CD40 agonist of the disclosure induces or enhances CD40 signaling, activates dendritic cells, and/or inhibits tumor growth. In some embodiments, CD40 signaling may be assessed using any suitable method, such as using CD40 reporter cells in vitro, e.g., reporter cells that emit a detectable signal upon activation of the CD40 signaling pathway. In some embodiments, CD40 signaling may be assessed using a HEK-Blue reporter assay, see, e.g., www.invivogen.com/hek-blue-cd401. In an exemplary HEK-Blue reporter assay, HEK293 reporter cells stably transfected with a CD40 gene, e.g., a human CD40 gene, and an NFκB-inducible secreted alkaline phosphatase (SEAP) construct are contacted with the CD40 agonist. The reporter cells respond to CD40 agonist binding by the production of a colorimetric readout. In some embodiments, dendritic cell activation may be assessed using any suitable method, such as by assessing levels of the CD86 activation marker expressed on one or more cells in vitro, e.g., using flow cytometry. In some embodiments, tumor growth inhibition may be assessed using any suitable method, such as using an in vivo mouse tumor model, e.g., as described in Example 2 herein.

Also provided herein are nucleic acids, e.g., isolated nucleic acids, having a nucleotide sequence encoding any of the CD40 agonists of the present disclosure. Such nucleic acids may encode an amino acid sequence of a CD40 ligand ectodomain, e.g., a human or murine CD40 ligand ectodomain as described above. In some embodiments, a nucleic acid of the disclosure encodes an amino acid sequence comprising three single-chain trimeric human CD40 ligand ectodomains, e.g., as described above. In other embodiments, a nucleic acid of the disclosure encodes an amino acid sequence comprising three single-chain trimeric human or murine CD40 ligand ectodomains, wherein the amino acid sequence of the three single-chain trimeric human CD40 ligand ectodomains is operably linked (for example, via a linker) to an amino acid sequence of a trimerization motif, such as a T4 fibritin trimerization motif, e.g., as described above. In some embodiments, a nucleic acid of the disclosure further encodes a signal peptide sequence, e.g., fused to the amino acid sequence of the three single-chain trimeric human CD40 ligand ectodomains and timerization motif, e.g., as described above.

Also provided herein are one or more vectors (e.g., cloning vectors or expression vectors) containing any of the nucleic acids of the disclosure, e.g. encoding any of the CD40 agonists of the present disclosure. Suitable vectors containing a nucleic acid sequence encoding any of the CD40 agonists of the present disclosure, or fragments thereof, include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR.322, pMB9, ColEl, pCRI, RP4, phage DNAs, and shuttle vectors such as pSA3 and pAT28. These and many other cloning vectors are available from commercial vendors such as BioRad, Strategene, and Invitrogen. Expression vectors generally are replicable polynucleotide constructs that contain a nucleic acid of the present disclosure. The expression vector may be replicable in the host cells either as episomes or as an integral part of the chromosomal DNA. Suitable expression vectors include but are not limited to plasmids, viral vectors, including adenoviruses, adeno-associated viruses, HSV viruses, e.g. HSV-1 or HSV-2, retroviruses, and cosmids. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually included, such as ribosome binding sites, translation initiation sites, and stop codons.

In some embodiments, a host cell containing any of the nucleic acids or vectors of the disclosure is also provided. In some embodiments, the host cell is an isolated host cell. An isolated cell is a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the host cell is eukaryotic, e.g., a Chinese Hamster Ovary (CHO) cell, human cells such as HELA cells, HEK293 cells, etc., or lymphoid cell (e.g., YO, NSO, Sp20 cell). Host cells of the present disclosure also include, without limitation, isolated cells, in vitro cultured cells, and ex vivo cultured cells.

A CD40 agonist of the disclosure may be produced using recombinant methods and compositions. In some embodiments, methods of making a CD40 agonist of the present disclosure are provided. In some embodiments, the methods include culturing a host cell of the present disclosure containing nucleic acid encoding a CD40 agonist of the disclosure, under conditions suitable for expression of the CD40 agonist. In some embodiments, the CD40 agonist is subsequently recovered from the host cell (or host cell culture medium). For recombinant production of a CD40 agonist of the present disclosure, nucleic acid encoding the CD40 agonist is isolated and inserted into one or more vectors for further cloning and/or expression in a host cell. Such nucleic acid may be readily isolated and sequenced using conventional procedures (e.g., by using oligonucleotide probes that are capable of binding specifically to sequences encoding the CD40 agonist). Vectors or nucleic acids of the disclosure can be introduced into a host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or nucleic acids will often depend on features of the host cell.

A CD40 agonist of the disclosure can be incorporated into a variety of formulations for therapeutic administration by combining the CD40 agonist with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The pharmaceutical composition can also include any of a variety of stabilizing agents, such as an antioxidant, for example. When the pharmaceutical composition includes a polypeptide, such as a CD40 agonist of the disclosure, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids. Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249:1527-1533 (1990).

### VI. Methods of Making Oncolytic Viruses

The oncolytic viruses (such as the oncolytic HSV) described herein may be prepared using any methods known in the art or as described herein. In some embodiments, the oncolytic virus (such as the oncolytic HSV) may be engineered (such as to comprise one or more of the expression cassettes described herein and/or to express one or more of the payload proteins described herein) by modifying a wild-type virus (such as a wild-type HSV-1) genome. Transgenes and/or expression cassettes, including as otherwise described herein, may be inserted in the native genome or replace native portions of the genome using recombinant cloning techniques well known in the art. Exemplary engineering methods are described herein at Examples 4-7. Engineered oncolytic virus genomes may be propagated in suitable cells and collected from cell media or collected from cell lysates. Purified virus may be titered using assays well known in the art. Viral titer may be expressed in terms of infectious viral units, such as plaque-forming units (pfu). The integrity and sequence of the viral genome may be assessed by techniques well known in the art, including whole-genome sequencing.

### VII. Pharmaceutical Compositions

Further provided herein are pharmaceutical compositions comprising any of the oncolytic viruses (such as any of the oncolytic HSV) described herein, and optionally a pharmaceutically acceptable excipient, carrier, and/or stabilizer. Pharmaceutical compositions can be prepared by mixing a oncolytic virus (such as any of the oncolytic HSV) described herein having a desired degree of purity with pharmaceutically acceptable carriers, excipients, and/or stabilizers.

The pharmaceutical compositions may be administered by any suitable route, including intratumoral or intravesical.

### IX. Methods of Use

Further provided are methods of treating cancer in an individual comprising administering to the individual a therapeutically effective amount of the oncolytic virus as described herein or a pharmaceutical composition comprising the oncolytic virus as described herein. Further provided are methods of killing tumor cells in an individual, the method comprising administering a therapeutically effective amount of the oncolytic virus as described herein or a pharmaceutical composition comprising the oncolytic virus as described herein. In some embodiments, the oncolytic virus comprises one or more of the expression cassettes described herein. In some embodiments, the oncolytic virus expresses one or more of the payload proteins described herein. In some embodiments, the oncolytic virus is the oncolytic HSV described herein. In some embodiments, the cancer to be treated is a solid tumor. In some embodiments, the cancer to be treated is a metastatic cancer. In some embodiments, the cancer to be treated is a recurrent cancer. In some embodiments, the cancer is regionally advanced.

In some embodiments, provided herein are methods of expressing genes in vivo, such as in a tumor microenvironment. In some embodiments, the methods comprise delivering a viral vector comprising the genes. In some embodiments, one or more of the genes cause an immunological response. In some embodiments, expression of one or more of the genes result in recruitment of dendritic cells to a tumor microenvironment. In some embodiments, one or more of the genes cause maturation of dendritic cells into licensed antigen-presenting cells (APC). In some embodiments, one or more of the genes enhance T-cell effector cytokine production. In some embodiments, one or more of the genes promotes CD4+T helper (Th)1 response that sustain cytotoxic CD8+ T cells. In some embodiments, one or more of the genes drives T-cell activation. In some embodiments, one or more of the genes promote depletion of regulatory T-cells (Tregs). In some embodiments, the genes are selected form the group consisting of a FLT3 ligand, a CD40 agonist, IL-1, and a CTLA-4 antagonist.

In some embodiments, administration of the oncolytic virus to an individual results in one or more therapeutic effects in the subject. In one embodiment, the one or more therapeutic effects is a reduction in the size of the tumor derived from the cancer. In one embodiment, the one or more therapeutic effects is lysing of tumor cells in the individual. In some embodiments, the oncolytic virus preferentially lyses tumor cells. To selectively lyse tumor cells, as used herein, means the oncolytic virus preferentially lyses tumor cells compared to neighboring non-tumor cells. It is understood that the oncolytic virus may still lyse neighboring healthy cells, but at a lower efficiency compared to target tumor cells. In one embodiment, the one or more therapeutic effects is decreased tumor size.

Administration of the oncolytic virus described herein or a pharmaceutical composition comprising the oncolytic virus described herein may result in an abscopal response in the subject. In an abscopal response, administration of the oncolytic virus or pharmaceutical composition to a first site (such as a primary tumor) may kill tumor cells at both the first site (a primary effect) and at one or more additional sites, such as a secondary site (such as distal tumor sites or metastatic tumor sites; an abscopal effect). Such an abscopal effect may be mediated by the immune response to the oncolytic virus at the first site (e.g., the site where the virus is originally delivered). Thus, in some embodiments, the oncolytic virus described herein, or a pharmaceutical composition comprising the oncolytic virus described herein, is administered at a first site, and the oncolytic virus triggers an immune response that results in killing of tumor cells at the second site, such as a distal tumor or metastasis. In some embodiments, administration of the oncolytic virus or a pharmaceutical composition comprising the oncolytic virus results in an immune response in the subject. In some embodiments, said immune response causes tumor growth inhibition at either or both of the first site (where the virus is delivered) and the second site. In some embodiments, the immune response is enhanced by expression of one or more payload proteins as described herein. Immunomodulatory payload protein expression following oncolytic virus treatment can result in dendritic cell infiltration into the tumor, dendritic cell, monocyte, and/or macrophage activation, T cell infiltration, T cell activation, T cell expansion, T regulatory cell depletion, NK cell infiltration, interferon-gamma production, and reduction of suppressive immune populations such as myeloid derived suppressor cells and tumor associated macrophages. In some embodiments, the oncolytic virus induces a sustained antitumor immune response. In some embodiments, the immune system develops a memory of tumor antigens and is able to recognize and ablate tumor cells several days, weeks, months, or later after administration.

In some embodiments, the oncolytic virus enhances T cell function. In some embodiments, the oncolytic virus depletes T regulatory cells (Tregs) in the tumor microenvironment. In some embodiments, the oncolytic virus recruits dendritic cells to the tumor microenvironment. In some embodiments, the oncolytic virus matures dendritic cells.

In the context of an oncolytic HSV, in some embodiments, the oncolytic HSV expresses an immediate-early US11. In some embodiments, the oncolytic HSV expresses both an immediate-early US11 and a native late US11. In some embodiments, the oncolytic HSV expresses UL49.5, which is a TAP inhibitor from bovine herpesvirus 1. In some embodiments, expression of UL49.5 inhibits TAP from transporting peptides into the lumen of the endoplasmic reticulum, thus impairing loading onto major histocompatibility complex (MHC) Class I molecules for display at the cell surface, reducing adaptive immune response to the virus. In some embodiments, this impairment of MHC Class I loading is specific for the targeted cells, wherein neighboring uninfected cells are not impaired for MHC Class I loading.

### EXEMPLARY EMBODIMENTS

The present disclosure may be better understood with reference to the following exemplary embodiments.

Embodiment 1. An oncolytic herpes simplex type 1 virus (HSV-1) comprising
a. a cassette integrated in one or both of the γ34.5 loci comprising in order, from upstream to downstream, a CMV promoter, a polynucleotide encoding hFLT3L, a P2A cleavage sequence, a polynucleotide encoding UL49.5, and a polyadenylation signal; and
b. another cassette integrated in the US10-12 locus comprising in order, from upstream to downstream, a polynucleotide comprising a variant US11 gene encoding native US11 protein, an additional polynucleotide encoding native US11 protein, a polynucleotide encoding US10 protein, a polyadenylation signal that is operably linked to the polynucleotide encoding the US10 protein, a CMV promoter, a polynucleotide encoding a CTLA-4 binding protein, a polyadenylation signal that is operably linked to the polynucleotide encoding the CTLA-4 binding protein, a polyadenylation signal that is operably linked to a polynucleotide encoding a CD40 agonist, a polynucleotide encoding a CD40 agonist, an AoHV1 promoter that controls expression of the CD40 agonist, an MMLV promoter that controls expression of an IL-12, a polynucleotide encoding an IL-12, and a polyadenylation signal that is operably linked to the polynucleotide encoding the IL-12, wherein
   the polynucleotide for hFLT3L encodes the amino acid sequence set forth in SEQ ID NO:71, the polynucleotide for UL49.5 encodes the amino acid sequence set forth in SEQ ID NO: 82, the polynucleotide for IL-12 encodes the amino acid sequence set forth in SEQ ID NO: 4, the polynucleotide for CD40 agonist encodes the amino acid sequence set forth in SEQ ID NO: 25, and the polynucleotide for CTLA-4 binding protein encodes the amino acid sequence set forth in SEQ ID NO: 50, the polynucleotide for variant US11 gene comprises the polynucleotide sequence set forth in SEQ ID NO: 204, the additional polynucleotide for US11 encodes the amino acid sequence set forth in SEQ ID NO:80, and the polynucleotide for US10 encodes the amino acid sequence set forth in SEQ ID NO:90.

2. The oncolytic HSV-1 of embodiment 1, wherein the bGH polyadenylation signal is SEQ ID NO: 102, the hGH polyadenylation signal is SEQ ID NO: 209, the GAPDH synthetic polyadenylation signal is SEQ ID NO: 213, the C2 transcriptional pause site is SEQ ID NO: 214, the hGT transcriptional pause site is SEQ ID NO: 215, and the hBG polyadenylation signal is SEQ ID NO: 216.

3. The oncolytic HSV-1 of claim 1, wherein and wherein expression of the variant US11 gene encoding a native US11 protein is under control of the native US12 immediate early promoter, the expression of the additional polynucleotide encoding native US11 protein is under control of its native promoter, and the expression of the polynucleotide encoding US10 protein is under control of its native promoter.

4. An oncolytic herpes simplex type 1 virus (HSV-1) comprising
a cassette integrated in one or both of the γ34.5 loci comprising in order, from upstream to downstream, a CMV promoter, a polynucleotide encoding hFLT3L, a P2A cleavage sequence, a polynucleotide encoding UL49.5, and a polyadenylation signal; and
another cassette integrated in the US10-12 locus comprising in order, from upstream to downstream, a polynucleotide comprising a variant US11 gene encoding a native US11 protein, an additional polynucleotide encoding native US11 protein, a polynucleotide encoding US10 protein, a polyadenylation signal that is operably linked to the polynucleotide encoding the US10 protein, a CMV promoter, a polynucleotide encoding a CTLA-4 binding protein, a polyadenylation signal that is operably linked to the polynucleotide encoding the CTLA-4 binding protein, a polyadenylation signal that is operably linked to a polynucleotide encoding a CD40 agonist, a polynucleotide encoding a CD40 agonist, an AoHV1 promoter that controls expression of the CD40 agonist, an MMLV promoter that controls expression of an IL-12, a polynucleotide encoding the IL-12, and a polyadenylation signal that is operably linked to the polynucleotide encoding the IL-12, wherein
the polynucleotide for hFLT3L encodes the amino acid sequence set forth in SEQ ID NO:71, the polynucleotide for UL49.5 encodes the amino acid sequence set forth in SEQ ID NO: 82, the polynucleotide for IL-12 encodes the amino acid sequence set forth in SEQ ID NO: 8, the polynucleotide for CD40 agonist encodes the amino acid sequence set forth in SEQ ID NO: 28, and the polynucleotide for CTLA-4 binding protein encodes the amino acid sequence set forth in SEQ ID NO: 56, the polynucleotide for variant US11 gene comprises the polynucleotide sequence set forth in SEQ ID NO: 204, the additional polynucleotide encoding US11 encodes the amino acid sequence set forth in SEQ ID NO:80, and the polynucleotide for US10 encodes the amino acid sequence set forth in SEQ ID NO:90.

5. The oncolytic HSV-1 of embodiment 4, wherein the bGH polyadenylation signal is SEQ ID NO: 102, the hGH polyadenylation signal is SEQ ID NO: 209, the GAPDH synthetic polyadenylation signal is SEQ ID NO: 213, the C2 transcriptional pause site is SEQ ID NO: 214, the hGT transcriptional pause site is SEQ ID NO: 215, and the hBG polyadenylation signal is SEQ ID NO: 216.

6. An oncolytic virus comprising one or more expression cassettes comprising a polynucleotide encoding IL-12, a polynucleotide encoding a CD40 agonist, and a polynucleotide encoding a CTLA-4 binding protein.

7. The oncolytic virus of embodiment 6, wherein the virus comprises backbone nucleic acid encoding one or more native viral proteins associated with viral replication and/or packaging.

8. The oncolytic virus of embodiment 6, wherein one or both native γ34.5 genes are inactivated by deletion, substitution, or insertion in the backbone nucleic acid.

9. The oncolytic virus of any one of embodiments 6-8, wherein a native US12 gene of the virus is inactivated by deletion, substitution, or insertion in the backbone nucleic acid.

10. The oncolytic virus of any one of embodiments 6-9, wherein the IL-12 is a heterodimer comprising a p35 subunit and a p40 subunit.

11. The oncolytic virus of embodiment 10, wherein the p35 subunit and/or the p40 subunit are human.

12. The oncolytic virus of embodiments 10 or 11, wherein the p35 subunit comprises the sequence of amino acids of SEQ ID NO:1.

13. The oncolytic virus of any one of embodiments 10-12, wherein the p40 subunit comprises the sequence of amino acids of SEQ ID NO:2.

14. The oncolytic virus of embodiment 10, wherein the p35 subunit and/or the p40 subunit are murine.

15. The oncolytic virus of embodiment 14, wherein the p35 subunit comprises the sequence of amino acids of SEQ ID NO:5.

16. The oncolytic virus of embodiments 14 or 15, wherein the p40 subunit comprises the sequence of amino acids of SEQ ID NO:6.

17. The oncolytic virus of any one of embodiments 10-16, wherein the p35 subunit and p40 subunit are connected by a peptide linker.

18. The oncolytic virus of embodiment 14, wherein the peptide linker comprises an amino acid sequence comprising glycine and serine residues.

19. The oncolytic virus of embodiment 18, wherein the linker comprises the amino acid sequence set forth in SEQ ID NO:3 or SEQ ID NO:7.

20. The oncolytic virus of any one of embodiments 6-19, wherein the CD40 agonist is a CD40 ligand.

21. The oncolytic virus of any one of embodiments 6-20, wherein the CD40 agonist comprises a CD40 ligand ectodomain.

22. The oncolytic virus of embodiment 21, wherein the CD40 agonist is a trimer of three single-chain trimeric CD40 ligand ectodomains.

23. The oncolytic virus of embodiments 21 or 22, wherein the CD40 ligand ectodomain is human.

24. The oncolytic virus of embodiment 23, wherein the CD40 ligand ectodomain comprises the amino acid sequence set forth in SEQ ID NO:20.

25. The oncolytic virus of embodiments 21 or 22, wherein the CD40 ligand ectodomain is murine.

26. The oncolytic virus of embodiment 24, wherein the CD40 ligand ectodomain comprises the amino acid sequence set forth in SEQ ID NO:26.

27. The oncolytic virus of any one of embodiments 22-26, wherein the CD40 agonist comprises a trimerization motif operably linked to each of the three single-chain trimeric CD40 ligand ectodomains.

28. The oncolytic virus of embodiment 27, wherein the trimerization motif is a T4 fibritin trimerization motif.

29. The oncolytic virus of embodiments 27 or 28, wherein the trimerization motif is linked to each of the three single-chain trimeric ectodomains of the CD40 agonist by a linker comprising glycine and serine residues.

30. The oncolytic virus of any one of embodiments 6-29, wherein the CTLA-4 binding protein is a CTLA-4 antibody or antigen binding fragment thereof.

31. The oncolytic virus of embodiment 30, wherein the CTLA-4 antibody or antigen binding fragment thereof is an scFv.

32. The oncolytic virus of embodiments 30 or 31, wherein the anti-CTLA-4 antibody or antigen binding fragment thereof specifically binds to human CTLA-4.

33. The oncolytic virus of any one of embodiments 30-32, wherein the anti-CTLA-4 antibody or antigen binding fragment is bivalent.

34. The oncolytic virus of any one of embodiments 31-33, wherein the anti-CTLA-4 scFv is fused to the N-terminus of a IgG1 constant domain.

35. The oncolytic virus of embodiment 34, wherein the human IgG1 is a variant human IgG1 comprising a C220S substitution, with numbering according to EU index numbering. 36. The oncolytic virus of any one of embodiments 30-35, wherein the anti-CTLA-4 antibody or antigen binding fragment thereof comprises:
a CDRH1 comprising the amino acid sequence set forth in SEQ ID NO:40;
a CDRH2 comprising the amino acid sequence set forth in SEQ ID NO:41;
a CDRH3 comprising the amino acid sequence set forth in SEQ ID NO:42
a CDRL1 comprising the amino acid sequence set forth in SEQ ID NO:43;
a CDRL2 comprising the amino acid sequence set forth in SEQ ID NO:44; and
a CDRL3 comprising the amino acid sequence set forth in SEQ ID NO:45.

37. The oncolytic virus of any one of embodiments 30-35, wherein the anti-CTLA-4 antibody or antigen binding fragment thereof comprises a variable heavy chain (VH) comprising the amino acid sequence set forth in SEQ ID NO:46 and a variable light chain (VL) comprising the amino acid sequence set forth in SEQ ID NO:47.

38. The oncolytic virus of embodiments 36 or 37, wherein the anti-CTLA-4 antibody or antigen binding fragment thereof comprises the sequence of amino acids of SEQ ID NO:50.

39. The oncolytic virus of embodiment 30, wherein the CTLA-4 antibody is a camelid antibody comprising an anti-CTLA-4 VHH.

40. The oncolytic virus of embodiment 39, wherein the VHH is fused to the heavy chain of a murine IgG2a Fc.

41. The oncolytic virus of embodiment 40, wherein the anti-CTLA-4 VHH comprises the amino acid sequence set forth in SEQ ID NO:54.

42. The oncolytic virus of any one of embodiments 6-41, wherein the one or more expression cassettes comprise nucleic acid encoding a FLT3 ligand (FLT3L).

43. The oncolytic virus of embodiment 42, wherein the FLT3L is human.

44. The oncolytic virus of embodiments 42 or 43, wherein the FLT3L is a homodimer.

45. The oncolytic virus of any one of embodiments 42-44, wherein the FLT3L comprises a signal peptide directing secretion to the plasma membrane.

46. The oncolytic virus of any one of embodiments 42-44, wherein the FLT3L comprises the amino acid sequence set forth in SEQ ID NO:71.

47. The oncolytic virus of any one of embodiments 6-46, wherein the one or more one or more expression cassettes further comprises a polynucleotide comprising a variant US11 gene.

48. The oncolytic virus of embodiment 47, wherein the polynucleotide comprising a variant US11 gene is human-codon-optimized compared to a native gene encoding US11.

49. The oncolytic virus of embodiments 47 or 48, wherein the variant US11 gene comprises the polynucleotide sequence of SEQ ID NO: 204.

50. The oncolytic virus of any one of embodiments 47-49, wherein the variant US11 gene is operably associated with an immediate-early promoter.

51. The oncolytic virus of embodiment 50, wherein the variant US11 gene is operably associated with the native US12 immediate-early promoter to express immediate-early US11 protein.

52. The oncolytic virus of any one of embodiments 6-51, wherein the oncolytic virus does not express granulocyte macrophage colony-stimulating factor (GM-CSF).

53. The oncolytic virus of any one of embodiments 6-52, comprising a native late US11 gene.

54. The oncolytic virus of any one of embodiments 6-53, further comprising a transporter associated with antigen processing (TAP) inhibitor.

55. The oncolytic virus of embodiment 54, wherein the TAP inhibitor is derived from herpesvirus 1 or herpes virus 2.

56. The oncolytic virus of embodiment 55, wherein the TAP inhibitor is derived from bovine herpes virus 1.

57. The oncolytic virus of embodiments 54 or 55, wherein the TAP inhibitor is UL49.5, US6, or ICP47.

58. The oncolytic virus of embodiment 57, wherein the TAP inhibitor is UL49.5

59. The oncolytic virus of any one of embodiments 54-58, wherein the TAP inhibitor is expressed as an immediate-early gene.

60. The oncolytic virus of any one of embodiments 42-59, wherein the expression cassette comprises a polynucleotide encoding the FLT3L.

61. The oncolytic virus of embodiment 60, wherein the expression cassette comprises polynucleotides encoding the TAP inhibitor.

62. The oncolytic virus of embodiment 61, wherein the expression cassette comprises a polynucleotide encoding a self-cleaving peptide.

63. The oncolytic virus of embodiment 62, wherein the polynucleotide encoding the self-cleaving peptide is positioned between the polynucleotide encoding the FLT3L and the polynucleotide encoding the TAP inhibitor.

64. The oncolytic virus of embodiments 62 or 63, wherein the self-cleaving peptide is P2A.

65. The oncolytic virus of embodiment 64, wherein P2A comprises the amino acid sequence of SEQ ID NO:91.

66. The oncolytic virus of any one of embodiments 6-65 wherein at least one of the native γ34.5 loci rendered functionally inactive by the insertion of the expression cassette.

67. The oncolytic virus of embodiment 66 wherein both native γ34.5 loci are replaced or substantially replaced by a copy of the expression cassette.

68. The oncolytic virus of any one of embodiments 61-67, wherein the one or more expression cassettes comprise a CMV promoter that regulates expression of the polynucleotide encoding the FLT3L and the polynucleotide encoding the TAP inhibitor.

69. The oncolytic virus of embodiment 68, wherein the expression cassette comprises a polyadenylation signal.

70. The oncolytic virus of embodiment 69, wherein the polyadenylation signal is a bovine growth hormone polyadenylation signal (BGHpA).

71. The oncolytic virus of any one of embodiments 63-70, wherein the expression cassette replaces all or substantially all of one or both of the γ34.5 loci of the long terminal repeat (TR_{L}) and comprises (i) the nucleic acid encoding the TAP inhibitor, (ii) nucleic acid encoding the self-cleaving peptide, (iii) the nucleic acid encoding the FLT3L; wherein the cassette is in the orientation relative to the unique long (UL) region of the genome of (i)-(ii)-(iii)-UL.

72. The oncolytic virus of embodiment 71, wherein the expression cassette replaces all or substantially all of both of the γ34.5 loci of the TR_{L}.

73. The oncolytic virus of any one of embodiments 56-63, wherein the expression cassette replaces all or substantially all of the γ34.5 locus of the internal long repeat (IRL) and comprises (i) the nucleic acid encoding the TAP inhibitor, (ii) the nucleic acid encoding the self-cleaving peptide, (iii) the nucleic acid encoding the FLT3L; wherein the cassette is in the orientation relative to the UL region of the genome of UL-(iii)-(ii)-(i).

74. The oncolytic virus of embodiment 73, wherein the expression cassette replaces all or substantially all of the IRL.

75. The oncolytic virus of any one of embodiments 6-74, wherein one of the expression cassettes is inserted at the native US10-US12 locus.

76. The oncolytic virus of embodiment 75, wherein the expression cassette comprises the polynucleotide encoding IL-12.

77. The oncolytic virus of embodiments 75 or 76, wherein the expression cassette comprises the polynucleotide encoding the CD40 agonist.

78. The oncolytic virus of any one of embodiments 75-77, wherein the expression cassette comprises the polynucleotide encoding the CTLA-4 binding protein.

79. The oncolytic virus of any one of embodiments 75-78, wherein the expression cassette comprises a polynucleotide encoding a US10 protein.

80. The oncolytic virus of embodiment 79, wherein the expression cassette comprises a polyadenylation signal positioned after the polynucleotide encoding the US10 protein.

81. The oncolytic virus of embodiment 80, wherein the polyadenylation signal is a human growth hormone polyadenylation signal (hGHpA).

82. The oncolytic virus of any one of embodiments 75-81, wherein the expression cassette comprises a native late US11 gene.

83. The oncolytic virus of any one of embodiments 75-82 wherein the expression cassette comprises a polynucleotide comprising a variant US11 gene.

84. The oncolytic virus of any one of embodiments 75-83, wherein the expression cassette does not express a US12 protein.

85. The oncolytic virus of any one of embodiments 78-84, wherein the expression cassette comprises a CMV promoter positioned upstream of the polynucleotide encoding the CTLA-4 binding protein.

86. The oncolytic virus of any one of embodiments 78-85, wherein the expression cassette comprises a polyadenylation signal positioned after the polynucleotide encoding the CTLA-4 binding protein.

87. The oncolytic virus of embodiment 86, wherein the polyadenylation signal is a polyadenylation signal derived from the human GAPDH gene.

88. The oncolytic virus of any one of embodiments 77-87, further comprising a polyadenylation signal positioned after the polynucleotide encoding the CD40 agonist.

89. The oncolytic virus of embodiment 88, wherein the polyadenylation signal positioned after the polynucleotide encoding the CD40 agonist is hBGpA.

90. The oncolytic virus of any one of embodiments 77-89, wherein the expression cassette comprises an AoHV1 promoter operably linked to the polynucleotide encoding the CD40 agonist.

91. The oncolytic virus of any one of embodiments 76-90, wherein the expression cassette comprises an MMLV promoter operably linked to the polynucleotide encoding the IL-12.

92. The oncolytic virus of any one of embodiments 76-91, wherein the expression cassette comprises a polyadenylation signal following the polynucleotide encoding IL-12.

93. The oncolytic virus of embodiment 92, wherein the polyadenylation signal following the polynucleotide encoding the IL-12 is a US9-10 pA.

94. The oncolytic virus of any one of embodiments 78-87, wherein the expression cassette comprises (i) the polynucleotide encoding the IL-12 protein, (ii) the nucleic acid encoding the CD40 agonist, (iii) and the polynucleotide encoding the CTLA-4 binding protein, wherein the expression cassette is in the orientation relative to the internal short repeat (IRS) region of the genome of IRS-(i)-(ii)-(iii).

95. The oncolytic virus of embodiment 94, wherein the expression cassette comprises (iv) a polynucleotide encoding a US10 protein, wherein the expression cassette is in the orientation relative to the IRS region of the genome of IRS-(i)-(ii)-(iii)-(iv).

96. The oncolytic virus of embodiment 95, wherein the cassette comprises (v) a polynucleotide encoding a US11 protein, wherein the expression is in the orientation relative to the IRS region of the genome of IRS-(i)-(ii)-(iii)-(iv)-(v).

97. The oncolytic virus of embodiment 96, wherein the cassette comprises (vi) nucleic acid comprising a variant US11 gene, wherein the expression cassette is in the orientation relative to the IRS region of the genome of IRS-(i)-(ii)-(iii)-(iv)-(v)-(vi).

98. The oncolytic virus of any one of embodiments 95-97, wherein the polynucleotide encoding the CD40 agonist is in the reverse orientation within the cassette relative to the polynucleotide encoding the IL-12 and the polynucleotide encoding the CTLA-4 binding protein.

99. The oncolytic virus of any one of embodiments 6-98, wherein the oncolytic virus exhibits increased T cell activation as assessed by an in vitro IL-2 secretion assay relative to an oncolytic virus lacking any one, any two, or all of the genes encoding the IL-12 protein, the CD40 agonist, and the CTLA-4 binding protein.

100. The oncolytic virus of any one of embodiments 1-100, wherein the virus is attenuated compared to a wild-type virus.

101. The oncolytic virus of any one of embodiments 42-99, wherein the oncolytic virus has increased abscopal efficacy relative to an oncolytic virus lacking any one, any two, or any three of the FLT3L, the IL-12, the CD40 agonist, and the CTLA-4 binding protein.

102. The oncolytic virus of any one of embodiments 1-100, wherein the virus is attenuated compared to a wild-type virus.

The oncolytic virus of any one of embodiments 1-101, wherein the virus is able to evade the human immune system.

103. A pharmaceutical composition comprising the oncolytic virus of any one of embodiments 1-102 and a pharmaceutically acceptable excipient.

104. An expression cassette comprising
a polynucleotide encoding a TAP inhibitor, and
a polynucleotide encoding a FLT3 ligand.

105. The expression cassette, of embodiment 104, comprising a polynucleotide encoding a self-cleaving peptide, wherein the polynucleotide encoding the self-cleaving peptide is located between the TAP inhibitor and the FLT3 ligand.

106. The expression cassette of embodiment 105, wherein the self-cleaving peptide is P2A.

107. The expression cassette of embodiment 106, wherein P2A comprises the amino acid sequence of SEQ ID NO:91.

108. The expression cassette of any one of embodiments 104-107, wherein the TAP inhibitor is derived from herpesvirus 1 or herpes virus 2.

109. The expression cassette of any one of embodiments 104-108, wherein the TAP inhibitor is derived from bovine herpes virus 1.

110. The expression cassette of any one of embodiments 104-107, wherein the TAP inhibitor is UL49.5, US6, or ICP47.

111. The expression cassette of embodiment 110, wherein the TAP inhibitor is UL49.5.

112. The expression cassette of embodiment 111, wherein the TAP inhibitor comprises the amino acid sequence set forth in SEQ ID NO:82.

113. The expression cassette of any one of embodiments 104-112, wherein the TAP inhibitor is expressed as an immediate-early gene.

114. The expression cassette of any one of embodiments 104-113, wherein FLT3L comprises the amino acid sequence set forth in SEQ ID NO:71.

115. The expression cassette of any one of embodiments 104-114, wherein the expression cassette comprises a CMV promoter that regulates expression of the polynucleotide encoding the FLT3L and the polynucleotide encoding the TAP inhibitor.

116. The expression cassette of any one of embodiments 104-115, wherein the expression cassette comprises a polyadenylation signal positioned after the polynucleotide encoding the FLT3L and the polynucleotide encoding the TAP inhibitor.

117. The expression cassette of embodiment 116, wherein the polyadenylation signal is a bovine growth hormone polyadenylation signal (BGHpA).

118. The expression cassette of embodiment 117, wherein the cassette comprises in order, from upstream to downstream, the CMV promoter, the polynucleotide encoding a FLT3L, the polynucleotide encoding the P2A peptide, the polynucleotide encoding the UL49.5 protein, and the BGHpA polyadenylation signal.

119. The expression cassette of embodiment 118, wherein the polynucleotide for the CMV promoter comprises the polynucleotide sequence set forth in SEQ ID NO: 107, the polynucleotide for hFLT3L encodes the amino acid sequence set forth in SEQ ID NO:71, the polynucleotide for P2A encodes the amino acid sequence set forth in SEQ ID NO:91, the polynucleotide for UL49.5 encodes the amino acid sequence set forth in SEQ ID NO: 82, and the polynucleotide for the BGHpA polyadenylation signal comprises the polynucleotide sequence set forth in SEQ ID NO: 102.

120. A modified HSV genome comprising the expression cassette of any one of embodiments 104-119.

121. The modified HSV genome of embodiment 120, wherein one or both γ34.5 loci of the modified HSV genome is replaced with the cassette.

122. An oncolytic virus comprising the expression cassette of any one of embodiments 104-119.

123. The oncolytic virus of embodiment 122, wherein one or both γ34.5 loci of the oncolytic virus are replaced with the expression cassette.

124. The oncolytic virus of embodiments 122 or 123, wherein both γ34.5 loci of the oncolytic virus are replaced with the expression cassette.

125. An expression cassette comprising:
a polynucleotide encoding an IL-12,
a polynucleotide encoding a CD40 agonist, and
a polynucleotide encoding a CTLA-4 binding protein.

126. The expression cassette of embodiment 125, wherein the IL-12 is a heterodimer comprising a p35 subunit and a p40 subunit.

127. The expression cassette of embodiment 126, wherein the p35 subunit and/or the p40 subunit are human.

128. The expression cassette of embodiments 126 or 127, wherein the p35 subunit comprises the sequence of amino acids of SEQ ID NO:1.

129. The expression cassette of any one of embodiments 126-128, wherein the p40 subunit comprises the sequence of amino acids of SEQ ID NO:2.

130. The expression cassette of embodiment 126, wherein the p35 subunit and/or the p40 subunit are murine.

131. The expression cassette of embodiment 130, wherein the p35 subunit comprises the sequence of amino acids of SEQ ID NO:5.

132. The expression cassette of embodiments 130 or 131, wherein the p40 subunit comprises the sequence of amino acids of SEQ ID NO:6.

133. The expression cassette of one of embodiments 126-132, wherein the p35 subunit and p40 subunit are linked by a peptide linker.

134. The expression cassette of embodiment 133, wherein the linker comprises an amino acid sequence comprising glycine and serine residues.

135. The expression cassette of embodiment 134, wherein the linker comprises the amino acid sequence set forth in SEQ ID NOs: 3 or 7.

136. The expression cassette of any one of embodiments 125-135, wherein the CD40 agonist is a CD40 ligand.

137. The expression cassette of any one of embodiments 125-136, wherein the CD40 agonist comprises a CD40 ligand ectodomain.

138. The expression cassette of embodiment 137, wherein the CD40 ligand is a trimer of three single-chain trimeric CD40 ligand ectodomains.

139. The expression cassette of embodiments 137 or 138, wherein the CD40 ligand ectodomain is human.

140. The expression cassette of embodiment 139, wherein the CD40 ligand ectodomain comprises the sequence of amino acids of SEQ ID NO:20.

141. The expression cassette of embodiments 137 or 138, wherein the CD40 ligand ectodomain is murine.

142. The expression cassette of embodiment 141, wherein the CD40 ligand ectodomain comprises the sequence of amino acids of SEQ ID NO:26.

143. The expression cassette of any one of embodiments 136-142, wherein the CD40 agonist comprises a trimerization motif operably linked to each of the three single-chain trimeric CD40 ligand ectodomains.

144. The expression cassette of embodiment 143, wherein the trimerization motif is a T4 fibritin trimerization motif.

145. The expression cassette of embodiments 143 or 144, wherein the trimerization motif is linked to each of the three single-chain trimeric ectodomains of CD40 agonist by a linker comprising glycine and serine residues.

146. The expression cassette of any one of embodiments 125-145, wherein the CTLA-4 binding protein is a CTLA-4 antibody or antigen binding fragment thereof.

147. The expression cassette of embodiment 146, wherein the CTLA-4 antibody or antigen binding fragment thereof is an scFv.

148. The expression cassette of embodiment 146 or 147, wherein the anti-CTLA-4 antibody or antigen binding fragment thereof specifically binds to human CTLA-4.

149. The expression cassette of any one of embodiments 146-148, wherein the anti-CTLA-4 antibody or antigen binding fragment is bivalent.

150. The expression cassette of any one of embodiments 147-149, wherein the anti-CTLA-4 scFv is fused to the N-terminus of a IgG1 constant domain.

151. The expression cassette of embodiment 150, wherein the human IgG1 is a variant human IgG1 comprising a C220S substitution, with numbering according to EU numbering.

152. The expression cassette of any one of embodiments 146-151, wherein the anti-CTLA-4 antibody or antigen binding fragment thereof comprises:
a CDRH1 comprising the sequence of amino acids of SEQ ID NO:40;
a CDRH2 comprising the sequence of amino acids of SEQ ID NO:41;
a CDRH3 comprising the sequence of amino acids of SEQ ID NO:42;
a CDRL1 comprising the sequence of amino acids of SEQ ID NO:43;
a CDRL2 comprising the sequence of amino acids of SEQ ID NO:44; and
a CDRL3 comprising the sequence of amino acids of SEQ ID NO:45.

153. The expression cassette of any one of embodiments 146-152, wherein the anti-CTLA-4 antibody or antigen binding fragment thereof comprises a variable heavy chain (VH) comprising the amino acid sequence set forth in SEQ ID NO:46 and a variable light chain (VL) comprising the amino acid sequence set forth in SEQ ID NO:47.

154. The expression cassette of any one of embodiments 146-153, wherein the anti-CTLA-4 antibody or antigen binding fragment thereof comprises the sequence of amino acids of SEQ ID NO:50.

155. The expression cassette of embodiment 146, wherein the CTLA-4 antibody is a camelid antibody comprising an anti-CTLA-4 VHH.

156. The expression cassette of embodiment 155, wherein the VHH is fused to the heavy chain of mouse IgG2a Fc.

157. The expression cassette of any one of embodiments 125-156, further comprising a polynucleotide encoding a US10 protein.

158. The expression cassette of any one of embodiments 125-157, wherein the expression cassette comprises a native late US11 gene.

159. The expression cassette of any one of embodiments 125-158, wherein the expression cassette comprises a variant polynucleotide encoding a US11 protein.

160. The expression cassette of any one of embodiments 125-159, wherein the expression cassette does not express US12 protein.

161. The expression cassette of any one of embodiments 125-160, wherein the expression cassette comprises a mCMV promoter positioned upstream from the polynucleotide encoding the CTLA-4 binding protein.

162. The expression cassette of any one of embodiments 125-161, wherein the expression cassette comprises a polyadenylation signal positioned after the polynucleotide encoding the CTLA-4 binding protein.

163. The expression cassette of embodiment 162, wherein the polyadenylation signal is a polyadenylation signal derived from the human GAPDH gene.

164. The expression cassette of any one of embodiments 125-163, further comprising a polyadenylation signal positioned after the polynucleotide encoding the CD40 agonist.

165. The expression cassette of embodiment 164, wherein the polyadenylation signal positioned after the polynucleotide encoding the CD40 agonist is hBGpA.

166. The expression cassette of any one of embodiments 125-165, wherein the expression cassette comprises an AoHV1 promoter operably linked to the polynucleotide encoding the CD40 agonist.

167. The expression cassette of any one of claims 125-166, wherein the expression cassette comprises an MMLV promoter operably linked to the polynucleotide encoding the IL-12.

168. The expression cassette of any one of embodiments 125-167, wherein the expression cassette comprises a polyadenylation signal following the polynucleotide encoding IL-12.

169. The expression cassette of embodiment 168, wherein the polyadenylation signal following the polynucleotide encoding the IL-12 is a US9-10 pA.

170. The expression cassette of any one of embodiments 157-169, wherein the expression cassette comprises a polyadenylation signal positioned after the polynucleotide encoding the US10 protein.

171. The expression cassette of embodiment 170, wherein the polyadenylation signal is a human growth hormone polyadenylation signal (hGHpA).

172. The expression cassette of any one of embodiments 160-171, wherein the expression cassette comprises, in order, from upstream to downstream, the polynucleotide comprising the variant US11 gene, the polynucleotide encoding the native late US11 protein, the polynucleotide encoding the US10 protein, the polynucleotide encoding the CTLA-4 binding protein, the polynucleotide encoding the CD40 agonist, and the polynucleotide encoding the IL-12.

173. The expression cassette of embodiment 168, wherein the expression cassette comprises, in order, from upstream to downstream, the polynucleotide comprising the variant US11 gene, the polynucleotide encoding the native late US11 protein, the polynucleotide encoding the US10 protein, a hGHpA polyadenylation sequence, a mCMV promoter, the polynucleotide encoding the CTLA-4 binding protein, a polyadenylation signal derived from the human GAPDH gene, a hBGpA polyadenylation sequence, the polynucleotide encoding the CD40 agonist, an AoHV1 promoter, an MMLV promoter, the polynucleotide encoding the IL-12, and a US9-10 pA polyadenylation sequence.

174. The expression cassette of embodiment 173, wherein the polynucleotide for the variant US11 gene comprises the polynucleotide sequence set forth in SEQ ID NO: 204, the polynucleotide encoding US11 protein encodes the amino acid sequence set forth in SEQ ID NO:80, the polynucleotide encoding US10 protein encodes the amino acid sequence set forth in SEQ ID NO:90, the polynucleotide for the mCMV promoter comprises the polynucleotide sequence set forth in SEQ ID NO: 107, the polynucleotide encoding the CTLA-4 binding protein encodes the amino acid sequence set forth in SEQ ID NO: 50, the polynucleotide for the polyadenylation signal derived from the human GAPDH gene comprises SEQ ID NO: 213, the polynucleotide for the hBGp polyadenylation signal comprises the polynucleotide sequence set forth in SEQ ID NO: 102, the polynucleotide for the CD40 agonist encodes the amino acid sequence set forth in SEQ ID NO: 25, the polynucleotide for the AoHV1 promoter comprises the polynucleotide sequence set forth in SEQ ID NO: 310, the polynucleotide for the MMLV promoter comprises the polynucleotide sequence set forth in SEQ ID NO: 311, the polynucleotide for the IL-12 encodes the amino acid sequence set forth in SEQ ID NO: 4, and the polynucleotide for the US9-10 pA polyadenylation sequence comprises SEQ ID NO: 314.

175. A modified HSV genome comprising the expression cassette of any one of embodiments 125-174.

176. The modified HSV genome of embodiment 175, wherein the expression cassette is integrated in the US10-12 locus of the modified HSV genome.

177. An oncolytic virus comprising the expression cassette of any one of embodiments 104-119 and the expression cassette of any one of embodiments 125-174.

178. The oncolytic virus of embodiment 177, wherein the virus is a herpes simplex virus (HSV).

179. The oncolytic virus of embodiment 178, wherein the virus is a HSV-1 or a HSV-2.

180. The oncolytic virus of any one of embodiments 177-179, wherein the virus is attenuated compared to a wild-type virus.

181. The oncolytic virus of any one of embodiments 177-180, wherein the virus is able to evade the human immune system.

182. A method of treating cancer in an individual comprising administering to the individual an effective amount of the oncolytic virus of any one of embodiments 1-102, 122-124, 177-181 or the pharmaceutical composition of embodiment 102 to the individual.

183. The method of embodiment 121, wherein cancer is regionally advanced cancer, metastatic cancer, or recurrent cancer.

184. The method of embodiments 182 or 183, wherein the cancer comprises a solid tumor.

185. A method of killing tumor cells in an individual comprising administering the oncolytic virus of any one of embodiments 1-102, 122-124, 177-181 or the pharmaceutical composition of embodiment 103 to the individual.

186. The method of embodiment 185, wherein the individual has tumor cells at first and second sites, wherein the oncolytic virus is administered at the first site, wherein the oncolytic virus causes an immune response at the first site that results in cell death of tumor cells at the second site.

187. The method of any one of embodiments 182-186, wherein an administration of the oncolytic virus to the individual results in an immune response in the individual.

188. The method of any one of embodiments 182-187, wherein the oncolytic virus causes tumor growth inhibition.

189. The method of any one of embodiments 182-188, wherein the oncolytic virus generates a sustained antitumor immune response.

190. The method of any one of embodiments 182-189, wherein the oncolytic virus preferentially lyses tumor cells.

191. The method of any one of embodiments 182-190, wherein the oncolytic virus enhances T cell effector functions and/or depletes Tregs in the tumor microenvironment.

192. The method of any one of embodiments 182-191, wherein the oncolytic virus recruits dendritic cells to the tumor microenvironment.

193. The method of any one of embodiments 182-192, wherein the oncolytic virus matures dendritic cells.

194. A method of producing an oncolytic comprising culturing a cell comprising the oncolytic virus of any one of embodiments 1-102, 122-124, 177-181, lysing the cell to produce a cell lysate, and purifying the oncolytic virus from the cell lysate.

195. A CD40 agonist protein comprising three single-chain trimeric CD40 ligand ectodomains, each fused to a trimerization motif, wherein the CD40 agonist protein is a trimer of trimers.

196. The CD40 agonist protein of embodiment 195, wherein the CD40 agonist is a CD40 ligand.

197. The CD40 agonist protein of embodiments 195 or 196, wherein the CD40 ligand ectodomain is human.

198. The CD40 agonist protein of any one of embodiments 195-197, wherein the CD40 ligand ectodomain comprises the sequence of amino acids of SEQ ID NO:20.

199. The CD40 agonist protein of any one of embodiments 195-198, wherein the CD40 ligand ectodomain is murine.

200. The CD40 agonist protein of embodiment 199, wherein the CD40 ligand ectodomain comprises the sequence of amino acids of SEQ ID NO:26.

201. The CD40 agonist protein of any one of embodiments 195-200, wherein the trimerization motif is a T4 fibritin trimerization motif.

202. The CD40 agonist protein of any one of embodiments 195-201, wherein the trimerization motif comprises the amino acid sequence set forth in SEQ ID NO:21.

203. The CD40 agonist protein of any one of embodiments 195-202, wherein the trimerization motif is linked to each of the three single-chain trimeric ectodomains of CD40 agonist by a peptide linker.

204. The CD40 agonist protein of embodiment 203, wherein the peptide liker comprises glycine and serine residues.

205. The CD40 agonist protein of embodiments 203 or 204, wherein the peptide linker comprises the amino acid sequence set forth in SEQ ID NOs: 23 or 27.

206. The CD40 agonist protein of any one of embodiments 195-205, wherein the CD40 agonist protein activates dendritic cells.

207. A polynucleotide encoding the CD40 agonist protein of any one of embodiments 195-206.

208. A vector comprising the polynucleotide of embodiment 207.

209. A host cell comprising the vector of embodiment 208.

210. A method of inhibiting tumor growth in an individual comprising administering an effective amount of the CD40 agonist protein of any one of embodiments 195-206, or a polynucleotide encoding the CD40 agonist protein of any one of embodiments 195-206 to the individual.

211. The method of any one of embodiments 182-193 and 210, wherein the individual is human.

212. An oncolytic virus corresponding to JP-OV-2.

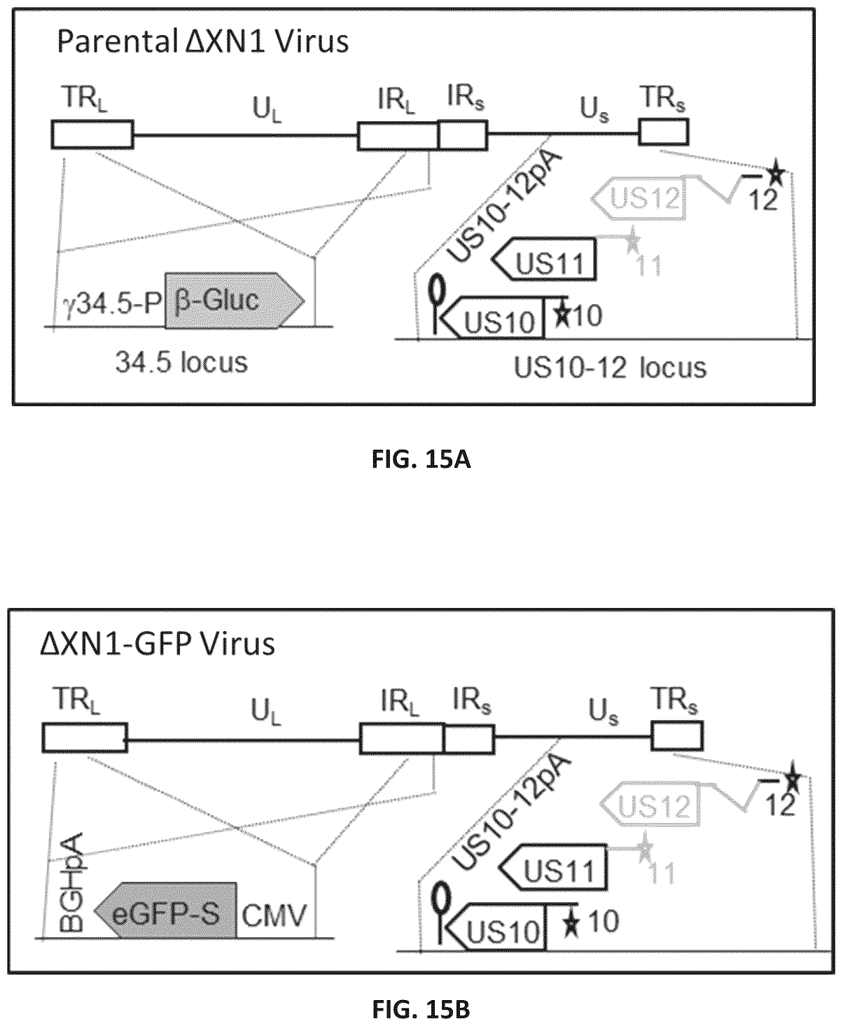

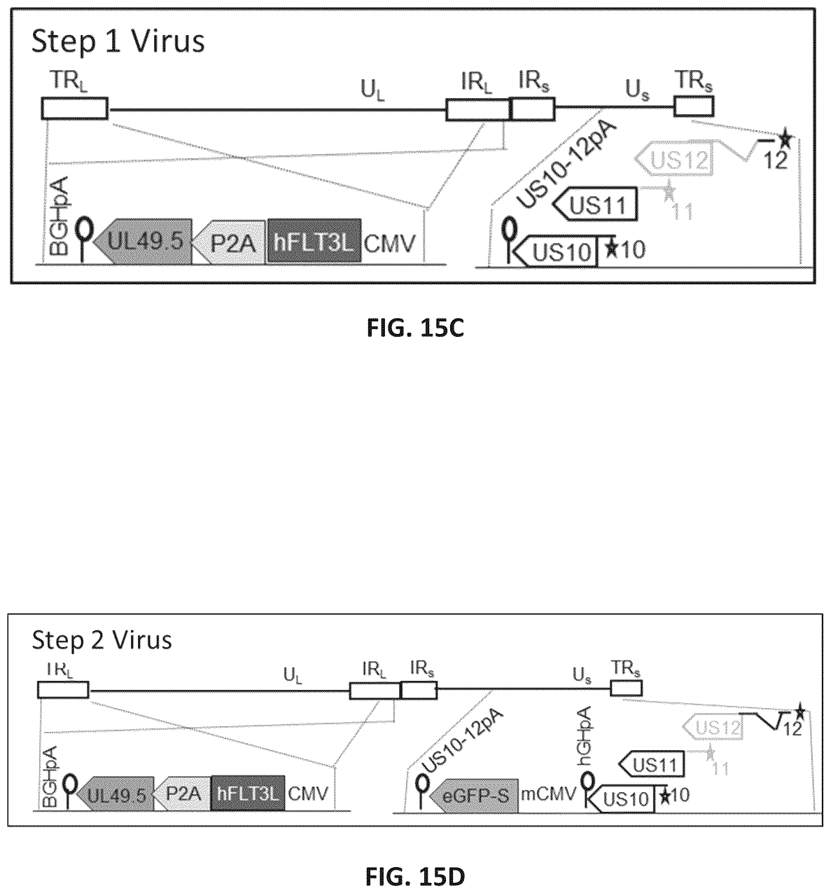

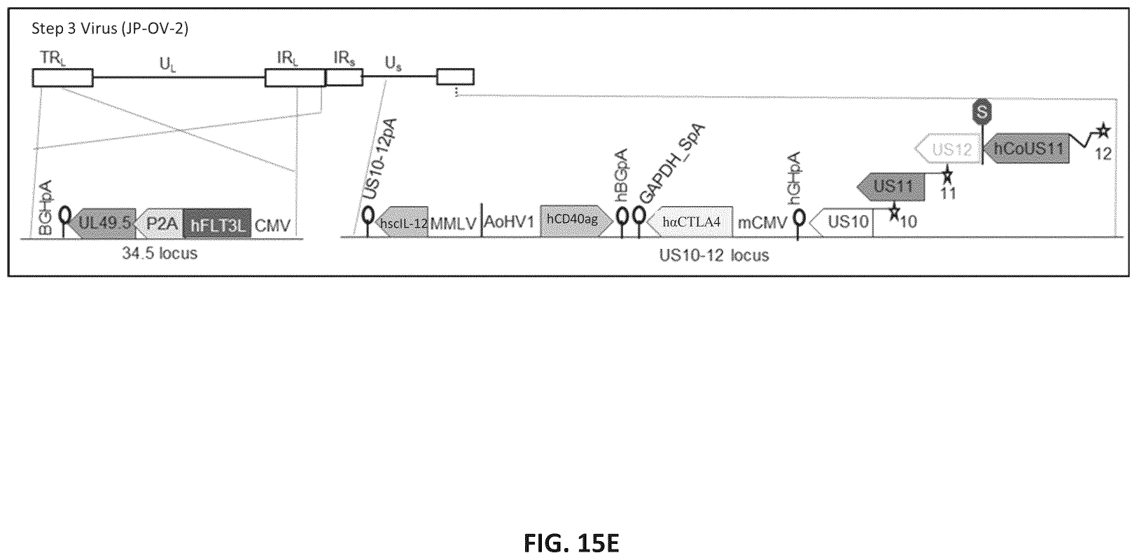

213. An oncolytic virus corresponding to the virus depicted in FIG. 15E (Step 3 Virus (JP-OV-2)).

### EXAMPLES

The presently disclosed subject matter will be better understood by reference to the following Examples, which are provided as exemplary of the invention, and not by way of limitation.

### Example 1: Engineering of an HSV-1 Oncolytic Virus with Innate and Adaptive Immune Stealth Functions

This Example describes the engineering of a herpes simplex virus Type 1 (HSV-1) oncolytic virus (OV) with stealth functions that enhance viral evasion of innate and adaptive anti-viral host responses.

### Engineering of Enhanced Innate Immune Stealth Functions

Previously developed HSV-1 OVs have employed several strategies to achieve partial attenuation of the virus while preserving sufficient viral replication in tumor cells. For example, Talimogene laherparepvec (a previously-reported genetically engineered oncolytic herpesvirus) is an HSV-1 OV that has an inactive γ34.5 gene (Δγ34.5), which encodes a neurovirulence factor, and has immediate-early (IE) expression of the US11 gene to improve viral replication. Deletion of the γ34.5 gene results in a highly attenuated virus that, while safe, also replicates poorly. IE-US11 expression is achieved by deleting the US12 gene, which causes the US11 gene to be driven by the US12 promoter, resulting in IE-US11 expression instead of the late (L) expression normally seen with the endogenous US11 promoter. IE-US11 expression has been shown to partially compensate for deletion of the γ34.5 gene, resulting in a virus that replicates better than the highly attenuated Δγ34.5 single mutants without reestablishing neurovirulence (Cassady et al. The herpes simplex virus US11 protein effectively compensates for the gammal(34.5) gene if present before activation of protein kinase R by precluding its phosphorylation and that of the alpha subunit of eukaryotic translation initiation factor 2. J Virol. 1998; 72(11):8620-8626; Mulvey et al. Regulation of eIF2alpha phosphorylation by different functions that act during discrete phases in the herpes simplex virus type 1 life cycle. J Virol. 2003; 77(20): 10917-10928; Mohr et al. A herpes simplex virus type 1 gamma34.5 second-site suppressor mutant that exhibits enhanced growth in cultured glioblastoma cells is severely attenuated in animals. J Virol. 2001; 75(11):5189-5196; and Taneja et al. Enhanced anti-tumor efficacy of a herpes simplex virus mutant isolated by genetic selection in cancer cells. Proc Natl Acad Sci USA. 2001; 98(15):8804-8808). However, IE-US11 expression by deletion of US12 eliminates L-US11 expression from its native promoter, resulting in decreased levels of US11 compared to a wild type (WT) virus at later timepoints in infection, which could render the virus vulnerable to inhibition by innate anti-viral responses as the infection cycle progresses.

To avoid the innate anti-viral response throughout the entire infection cycle, an HSV-1 OV was engineered to include two copies of the US11 gene. The first copy of the US11 gene was a codon-optimized IE-US11 under the control of the US12 promoter, which, as discussed above, partially compensates for deletion of the γ34.5 gene to enable efficient viral replication in tumor cells. The second copy of the US11 gene was an endogenous L-US11 gene under the endogenous US11 promoter, which was intended to protect the virus from protein kinase R (PKR)-mediated shutdown of translation throughout the entire temporal viral gene expression cycle.

### Engineering of Enhanced Adaptive Immune Stealth Functions

As discussed above, previously developed HSV-1 OVs, such as a previously-reported genetically engineered oncolytic herpesvirus, include a deletion of the US12 gene to drive IE-US11 expression from the US12 promoter. US12 encodes ICP47, which is a transporter associated with antigen processing (TAP) inhibitor that normally prevents the display of viral antigens on the cell surface. Removal of ICP47 may increase the presentation of viral antigens, leading to rapid clearance of virus-infected cells by anti-viral T cells, and limiting the effectiveness of the OV. Thus, deletion of US12 may affect viral replication and susceptibility to the host adaptive immune system in vivo (Pourchet A, Fuhrmann S R, Pilones K A, et al. CD8(+) T-cell immune evasion enables oncolytic virus immunotherapy. EBioMedicine. 2016; 5:59-67).

To enhance adaptive immune evasion, an HSV-1 OV was engineered to express UL49.5, a TAP inhibitor from bovine herpesvirus 1. Restoring TAP inhibitor activity to the virus may prevent premature clearance of infected cells, enabling virus persistence through multiple rounds of virus replication, leading to a potentially greater anti-cancer effect.

### Results

Engineered Innate Stealth Function Resulted in Sustained High Levels of US11 Expression and Lower Phosphorylated eIF2α

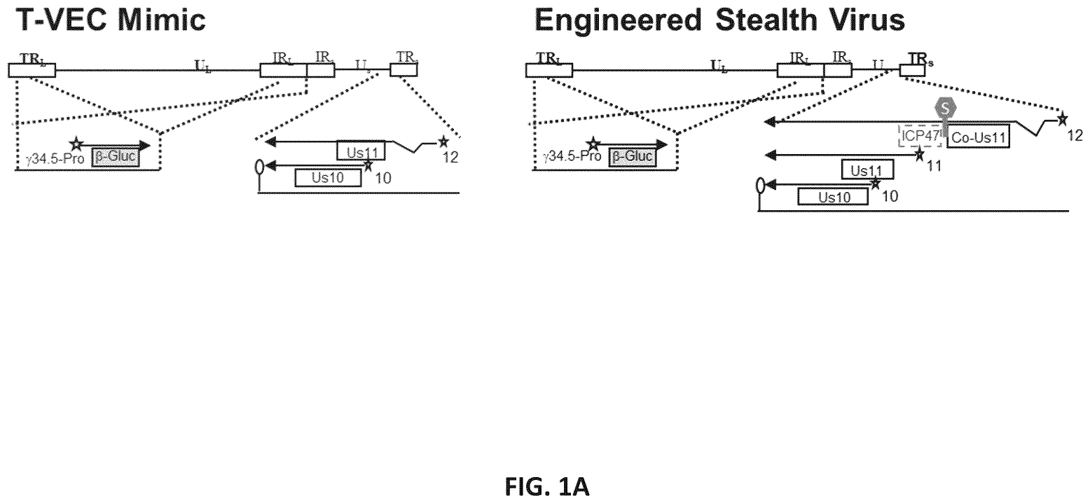

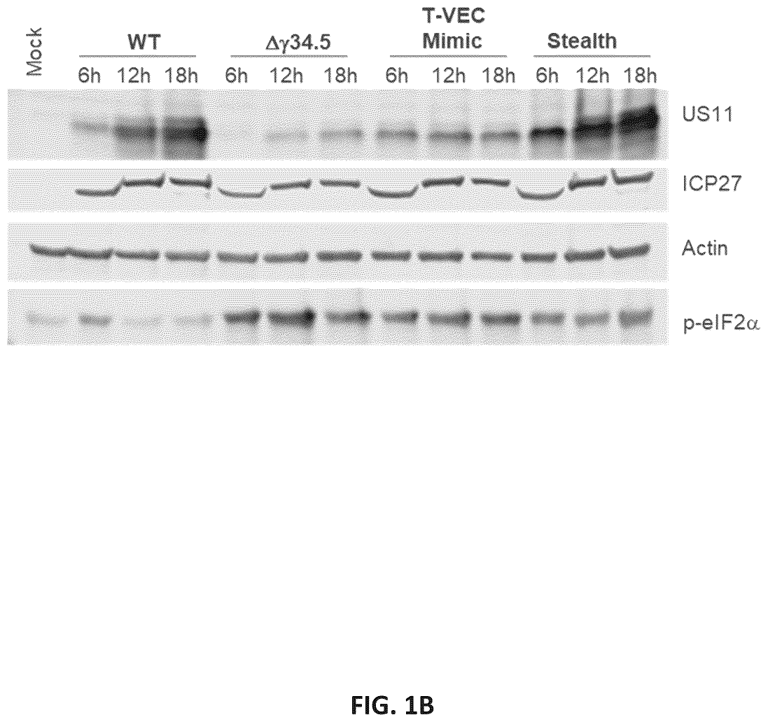

US11 and phosphorylated (p)-eIF2α levels were assessed by Western blot in A549 human lung cancer cells infected (MOI=5) with WT HSV-1, a Δγ34.5 HSV-1 virus, an engineered Stealth virus expressing both IE-US11 and L-US11 (as described above), and a virus mimicking a previously-reported genetically engineered oncolytic herpesvirus (FIG. 1A; hereinafter “mimic virus”). The previously-reported oncolytic herpesvirus was engineered to: (i) delete both copies of the ICP34.5 neurovirulence factor gene, (ii) delete the ICP47 gene, (iii) translocate the US11 gene to control of an early promoter, and (iv) comprise two copies of the GM-CSF gene. The mimic virus has a genetic architecture that is similar to the previously-reported oncolytic herpesvirus except that it does not comprise a GM-CSF gene. p-eIF2α is part of the host PKR pathway, and prevents translation initiation to block the production of viral proteins.

As shown in FIG. 1B, WT HSV-1 produced high levels of US11, especially at later infection timepoints (i.e., 12 and 18 hours post infection), which prevented the accumulation of p-eIF2α at those late times post-infection. On the other hand, highly attenuated Δγ34.5 HSV-1 showed high levels of p-eIF2α accumulation and consequently low levels of US11, since expression of late proteins is blocked by the inability of this virus to counteract the PKR response. A previously-reported genetically engineered oncolytic herpesvirus mimic virus, which only expresses IE-US11, showed moderate, steady levels of US11 accumulation, as well as moderate levels of p-eIF2α. In contrast, the engineered Stealth virus expressing both IE- and L-US11 showed the highest levels of US11 accumulation, slightly superior to the WT virus, and consequently demonstrated lower levels of p-eIF2α, however to a higher extent than WT virus. p-eIF2α was not completely blocked by the engineered Stealth virus. Thus, the Stealth virus is partially attenuated and would be restricted to tumor cells, which are typically impaired in anti-viral signaling, and therefore susceptible to the virus replication, while normal cells with a fully intact response are resistant.

The results described above demonstrated that the engineered Stealth virus expressing both IE- and L-US11 resulted in sustained high levels of US11 expression and lower p-eIF2α levels to enable enhanced translation of virally-expressed proteins.

### Engineered Adaptive Stealth Function Resulted in Reduced Display of MHC-Viral Peptides

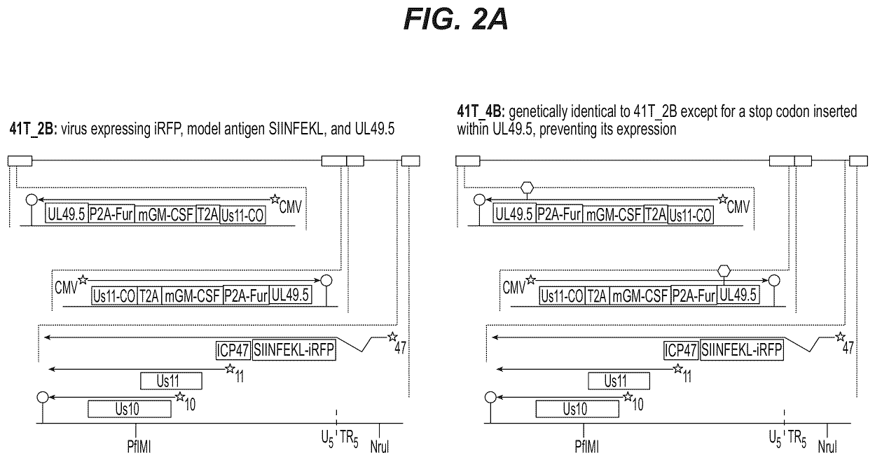

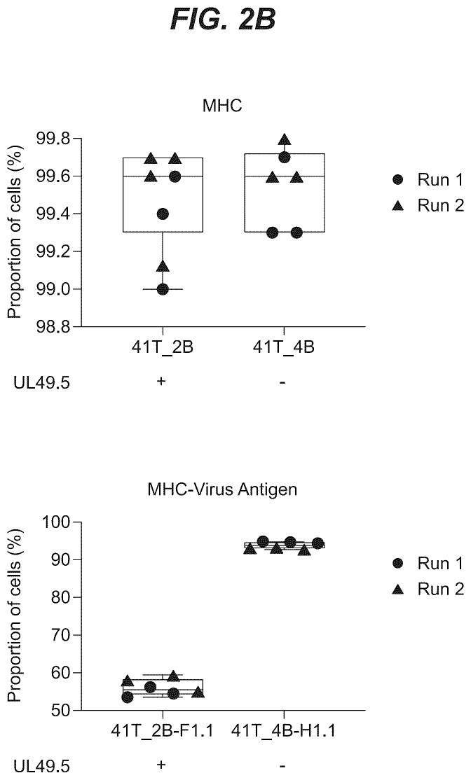

The display of major histocompatibility complex (MHC)-viral antigens on the surface of cells was assessed in MB49 cells infected (MOI=10) with an HSV-1 virus expressing a model antigen (SIINFEKL (SEQ ID NO: 319)) and UL49.5, or an equivalent virus lacking expression of UL49.5 (FIG. 2A).

As shown in FIG. 2B, TAP inhibition by expression of UL49.5 decreased the surface display of the model antigen (SIINFEKL (SEQ ID NO: 319)) in infected cells without changing cell surface levels of MHC. The odds ratio was 1.098 for MHC expression and 11.803 for MHC-SIINFEKL (“SIINFEKL” disclosed as SEQ ID NO: 319) presentation.

The observed reduction of MHC-viral peptides in cells infected with virus expressing UL49.5 suggested that TAP inhibition may reduce detection of infected cells by cytotoxic T lymphocytes (CTLs), which would otherwise lead to rapid clearance of infected cells, thereby enabling the virus to produce its desired long-term effects.

In sum, the results described in this Example showed that expression of both IE- and L-US11 and TAP inhibition through expression of UL49.5 resulted in innate and adaptive immune Stealth functions, respectively, that would enable an HSV-1 OV to have enhanced translation of virally-expressed proteins and to escape immune surveillance.

### Example 2: Selection and Engineering of Immunomodulatory Payload Proteins for Expression in an HSV-1 Oncolytic Virus

This Example describes the selection and engineering of immunomodulatory payload proteins that synergize with HSV-1 OV-induced cell death to generate potent anti-tumor immune responses.

### Payload Selection and Engineering

### CTLA-4 Antagonist

In order to select the best payload molecule to antagonize the CTLA-4 immune checkpoint pathway, a number of designs were tested in a reporter assay measuring the ability of a given protein construct to block the interaction of CTLA-4 and CD80:CD86. Reporter luminescence (RLU) was detected upon increasing concentrations of test anti-CTLA-4 molecules, with increasing RLU indicating the ability to block the interaction of CTLA-4 and CD80:CD86.

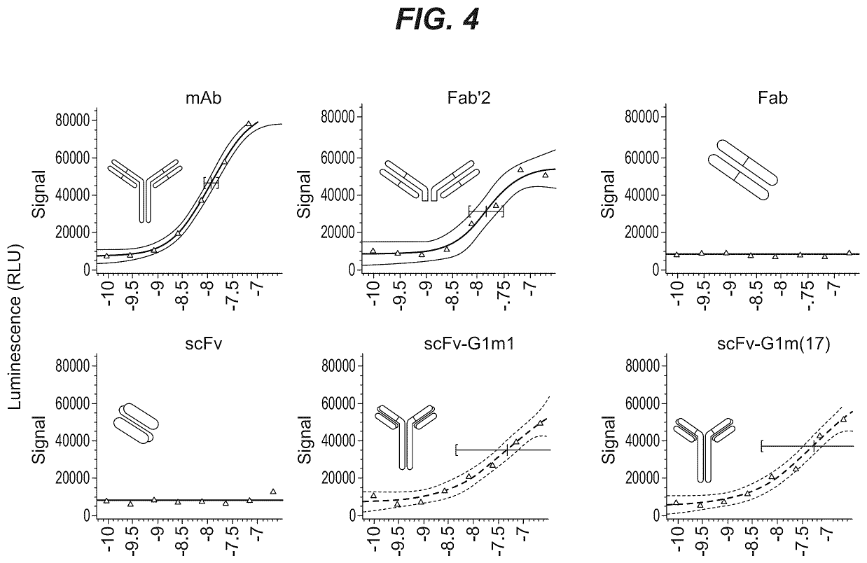

As shown in FIG. 4, it was observed that molecules with bivalent CTLA-4 binding (i.e., the anti-CTLA-4 mAb, Fab′2, scFv-G1m1, and scFv-G1m(17)) were able to block the interaction of CTLA-4 and CD80:CD86, whereas monovalent molecules (i.e., anti-CTLA-4 Fab and scFv) showed no blocking ability. These results demonstrated that, unexpectedly, the CTLA-4 antagonist payload should have bivalent CTLA-4 binding activity.

Next, the optimal strategy for providing a bivalent anti-CTLA-4 antagonist in the context of the three other payloads described above was determined. To induce strong CD40 agonist activity on APCs, oligomerization of the CD40 receptor (e.g., induced by a CD40 agonist payload) is crucial to sustain maximal nuclear factor kappa-light-chain-enhancer of activated B cells (NFκB) signaling (Vom Berg et al. Intratumoral IL-12 combined with CTLA-4 blockade elicits T cell-mediated glioma rejection. J Exp Med. 2013; 210(13):2803-2811). One possibility to promote oligomerization of the CD40 receptor was to use a CD40 agonist with an Fc region to promote dimerization. However, only one payload could have an Fc region to avoid the risk of different payloads (e.g., an anti-CTLA-4 antagonist and a CD40 agonist) heterodimerizing through the Fc region. Therefore, the requirement for an Fc region in bivalent CTLA-4-binding molecules was tested in MC38-5AG tumors in human CTLA-4 knock-in mice by intratumoral (IT) injection to mimic expression from an OV. Briefly, human CTLA-4 knock-in mice were implanted with 5×10⁵ MC38-5AG cells and randomized when tumors were about 100 mm³. 20 μg of negative control isotype antibody, positive control anti-CTLA mAb, Fc-containing scFv anti-CTLA4-Fc, or molar equivalent of Fc-less Ipi-Fab tandem-scFv were injected IT biweekly for a total of 4 treatments.

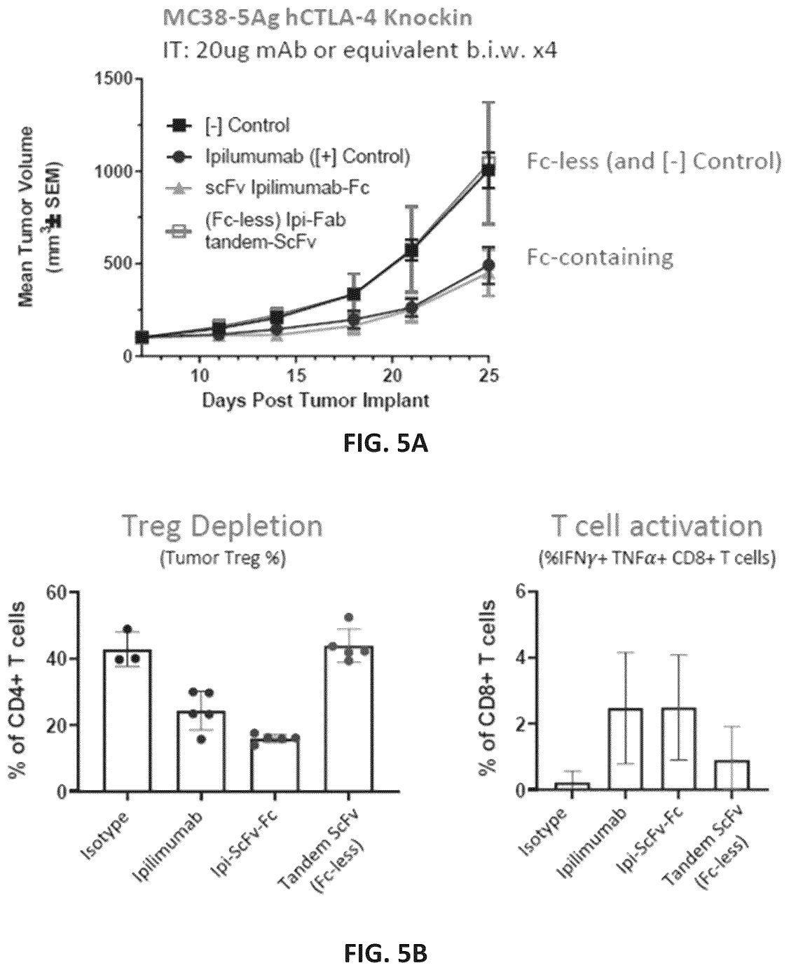

As shown in FIG. 5A, compared to negative control isotype antibody treatments, the Fc-containing anti-CTLA-4 molecules (i.e., positive control anti-CTLA4 monoclonal antibody and scFv anti-CTLA4-Fc) displayed anti-tumor activity, while the bivalent Fc-less construct (i.e., Ipi-Fab tandem-scFv) did not exhibit anti-tumor activity. In addition, as shown in FIG. 5B, an analysis of tumors obtained from the treated mice showed that the Fc-containing anti-CTLA-4 molecules were able to deplete Tregs and to expand tumor-specific CD8 T-cell effector function in the TME as compared to isotype control treatment, whereas the bivalent Fc-less construct did not. These results showed that, unexpectedly, the CTLA-4 antagonist payload should have an active Fc region, in addition to being bivalent, as discussed above.

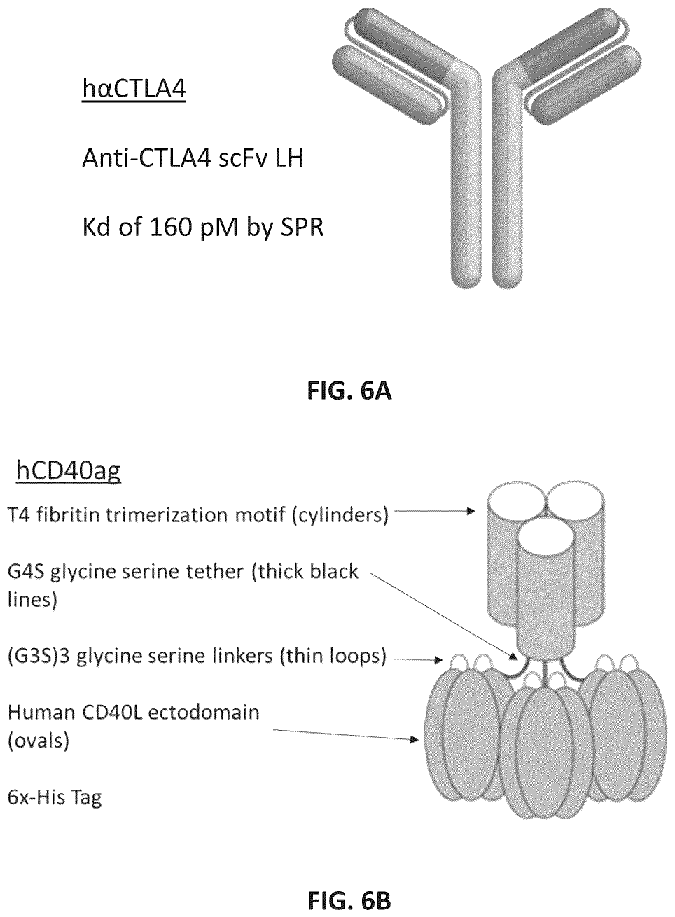

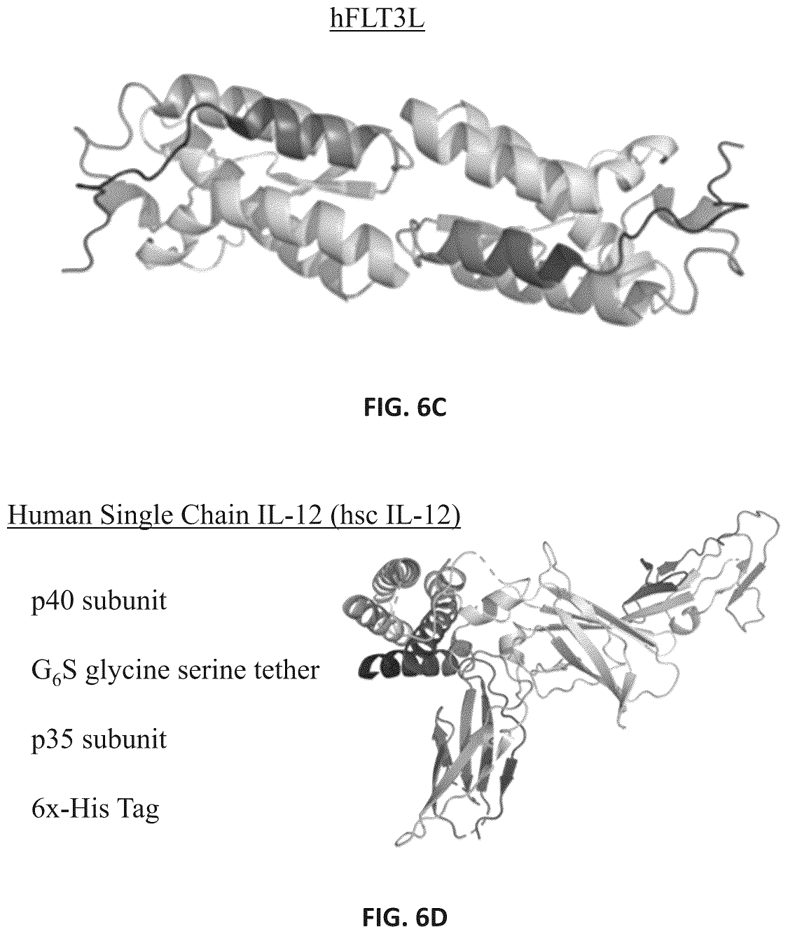

Based on the results described above, an anti-CTLA-4 antagonist payload protein, termed hαCTLA-4, was designed as an anti-CTLA-4 single-chain variable fragment (scFv) fused to the N-terminus of the heavy chain of human IgG1_G1m(17) (FIG. 6A). hαCTLA-4 includes a mutation of C220 to serine in the hinge of the Fc constant region to abolish disulfide formation between CH1 and CL (Frangione et al. Structural studies of immunoglobulin G. Nature. 1969; 221(5176):145-148; and Tam et al. Antibodies (Basel). 2017; 6(3):12). hαCTLA-4 has a dissociation constant K_{d} for CTLA-4 of 160 pM, assessed by surface plasmon resonance (SPR).

### CD40 Agonist

As discussed above, it was discovered that the anti-CTLA-4 antagonist payload required a functional Fc region. Therefore, CD40 agonist constructs that would not heterodimerize with the anti-CTLA-4 antagonist were engineered and evaluated.

Two alternative CD40 agonist constructs were designed: hCD40ag, which is an Fc-less trimer of CD40L trimer bundles, and hCD40ag2, which includes bivalent CD40L trimer bundles connected to an Fc region designed to disfavor heterodimerization with huIgG1.

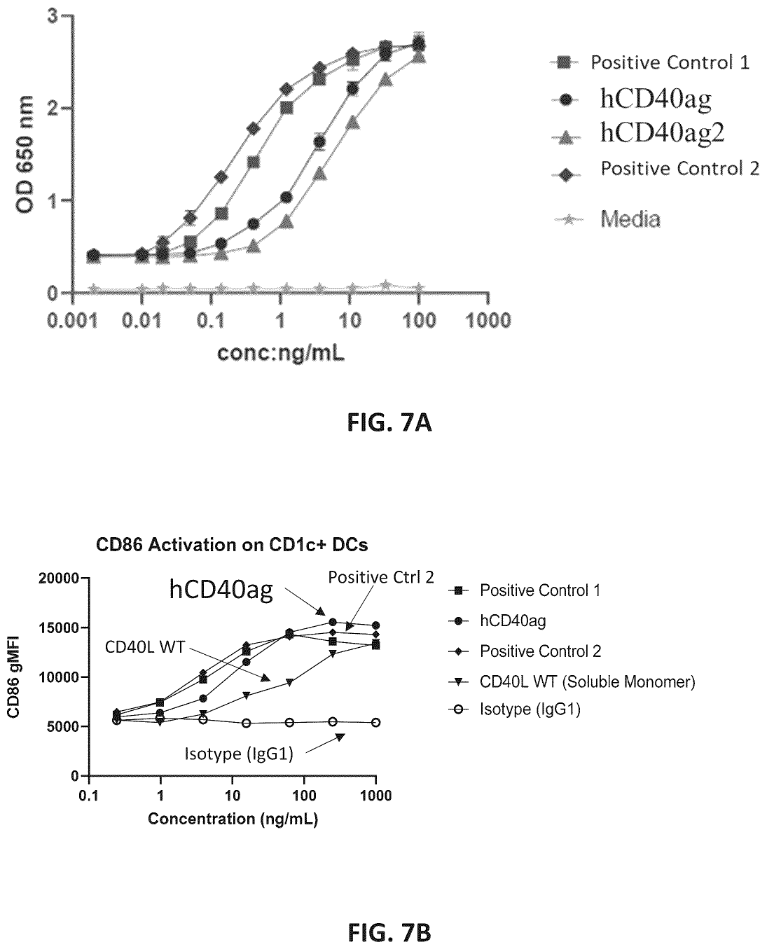

hCD40ag and hCD40ag2 were first tested for their ability to induce CD40 signaling using reporter cells that emit signal upon activation of the CD40 pathway. Briefly, CD40 reporter cells were incubated with increasing concentrations of both Fc-containing and alternative Fc constructs. As shown in FIG. 7A, both hCD40ag and hCD40ag2 had CD40 agonist activity, as did two positive controls.

hCD40ag and hCD40ag2 were further tested for their ability to activate DCs, assessed by levels of the CD86 activation marker. Briefly, Primary DCs were incubated overnight with increasing concentrations of Fc-containing and alternative Fc constructs. Following incubation, cells were evaluated for increased CD86 DC activation marker expression by flow cytometry. As shown in FIG. 7B, the Fc-less hCD40ag CD40 agonist activated DCs to express higher levels of the CD86 activation marker in a dose-dependent manner.

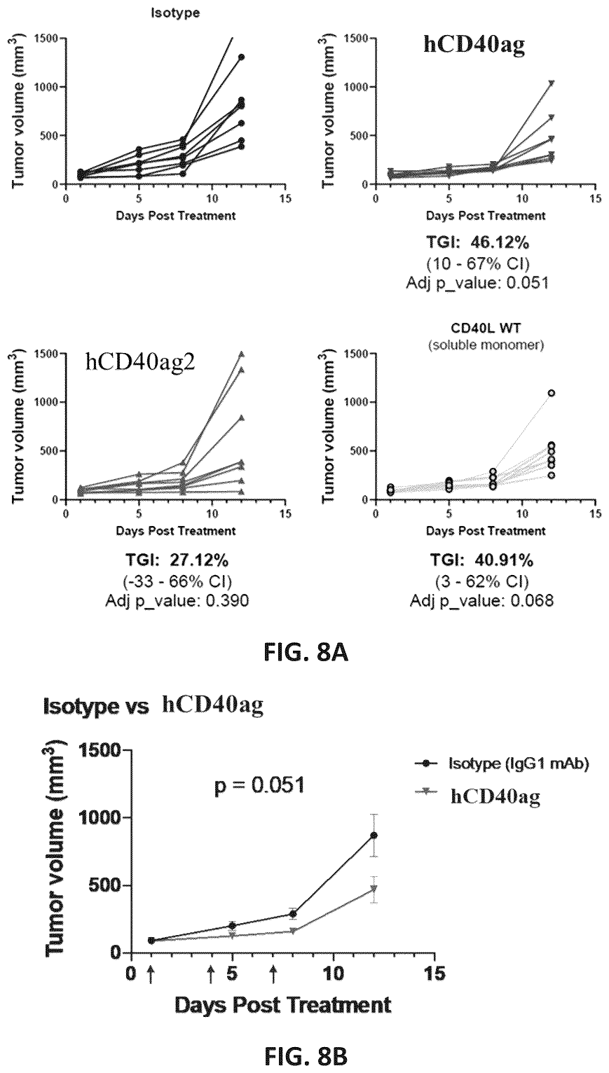

Next, the CD40 agonist constructs were tested for anti-tumor activity in vivo using MC38-5AG tumors in human CD40 knock-in mice. Briefly, human CD40 knock-in mice were implanted with 5×105 MC38-5AG cells and randomized when tumors were ˜100 mm³. 20 μg of negative control isotype antibody, hCD40ag, hCD40ag2, or molar equivalent of CD40 agonist (CD40L) WT monomer were injected intratumorally (IT) every 4 days for 3 treatments. Tumor growth inhibition and statistical analyses were performed by InVivoLDA Version 4.8. As shown in FIGS. 8A-8B, compared to isotype control, hCD40ag, hCD40ag2, and WT CD40 agonist (CD40L) monomer displayed tumor growth inhibition (TGI) of 46.12%, 27.12%, and 40.91%, respectively. Strong antitumor activity was observed during the treatment period with the Fc-less construct hCD40ag, and statistical significance reached a p-value of 0.051. The combined in vitro and in vivo activity led to the selection of hCD40ag as the CD40 agonist payload.

As shown in FIG. 6B, hCD40ag, is a trimerized, C-terminal fusion protein containing trimeric bundles of human CD40L ectodomains (Uniprot: P29965) that bind to the CD40 receptor protein on APCs to promote anti-cancer activity. hCD40ag has the 27 amino acid trimerization motif from the fibritin protein of T4 phage (Uniprot: P10104) fused with a glycine/serine linker to the N-terminal CD40L ectodomain trimer bundles. The CD40L ectodomain trimer bundles consist of individual CD40L protomers fused together by glycine/serine linkers from the C-terminus of one protomer to the N-terminus of the subsequent protomer to form single polypeptide trimeric bundles. hCD40ag was designed to promote stable and predictable oligomerization of CD40L without the need for an Fc region.

### FLT3L

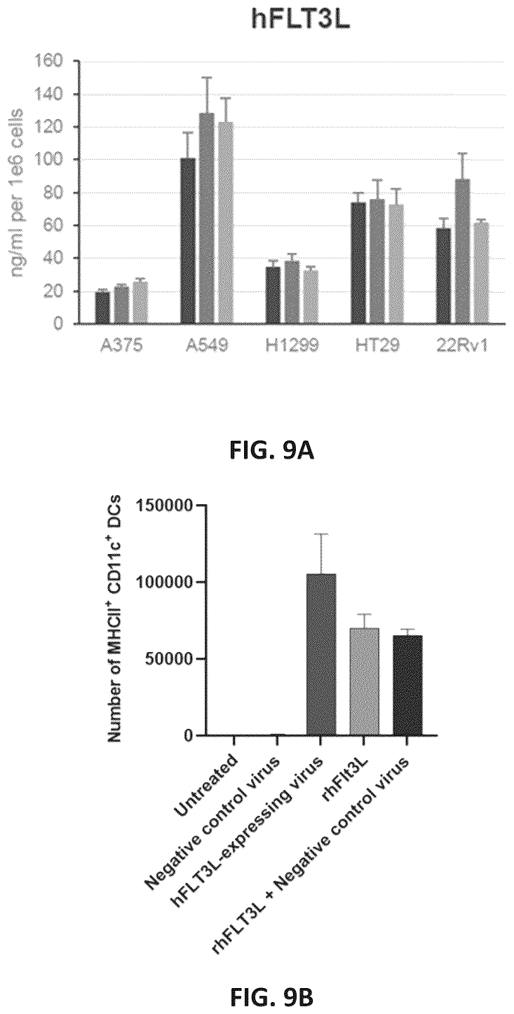

Full-length FLT3L is membrane-bound and undergoes proteolytic cleavage to become a soluble growth factor (Horiuchi et al. Ectodomain shedding of FLT3 ligand is mediated by TNF-alpha converting enzyme. J Immunol. 2009; 182(12): 7408-14). Therefore, it was first determined whether virally-expressed FLT3L is processed into soluble FLT3L. Briefly, several human tumor cell lines (A375, A549, H1299, HT29, and 22Rv1) were infected with three viruses expressing human FLT3L (hFLT3L). As shown in FIG. 9A, soluble hFLT3L was detected in the supernatant in every case, showing that cleavage and shedding of virally-expressed hFLT3L occurs.

Next, full-length WT hFLT3L protein was tested for bioactivity using a DC differentiation assay. Briefly, differentiation of murine DCs (MHCII⁺CD11c⁺) from bone marrow was assessed by flow cytometry upon treatment with recombinant human FLT3L (rhFLT3L), or the supernatant of Vero cells infected (MOI=1) with either an HSV-1 virus expressing hFLT3L or a negative control virus with similar architecture, but which did not express hFLT3L. The amount of hFLT3L in the supernatant was determined by enzyme-linked immunosorbent assay (ELISA). The activity of rhFLT3L was assessed alone or spiked in the supernatant from cells infected with the negative control virus. As shown in FIG. 9B, bone marrow cells differentiated into MHCII⁺CD11c⁺ DCs after exposure to supernatant from cells infected with virus encoding hFLT3L, but not with virus not expressing hFLT3L. This differentiation was similar to, but slightly higher than what was observed with an equal amount of recombinant hFLT3L. This experiment also showed that virally-expressed hFLT3L was cross-reactive with the mouse FLT3 receptor. See O'Keeffe, Blood. 2002; 99(6):2122-2130.

Based on the above results, the full-length human WT FLT3L (hFLT3L) protein (Uniprot: P49771; FIG. 6C) was selected as the FLT3L payload.

### IL-12

A human single chain IL-12 (hscIL-12) construct was engineered to promote stable and predictable heterodimerization of the p40 and p35 subunits of IL-12 to generate functionally active IL-12. As shown in FIG. 6D, the hscIL-12 construct includes the human p40 subunit, also known as IL-12 subunit beta from IL-12, fused with a linker to the N-terminus of human p35, also known as IL-12 subunit alpha.

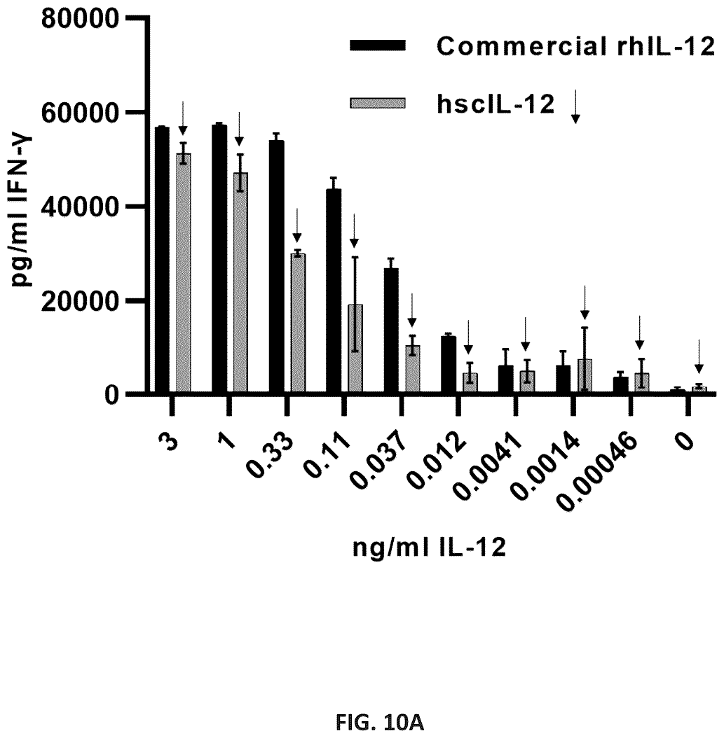

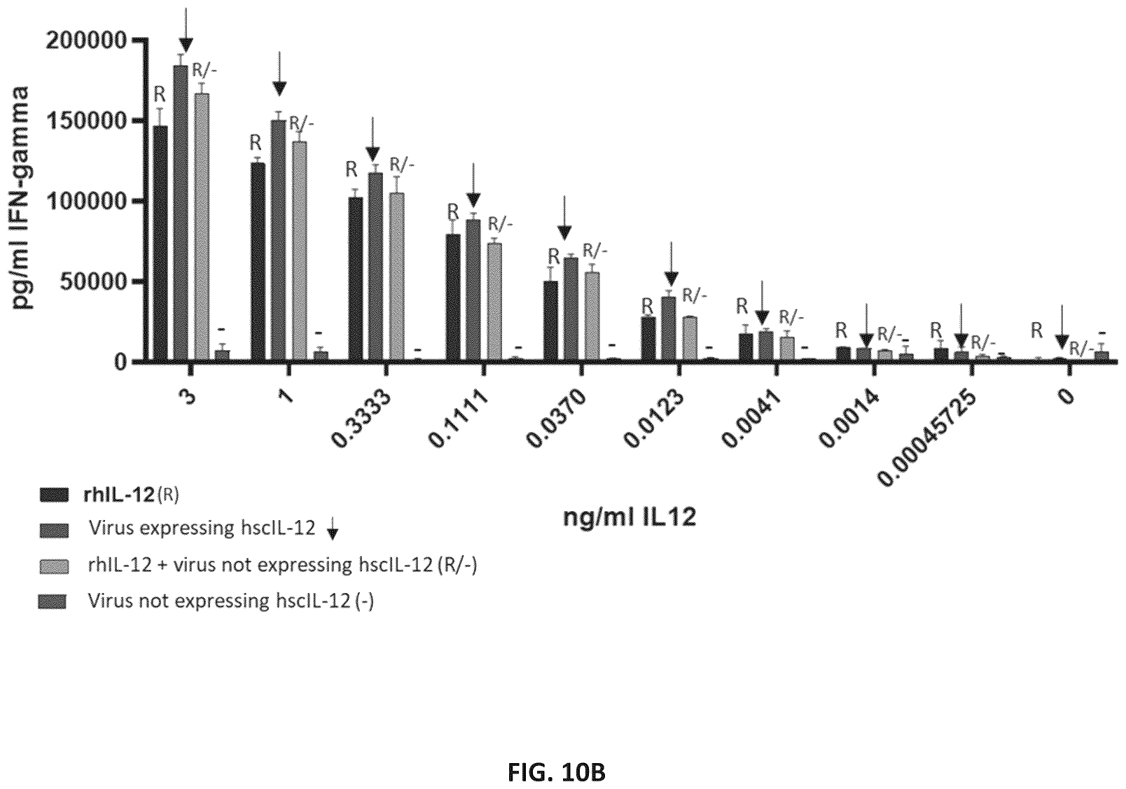

The hscIL-12 construct was tested for bioactivity based on its ability to induce IFNγ production by human peripheral blood mononuclear cells (PBMCs). Briefly, human PBMCs were treated for 7 days with serial dilutions of recombinant hIL-12 (2 subunits) or hscIL-12. Levels of IFNγ were measured by ELISA. As shown in FIG. 10A, human PBMCs produced IFNγ after treatment with purified hscIL-12. The hscIL-12 appeared to be slightly less bioactive than recombinant WT IL-12 (rhIL-12). However, the purified hscIL-12 used in this assay included a polyhistidine tag added for easier purification, which could be the cause of the decreased activity, and which would not be included for expression in an HSV-1 OV. This was supported by data for the bioactivity of virally-encoded hscIL-12, which is untagged, and which was shown to have bioactivity (assessed by induction of IFNγ production by PBMCs) that was identical to the recombinant molecule (FIG. 10B).

### Synergy Between Payloads

To assess whether the selected payloads synergized to produce better effector T-cell responses than each individual payload alone, a T-cell activation assay was performed in the presence of the anti-CTLA-4 antagonist payload (hαCTLA-4), the CD40 agonist payload (hCD40ag), and the IL-12 payload (hscIL-12) as single agents, in double combinations, or in triple combinations. Briefly, human PBMCs (2×105 cells) were incubated for 5 days in the presence of 100 ng/mL SEB superantigen and the payloads as single agents, or as double or triple combinations. Supernatants were harvested on day 5, and IL2 secretion (a measure of T cell activation) in the supernatant was quantified by Meso-Scale Discovery (MSD).

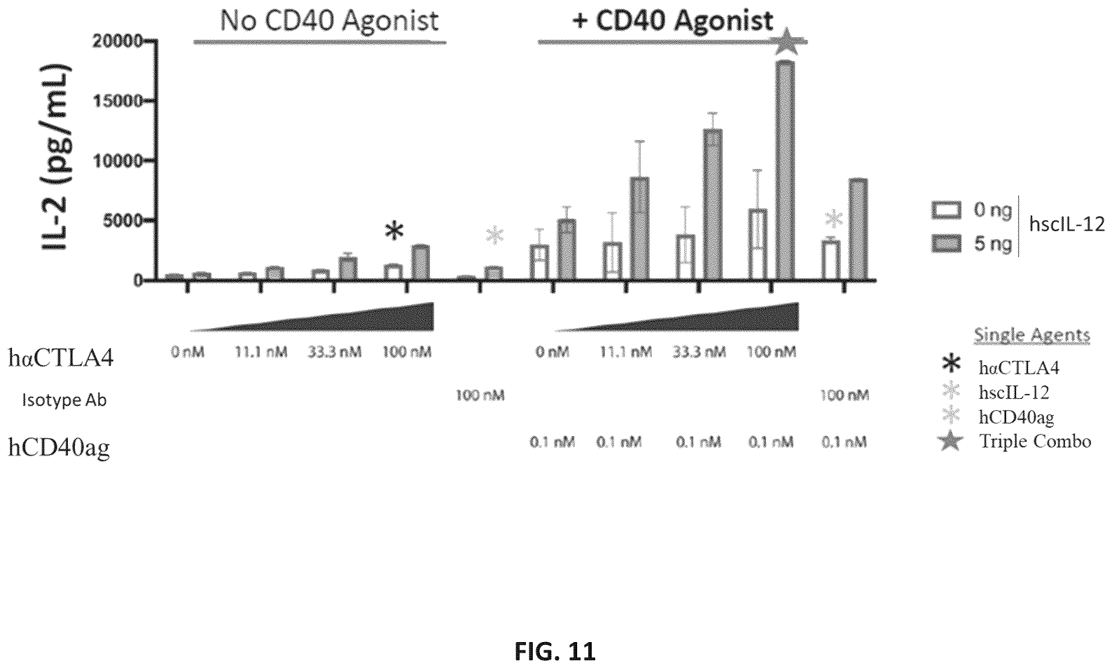

As shown in FIG. 11, the cells were incubated with a fixed concentration of T-cell-activating super antigen in the presence of increasing concentrations of the anti-CTLA-4 antagonist payload (hαCTLA-4) alone, or the double combination of hαCTLA-4 and the IL-12 payload (hscIL-12) (FIG. 11, left). In addition, the cells were exposed to a fixed concentration of the CD40 agonist payload (hCD40ag), combined with increasing concentrations of hαCTLA-4, or hscIL-12 and increasing concentrations of hαCTLA-4 (FIG. 11, right). The highest levels of T-cell activation were observed in the triple combination of hCD40ag, hscIL-12, and the dose-dependent increase in hαCTLA-4, showing that the combination of the hCD40ag, hscIL-12 and hαCTLA-4 payloads has a synergistic effect on T-cell activation.

### Example 3: Immunomodulatory Payload Expression Cassette Engineering

This Example describes the engineering, testing and selection of an HSV-1 OV expression cassette for the expression of the anti-CTLA-4 antagonist (hαCTLA-4), CD40 agonist (hCD40ag), and IL-12 (hscIL-12) payloads selected as described in Example 2 herein.

The cassette was designed to be as compact as possible, while also having strong output. A compact cassette could be obtained by designing a polycistronic expression cassette using a combination of 2A self-cleaving peptides or internal ribosome entry sites (IRES) capable of driving the expression of all 3 payloads from a single transcriptional unit. However, 2A self-cleaving peptides are not usually 100% cleaved, and the 2A peptide is retained on the protein located upstream of the polypeptide unless an extra protease site is engineered upstream of the 2A peptide. Moreover, all the payloads are extracellularly excreted, raising the possibility that the cotranslational processing of the 2A peptides could interfere with the translocation of the secreted polypeptide chains to the endoplasmic reticulum lumen. In addition, while IRES elements enable the independent translation of multiple polypeptides from a single mRNA, they are not as compact as 2A peptides (hundreds of nucleotides versus ˜60 nucleotides), and are usually not as efficient for translation as a bona fide mRNA 5′ untranslated region (5′ UTR), leading to a translational gradient of expression levels along the polycistronic mRNA transcript. Therefore, compactness of the cassette was sacrificed in favor of a cassette with 3 independent transcriptional (promoter, gene, and polyA) units.

### Promoter and PolyA Selection

### Promoters

The choice of promoters to drive the expression of the payloads was based on two main factors. First, since the human cytomegalovirus (hCMV) IE promoter was previously selected to drive expression of genes within the HSV-1 γ34.5 locus (hFLT3L-P2A-UL49.5 expression, as discussed in greater detail in Example 4, below), this promoter, or any other promoter derived from it (e.g., CAG promoter or other CMV hybrid promoters), could not be used again in order to avoid sequence homology and potential subsequent viral DNA recombination. To drive independent expression of each of the three payloads, three promoters from the following four strong short constitutive viral promoters were selected:
The major IE promoter from the murine cytomegalovirus (mCMV, 536 bp). The mCMV IE promoter is comparable in strength and kinetics in most human cell lines and superior in mouse cells to the hCMV IE promoter.
The AoHV-1 IE promoter from Aotine betaherpesvirus 1 (AoHV-1, 482 bp). This short promoter was shown to be potent and able to drive transgene expression from a viral vector (US Patent Application Pub. No. 20180223314A1). Moreover, it also has limited sequence identity with the mCMV and the hCMV promoters.
The MMLV 5′ long terminal repeat (LTR) from the Moloney murine leukemia virus (MMLV, 453 bp).
A bidirectional promoter derived from the mCMV IE promoter (Pbidir3, 1,459 bp). This promoter was based on the endogenous bidirectional IE promoter from mCMV and was shown to have strong and balanced expression from both directions of the promoter in viral vectors (US Patent Application Pub. No. 20180135075A1).

### PolyA Elements

It was determined that the 3′ most transcriptional unit in the cassette would use the endogenous US9-10 polyA element to drive cleavage and polyadenylation of its mRNA. This poly A element is naturally located at the 3′end of the US10 locus within the WT HSV-1 genome.

For the 2 remaining polyadenylation signals in the cassette, three 3 strong short polyadenylation signals commonly used in expression vectors were chosen:
The simian vacuolating virus 40 polyA (SV40 pA, 122 bp).
The human beta globin polyA (hBGpA, chr11:5,225,203-5,225,597, GRCh38/hg38, 395 bp).
The rabbit beta globin polyA (rBGpA, chr1: 146,236,955-146,237,053, Broad/oryCun2, 99 bp).

In addition, a fourth polyadenylation signal was generated, containing a short synthetic polyadenylation signal composed of the human glyceraldehyde 3phosphate dehydrogenase (GAPDH) 3′ UTR (chr12:6,538,171-6,538,347, GRCh38/hg38, 117 bp), a strong synthetic poly A signal (62 bp; see, Levitt et al. Definition of an efficient synthetic poly(A) site. Genes Dev. 1989; 3(7): 1019-25), and the RNA polymerase II transcriptional pause signal from the human alpha2 globin gene (chr16:174,014-174,105, GRCh38/hg38, 92 bp). This fourth polyadenylation signal was termed GAPDH_SPA (331 bp).

Finally, because the expression cassette was designed to be compact, it was important to avoid read-through transcription that could interfere with downstream transcriptional units. Therefore, RNA Polymerase II transcriptional pause signals were included between the transcriptional units. These elements were intended to stop RNA Polymerase II transcription shortly after the polyadenylation signals and avoid RNA Polymerase II read-through. Two RNA Polymerase II transcriptional pause signals were selected:
Human complement C2 protein terminator (C2, chr6:31,945,677-31,945,837, GRCh38/hg38, 161 bp; see, Ashfield et al. MAZ-dependent termination between closely spaced human complement genes. EMBO J. 1994; 13(23):5656-67).
Human Gastrin terminator (hGT, chr17:41,716,132-41,716,204, GRCh38/hg38, 73 bp).

The two selected RNA Polymerase II transcriptional pause signals have also been reported to increase gene expression by improving mRNA 3′ end processing (Kim et al. Improved mammalian expression systems by manipulating transcriptional termination regions. Biotechnol Prog. 2003; 19(5):1620-2).

### Cassette Engineering and Testing

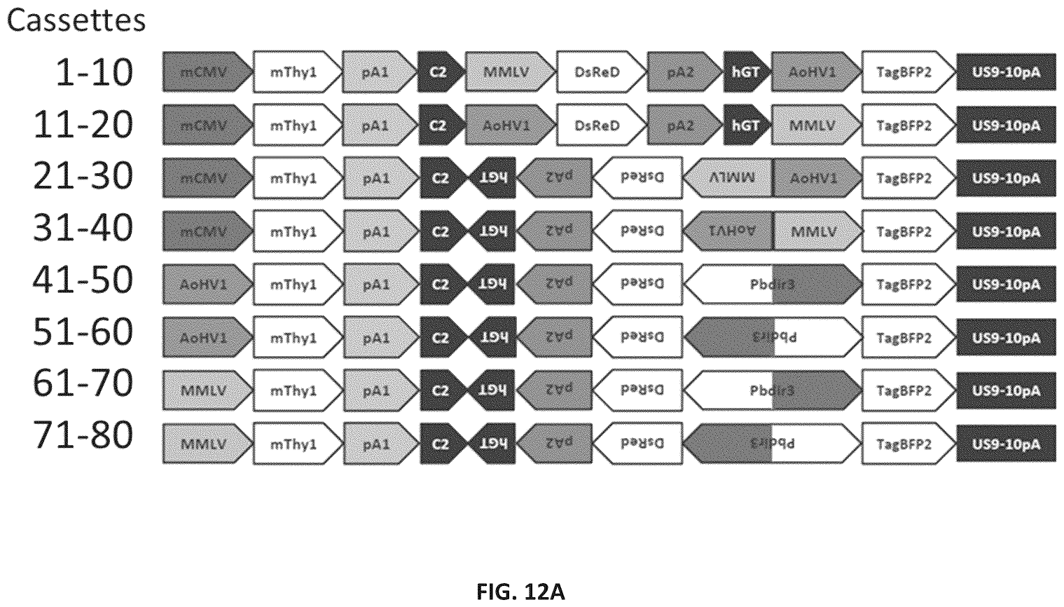

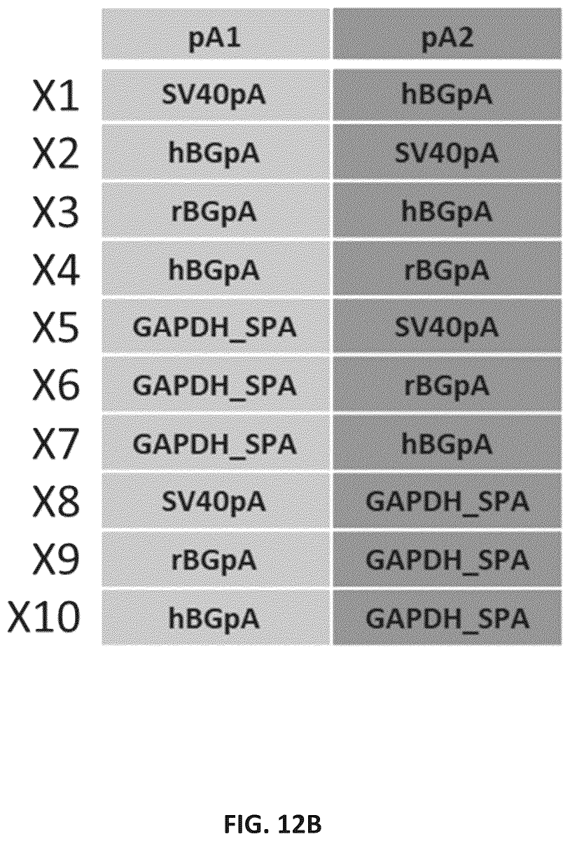

To select the optimal expression cassette for the three independent transcriptional units for expression of the anti-CTLA-4 antagonist (hαCTLA-4), CD40 agonist (hCD40ag), and IL-12 (hscIL-12) payloads, a library of 80 promoter and polyA combinations was built using the elements described above (FIGS. 12A-12B).

As shown in FIG. 12A, eight different promoter combinations and orientations were designed. Then, each of those eight combinations was paired with ten different poly A pairs (FIG. 12B), yielding 80 unique expression cassettes. As discussed above, the third polyA signal for the 3′ most transcriptional unit was fixed (US9-10 polyA).

To avoid the cumbersome quantification of each immunomodulatory payload within the cassettes, a reporter system was initially used to screen the 80 designed cassettes. The immunomodulatory payloads were replaced by 2 fluorescent proteins (i.e., DsRed and TagBFP2) and a plasma membrane marker easily detectable by flow cytometry (i.e., mThy1.1). Those markers were chosen to allow for a simplified screening approach in one step using flow cytometry with minimum spill over and need for compensation:
TagBFP2 (Ex 399 nm/Em 454 nm).
DsRed (Ex 558 nm/Em 583 nm).
LIVE/DEAD Fixable Dead Cell Stain (Ex 633-635 nm/Em 660 nm).
mThy1.1 detected by an APC-vio770 labelled antibody (Ex 652 nm/Em 775 nm).

To screen the library of cassettes, individual cassettes were synthetized and cloned into pUC57 plasmid vectors. The initial screen was carried out by transient transfection of the plasmids in HEK293T cells. Transient transfections rarely produce a normal distribution of the overexpressed markers by flow cytometry, making it difficult to quantify absolute expression levels using standard readouts such as median fluorescent intensity, mean fluorescent intensity, or geometric mean. Instead, the percentage of positive cells for each pair of reporters (TagBFP2+DsRed, DsRed+mThy1.1, and TagBFP2+mThy1.1) was calculated and compared to a mock transfection (empty plasmid). This analysis quickly identified 3 sets of cassettes with a robust signal for each reporter transcriptional unit: Cassettes 11-20, 31-40, and 71-80 (Table 1).

To further narrow the expression cassette selection, the expression of individual reporters was then evaluated by Western blot for Cassettes 11-20, 3140, and 71-80 transiently-transfected in HEK293T (Table 1). This Western blot analysis enabled narrowing the selection to 6 candidate cassettes showing a balanced expression of all 3 reporters: Cassettes 14, 17, 37, 38, 72, and 75. These 6 cassettes were then screened by Western blot for reporter expression after transient transfection in 2 different tumor cell lines (i.e., A549 and H1299) to factor in possible differences of promoter activity depending on cell type. This analysis showed that reporter payload expression from the MMLV promoter was the most affected by cell line used, while mCMV expression was the most stable, and that these variations were independent from the cassette architecture. Based on these results, one cassette from each set was retained: Cassettes 17, 37, and 75.

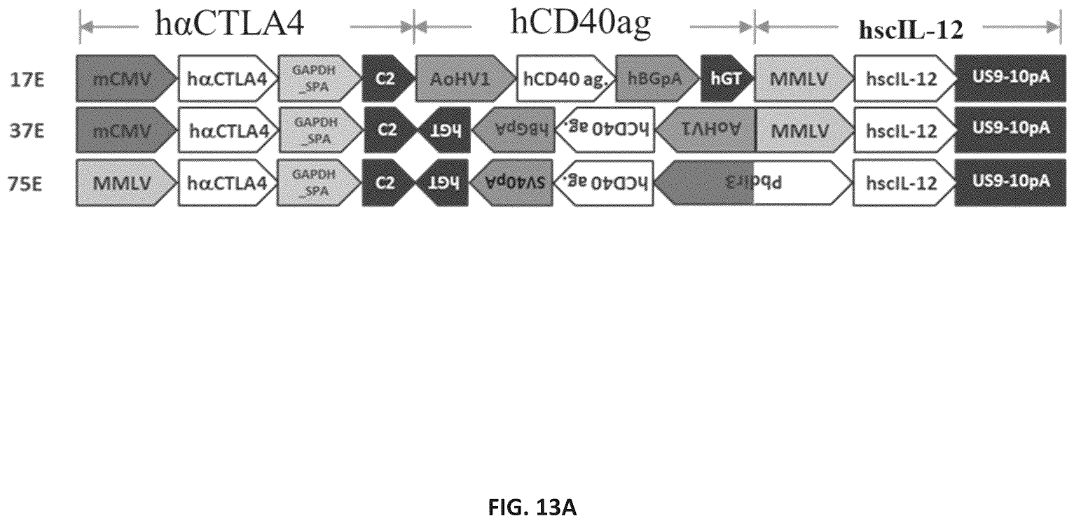

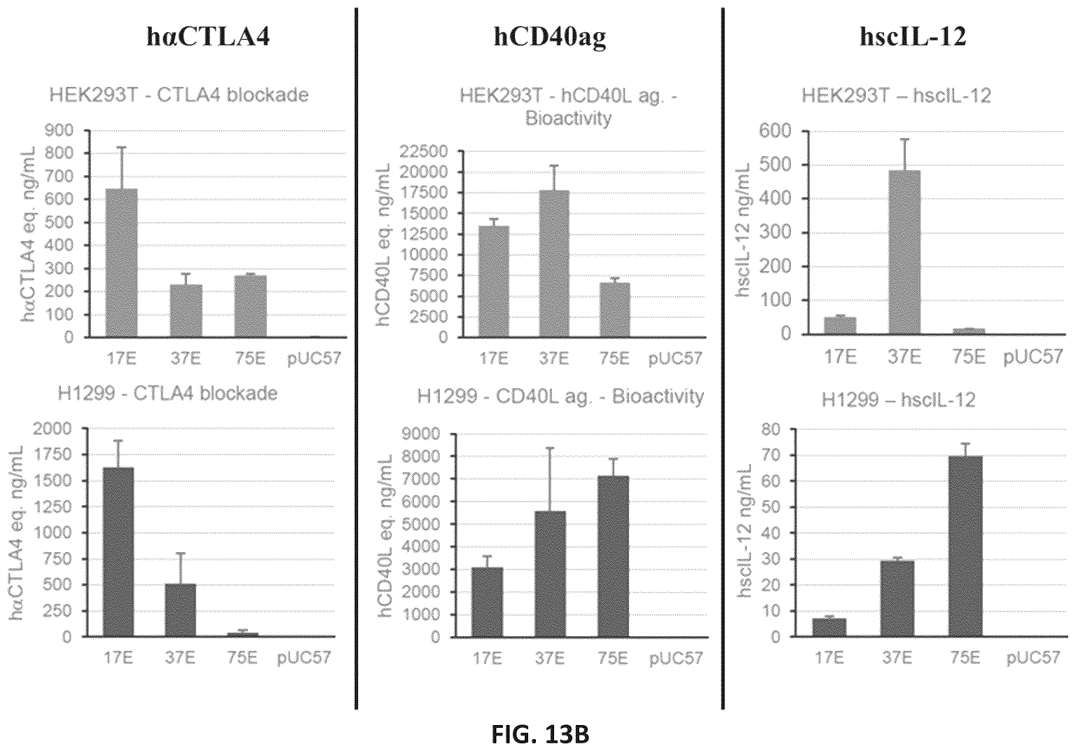

The final three cassettes were tested in the context of the immunomodulatory payloads: the anti-CTLA-4 antagonist (hαCTLA-4), CD40 agonist (hCD40ag), and IL-12 (hscIL-12) payloads. The selected cassette architectures with the immunomodulatory payloads (Table 1 and FIG. 13) were synthetized and cloned into pUC57 plasmid vectors:
Cassette 17E encodes the same promoter/polyA architecture as Cassette 17; hαCTLA-4 replaces mThy1.1; hCD40ag replaces DsRed; hscIL-12 replaces TagBFP2.
Cassette 37E encodes the same promoter/polyA architecture as Cassette 37; hαCTLA-4 replaces mThy1.1; hCD40ag replaces DsRed; hscIL-12 replaces TagBFP2.
Cassette 75E encodes the same promoter/polyA architecture as Cassette 75; hαCTLA-4 replaces mThy1.1; hCD40ag replaces DsRed; hscIL-12 replaces TagBFP2.

After transient transfection of each cassette in HEK293T and H1299 cells, the expression of each payload in the culture medium of the transfected cells was quantified (FIG. 13B). Cassette 75E showed the lowest expression of 2 payloads (i.e., hCD40ag and hscIL-12) in HEK293T cells and a critically low expression of the third payload (i.e., hαCTLA-4) in H1299 cells. The 17E and 37E cassettes showed more balanced expression of the payloads. Based on these results, cassettes 17E and 37E were selected to be cloned and tested in the context of an OV.

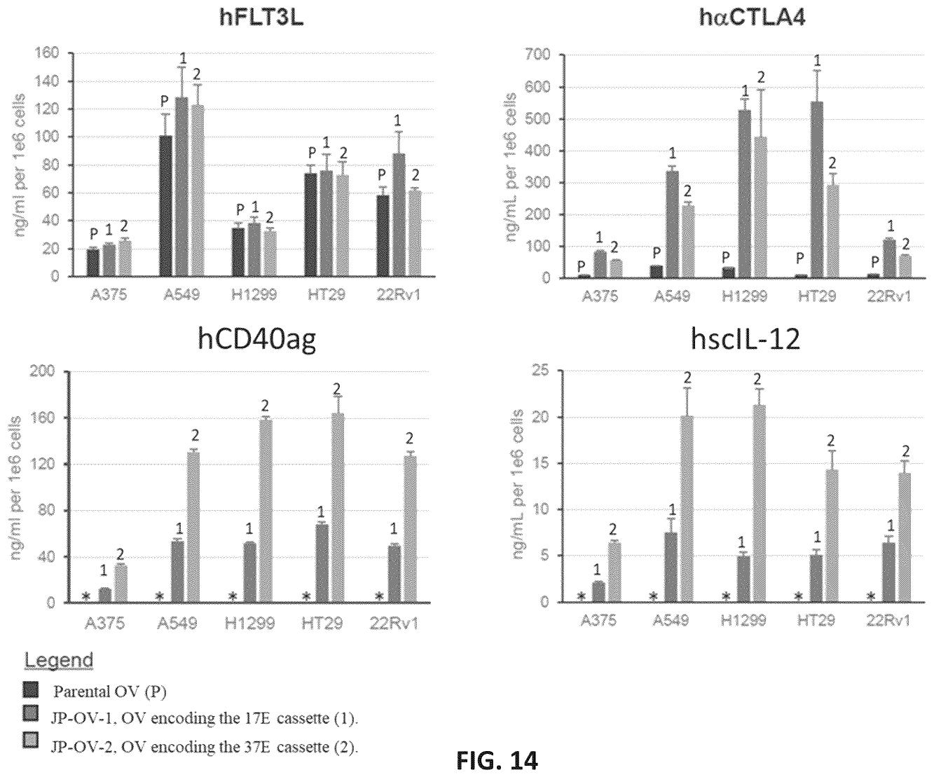

Expression cassettes 17E and 37E were subsequently subcloned into targeting vectors for recombination with a parental HSV-1 virus to obtain the viruses JP-OV-1 and JP-OV-2, respectively (Table 1; see Example 4, below, for a detailed description of the construction of these viruses, in particular of JP-OV-2). Immunomodulatory payload expression from each virus was assessed on five tumor cell lines (i.e., A375, A549, H1299, HT29, and 22Rv1) known to be permissive for HSV-1 replication, and covering four different tumor types. Briefly, cancer cells were infected with the parental virus, JP-OV-1 (encoding cassette 17E), or JP-OV-2 (encoding cassette 37E) at a multiplicity of infection (MOI) of 1. After 24 hours, culture supernatants were collected and quantified by enzyme-linked immunosorbent assay (ELISA) for the expression of the hαCTLA-4, hCD40ag, and hscIL-12 payloads, as well as the hFLT3L payload (encoded within the HSV-1 γ34.5 locus, including in the parental virus; see Example 4, below). Since the infection was carried out at MOI=1 on near-confluent cell culture plates (for optimum infection conditions), the number of cells infected varied by cell line. To account for this factor, the payload concentration was expressed as ng/mL per 106 cells. As shown in FIG. 14, expression of hFLT3L, although variable between cell lines, was comparable between the three viruses for each cancer cell line infected. This illustrated the inherent variability of constitutive viral promoters depending on infected cell type. More importantly, the consistent hFLT3L expression between the three viruses demonstrated that the introduction of the 3-payload expression cassette (i.e., the 17E or 37E cassettes) had no impact on hFLT3L expression from the γ34.5 locus. The expression of the CD40 agonist (hCD40ag) and IL-12 (hscIL-12) payloads was consistently higher in the JP-OV-2-infected cell supernatants compared to JP-OV-1-infected cell supernatants across all cancer cell lines tested. Finally, the expression of the anti-CTLA-4 antagonist (hαCTLA-4) payload was slightly lower from JP-OV-2-infected cell supernatants compared to JP-OV-1-infected cell supernatants across all cancer cell lines tested. Based on these results, JP-OV-2, which showed robust expression of all payloads over the panel of cancer cell lines was selected as the HSV-1 OV candidate, as described in detail in Examples 4-5, below.

A summary of the screening steps described above is provided in Table 1.

TABLE 1
Summary of the screening steps leading to the selection of the payload
expression cassette and HSV-1 oncolytic virus candidate.

| | | | | |
|---|---|---|---|---|
| | | | Cassettes | Cassettes |
| Payloads | Screen | Cell lines | tested | selected |

Reporter cassettes

| | | | | |
|---|---|---|---|---|
| TagBFP2, | Flow | HEK293T | 1 to 80 | 11-20, 31- |
| DsRed, | cytometry | | | 40, 71-80 |
| mThy1.1 | | | | |
| TagBFP2, | Western | HEK293T | 11-20, 31- | 14, 17, 37, |
| DsRed, | blot | | 40, 71-80 | 38, 72, 75 |
| mThy1.1 | | | | |
| TagBFP2, | Western | A549, H1299 | 14, 17, 37, | 17, 37, 75 |
| DsRed, | blot | | 38, 72, 75 | |
| mThy1.1 | | | | |

Immunomodulatory payload expressing cassettes

| | | | | |
|---|---|---|---|---|
| hαCTLA-4, | ELISA | HEK293T, | 17E, 37E, | 17E and |
| hCD40ag, | bioactivity | H1299 | 75E | 37E |
| hscIL-12 | | | | |

Immunomodulatory payload expressing virus

| | | | | |
|---|---|---|---|---|
| hαCTLA-4, | ELISA | A375, A549, | JP-OV-1 | 37E |
| hCD40ag, | | H1299, HT29, | (encoding | |
| hscIL-12 | | 22Rv1 | cassette 17E) | |
| | | | JP-OV-2 | |
| | | | (encoding | |
| | | | cassette 37E) | |

### Example 4: Construction of an HSV-1 Oncolytic Virus with Stealth Immune Functions and Synergistic Immunomodulatory Payloads

This Example describes the construction of JP-OV-2, an HSV-1 OV with the innate and adaptive immune stealth functions as described in Example 1 herein (i.e., IE- and L-US11 expression, and TAP inhibition through expression of UL49.5), and expressing the immunomodulatory payloads as described in Examples 2 and 3 herein (i.e., hFLT3L, hαCTLA-4, hCD40ag, and hscIL-12).

### Recombinant Virus Engineering

Recombinant HSV-1 viruses were generated using homologous recombination in Vero cells adapted to proliferate on serum-free media (VSF cells) by co-transfecting viral DNA and linearized target vectors. Target vectors were designed to harbor virus homologous DNA sequences designed to target the γ34.5 or US10-12 loci flanking the DNA encoding the transgenes to insert within the selected locus. The methodology used for each step of engineering was based on insertion followed by replacement of a reporter gene (i.e., green fluorescent protein (GFP)) with transgenes within the γ34.5 or US10-12 loci of the virus. Insertion/replacement of a reporter gene within the viral genome enabled visual selection of recombinant virus plaques. Recombinant viruses were identified and selected based on loss or gain of the reporter gene, followed by polymerase chain reaction (PCR) screening for the transgene cassette. Recombinant clones (4-8 clones per transfection) were subjected to 3 to 4 rounds of plaque purification and PCR confirmation. Following final clone selection, release assays were performed on recombinant clones, from which one final recombinant virus was selected and advanced for further characterization and/or for generating intermediate viruses such as Step 2 virus, as described in detail below.

### Multi-Step Virus Construction

JP-OV-2 was constructed in four steps using a parental HSV-1 virus termed ΔXN1. The parental ΔXN1 virus is an attenuated virus deficient for the γ34.5 and US12 genes, and was constructed using the WT HSV-1 Patton strain (Mulvey et al. A herpesvirus ribosome-associated, RNA-binding protein confers a growth advantage upon mutants deficient in a GADD34-related function. J Virol. 1999; 73(4):3375-3385). ΔXN1 expresses β-glucuronidase from the γ34.5 locus, and the deletion of the US12 gene licenses the virus to express US11 from the IE US12 promoter (FIG. 15A).

The first step for the construction of JP-OV-2 was to replace the β-glucuronidase cassette within the γ34.5 locus of ΔXN1 with an enhanced green fluorescent protein (eGFP) cassette, generating ΔXN1-GFP (FIG. 15B).

The second step for the construction of JP-OV-2 was to further engineer the ΔXN1-GFP virus to replace the eGFP cassette with a hFLT3L-P2A-UL49.5 cassette for the expression of the hFLT3L payload and UL49.5, thereby generating the Step 1 virus (FIG. 15C) The hFLT3L-P2A-UL49.5 cassette is present in two copies in the viral genome (one copy in each of TR_{L} and IR_{L}).

The third step for the construction of JP-OV-2 was to prepare the US10-12 locus for further payload insertion. A GFP reporter cassette (eGFP) was inserted within the US10-12 locus of the Step 1 virus by recombining the Step 1 virus viral DNA with a pTVTRsD1210 pA_hGHmCMVeGFPS target vector. From this recombination and subsequent purification/selection steps, Step 2 virus was selected (FIG. 15D). The Step 2 virus encodes the hFLT3L-P2A-UL49.5 cassette in the γ34.5 locus, and the GFP cassette in the US10-12 locus.

In the fourth and final step to construct JP-OV-2, the US1012 locus in the Step 2 virus was further engineered to replace the eGFP cassette by the 37E cassette as described in Example 3 herein, which encodes the anti-CTLA-4 antagonist (hαCTLA-4), CD40 agonist (hCD40ag), and IL-12 (hscIL-12) payloads. The Step 2 virus DNA was co-transfected with a pTVTRsh11CO10 pA_37 E target vector, which is the 37E cassette with viral DNA flanking sequences, to replace the GFP cassette, thus generating the final Step 3 virus (FIG. 15E). To obtain the final virus clone, recombinant virus plaques, which displayed loss of GFP expression, were identified and discriminated from GFP-positive non-recombinant virus plaques by visual screening using fluorescence microscopy. Clonally recombinant viruses were isolated through four rounds of plaque purification, and the recombination status of the clones was confirmed at each round of plaque purification using microscopy and PCR assays. Master seed stocks (MSS), which is the initial ˜60 mL virus stock from plaque-purified viruses, were prepared from four highly purified (through successive plaque purification) recombinant clones, and the fitness of the recombinant clones was assessed by a series of release assays (see below). The clones selected for further analysis were Step 3 clone 1, Step 3 clone 2, Step 3 clone 3, and Step 3 clone 4. As described in detail in Example 5, below, the Step 3 clone 3 virus was selected as the final viral clone, termed JP-OV-2.

### Example 5: Selection and Validation of JP-OV-2, an Oncolytic HSV-1 with Stealth Immune Functions and Synergistic Immunomodulatory Payloads

As discussed in Example 4, above, viral engineering and construction resulted in four virus clones containing the final viral genome construct for expression of the innate and adaptive immune stealth functions as described in Example 1 herein (i.e., IE- and L-US11 expression, and TAP inhibition through expression of UL49.5), and the immunomodulatory payloads as described in Examples 2 and 3 herein (i.e., hFLT3L, hαCTLA-4, hCD40ag, and hscIL-12). The four clones were Step 3 clone 1, Step 3 clone 2, Step 3 clone 3, and Step 3 clone 4 (see, Example 4, above). This Example describes the selection and validation of the final HSV-1 OV clone, Step 3 clone 3, which was termed JP-OV-2.

### Genomic Integrity

Presence of transgene cassettes in MSS stocks of the Step 3 recombinant clones was confirmed by PCR assay (data not shown). Next, the genome integrity at the γ34.5 and US10-12 loci of the recombinant viruses was confirmed by Southern blotting analysis using viral DNA isolated from Vero cells infected with MOI=1 at 24 hours post infection, the results of which confirmed that the intended recombination events had occurred, and that the desired recombinant viruses were properly engineered (data not shown).

Following confirmation of the DNA integrity, the expression of the stealth immune function proteins (i.e., UL49.4 and US11) and the four payload proteins (i.e., hFLT3L, hαCTLA-4, hCD40ag, and hscIL-12) was confirmed by Western blot and ELISA, as described below.

### Transgene Expression

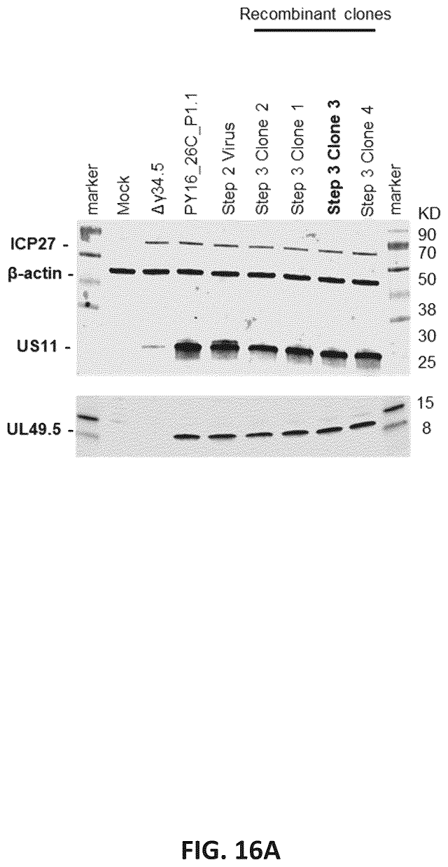

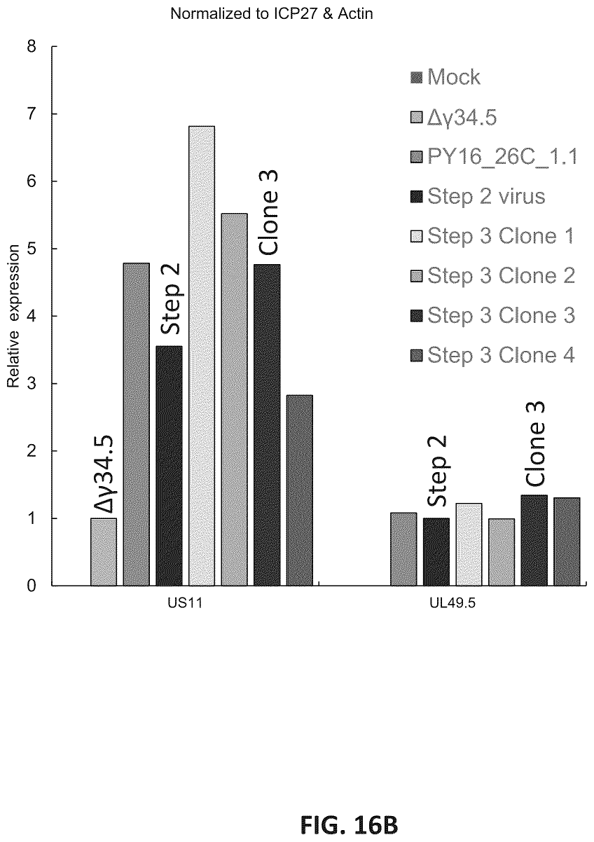

Expression of immune stealth function proteins UL49.5 and US11 was analyzed following infection of Vero cells with MOI=5. At 6 hours post-infection, proteins from cell lysates (˜6×104 cells) were resolved on sodium dodecyl sulfate-polyacrylamide gel electrophoresis (SDS-PAGE), electroblotted to polyvinylidene fluoride (PVDF) membrane, and immunoblotted with anti-UL49.5 and anti-US11 antibodies, as well as control viral protein (i.e., anti-ICP27) and control cell loading (i.e., anti-βactin) antibodies (FIGS. 16A-16B). The Step 3 clone 3, Step 2, and PY16_26C_P1.1 which is an oncolytic HSBV-1 virus with UL49.5, FLT3L and a CD40 angonist integrated at the γ34.5 locus recombinant viruses constitutively expressed similar levels of UL49.5 and US11 proteins, but to about a 4× greater extent for US11 compared to the control Δγ34.5 virus, which also does not express UL49.5.

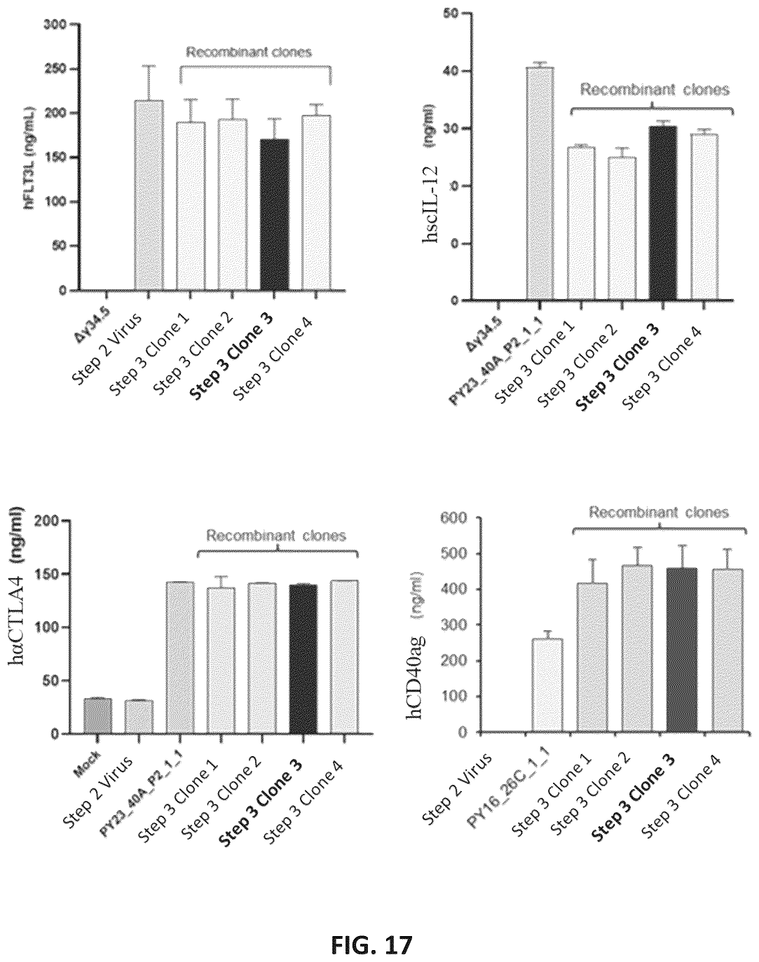

Expression of the four immunomodulatory payloads was analyzed by infecting Vero cells at MOI=1 with Step 3 virus clones 1-4. At 30 hours post-infection, cell culture supernatants were harvested, centrifuged to remove cell debris, and stored at 80° C. The hFLT3L, hαCTLA-4, hCD40ag, and hscIL-12 payload proteins from the harvested supernatants were quantitated using ELISA. The Δγ34.5 virus served as negative control virus for hFLT3L and hscIL-12 expression, and the Step 2 virus served as parental control virus not encoding the hαCTLA-4, hCD40ag, and hscIL-12 payloads (i.e., not containing the 37E cassette) within the US1012 locus. PY23_40A_P2_1_1, which has the same γ34.5 expression cassette for UL49.5 and FLT3L but only expresses IL-12 and anti-CTLA-4 from the US10-12 locus served as positive control for hscIL-12 and hαCTLA-4 expression. PY16_26C_1_1 served as positive control for hCD40ag expression, assessed by ELISA. Using a standard analyte concentration curve, it was determined that Step 3 clone 3 virus expressed about 170 ng/ml hFLT3L, about 30 ng/ml hscIL-12, about 140 ng/ml hαCTLA-4, and about 450 ng/ml hCD40ag under the tested conditions (FIG. 17).

### Replication and Cytotoxicity

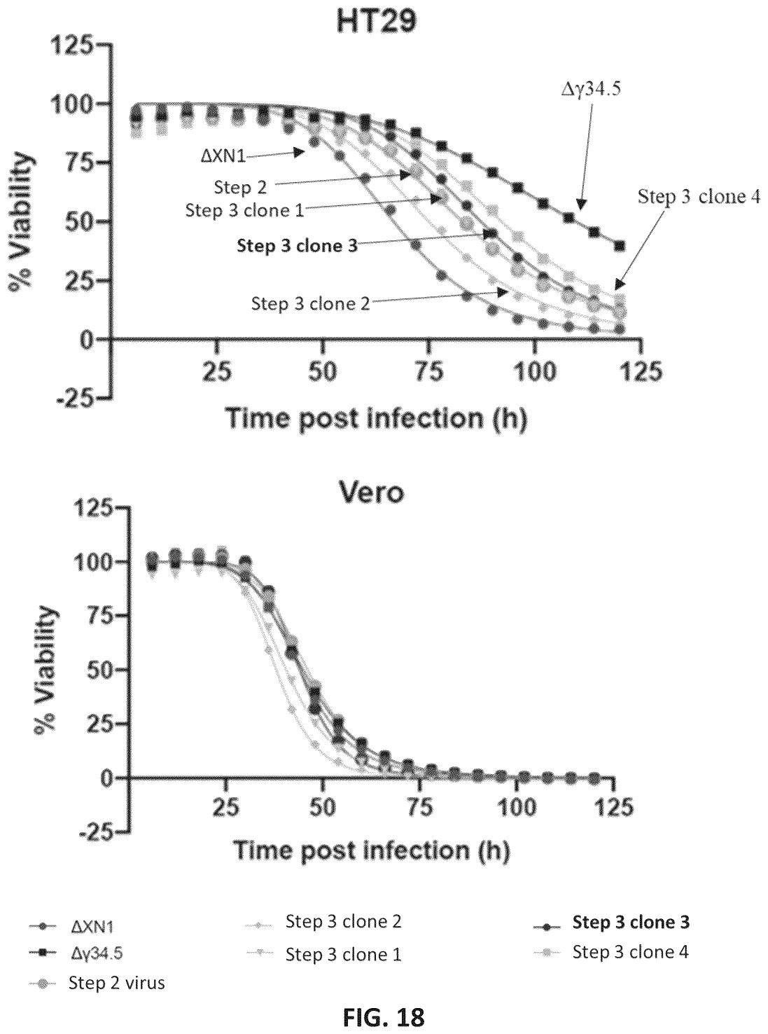

Next, the replication of Step 3 virus clones 1˜4 was assessed by infecting HT29 and Vero cells at MOI=0.01. The cytopathic effect produced by each virus was monitored using real-time cell analysis with the xCelligence system every 6 hours post infection. Virus-induced cytotoxicity was measured based on cell viability, and 50% effective time (ET₅₀) value was calculated from the slope of the normalized cell viability curves. To account for differences in titers between the viruses, the ET₅₀ values of HT29 were normalized to the ET₅₀ values of Vero cells. As shown in FIG. 18, Step 3 clone 3 virus displayed higher cytotoxicity on the HT29 cell line, with an ET₅₀ value of 87.34 hours compared to control virus Δγ34.5, which displayed an ET₅₀ value of 109.8 hours (FIG. 18 and Table 2). In addition, Step 3 clone 3 virus induced slightly more cytotoxicity compared to the other Step 3 clones tested

TABLE 2
Cytotoxicity and cell viability of virus-infected cells.

| | | | | Step | Step | Step | Step |
|---|---|---|---|---|---|---|---|
| | | | | 3 | 3 | 3 | 3 |
| | | | Step | Clone | Clone | Clone | Clone |
| ET₅₀ | | | 2 | 1 | 2 | 3 | 4 |
| (hours) | ΔXN1 | Δγ34.5 | Virus | Virus | Virus | Virus | Virus |
| Vero cells | 44.1 | 44.92 | 46.08 | 40.58 | 38.02 | 44.07 | 44.38 |
| HT29 cells | 66.73 | 109.8 | 82.93 | 83.23 | 75.1 | 87.34 | 92.22 |
| Normalized | 1.51 | 2.44 | 1.8 | 2.05 | 1.98 | 1.98 | 2.08 |
| (HT29/Vero) | | | | | | | |

ET₅₀, 50% effective time.

Based on all of the results described above, the Step 3 clone 3 virus was selected as the final clone, and named JP-OV-2.

The viral genome of JP-OV-2 was sequenced using the Pacbio Sequel systems SMRT Cell long-read whole-genome sequencing method and annotated with a reference sequence derived from parental virus genome and transgene cassette. The sequences of transgene cassettes matched the reference sequence with no detected mutations, confirming the genetic identity of the engineered transgenes (data not shown). Whole-genome sequencing indicated the presence of a missense mutation in the viral DNA polymerase catalytic subunit UL30 gene of JP-OV-2. The observed missense mutation, 1193Pro>Ser, is localized outside of the enzyme's active site and should not interfere with UL30 activity. To confirm the absence of detrimental effect caused by the mutation on UL30 activity, replication and sensitivity to acyclovir (which targets UL30) were assessed and compared to that of parental virus (i.e., Step 2 virus, which is WT for UL30). It was demonstrated that both viruses replicated similarly and were similarly sensitive to acyclovir, confirming that the UL30 1193Pro>Ser mutation within JP-OV-2 had no effect on virus replication and sensitivity to acyclovir (data not shown).

### Genetic and Functional Stability of JP-OV-2

The genetic stability of JP-OV-2 was assessed in duplicate experiments (biological replicates) over 8 cycles of in vitro replication. Eight cycles of replication was chosen to adhere to the strictest guidelines on genetic stability for viral vaccine seed lots requiring demonstration of genotype and phenotype stability for a minimum of 5 cycles beyond the final product (United States Pharmacopeia (USP). Chapter 1235: Vaccines for human use—general considerations). The 8 replication cycles mimic the number of virus replication cycles from the master virus seed stock (MVS) production (Cycle 1), working stock production (Cycle 2), clinical stock production (Cycle 3), and 5 additional replication cycles. Briefly, each replication cycle consisted of multiple rounds of virus replication over 3 days following serum-free Vero cell infection at a low MOI of 0.01. Infected cells and supernatant were harvested, virus was released using the freeze/thaw method, and virus titer was determined by plaque assay for subsequent virus replication cycles.

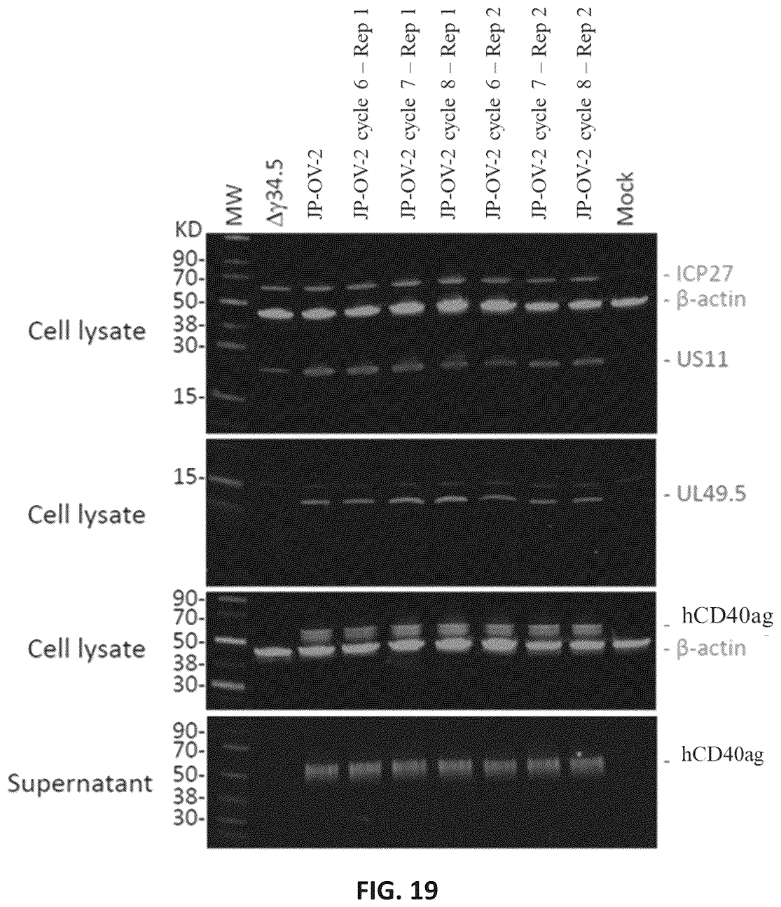

The expression of the ICP27, UL49.5, US11, and hCD40ag proteins was assessed by Western blot in total cell extracts of serum-free Vero cells 24 hours after infection at a MOI=5 with JP-OV-2 Cycle 6, 7, and 8 viruses, and compared with cell extracts of serum-free Vero cells infected in the same conditions with Δγ34.5 (negative control) and an early-passage JP-OV-2 virus. Expression of hCD40ag was also analyzed in the culture supernatant by the same method. No significant changes in protein expression were observed by Western blot analysis between all replication cycles, suggesting stability of virus replication for key proteins during up to 8 cycles of virus replication (FIG. 19).

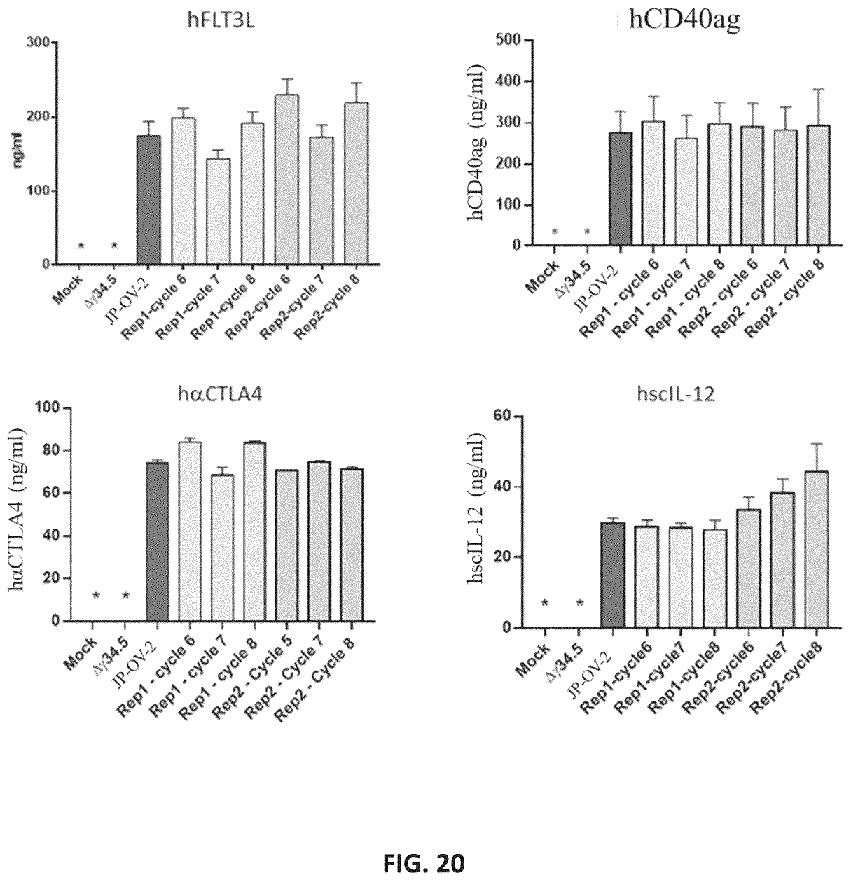

The expression of the hFLT3L, hαCTLA-4, hCD40ag, and hscIL-12 payload proteins was assessed by ELISA in culture supernatants of Vero cells 24 hours after infection at a MOI=1 with JP-OV-2 replication Cycle 6, 7, and 8 viruses, and compared with culture supernatants of serum-dependent Vero cells infected in the same conditions with Δγ34.5 (negative control) and an early passage of JP-OV-2. As shown in FIG. 20, quantification of the immunomodulatory payloads in the culture supernatant did not show any significant changes in protein expression between early-passage JP-OV-2 and following multiple replication cycles (Cycle 6, 7, and 8 in replicates).

Genetic stability of the individual payloads (i.e., hFLT3L, hαCTLA-4, hCD40ag, and hscIL-12) was assessed by Sanger sequencing. Briefly, the DNA sequence corresponding to the open reading frame and flanking sequences of each payload was amplified by PCR using JP-OV-2 replication Cycle 8 duplicate viruses as templates. These PCR products were sequenced by the Sanger method using a set of 10 primers (5 primers in each direction) to obtain optimum base coverage. Overall, each nucleotide in the open reading frame of each payload was read at least twice in each direction. No mutations were detected in either replicate, confirming the stability of JP-OV-2 immunomodulatory payload nucleotide sequence over 8 cycles of replication.

Genome integrity of the payload expression cassettes within the Δγ34.5 and US10-12 loci was individually assessed by Southern blotting using viral DNA extracted from serum-dependent Vero cells infected with JP-OV-2 after replication Cycles 6, 7, and 8, and compared with viral DNA extracted from serum-dependent Vero cells infected in the same conditions with an early passage of JP-OV-2 (early-passage control), as well as with Δγ34.5 and Step 2 viruses (negative controls for transgene expression). For both loci, DNA fragment patterns for JP-OV-2 following Cycles 6, 7, and 8 of replication were identical to early-passage JP-OV-2 in two replicates, confirming the macroscopic integrity of the expression cassettes over 8 cycles of replication (data not shown).

Finally, the full-genome integrity of JP-OV-2 after 8 replication cycles was further assessed by whole-genome sequencing using Pacbio Sequel SMRT Cell long-read whole-genome sequencing. Reads were mapped to a reference genome for JP-OV-2 as previously determined by whole-genome sequencing and analyzed for sequence variants (single-nucleotide polymorphisms (SNPs), short insertions/deletions) and structural variants (large insertions/deletions). Whole-genome sequencing confirmed that no DNA segment within each payload was lost, and that no mutations were detected over the 8 cycles of replication. Moreover, no losses, insertions, deletions, or mutations were detected in any of the endogenous open reading frames of the viruses in comparison with the reference sequence for JP-OV-2 (data not shown).

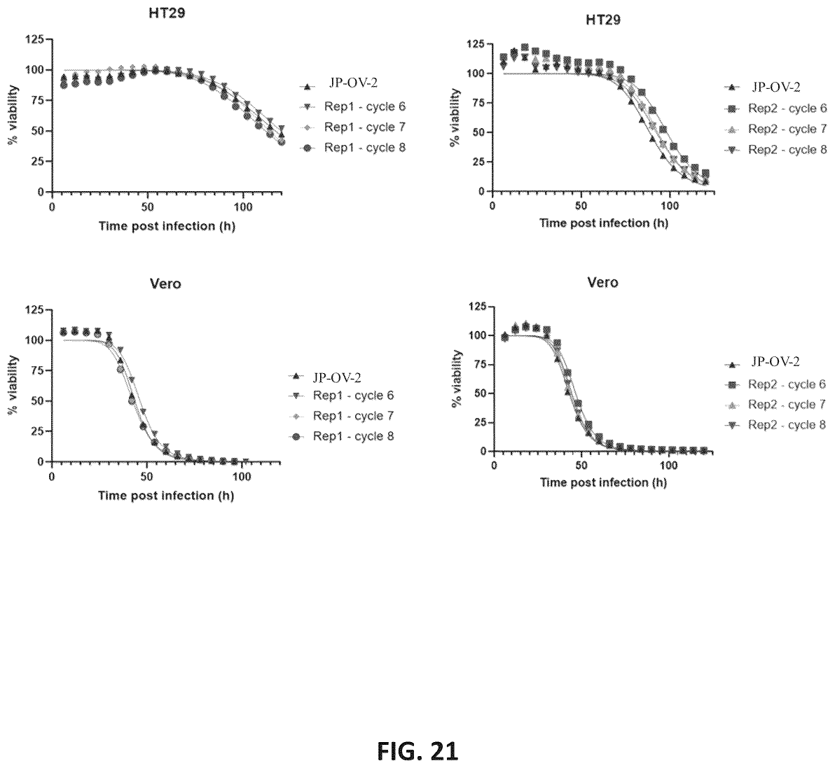

As an overall and biological measure of the functional stability of JP-OV-2, virus-induced cancer cytotoxicity was tested after replication Cycles 6, 7, and 8 and compared to that of an early-passage JP-OV-2 (early-passage control). Virus cytotoxicity over time was measured via xCelligence on human colorectal adenocarcinoma cell line HT29, which is relatively resistant to HSV-1 replication. This HSV-1 replication stringency enhances potential minor differences in viral fitness. As shown in FIG. 21, virus samples (from two replicates) collected after all late replication cycles (i.e., Cycles 6, 7, and 8) replicated similarly to the early-passage control. Vero cells, which are highly permissive, were used as a control to confirm that equal amounts of all viruses were used in the assay. While virus-induced HT29 cytotoxicity was similar between viruses collected after few or multiple replication cycles within a given assay, a difference in overall cytotoxicity was observed between independent assays. This variation is common with the xCelligence technique, which is highly dependent on cell density and general cell health (which varies between experiments) and is not relevant to virus replication.

In sum, the results described above demonstrated that JP-OV-2 was genetically stable over at least 8 cycles of replication in vitro.

### Sensitivity to Acyclovir

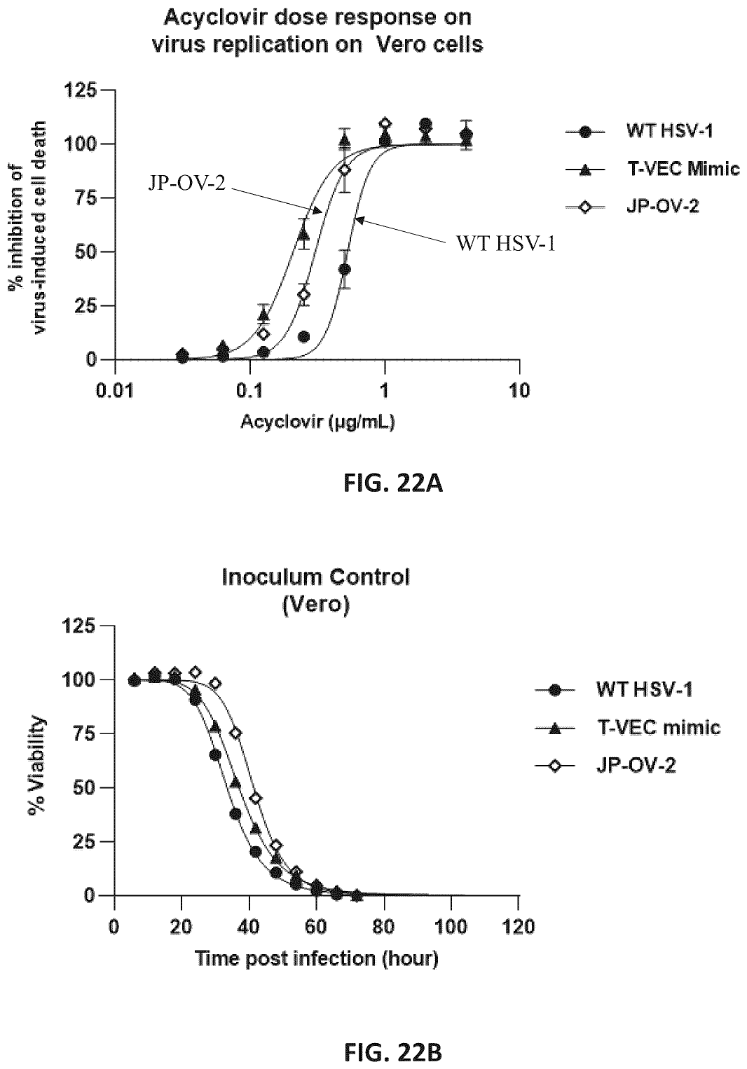

OV therapy with HSV-1 offers the advantage of the availability of anti-viral agents, such as acyclovir, in the instance of an unexpected event occurring during treatment therapy. The sensitivity of JP-OV-2 was therefore assessed and compared to that of WT HSV-1 and a previously-reported genetically engineered oncolytic herpesvirus mimic, which has a genetic architecture similar to a previously-reported genetically engineered oncolytic herpesvirus, but expresses murine GM-CSF. As shown in FIGS. 22A-22B, both JP-OV-2 and a previously-reported genetically engineered oncolytic herpesvirus mimic demonstrated slightly enhanced sensitivity to acyclovir compared to that of WT HSV-1, which is consistent with the partial attenuation of both OVs. These data demonstrated that JP-OV-2 is highly sensitive to anti-HSV agents such as acyclovir, which could be used in the unexpected event of uncontrolled HSV-1 replication.

### Maintenance of IFN Response

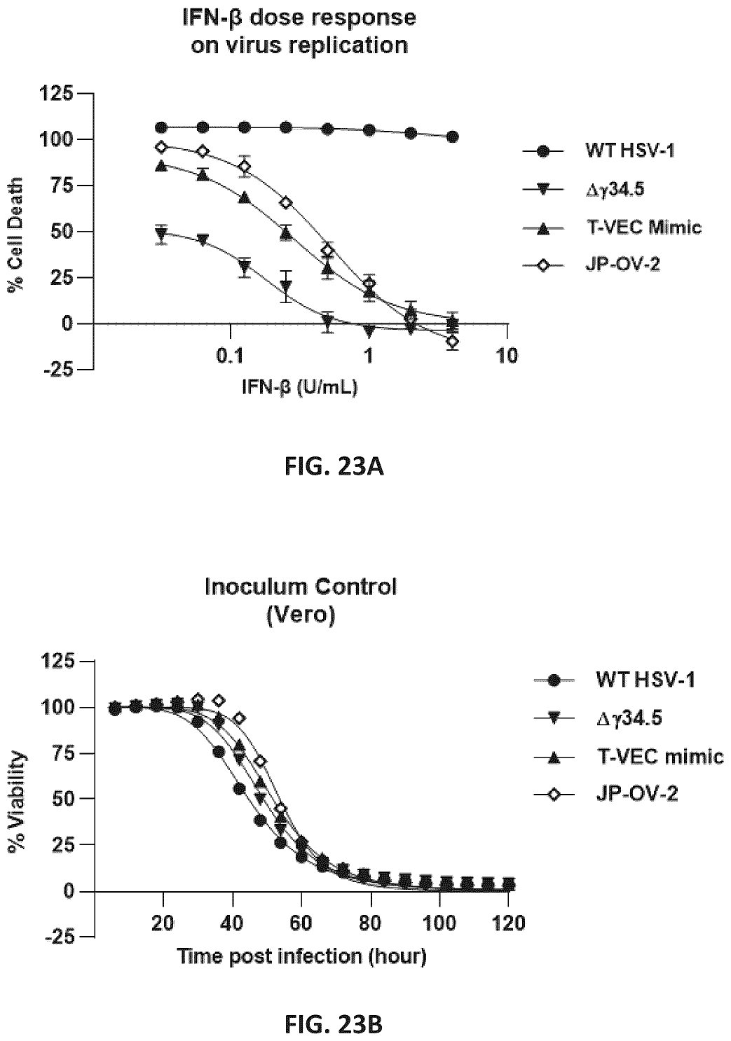

As discussed above, JP-OV-2 is designed to replicate in tumor cells, and not (or to a lesser extent) in normal cells due to its reduced ability to overcome innate anti-viral responses, which are known to be attenuated in cancer cells. Therefore, the sensitivity of JP-OV-2 to Type I IFN, a key mediator of the innate anti-viral response, was tested and compared to that of Δγ34.5 and a previously-reported genetically engineered oncolytic herpesvirus mimic. As shown in FIGS. 23A-23B, JP-OV-2 showed an intermediate level of sensitivity to IFN. It was more resistant than the highly attenuated Δγ34.5 virus, but was considerably more sensitive than WT HSV-1. While JP-OV-2 resistance to IFN was similar to the a previously-reported genetically engineered oncolytic herpesvirus mimic, an improvement was observed for JP-OV-2 over the previously-reported genetically engineered oncolytic herpesvirus mimic, consistent with the innate stealth functions encoded by JP-OV-2. These results aligned with the concept of cancer-selective replication of OVs, supporting that JP-OV-2 would efficiently replicate in tumor cells with aberrant anti-viral innate response, but not in normal cells with an intact IFN response.

### Viral Replication and In Vivo Tumor Growth Inhibition

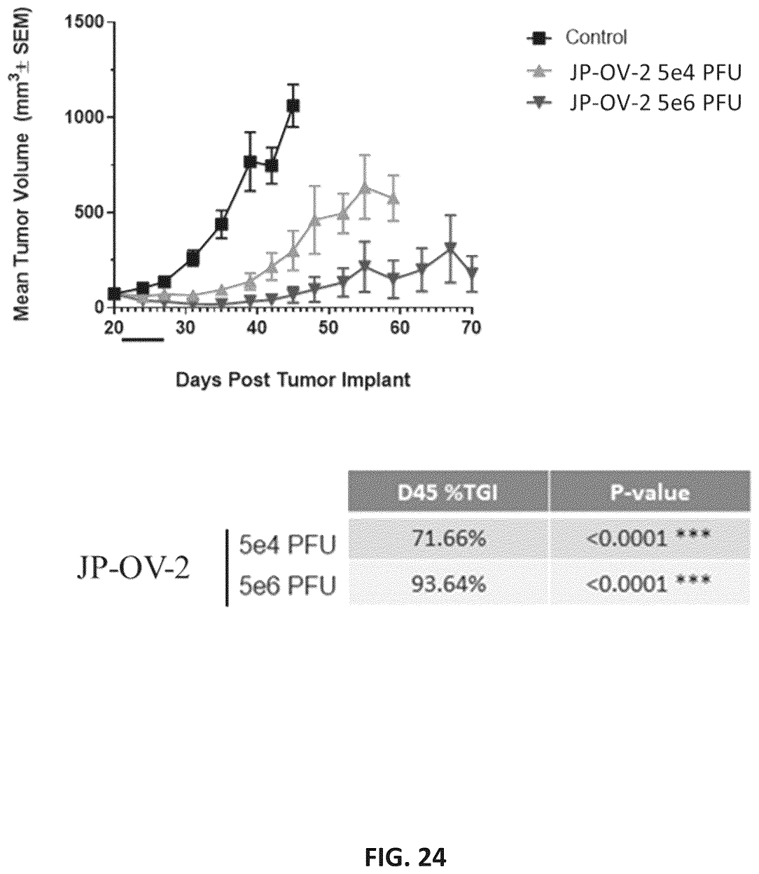

Viral replication of JP-OV-2 was assessed in vivo in a human xenograft tumor model using the human lung cancer H1299 tumors. Use of a human xenograft model allows for virus replication and direct tumor killing due to viral replication. However, payload activity cannot be assessed since human xenograft studies are performed in athymic Nude mice, which lack an intact immune system. Tumor growth inhibition (TGI) following IT injection of JP-OV-2 would indicate viral replication is occurring in the tumor. Two different doses of JP-OV-2 (5×104 and 5×106 PFU/injection) were administered to assess direct tumor cell killing. As shown in FIG. 24, the low dose of JP-OV-2 (5×104 PFU/injection) was efficacious, with TGI of 71.7% compared to vehicle control. The high dose (5×106 PFU/injection) resulted in 93.6% TGI, with 4 out of 10 animals cured by the end of the study.

### Bioactivity of Payloads Expressed from JP-OV-2

hFLT3L

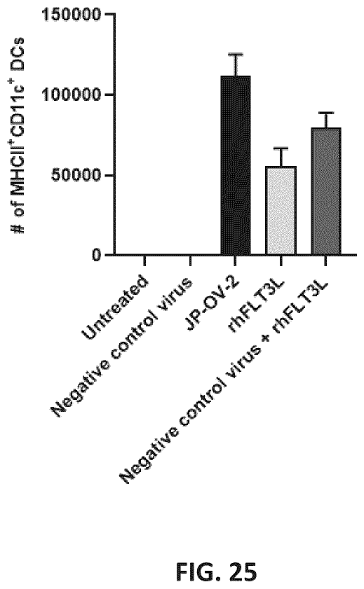

Bioactivity of the hFLT3L payload expressed by JP-OV-2 was assessed using the DC differentiation assay described in Example 2 herein. As shown in FIG. 25, mouse bone marrow cells were differentiated into MHCII⁺CD11c⁺ DCs after exposure to supernatant from cells infected with JP-OV-2, but not supernatant of a negative control virus that does not express hFLT3L. A slight enhancement in DC differentiation was observed for virally-produced hFLT3L as compared to recombinant hFLT3L (after confirmation of similar concentrations of hFLT3L by ELISA). Unexpectedly, spiking the supernatant from the negative control virus with recombinant hFLT3L also led to an increase in activity, suggesting that some viral components may synergize with hFLT3L to further enhance DC differentiation.

hscIL-12

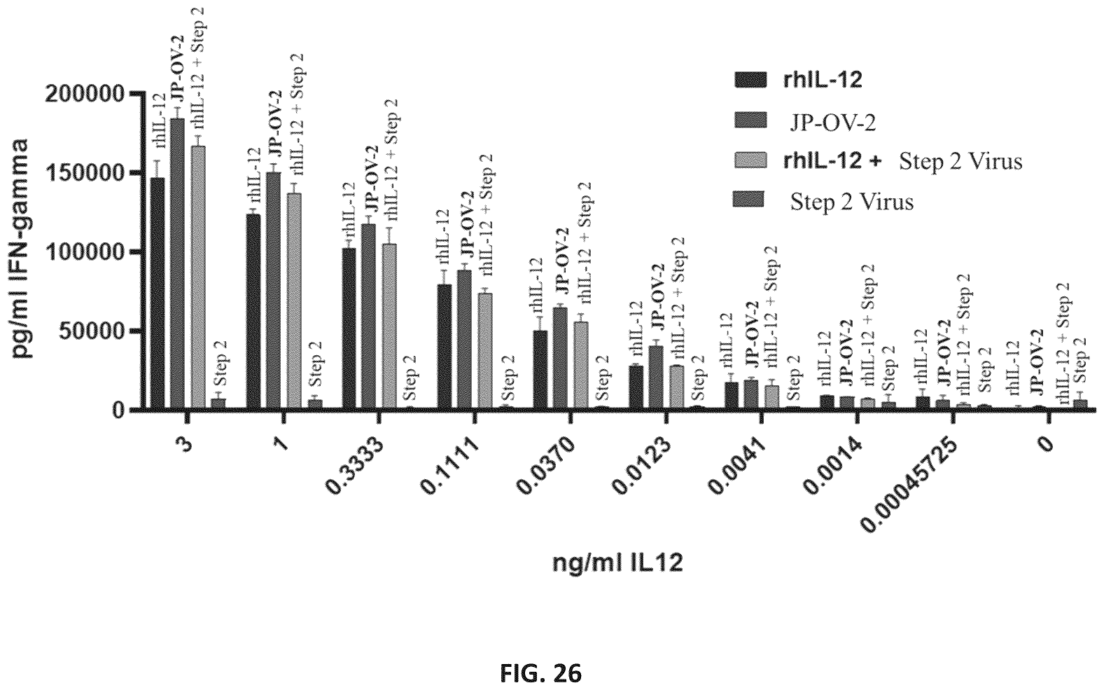

Bioactivity of the hscIL-12 payload produced by JP-OV-2 was assessed based on production of IFNγ by human PBMCs. Briefly, IFNγ produced by human PBMCs was assessed by ELISA upon treatment with suboptimal PHA/PMA stimulation for T cell activation and then recombinant hIL-12 (rhIL-12), the supernatant of Vero cells infected (MOI=1) with JP-OV-2 or Step 2 virus lacking hscIL-12, or rhIL-12 spiked in the supernatant of Vero cells infected with Step 2 virus. The amount of hIL-12 in the supernatant was determined by ELISA. As shown in FIG. 26, human PBMCs produced IFNγ after treatment with supernatant from cells infected with JP-OV-2 but not Step 2 virus, which does not express hscIL-12. Virally-produced hscIL-12 was equally bioactive as recombinant hIL-12, and the activity of recombinant hIL-12 was not affected by supernatant from the Step 2 virus.

### Example 6: Design and Engineering of a Mouse Surrogate Virus for JP-OV-2, an HSV-1 Oncolytic Virus with Stealth Immune Functions and Synergistic Immunomodulatory Payloads

This Example describes the design and engineering of a mouse surrogate virus for JP-OV-2, an HSV-1 OV as described in Examples 3-5, above.

Testing HSV-1 OVs pre-clinically in murine models is complex and challenging. As a human virus, HSV-1 is well adapted to replicate in human cells and interact with human proteins. However, since mice are not a natural host for this human virus, its interactions with mouse protein homologs are in many cases less efficient, or less or non-functional, resulting in reduced human OV replication in mouse cells. In addition, unlike human tumors, which generally have abnormal innate anti-viral signaling pathways, mouse tumors often have an intact response (Schreiber et al. The lytic activity of VSV-GP treatment dominates the therapeutic effects in a syngeneic model of lung cancer. Br J Cancer. 2019; 121(8):647-658), leading to further reduced human OV replication in mouse cells. As a result, HSV-1-based OVs do not generally replicate well in syngeneic mouse cancer models, resulting in decreased efficacy, due to both a reduction in direct tumor cell lysis as well as lower payload expression. In addition, mouse cross-reactive payloads must be used in mouse syngeneic models, both for IT payload injection as well as viral expression, or alternatively, knock-in mice must be used to express the human targets for human payloads.

Given the challenges and experimental caveats for testing an HSV-1 OV in vivo, a combination of strategies was used for testing JP-OV-2. As described below, several studies were performed using direct injection of mouse surrogate payloads, or using human payloads and knock-in mice for the relevant human gene. In addition, a human HSV-1 OV encoding mouse surrogate payloads was engineered, referred to as mJP-OV-2. As described in detail below, this mouse surrogate virus has an identical viral backbone to JP-OV-2, but the genes encoding the hCD40ag, hscIL-12, and hαCTLA-4 payloads were replaced with mouse surrogate versions, while the genetic architecture, immune stealth components, and expression cassettes remained unchanged. In addition, the hFLT3L payload was kept unchanged in the γ34.5 locus because this payload is cross-reactive in mouse.

### Mouse Surrogate Payload Design and Testing

A mouse surrogate virus identical to JP-OV-2, but with mouse bioactive payloads, was constructed. The mouse surrogate virus was termed mJP-OV-2. To ensure that the mouse surrogate payloads chosen for the mouse surrogate virus had bioactivity comparable to the corresponding human payloads expressed from JP-OV-2, the bioactivity of the mouse and human payloads was compared head-to-head in the same assay when possible.

### Mouse CD40 Agonist Payload

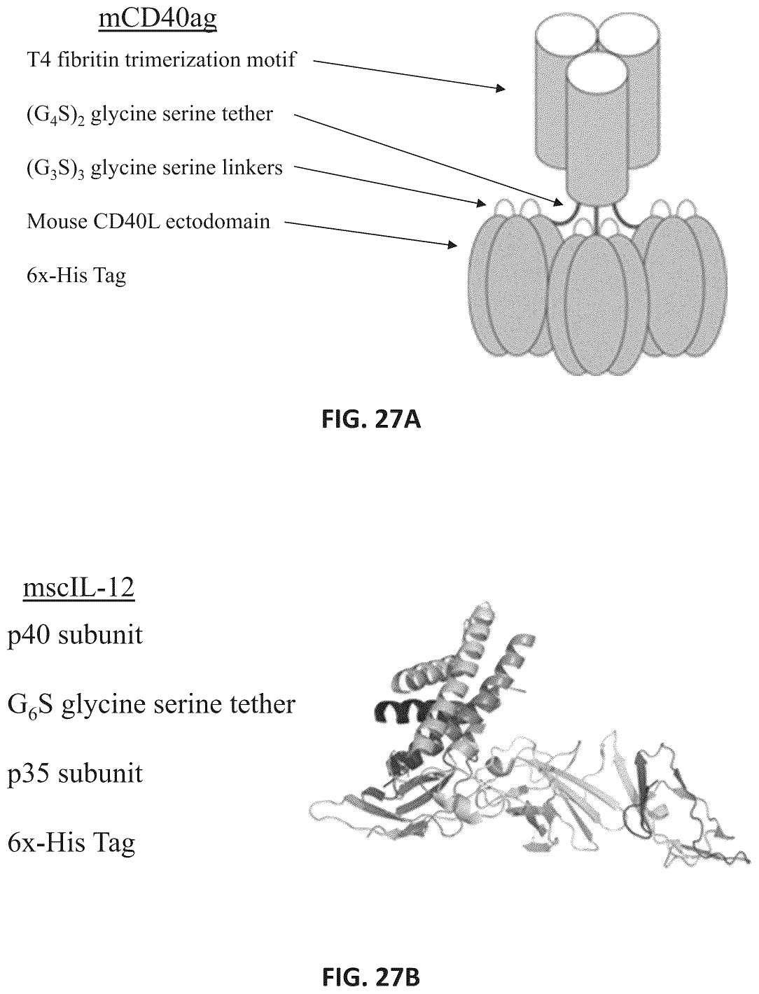

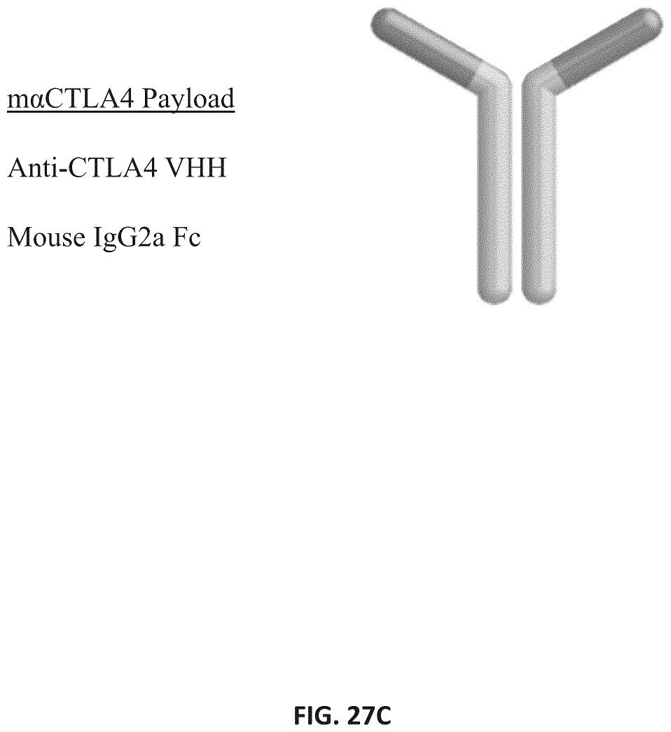

As shown in FIG. 27A, similar to the design of the human CD40 agonist in JP-OV-2 (i.e., the hCD40ag payload), the mouse surrogate CD40 agonist, termed “mCD40ag,” is a trimerized, C-terminal fusion protein consisting of trimeric bundles of mouse CD40L ectodomains (Uniprot: P27548) that bind to the CD40 receptor protein (Uniprot: P27512) on APCs to promote anti-cancer activity. The molecule features the 27-amino-acid trimerization motif from the fibritin protein of T4 phage (Uniprot: P10104) fused with a glycine/serine linker to the N-terminus CD40L ectodomain bundles. The CD40L ectodomain trimer bundles consist of individual CD40L protomers fused together by glycine/serine linkers from the C-terminus of one protomer to the N-terminus of the subsequent protomer to form single polypeptide trimeric bundles. This molecule was designed to promote stable and predictable oligomerization of CD40L without the need for mouse IgG2a Fc. A 6-mer polyhistidine sequence was added to the Cterminus of the final CD40L protomer to aid in purification of recombinant protein by Ni-NTA resin, however this feature is not present in the final virus construct.

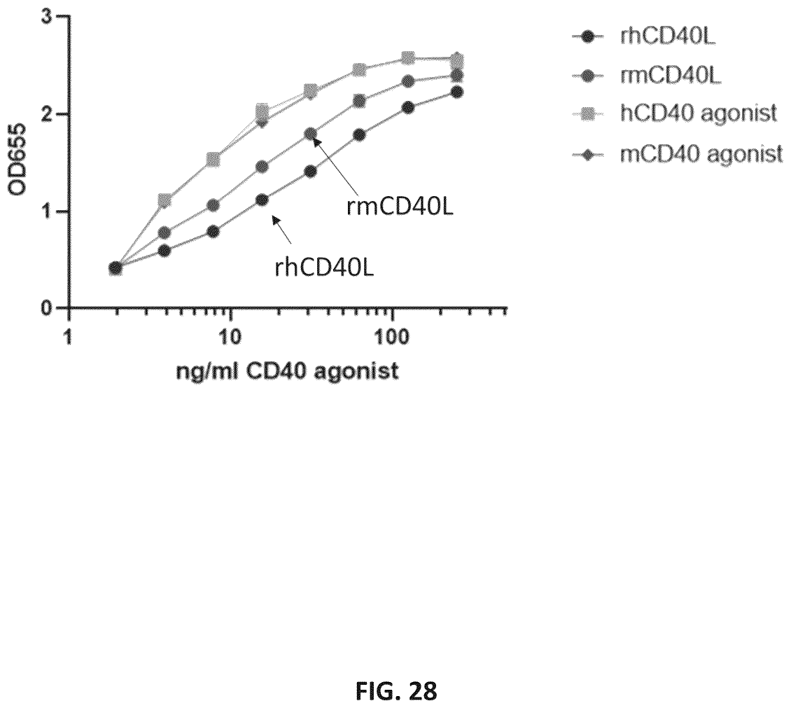

The bioactivity of purified hCD40ag was compared to purified mCD40ag in a HEK-Blue reporter assay. This assay consists of HEK293 cells stably transfected with the human CD40 gene and an NFκB-inducible secreted alkaline phosphatase (SEAP) construct. The reporter cells respond to CD40 agonist binding by the production of a colorimetric readout. In this assay, the bioactivity of the mouse and human CD40 agonists is measured following binding to the stably expressed human CD40 receptor. Mouse CD40L is cross-reactive in this assay, and recombinant mouse CD40L and human CD40L demonstrated similar bioactivity. As shown in FIG. 28, the hCD40ag and mCD40ag payloads showed almost identical bioactivity, supporting the choice of mCD40ag as the surrogate CD40 agonist payload in the surrogate virus.

### IL-12 Payload

Similar to the design of the hscIL-12 payload protein in JP-OV-2, the mouse surrogate was designed as a mouse IL-12 fusion protein featuring the mouse p40 subunit, also known as IL-12 subunit beta (Uniprot: P43432), from IL-12 fused with a glycine/serine linker to the N-terminus of mouse p35, also known as IL-12 subunit alpha (Uniprot: P43431) (FIG. 27B). This molecule was designed to promote stable and predictable heterodimerization of the p40 and p35 subunits to generate functionally active IL-12, and to promote anti-cancer immunity responses by activating T cells and NK cells in the TME. A 6-mer polyhistidine sequence, C-terminal of the p35 subunit, was added to aid in purification of recombinant protein by Ni-NTA resin. However, this purification tag is not present in the final virus construct.

The purified hscIL-12 payload was tested against the mouse surrogate payload, mscIL-12, in a hIL-12 receptor HEK-Blue reporter assay. This assay uses HEK293 reporter cells stably transfected with the human IL-12 receptor gene and an NF-κB-inducible secreted alkaline phosphatase (SEAP). The reporter cells respond to IL-12 binding by the production of a colorimetric readout. The bioactivity of mouse and human IL-12 is measured following binding to stably expressed human IL-12 receptor. Mouse IL-12 is cross-reactive in this assay.

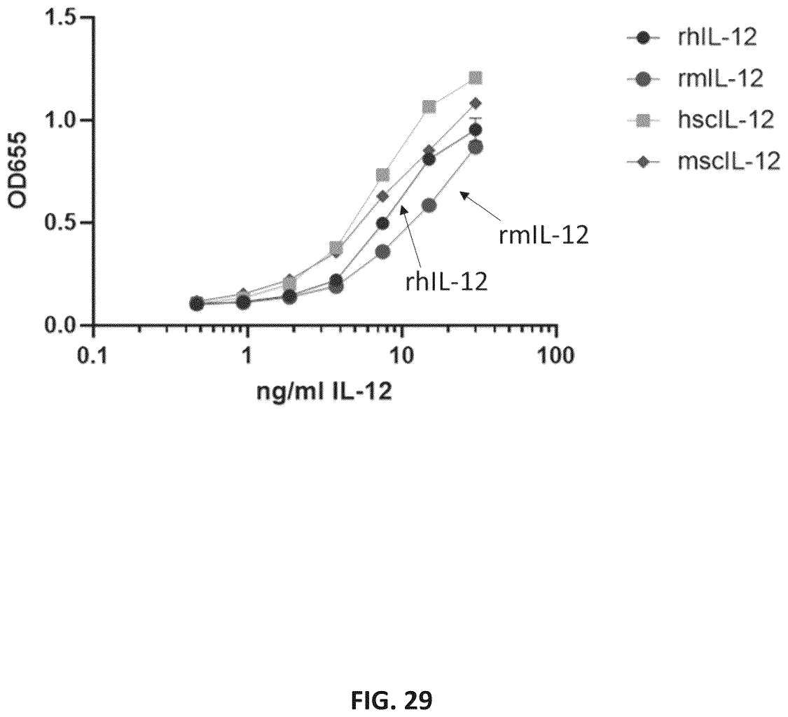

As shown in FIG. 29, recombinant WT mouse IL-12 was slightly less bioactive compared to WT human IL-12. The same pattern was observed with the human and mouse payloads: mscIL-12 was slightly less active than hscIL-12. However, the bioactivity was similar between the human and mouse payloads, and endorses the selection of the mscIL-12 surrogate payload.

### CTLA-4 Antagonist Payload

The mouse surrogate for hαCTLA-4, the human anti-CTLA-4 antagonist, was termed mαCTLA-4 and is an IgG2a antibody that binds to mouse CTLA-4 (Uniprot ID: P09793) on T cells. The molecule comprises an anti-CTLA-4 VHH fused to the heavy chain of mouse IgG2a Fc (FIG. 27C).

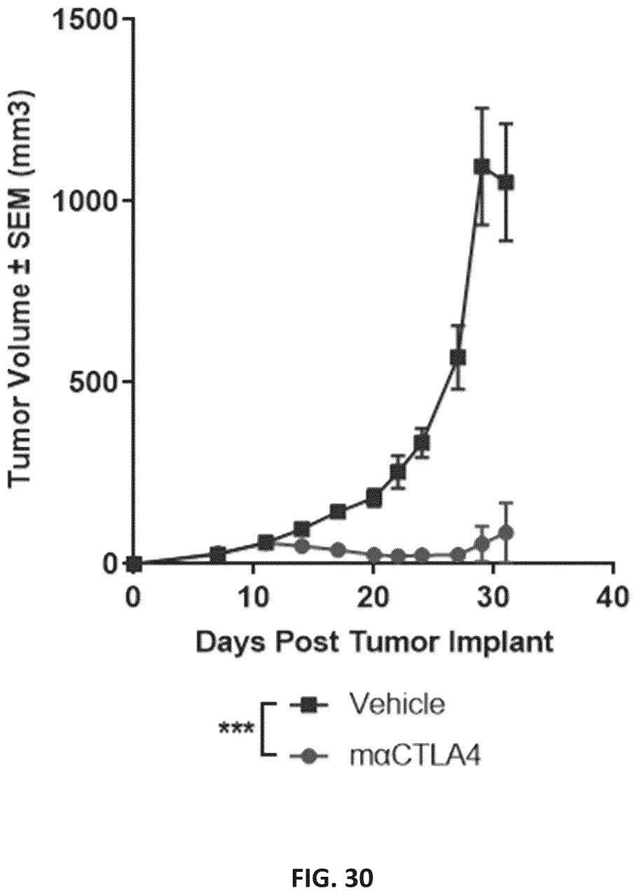

The bioactivity of the mαCTLA-4 surrogate payload was assessed by injection of purified mαCTLA-4 in a single MC38-5AG tumor in WT mice. Briefly, WT mice were implanted with single MC38-5AG tumors and treated IT once every 3 days for 4 doses with purified mαCTLA-4 payload. As shown in FIG. 30, mαCTLA-4 demonstrated anti-tumor activity comparable to the results shown in FIG. 5A, which used anti-human CTLA-4 molecules in human CTLA-4 knock-in animals, although these studies were performed in different animal models.

hFLT3L Payload

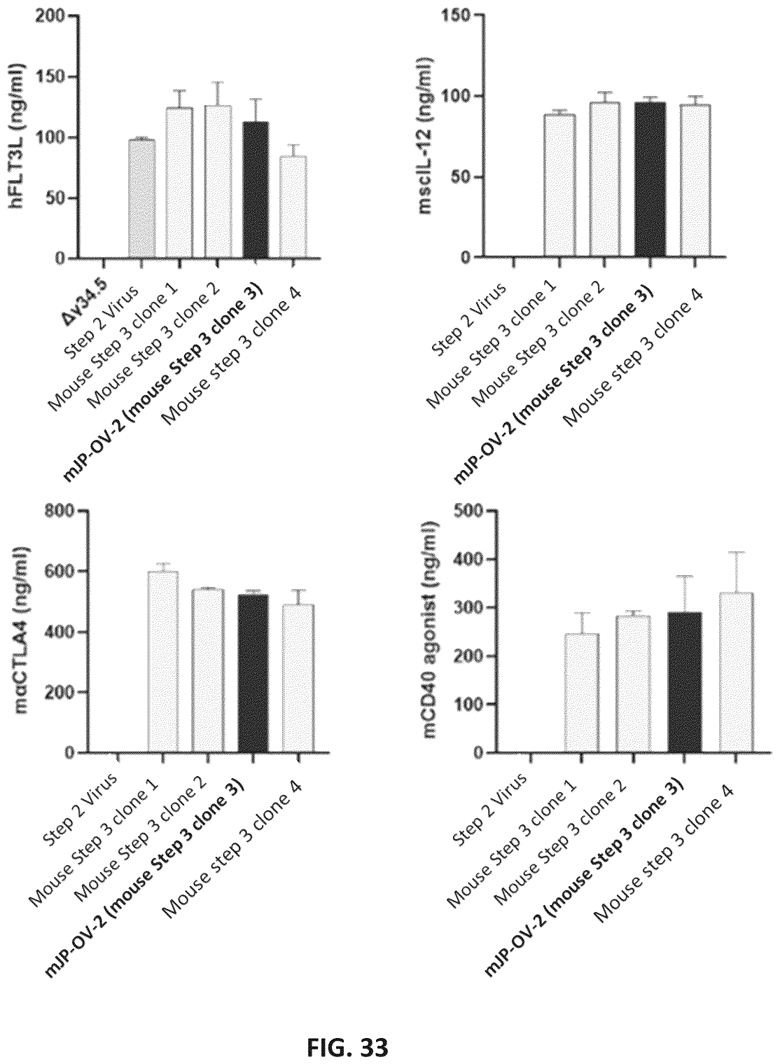

The hFLT3L protein is cross-reactive in mouse. Therefore, a bioactivity comparison was not necessary since hFLT3L was similarly expressed from both JP-OV-2 and the mouse surrogate virus (mJP-OV-2). See, for example, FIG. 17 and FIG. 33 (discussed in detail below).

### Mouse Surrogate Virus Construction

To construct mJP-OV-2, the mouse surrogate virus, the eGFP cassette within the US10-12 locus of the Step 2 virus as described in Example 4, above, was replaced by a cassette encoding the mαCTLA-4, mCD40ag, and mscIL-12 payloads, while conserving an identical promoter and polyA architecture. The mouse surrogate expression cassette was termed m37E. This engineering step was identical to the one leading from Step 2 virus to JP-OV-2 as described in Example 4, above (see, FIGS. 15D-15E).

The fourth payload, hFLT3L, located in the γ34.5 locus of the Step 2 virus, is cross-reactive in mice (O'Keeffe et al. Effects of administration of progenipoietin 1, Flt-3 ligand, granulocyte colony-stimulating factor, and pegylated granulocyte-macrophage colony-stimulating factor on dendritic cell subsets in mice. Blood. 2002; 99(6):2122-30) and was conserved.

The human codon-optimized IE copy of US11 located at the 3′end of the cassette (hCoUS11, FIG. 15E) was also conserved and was not codon-optimized for murine expression due to similar codon usage frequency between mice and humans.

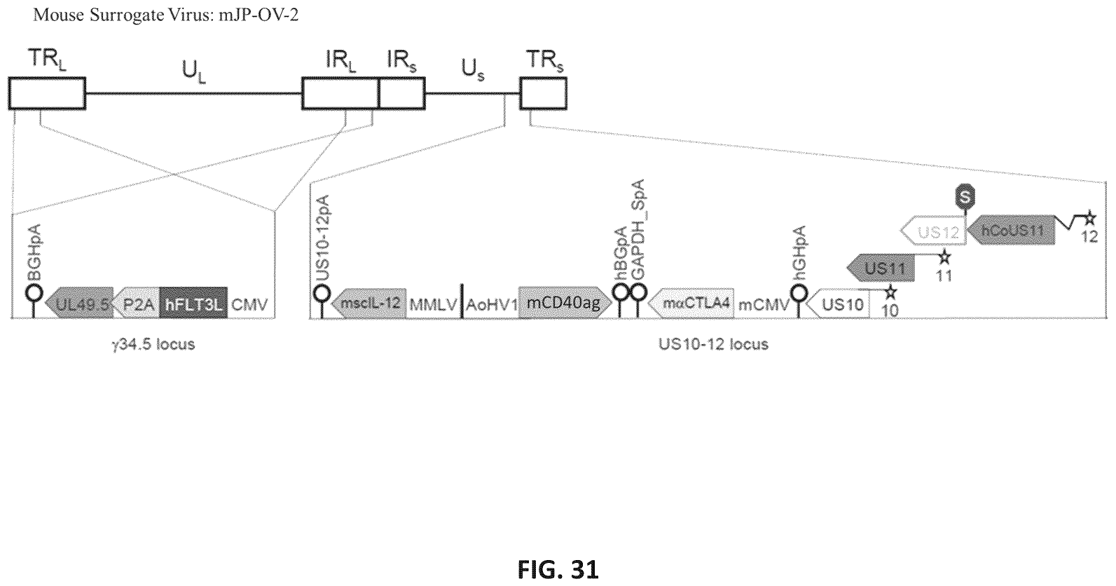

The genome architecture of the mouse surrogate virus, mJP-OV-2, is provided in FIG. 31.

Briefly, Step 2 viral DNA was co-transfected with pTVTRsh11CO10 pA_m37E target vector (m37E cassette with viral DNA flanking sequences) to replace the eGFP cassette. Recombinant virus plaques, which displayed loss of GFP expression, were identified and discriminated from GFP-positive non-recombinant virus plaques by fluorescence microscopy. Recombinant viral clones were isolated through 3 rounds of plaque purification, and recombinant clones were confirmed at each round of plaque purification using microscopy and PCR assays. Four recombinant clones were selected for further characterization: mouse Step 3 clone 1, mouse Step 3 clone 2, mouse Step 3 clone 3, and mouse Step 3 clone 4.

Master seed stocks (MSS) were prepared, and the fitness of the different clones was assessed with the same series of release assays as for JP-OV-2 as described above. Mouse Step 3 clone 3, was selected as the final mouse surrogate virus, termed mJP-OV-2, as described in detail below.

### Genomic Integrity

The presence of the transgene cassettes in MSS stocks was confirmed by PCR assay (data not shown). To confirm the genome integrity at the γ34.5 and US10-12 loci of the recombinant viruses, Southern blotting analysis was performed using viral DNA isolated from infected Vero cells (MOI=1) at 24 hours post infection. The results confirmed the integrity of the γ34.5 and US1012 loci (data not shown).

### Stealth and Payload Protein Expression

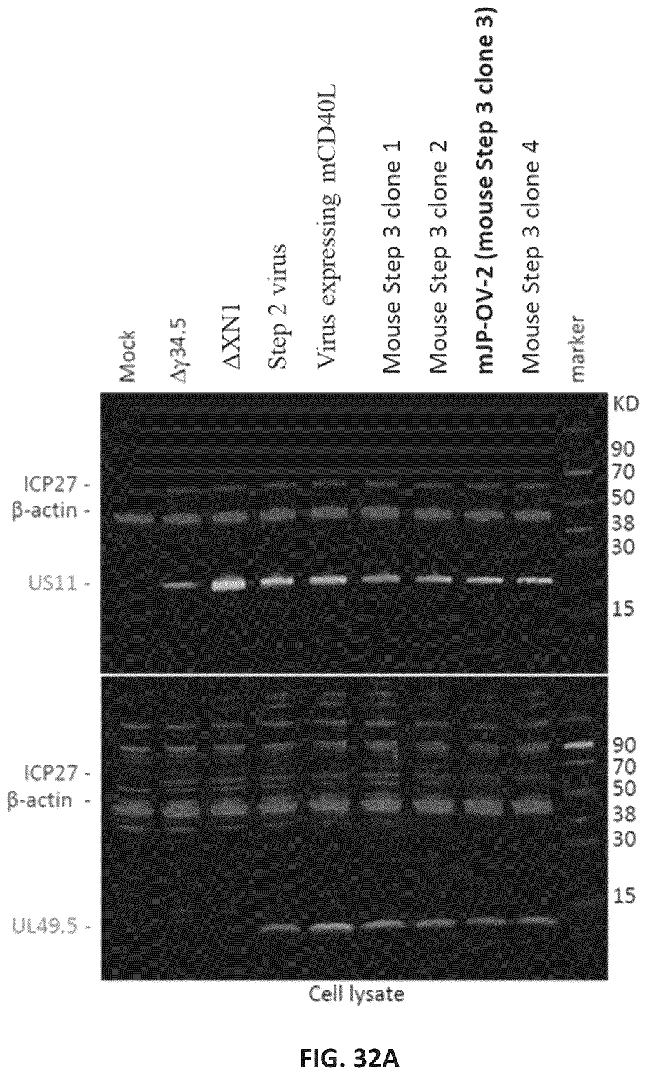

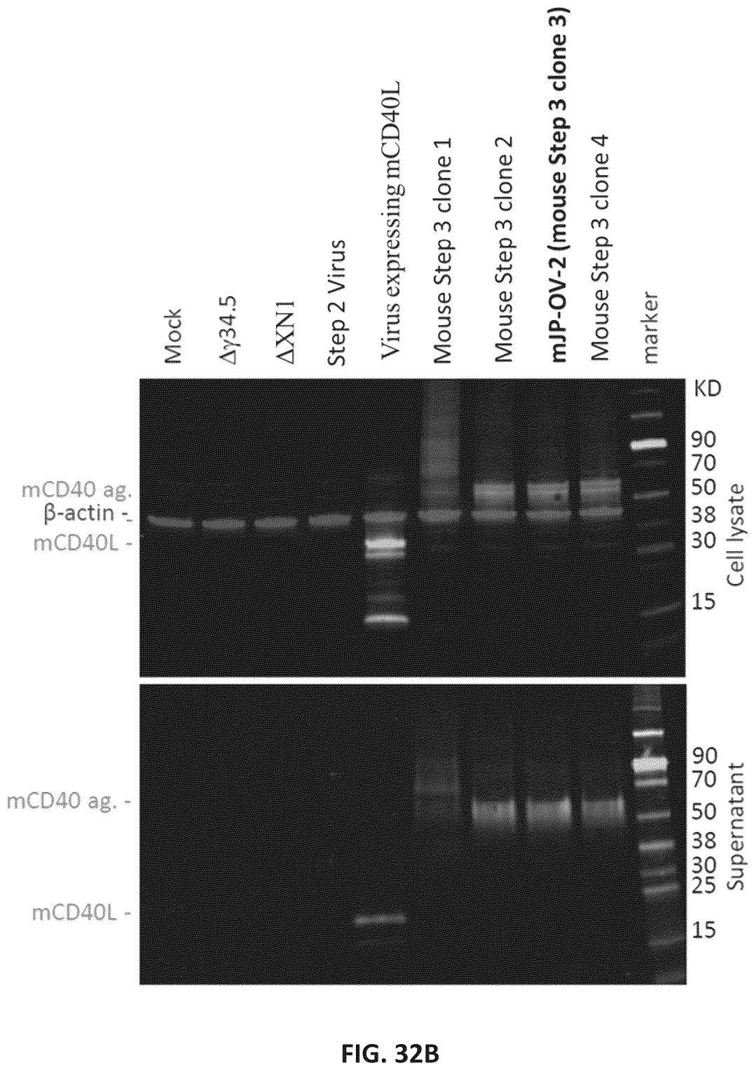

The expression of the two immune stealth function proteins (i.e., UL49.5 and US11), and of the mCD40ag payload was assessed by Western blot using cells infected by the four mouse surrogate virus clones. Briefly, protein expression was analyzed 6 hours following the infection of Vero cells with the four mouse surrogate virus clones at MOI=5. The Δγ34.5 virus was used as a negative control for IE expression of US11, the ΔXN1 virus was used as a negative control for UL49.5 expression, Step 2 virus is the parental virus and was used as a negative control for mCD40ag expression, and a virus that contains a cassette in the γ34.5 locus expressing mCD40L was used as a positive control for mCD40ag expression. ICP27, an IE viral protein, was used as an infection control and is present in all tested viruses. As shown in FIG. 32A, immunoblotting of cell lysates from infected cells with anti-US11 and anti-UL49.5 antibodies confirmed the expression of both immune stealth function proteins in mJP-OV-2 at levels comparable with the parental Step 2 virus. Immunoblotting of whole-cell lysates and supernatants of infected cells with anti-mCD40L antibodies also showed the expression of an immunoreactive band slightly above the 50 kDa marker for mJP-OV-2 that was compatible with the 54.3 kDa theoretical molecular weight of the mCD40ag monomer (FIG. 32B).

Expression of the four secreted payloads was assessed for the mouse surrogate virus mJP-OV-2. Briefly, Vero cells were infected (MOI=1) for 24 hours with control Δγ34.5 virus, precursor Step 2 virus, and the 4 plaque-purified mouse surrogate virus clones selected during the construction of the mouse surrogate virus. Supernatants of infected cells were analyzed by ELISA. As shown in FIG. 33, the levels of the hFLT3L, mscIL-12, and mCD40ag payloads expressed by mJP-OV-2 were all highly comparable to what was observed for the human payloads from JP-OV-2 (FIG. 17), particularly considering that minor differences in cell density and time points can impact expression levels, and quantification can be affected by the required differences in the ELISA kits used for mouse versus human payloads. The mouse anti-CTLA-4 antagonist payload (mαCTLA-4) did show about 4-fold higher expression in mJP-OV-2, as compared to the human payload (hαCTLA-4) expression from JP-OV-2, which is consistent with the generally improved expression of a VHH compared to an scFv-Fc. However, this difference in expression was a relatively minor difference. Therefore, expression of all payloads from mJP-OV-2 was considered approximately equivalent to JP-OV-2.

### Cytotoxicity

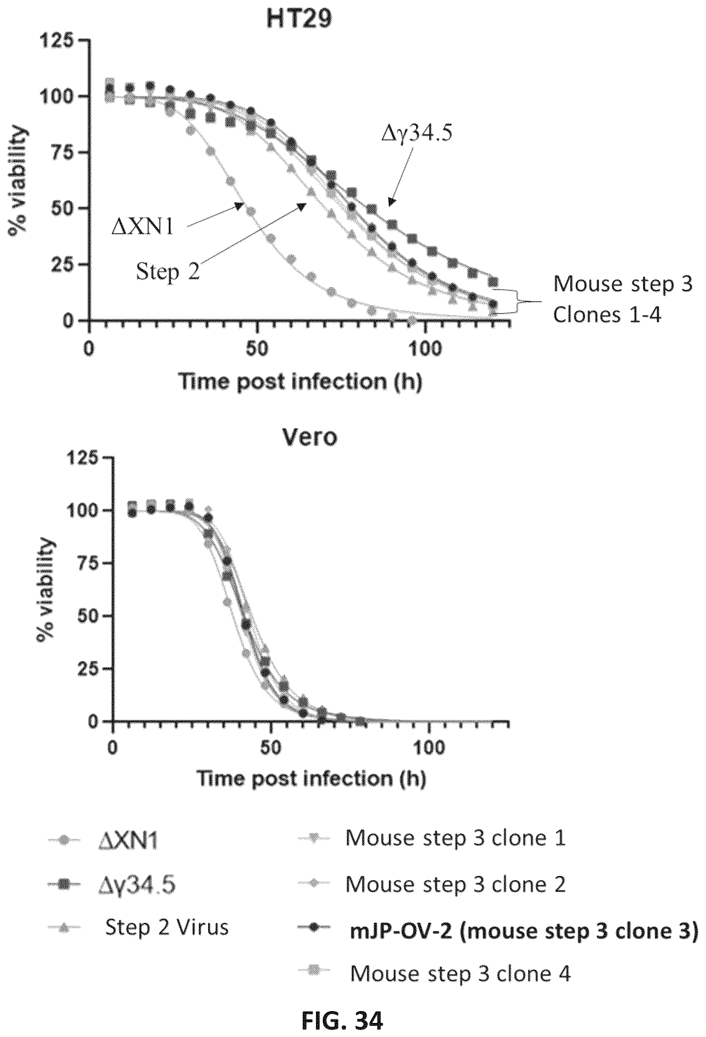

Mouse surrogate virus cytotoxicity over time was measured via xCelligence, as described above in Example 5 (see, FIG. 18). As shown in FIG. 34, on stringent HT29 cells, mJP-OV-2 replicated approximately equally to parental Step 2 virus. Control Vero cells indicated that equivalent amounts of all viruses were added to the assays.

### Final Selection and Sequencing of mJP-OV-2

Based on the above results, the mouse Step 3 clone 3 virus, mJP-OV-2, was selected as the final mouse surrogate virus clone.

mJP-OV-2 was sequenced using the Pacbio Sequel SMRT Cell long-read whole-genome sequencing method, and annotated with reference sequences derived from parental virus genome and transgene cassette. Overall, the long read whole-genome sequencing did not identify any structural variants, large insertions, or deletions of concern in mJP-OV-2, confirming the overall integrity of the mouse surrogate virus genome. The sequencing detected the following mutation in an open read frame of the virus: base 72885C->CTGGGGCTGGGGT (“CTGGGGCTGGGGT” disclosed as SEQ ID NO: 321). This 12-nucleotide in-frame insertion is in a G-rich repeated region coding for a Q/P-repeat in the UL36 protein. It brings the Q/P repeat number from 21 in the Step 2 virus to 23 in mJP-OV-2. This region has been reported to be variable in length for HSV-1 and HSV2 (Colgrove et al. History and genomic sequence analysis of the herpes simplex virus 1 KOS and KOS1.1 sub-strains. Virology. 2016; 487:215-21; and Colgrove et al. Genomic sequences of a low passage herpes simplex virus 2 clinical isolate and its plaque-purified derivative strain. Virology. 2014; 450-451:140-5). It is of unknown consequence but most likely benign.

### Example 7: Functional Testing of a Mouse Surrogate Virus for JP-OV-2, an HSV-1 Oncolytic Virus with Stealth Immune Functions and Synergistic Immunomodulatory Payloads

This Example describes the results of experiments that assessed the functionality of mJP-OV-2, which is a mouse surrogate virus for JP-OV-2, as described above in Example 6.

### Bioactivity of Payloads Expressed by the Mouse Surrogate Virus

The bioactivity of the mCD40ag and mscIL-12 mouse surrogate payloads when expressed by mJP-OV-2, the mouse surrogate virus, was assessed in vitro.

mCD40ag Bioactivity

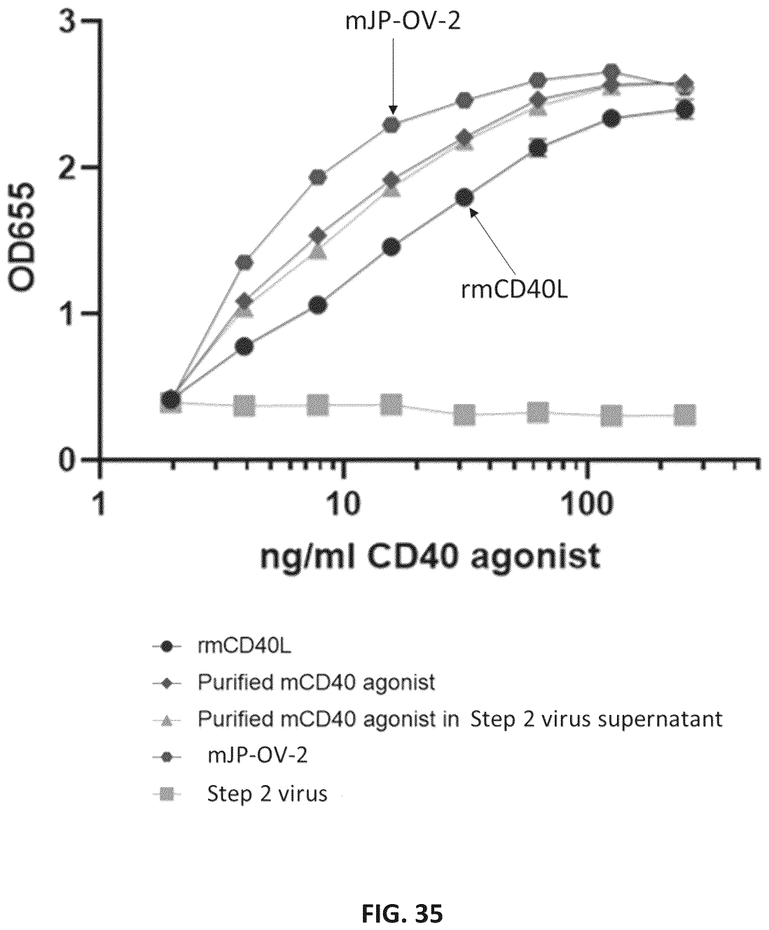

The bioactivity of the mCD40ag mouse surrogate protein was assessed using an hCD40 receptor HEK-Blue reporter assay (see, Example 6, above). The activity of the mCD40ag mouse surrogate protein from supernatant media of Vero cells infected (MOI=1) with mJP-OV-2 was compared to that of recombinant WT mouse CD40L (rmCD40L) or purified mCD40ag mouse surrogate protein diluted in either media or supernatant from cells infected with the negative control Step 2 virus. As shown in FIG. 35, the mCD40ag mouse surrogate protein expressed from the mouse surrogate virus was bioactive, and was more potent than commercially available recombinant mCD40L, and slightly more potent than purified mCD40ag mouse surrogate protein. The supernatant of cells infected with mCD40L-null virus (Step 2 virus) was inactive. Purified mCD40ag mouse surrogate protein showed similar activity when diluted in media or when spiked into Step 2 virus supernatant, demonstrating that the increase in mCD40ag mouse surrogate protein bioactivity from the supernatant of cells infected with mJP-OV-2 was not related to any contribution of infected cell supernatant.

mscIL-12 Bioactivity

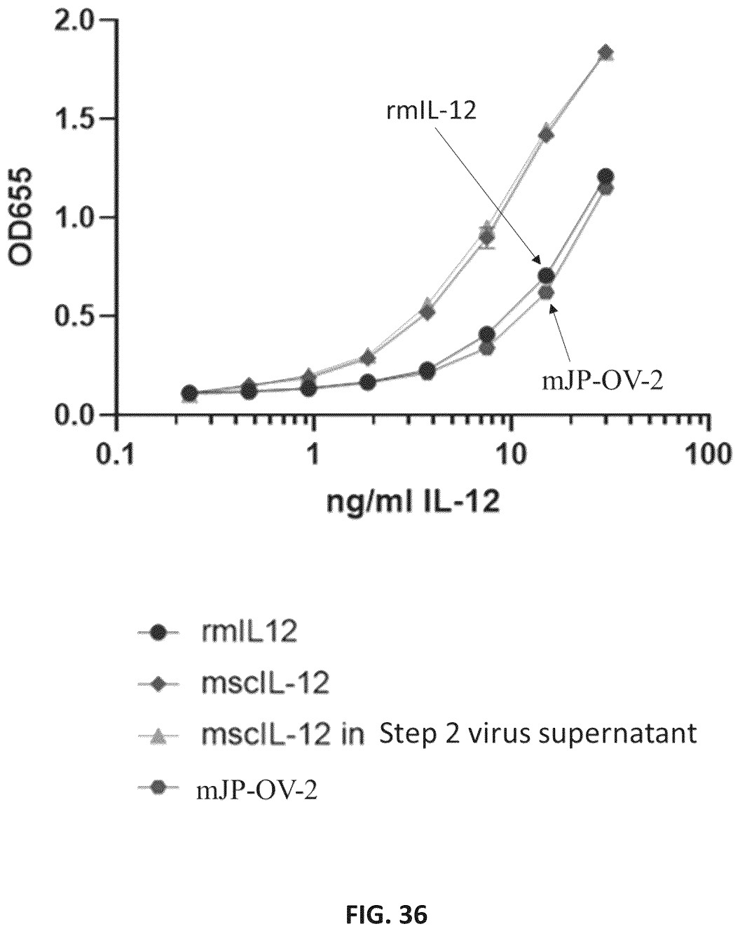

The bioactivity of the mscIL-12 mouse surrogate payload protein was assessed using an hIL-12 receptor HEK-Blue reporter assay (see, Example 6). The activity of the mscIL-12 mouse surrogate protein from cells infected with mJP-OV-2 was compared to that of commercially available recombinant WT mIL-12 or purified mscIL-12 mouse surrogate protein diluted in either media or supernatant from cells infected with negative control Step 2 virus. The amount of mscIL-12 protein in the supernatant was quantified by ELISA. As shown in FIG. 36, mscIL-12 protein from cells infected with mJP-OV-2 had similar bioactivity to an equal amount of recombinant WT mIL-12. Purified mscIL-12 showed higher bioactivity in this assay as compared to mscIL-12 expressed from mJP-OV-2, which does not appear to be linked to the contribution of the infected cell supernatant, since spiking purified mscIL-12 into the negative control supernatant did not change the bioactivity. The difference in activity may result from the polyhistidine tag on purified mscIL-12, which may have affected quantification or bioactivity, and which is not present on the untagged mscIL-12 expressed by mJP-OV-2. Thus, it was demonstrated that mJP-OV-2 expresses bioactive mscIL-12.

The bioactivity of the mαCTLA-4 mouse surrogate payload was not tested further for virus-produced mαCTLA-4 due to the lack of a suitable in vitro assay. However, the ELISA experiment shown in FIG. 33 demonstrates that mαCTLA-4 expressed from mJP-OV-2 was active and capable of binding immobilized mCTLA-4 used in the ELISA.

The hFLT3L protein expressed by mJP-OV-2 was not tested further because the hFLT3L-expressing construct in mJP-OV-2 (gene and promoter sequence, as well as viral locus) was identical to that of the JP-OV-2 virus, which was previously shown to be bioactive (see, FIG. 25). Further, as described above, the sequence of this region in mJP-OV-2 was confirmed via Pacbio sequencing.

### In Vivo Synergy and Functionality of the Mouse Surrogate Payloads

### Synergy of Mouse Surrogate Payloads

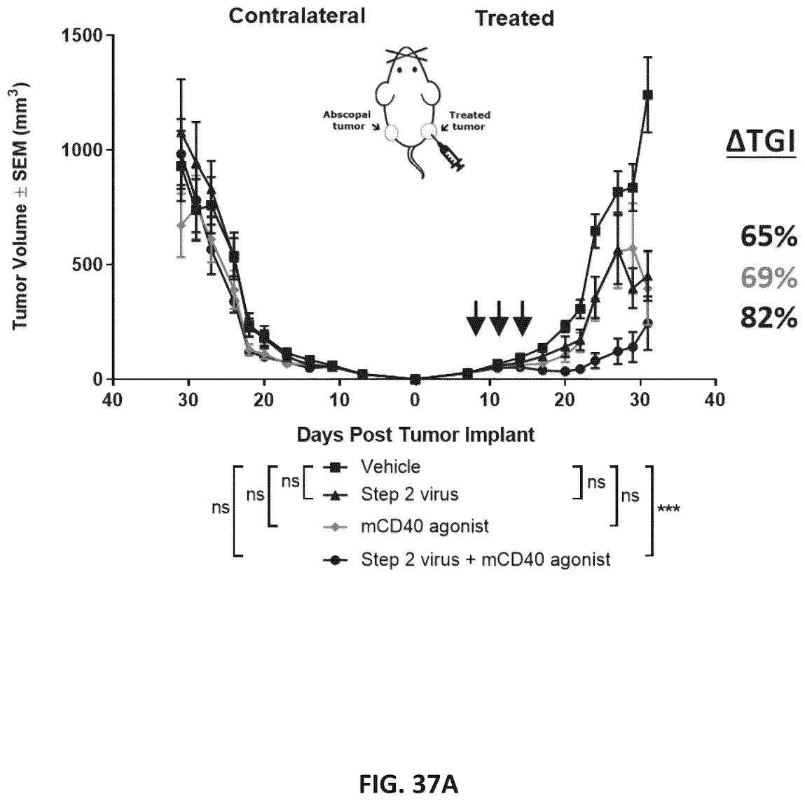

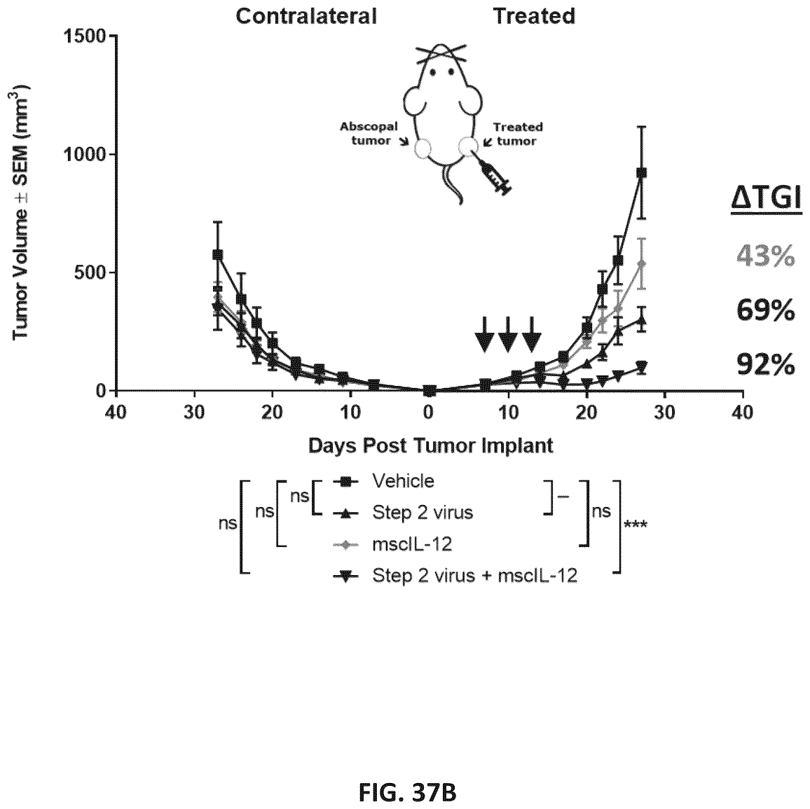

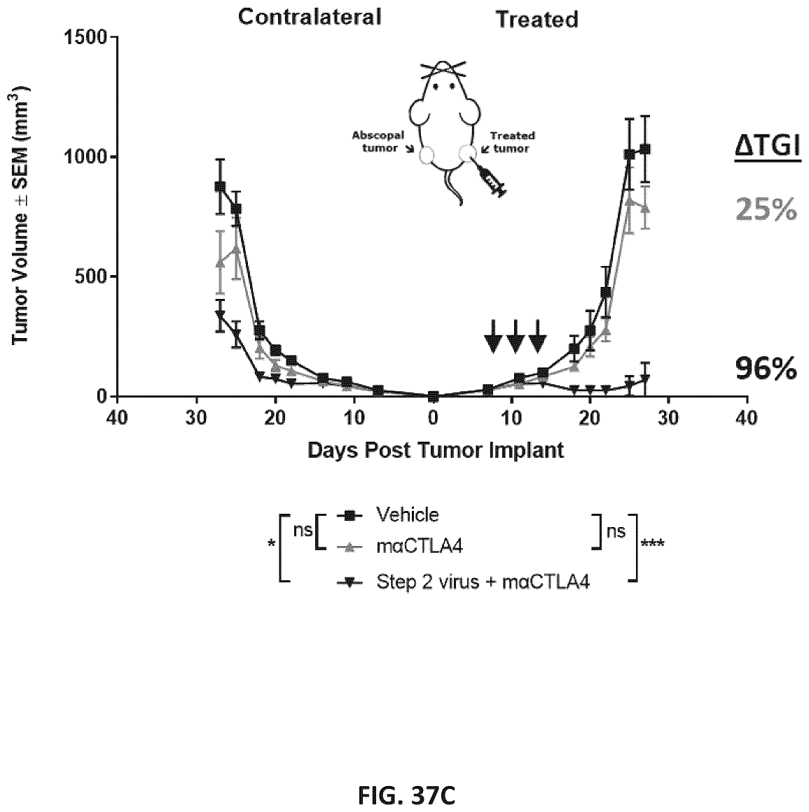

The synergistic activity of the payloads encoded by mJP-OV-2 was first tested using commercially available and/or purified payload proteins combined with a hFLT3L-expressing virus (Step 2 virus) in MC38-5AG mouse syngeneic tumors. The payloads used were FGK4.5 (an anti-mouse CD40 agonist antibody), 9H10 (a monoclonal mouse anti-CTLA-4 antibody), both commercially available, as well as the purified mscIL-12 mouse surrogate payload. In this experiment, the payload proteins and Step 2 virus were intratumorally (IT) injected in only one (right side) of two bilateral tumors in mice. An abscopal effect was defined as efficacy against the untreated tumor (left side), as a demonstration of anti-tumor immune cell activity. As shown in FIGS. 37A-37C, each of the Step 2 virus (hFLT3L-expressing virus), the mouse CD40 agonist antibody, the purified mscIL-12, and the mouse anti-CTLA-4 antibody alone demonstrated growth control in the treated tumor. The combination of the Step 2 virus with the mouse CD40 agonist antibody, mscIL-12, or the mouse anti-CTLA-4 antibody demonstrated superior treated tumor growth control compared to each agent alone. However, combinations of Step 2 virus and each single payload were unable to control the untreated, contralateral, tumor growth, with the exception of the combination of Step 2 virus and the mouse anti-CTLA-4 antibody, which demonstrated a moderate abscopal effect (FIG. 37C).

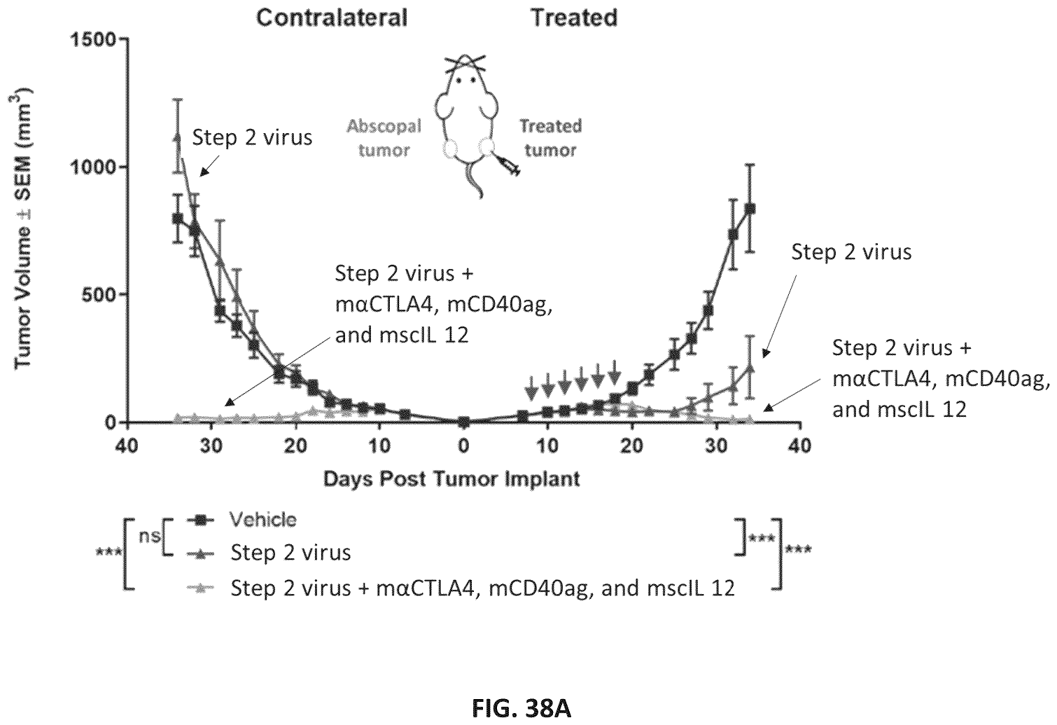

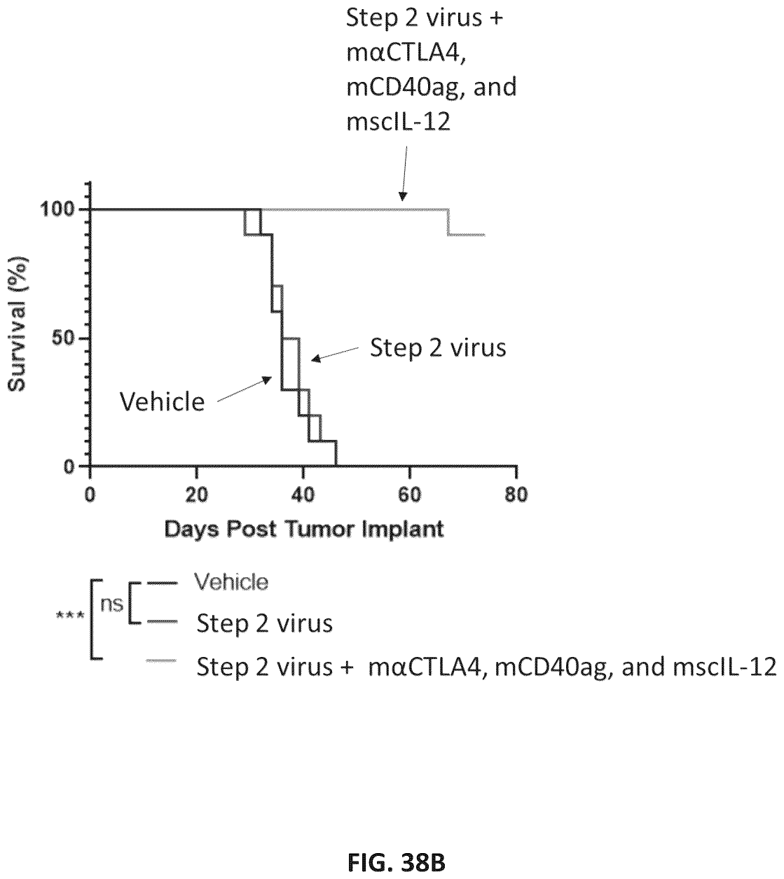

To further characterize the efficacy of the hFLT3L-expressing OV in combination with 3 purified mouse payloads, Step 2 virus (expressing hFLT3L) was simultaneously IT dosed with all three remaining mouse surrogate payloads in the right tumor of a bilateral MC38-5AG syngeneic model. In this experiment, the mouse payloads used were those actually encoded by the surrogate virus mJP-OV-2, i.e., the mαCTLA-4, mCD40ag, and mscIL-12 payloads. Each payload protein was IT administered as purified protein in combination with Step 2 virus. As shown in FIGS. 38A-38B, Step 2 virus alone showed significant control of treated tumor growth, with further improved efficacy when simultaneously injected with the three mouse purified payloads (although this difference was not statistically significant). Step 2 virus injected alone was unable to control untreated tumor growth. In contrast, strong abscopal efficacy was observed when Step 2 virus was combined with the three purified mouse payloads. Survival analysis indicated that 9 of 10 mice treated with the combination of Step 2 virus and payloads were cured, with no detectable tumor on either side (FIG. 38B).

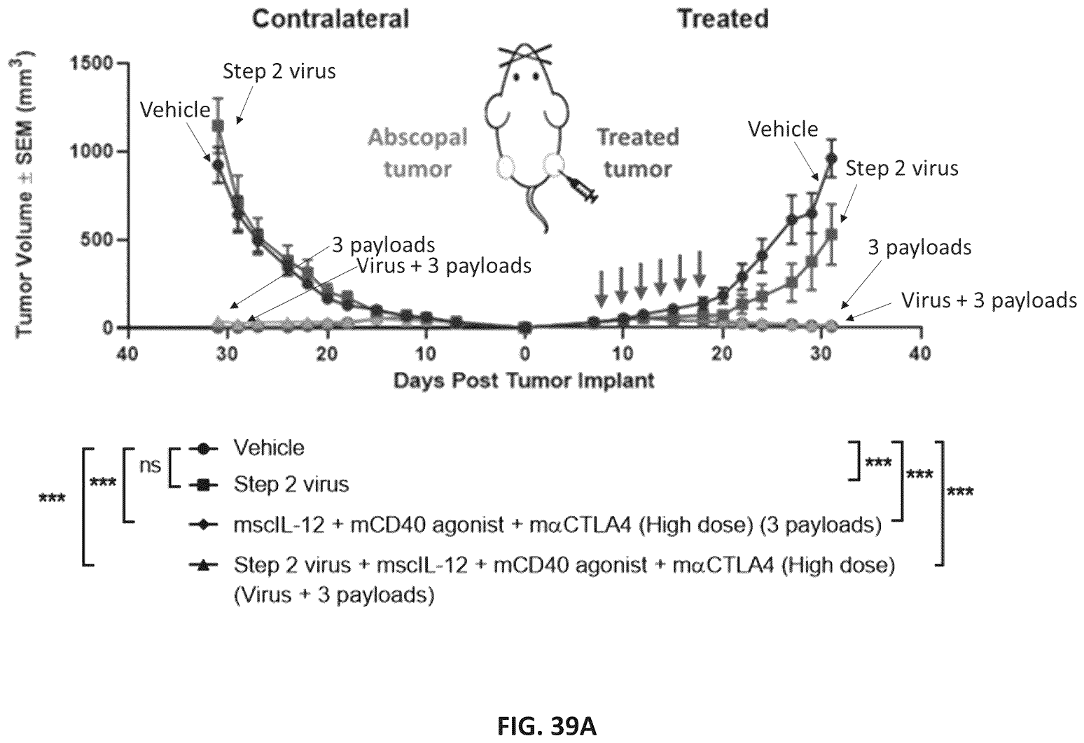

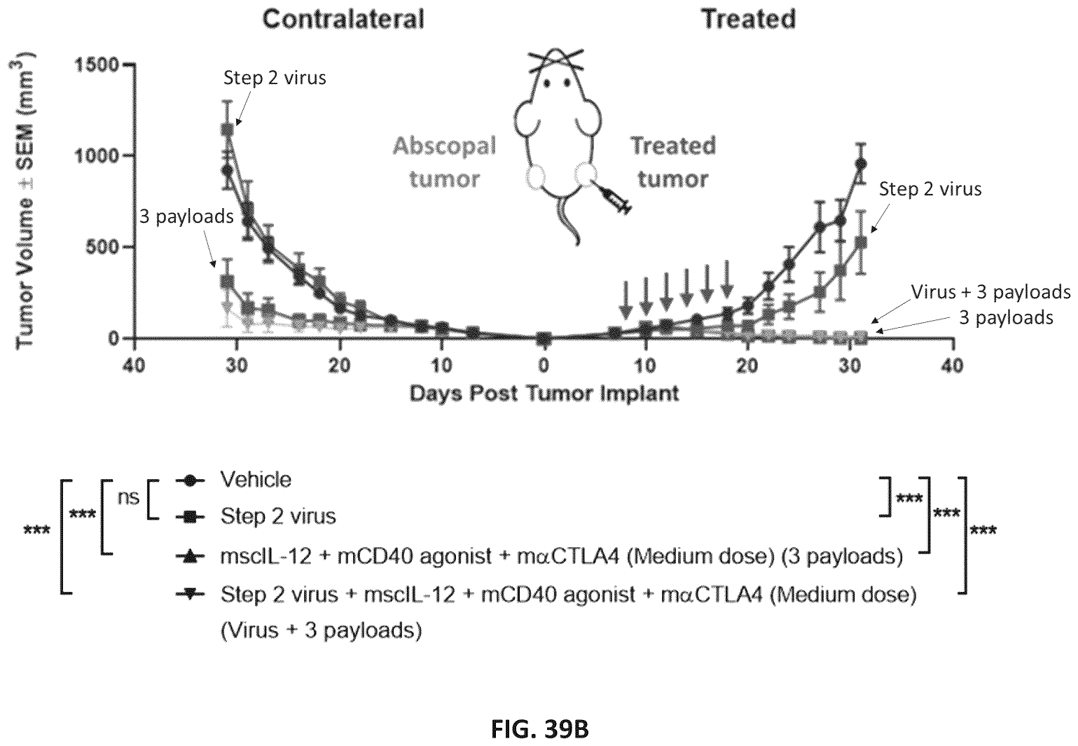

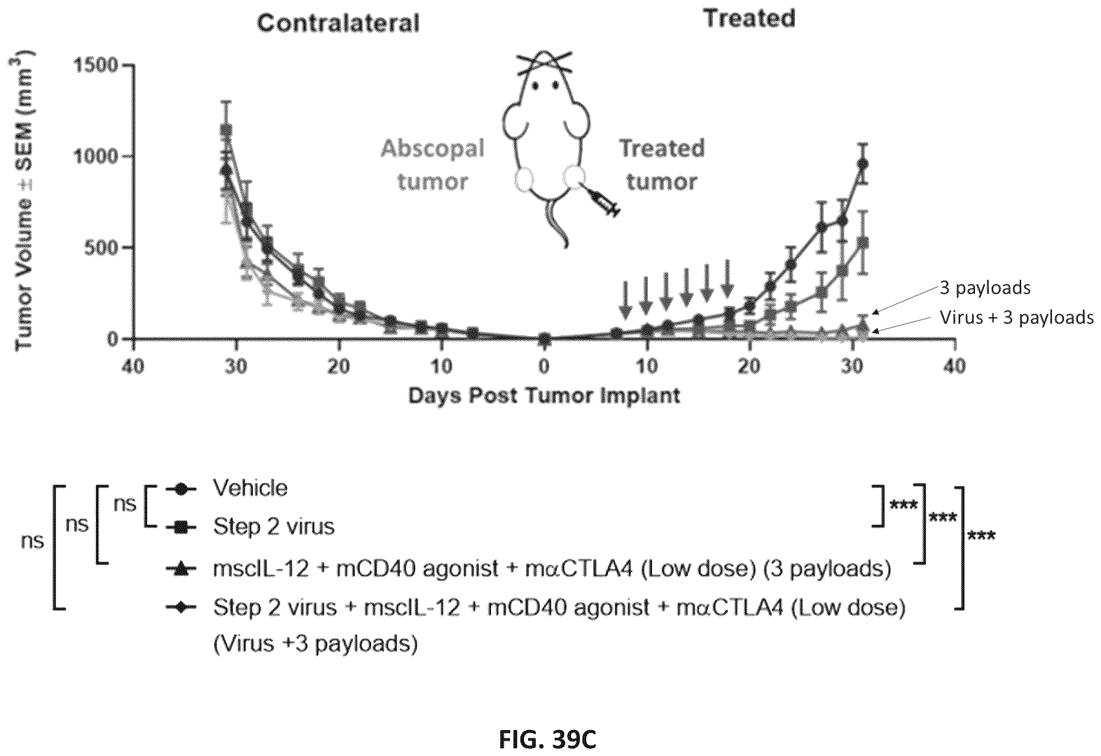

To determine the minimal quantity of mouse payloads required for abscopal efficacy, a payload dose-response experiment was performed, combined with Step 2 virus (i.e., hFLT3L-expressing OV). Step 2 virus (expressing hFLT3L) was simultaneously dosed in a bilateral MC38-5AG syngeneic model with all three remaining mouse surrogate payloads (i.e., the mαCTLA-4, mCD40ag, and mscIL-12 payloads). mCD40ag and mαCTLA-4 were tested at 3 different doses. The High dose was 13 μg for mCD40ag and 10.8 μg for mαCTLA-4, with 10-fold (Medium dose) and 100-fold (Low dose) dilutions for additional doses. Step 2 virus and mscIL-12 were given at 5×10⁶ PFU and 50 ng, respectively. Payloads without virus infection were also tested. As shown in FIGS. 39A-39C, a significant tumor growth inhibition (TGI) effect was observed upon treatment with virus alone for the treated tumor. An increased treated tumor TGI effect was observed with simultaneous IT administration of the three payloads, with or without the virus injection, at all payload doses tested. As for the contralateral tumors, injection of virus alone demonstrated no beneficial effect. At the highest payload concentration (High dose), a strong abscopal response for the payloads was seen with or without virus treatment (FIG. 39A). At the Medium payload dose, a strong abscopal response was also observed, albeit slightly less robust than that observed with the High dose payloads (FIG. 39B). While the High and Medium payload IT injections without virus co-injection demonstrated abscopal efficacy, at the Medium payload dose, an improved abscopal response was observed upon virus co-injection (FIG. 39B; not significant). At the Low payload dose, no statistically significant abscopal response was observed for the payloads, with or without virus co-administration (FIG. 39C). The contribution of Step 2 virus to the abscopal efficacy of payload administration was relatively minor in these experiments. This was unsurprising given that HSV-1, here Step 2 virus, replicates poorly in mouse cancer cells. Overall, these results demonstrated that treatment with lower purified payload concentrations (i.e., Medium dose) led to strong and significant abscopal TGI effect. In addition, the results also strongly supported the choice of the specific payloads that oncolytic viruses JP-OV-2 and mJP-OV-2 are engineered to express, as they demonstrated a strong synergistic anti-tumor immune response.

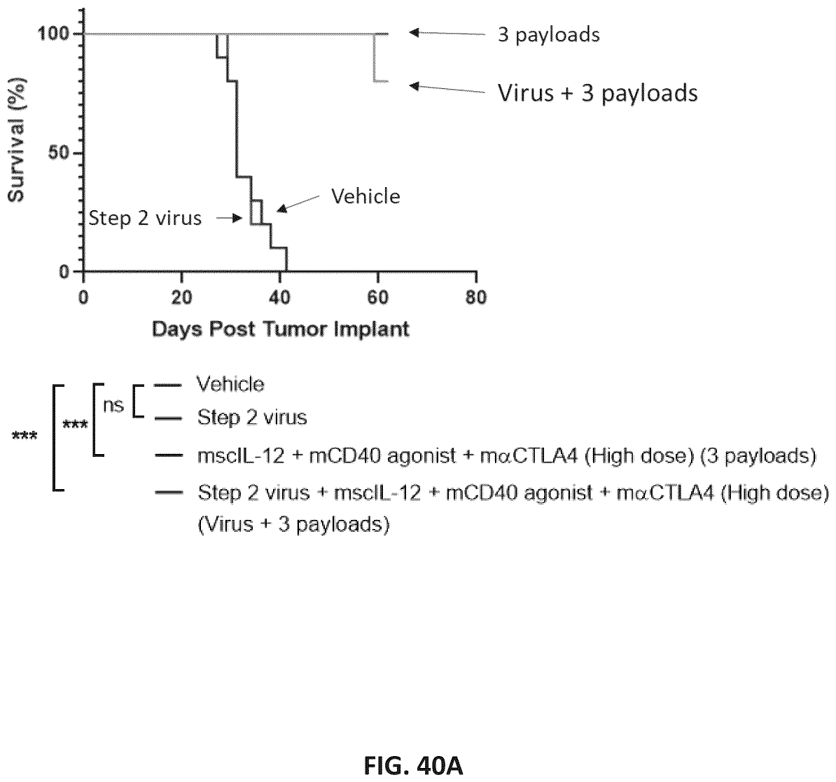

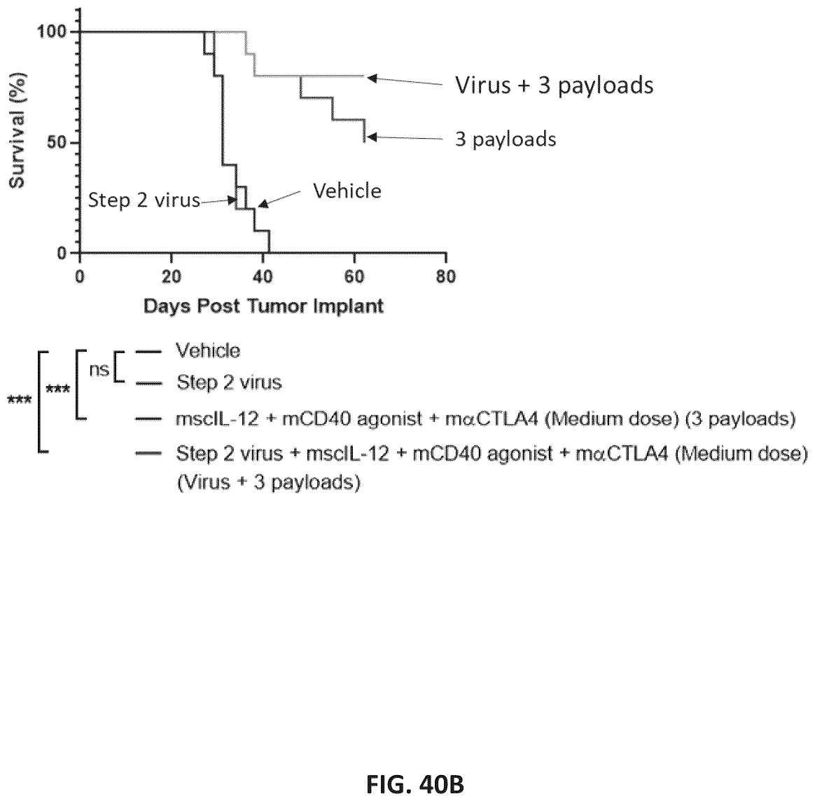

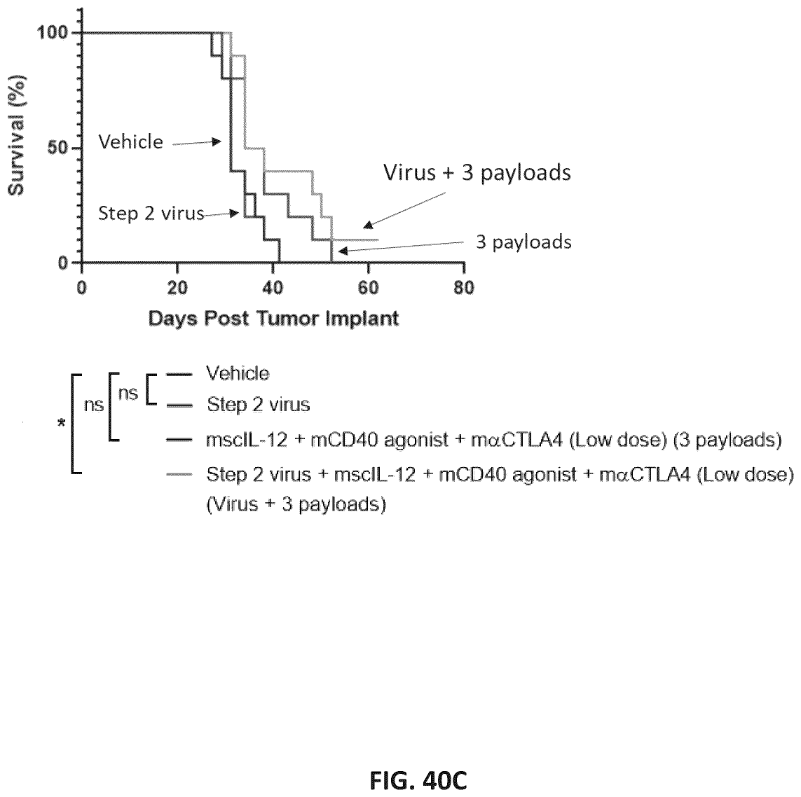

A survival analysis was carried out on the mice used in the experiments shown in FIGS. 39A-39C. As shown in FIGS. 40A-40C, the High dose of payloads significantly improved overall survival, with 7 of 10 and 9 of 10 animals cured (with and without Step 2 virus treatment, respectively), with no detectable tumor on either side (FIG. 40A). The Medium dose also produced a significant survival advantage, with 7 of 10 and 4 of 10 complete cures with and without Step 2 virus, respectively (FIG. 40B). The Low dose only produced a significant effect on survival when combined with Step 2 virus, and no cures were observed with or without virus treatment (FIG. 40C).

Overall, the results described above established the concentration of IT payloads required for abscopal efficacy, and also assessed the efficacy of payloads at concentrations most likely higher than that generated upon surrogate virus infection of MC38-5AG mouse tumors, where HSV-1 replication is poor. These data demonstrated that increased payload concentration resulted in durable cures, with High or Medium doses most likely representative of payload expression levels by oncolytic virus JP-OV-2 upon human tumor infection.

### Systemic Anti-Tumor Memory Immune Responses

To test for the formation of a systemic anti-tumor memory immune response, a re-challenge study was designed to test mice that had been cured of bilateral MC38-5AG tumors by the combination of Step 2 virus (which expresses the hFLT3L payload) and the mαCTLA-4, mCD40ag, and mscIL-12 payloads.

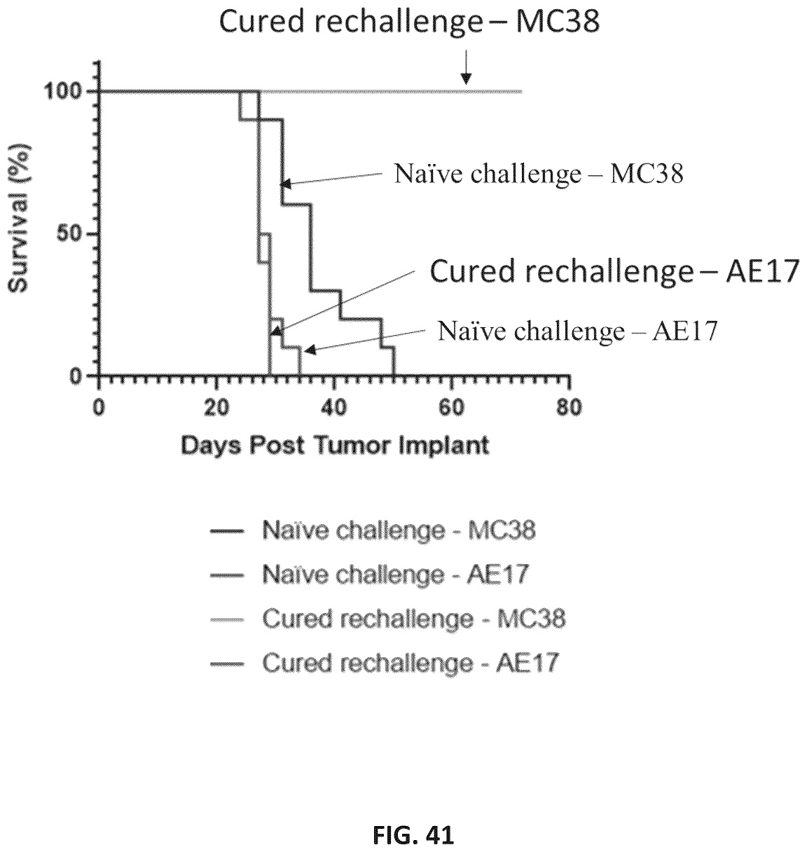

Naïve mice were challenged with single MC38-5AG (n=10) or AE17 (n=10) tumors. All naïve mice grew tumors that eventually reached the tumor burden criteria for sacrifice (FIG. 41). Mice that had been previously cured were also challenged with either MC38-5AG (n=5) or AE17 (n=4) tumors. While all previously cured mice challenged with AE17 grew tumors, cured mice were completely protected from re-challenge with MC38-5AG tumors (FIG. 41). These results showed that treatment with Step 2 virus (which expresses the hFLT3L payload) in combination with all other payloads (i.e., the mαCTLA-4, mCD40ag, and mscIL-12 payloads) produced a durable, specific, and protective anti-tumor response.

### In Vivo Efficacy of Mouse Surrogate Virus

### Anti-Tumor Efficacy and Survival

The efficacy of the mouse surrogate virus, mJP-OV-2, was assessed in a bilateral MC38-5AG syngeneic tumor model. Two dosing regimens were compared: a once every 3 days for 3 doses (q3dx3) regimen traditionally used to test OVs, as well as a more frequent once every other day for 6 doses (q2dx6) regimen previously used for the assessment of IT-injected purified payloads. mJP-OV-2 efficacy was compared to that of a similar virus with no payloads, referred to as unarmed backbone virus.

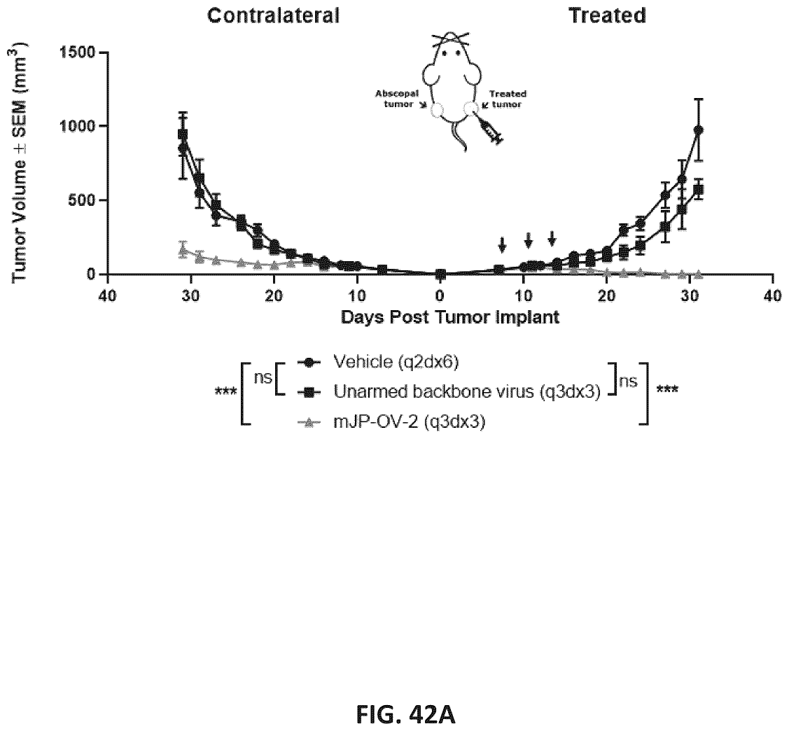

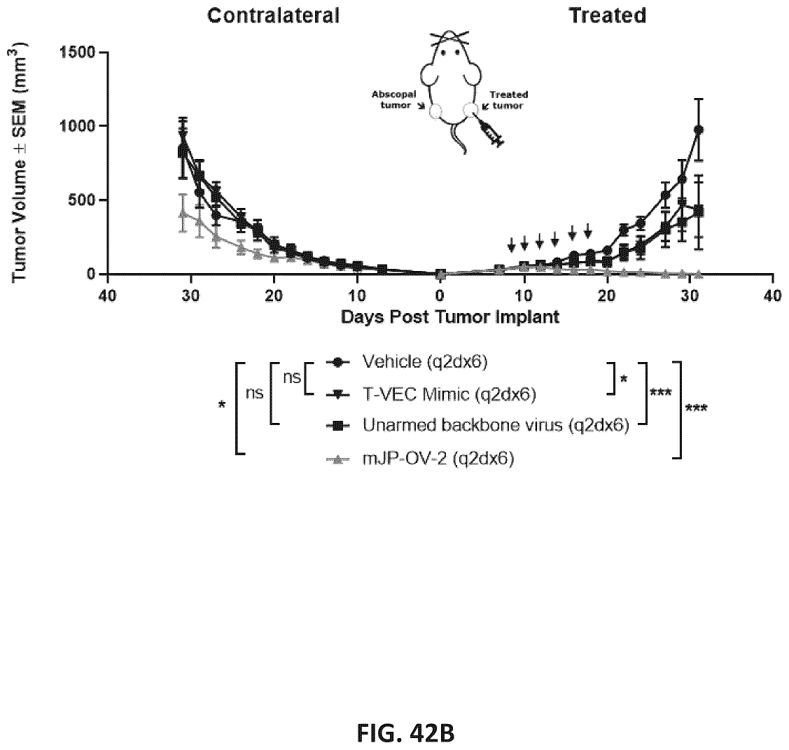

As shown in FIGS. 42A-42B, under both dosing schedules, the surrogate virus showed a strong effect on treated tumors, which was far superior to that of unarmed backbone OV. A significant TGI effect was also demonstrated for the untreated, contralateral tumor upon treatment with mJP-OV-2 under both treatment schedules tested. In contrast, no abscopal effect was observed with the unarmed backbone virus under both dosing schedules. Interestingly, the traditional q3dx3 dosing regimen was more efficacious than the q2dx6 regimen. A previously-reported genetically engineered oncolytic herpesvirus mimic, which is a close analog of a previously-reported genetically engineered oncolytic herpesvirus but expressing murine GM-CSF instead of human GM-CSF, was also compared to mJP-OV-2 in this syngeneic mouse model. Using the q2dx6 dosing schedule, the previously-reported genetically engineered oncolytic herpesvirus mimic demonstrated similar efficacy to the unarmed backbone virus, and inferior efficacy to mJP-OV-2 on both treated and contralateral tumors.

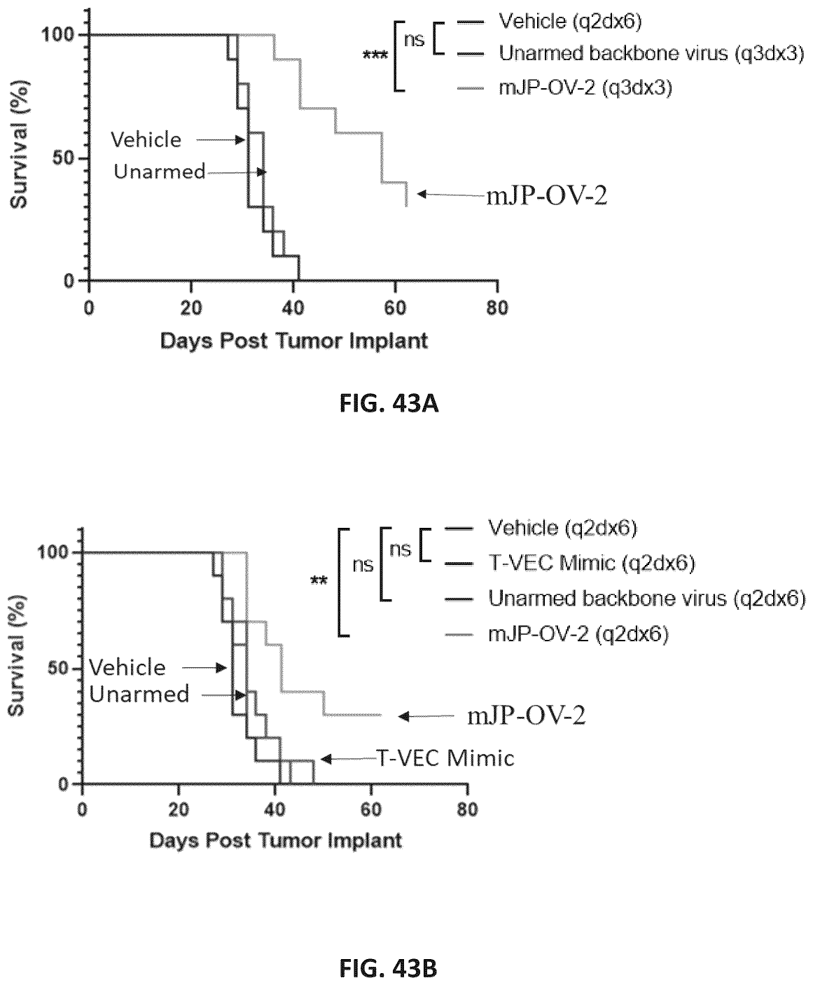

A survival analysis of the mice used in the experiments described above demonstrated that mJP-OV-2 significantly enhanced overall survival with both dosing schedules, with complete cures of 3 of 10 animals with both the q2dx6 and q3dx3 regimens (FIGS. 43A-43B). Neither the unarmed backbone virus nor the previously-reported genetically engineered oncolytic herpesvirus mimic significantly improved survival or produced cures. Although fewer cures were observed-with surrogate virus mJP-OV-2 compared to Step 2 virus combined with purified surrogate payloads (see, FIGS. 38B and 40A-40C), this was consistent with the reduced payload concentration produced by virus expression in MC38-5AG tumors as a result of poor HSV-1 replication in murine cells. Thus, it is important to consider that mouse tumor models may underrepresent the efficacy of the human oncolytic virus JP-OV-2 in human tumors due to the attenuated OV replication in murine compared to human cancer cells.

Overall, these results demonstrated abscopal efficacy for mJP-OV-2 that was greatly superior to that of a previously-reported genetically engineered oncolytic herpesvirus mimic. Based on these data, and knowing that HSV-1 replication, and thus payload expression, is attenuated in this murine syngeneic model compared to that in human tumors, infection of human tumors with oncolytic virus JP-OV-2 may lead to clinical efficacy superior to that observed for the mouse surrogate virus, mJP-OV-2, against murine tumor models.

### In Vivo Payload Protein Expression

Since the efficacy of human oncolytic viruses, such as JP-OV-2, cannot be evaluated in immunocompetent animal models (as JP-OV-2 encodes human payloads), its potential efficacy can be extrapolated from the level of payload expression upon infection of human tumors, compared to payload expression from the efficacious mouse surrogate virus (mJP-OV-2) upon infection of murine tumors in an immunocompetent mouse animal model.

Accordingly, mouse surrogate payload expression from mJP-OV-2 was determined following one IT injection of mJP-OV-2 virus in mouse MC38-5AG tumors, and compared to that of one JP-OV-2 injection in human H1299 tumors in a mouse xenograft model. Tumors were harvested at different timepoints post virus injection, homogenized, and each payload was quantitated via ELISA.

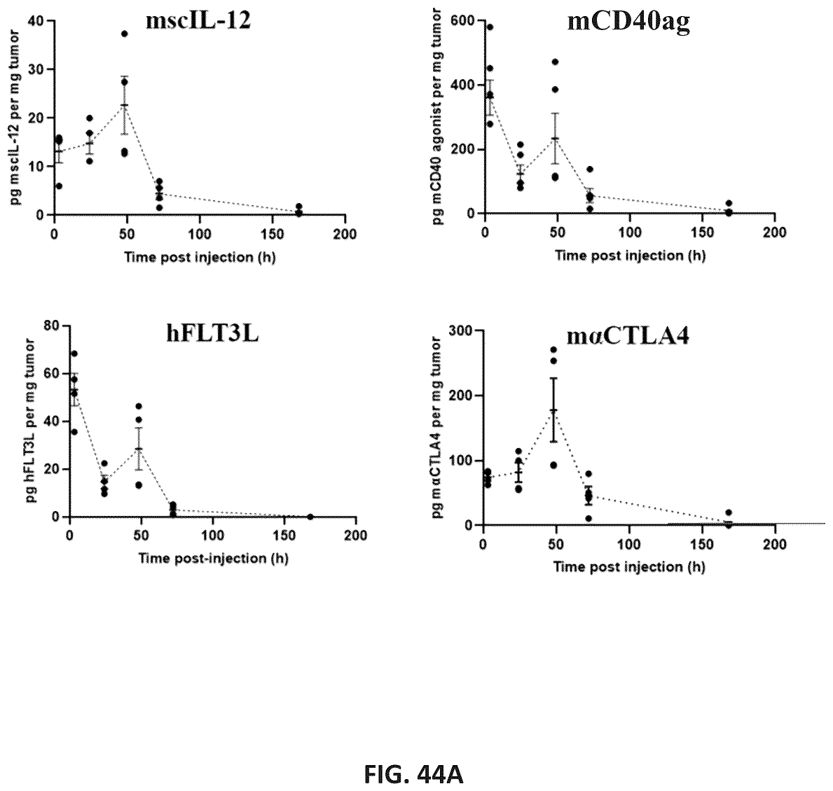

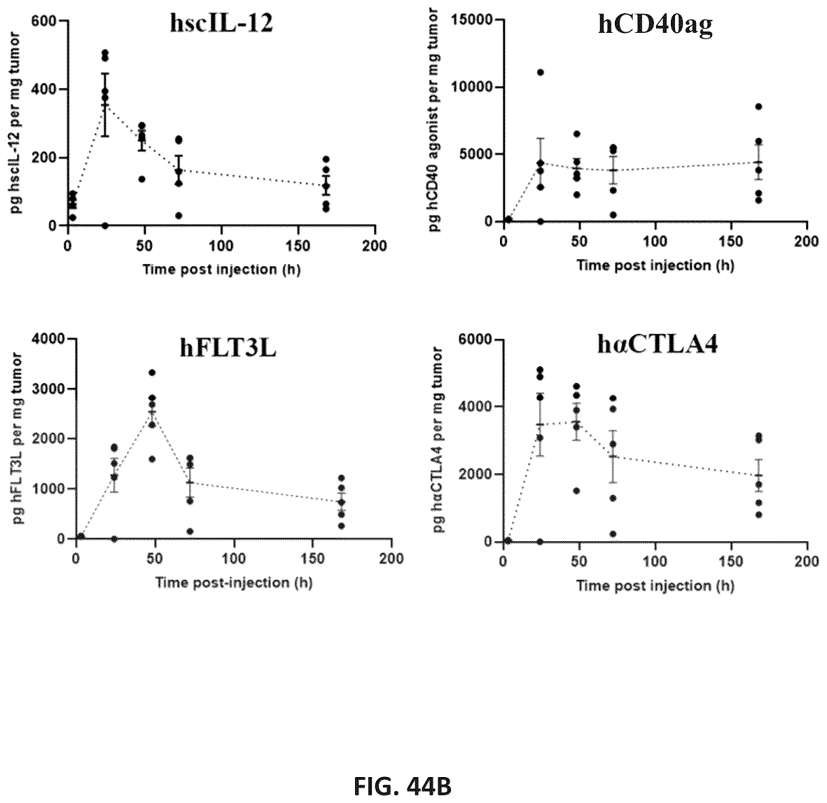

As shown in FIG. 44A, upon infection of MC38-5AG mouse tumors with mJP-OV-2, all payloads were expressed and reached an expression peak at around 48 hours post infection, returning to baseline expression levels by 7 days post infection. This was consistent with the poor replication of this human OV in murine models. Although expression levels were relatively low, it was demonstrated that treatment with mJP-OV-2 in this model produced abscopal efficacy, as discussed above. Therefore, this level of payload expression was sufficient to produce a biological effect.

As shown in FIG. 44B, upon infection of H1299 human tumors with JP-OV-2, all payloads were expressed, and around 24 to 48 hours post-infection, each reached an expression peak considerably higher than what was observed for the corresponding payloads after MC38-5AG infection with mJP-OV-2. Payload expression was also sustained until Day 7 post-infection in all cases (the last day of measurement), which was consistent with continued virus replication in human tumor cells.

The results shown in FIGS. 44A-44B are summarized and quantified in Table 3.

TABLE 3
Total and average payload expressed by JP-
OV-2 in H1299 xenograft tumor models, or by
mJP-OV-2 in MC38-5AG mouse tumor models.

| JP-OV-2 in Human H1299 | | | mJP-OV-2 in Mouse MC38-5AG | | |
|---|---|---|---|---|---|
| | AUCₗₐₛₜ | C_{average} | | AUCₗₐₛₜ | C_{average} |
| Analyte | (pg · h/mg) | (pg/mg) | Analyte | (pg · h/mg) | (pg/mg) |
| hscIL-12 | 30,235.59 | 179.97 | mscIL-12 | 1,336.14 | 7.95 |
| hαCTLA- | 409,524.08 | 2,437.64 | mαCTLA- | 9,986.39 | 59.44 |
| 4 | | | 4 | | |
| hCD40ag | 634,472.82 | 3,776.62 | mCD40ag | 19,166.28 | 114.08 |
| hFLT3L | 193,792.79 | 1,153.53 | hFLT3L | 1,684.78 | 10.03 |

AUCₗₐₛₜ, area under the plasma concentration - time curve from time zero to time corresponding to last quantifiable concentration; C, payload expression per hour; NCA, non-compartmental analysis.
Payload expression data was performed with Phoenix WinNolin using sparse sampling NCA analysis; total payload expression (pg per mg of tumor for duration of the study) is represented by AUCₗₐₛₜ (pg · h/mg) and average payload expression per hour (C_{average} (pg/mg)) is derived from AUCₗₐₛₜ/168 hours. All 4 payloads were found to be expressed higher in H1229 compared to MC38-5AG, being approximately 23-, 41-, 33-, and 115-fold higher for IL-12, αCTLA-4, CD40ag, and FLT3L, respectively.

Given that the levels and duration of payload expression for JP-OV-2 in human tumors was higher than the levels and duration of payload expression shown to produce biological efficacy with mJP-OV-2 in mouse tumors, the results described above suggest that JP-OV-2 possesses equal or better activity.

### Sequences

TABLE 4
Single Chain IL-12 Payload Amino Acid Sequences.

| Description | Sequence | SEQ ID NO |
|---|---|---|
| [[OVTSW24]] | RNLPVATPDPGMFPCLHHSQNLLRAVSNMLQKA | 1 |
| p35 subunit of human IL- | RQTLEFYPCTSEEIDHEDITKDKTSTVEACLPL | |
| 12 in human single chain | ELTKNESCLNSRETSFITNGSCLASRKTSFMMA | |
| (sc) IL-12 payload. | LCLSSIYEDLKMYQVEFKTMNAKLLMDPKRQIF | |
| | LDQNMLAVIDELMQALNFNSETVPQKSSLEEPD | |
| | FYKTKIKLCILLHAFRIRAVTIDRVMSYLNAS | |
| [[OVTSW24]] | MCHQQLVISWFSLVFLASPLVAIWELKKDVYVV | 2 |
| p40 subunit of human IL- | ELDWYPDAPGEMVVLTCDTPEEDGITWTLDQSS | |
| 12 in human scIL-12 | EVLGSGKTLTIQVKEFGDAGQYTCHKGGEVLSH | |
| payload. | SLLLLHKKEDGIWSTDILKDQKEPKNKTFLRCE | |
| | AKNYSGRFTCWWLTTISTDLTFSVKSSRGSSDP | |
| | QGVTCGAATLSAERVRGDNKEYEYSVECQEDSA | |
| | CPAAEESLPIEVMVDAVHKLKYENYTSSFFIRD | |
| | IIKPDPPKNLQLKPLKNSRQVEVSWEYPDTWST | |
| | PHSYFSLTFCVQVQGKSKREKKDRVFTDKTSAT | |
| | VICRKNASISVRAQDRYYSSSWSEWASVPCS | |
| [[OVTSW24]] | GGGGGGS | 3 |
| G₆S glycine serine tether | | |
| in human scIL-12 | | |
| payload. | | |
| [[OVTSW24]] | MCHQQLVISWFSLVFLASPLVAIWELKKDVYVV | 4 |
| Single chain human IL- | ELDWYPDAPGEMVVLTCDTPEEDGITWTLDQSS | |
| 12. | EVLGSGKTLTIQVKEFGDAGQYTCHKGGEVLSH | |
| | SLLLLHKKEDGIWSTDILKDQKEPKNKTFLRCE | |
| | AKNYSGRFTCWWLTTISTDLTFSVKSSRGSSDP | |
| | QGVTCGAATLSAERVRGDNKEYEYSVECQEDSA | |
| | CPAAEESLPIEVMVDAVHKLKYENYTSSFFIRD | |
| | IIKPDPPKNLQLKPLKNSRQVEVSWEYPDTWST | |
| | PHSYFSLTFCVQVQGKSKREKKDRVFTDKTSAT | |
| | VICRKNASISVRAQDRYYSSSWSEWASVPCSGG | |
| | GGGGSRNLPVATPDPGMFPCLHHSQNLLRAVSN | |
| | MLQKARQTLEFYPCTSEEIDHEDITKDKTSTVE | |
| | ACLPLELTKNESCLNSRETSFITNGSCLASRKT | |
| | SFMMALCLSSIYEDLKMYQVEFKTMNAKLLMDP | |
| | KRQIFLDQNMLAVIDELMQALNFNSETVPQKSS | |
| | LEEPDFYKTKIKLCILLHAFRIRAVTIDRVMSY | |
| | LNAS | |
| [[OVTSW25]] | RVIPVSGPARCLSQSRNLLKTTDDMVKTAREKL | 5 |
| p35 subunit in mouse | KHYSCTAEDIDHEDITRDQTSTLKTCLPLELHK | |
| scIL-12 payload. | NESCLATRETSSTTRGSCLPPQKTSLMMTLCLG | |
| | SIYEDLKMYQTEFQAINAALQNHNHQQIILDKG | |
| | MLVAIDELMQSLNHNGETLRQKPPVGEADPYRV | |
| | KMKLCILLHAFSTRVVTINRVMGYLSSA | |
| [[OVTSW25]] | MCPQKLTISWFAIVLLVSPLMAMWELEKDVYVV | 6 |
| p40 subunit in mouse | EVDWTPDAPGETVNLTCDTPEEDDITWTSDQRH | |
| scIL-12 payload. | GVIGSGKTLTITVKEFLDAGQYTCHKGGETLSH | |
| | SHLLLHKKENGIWSTEILKNFKNKTFLKCEAPN | |
| | YSGRFTCSWLVQRNMDLKFNIKSSSSSPDSRAV | |
| | TCGMASLSAEKVTLDQRDYEKYSVSCQEDVTCP | |
| | TAEETLPIELALEARQQNKYENYSTSFFIRDII | |
| | KPDPPKNLQMKPLKNSQVEVSWEYPDSWSTPHS | |
| | YFSLKFFVRIQRKKEKMKETEEGCNQKGAFLVE | |
| | KTSTEVQCKGGNVCVQAQDRYYNSSCSKWACVP | |
| | CRVRS | |
| [[OVTSW25]] | GGGGSGGGGSGGGGS | 7 |
| Glycine serine tether in | | |
| mouse scIL-12 payload. | | |
| [[OVTSW25]] | MWELEKDVYVVEVDWTPDAPGETVNLTCDTPEE | 8 |
| Single chain mouse IL-12 | DDITWTSDQRHGVIGSGKTLTITVKEFLDAGQY | |
| | TCHKGGETLSHSHLLLHKKENGIWSTEILKNFK | |
| | NKTFLKCEAPNYSGRFTCSWLVQRNMDLKFNIK | |
| | SSSSSPDSRAVTCGMASLSAEKVTLDQRDYEKY | |
| | SVSCQEDVTCPTAEETLPIELALEARQQNKYEN | |
| | YSTSFFIRDIIKPDPPKNLQMKPLKNSQVEVSW | |
| | EYPDSWSTPHSYFSLKFFVRIQRKKEKMKETEE | |
| | GCNQKGAFLVEKTSTEVQCKGGNVCVQAQDRYY | |
| | NSSCSKWACVPCRVRSGGGGSGGGGSGGGGSRV | |
| | IPVSGPARCLSQSRNLLKTTDDMVKTAREKLKH | |
| | YSCTAEDIDHEDITRDQTSTLKTCLPLELHKNE | |
| | SCLATRETSSTTRGSCLPPQKTSLMMTLCLGSI | |
| | YEDLKMYQTEFQAINAALQNHNHQQIILDKGML | |
| | VAIDELMQSLNHNGETLRQKPPVGEADPYRVKM | |
| | KLCILLHAFSTRVVTINRVMGYLSSA | |
| [[OVTSW24]] | IWELKKDVYVVELDWYPDAPGEMVVLTCDTPEE | 9 |
| p40 subunit of human IL- | DGITWTLDQSSEVLGSGKTLTIQVKEFGDAGQY | |
| 12 in human scIL-12 | TCHKGGEVLSHSLLLLHKKEDGIWSTDILKDQK | |
| payload without signal | EPKNKTFLRCEAKNYSGRFTCWWLTTISTDLTF | |
| peptide sequence. | SVKSSRGSSDPQGVTCGAATLSAERVRGDNKEY | |
| | EYSVECQEDSACPAAEESLPIEVMVDAVHKLKY | |
| | ENYTSSFFIRDIIKPDPPKNLQLKPLKNSRQVE | |
| | VSWEYPDTWSTPHSYFSLTFCVQVQGKSKREKK | |
| | DRVFTDKTSATVICRKNASISVRAQDRYYSSSW | |
| | SEWASVPCS | |
| [[OVTSW24]] | IWELKKDVYVVELDWYPDAPGEMVVLTCDTPEE | 10 |
| Single chain human IL-12 | DGITWTLDQSSEVLGSGKTLTIQVKEFGDAGQY | |
| without signal peptide. | TCHKGGEVLSHSLLLLHKKEDGIWSTDILKDQK | |
| | EPKNKTFLRCEAKNYSGRFTCWWLTTISTDLTF | |
| | SVKSSRGSSDPQGVTCGAATLSAERVRGDNKEY | |
| | EYSVECQEDSACPAAEESLPIEVMVDAVHKLKY | |
| | ENYTSSFFIRDIIKPDPPKNLQLKPLKNSRQVE | |
| | VSWEYPDTWSTPHSYFSLTFCVQVQGKSKREKK | |
| | DRVFTDKTSATVICRKNASISVRAQDRYYSSSW | |
| | SEWASVPCSGGGGGGSRNLPVATPDPGMFPCLH | |
| | HSQNLLRAVSNMLQKARQTLEFYPCTSEEIDHE | |
| | DITKDKTSTVEACLPLELTKNESCLNSRETSFI | |
| | TNGSCLASRKTSFMMALCLSSIYEDLKMYQVEF | |
| | KTMNAKLLMDPKRQIFLDQNMLAVIDELMQALN | |
| | FNSETVPQKSSLEEPDFYKTKIKLCILLHAFRI | |
| | RAVTIDRVMSYLNAS | |
| [[OVTSW25]] | MWELEKDVYVVEVDWTPDAPGETVNLTCDTPEE | 11 |
| p40 subunit in mouse | DDITWTSDQRHGVIGSGKTLTITVKEFLDAGQY | |
| scIL-12 payload without | TCHKGGETLSHSHLLLHKKENGIWSTEILKNFK | |
| signal peptide. | NKTFLKCEAPNYSGRFTCSWLVQRNMDLKFNIK | |
| | SSSSSPDSRAVTCGMASLSAEKVTLDQRDYEKY | |
| | SVSCQEDVTCPTAEETLPIELALEARQQNKYEN | |
| | YSTSFFIRDIIKPDPPKNLQMKPLKNSQVEVSW | |
| | EYPDSWSTPHSYFSLKFFVRIQRKKEKMKETEE | |
| | GCNQKGAFLVEKTSTEVQCKGGNVCVQAQDRYY | |
| | NSSCSKWACVPCRVRS | |
| [[OVTSW25]] | MWELEKDVYVVEVDWTPDAPGETVNLTCDTPEE | 12 |
| Single chain mouse IL-12 | DDITWTSDQRHGVIGSGKTLTITVKEFLDAGQY | |
| without signal peptide. | TCHKGGETLSHSHLLLHKKENGIWSTEILKNFK | |
| | NKTFLKCEAPNYSGRFTCSWLVQRNMDLKFNIK | |
| | SSSSSPDSRAVTCGMASLSAEKVTLDQRDYEKY | |
| | SVSCQEDVTCPTAEETLPIELALEARQQNKYEN | |
| | YSTSFFIRDIIKPDPPKNLQMKPLKNSQVEVSW | |
| | EYPDSWSTPHSYFSLKFFVRIQRKKEKMKETEE | |
| | GCNQKGAFLVEKTSTEVQCKGGNVCVQAQDRYY | |
| | NSSCSKWACVPCRVRSGGGGSGGGGSGGGGSRV | |
| | IPVSGPARCLSQSRNLLKTTDDMVKTAREKLKH | |
| | YSCTAEDIDHEDITRDQTSTLKTCLPLELHKNE | |
| | SCLATRETSSTTRGSCLPPQKTSLMMTLCLGSI | |
| | YEDLKMYQTEFQAINAALQNHNHQQIILDKGML | |
| | VAIDELMQSLNHNGETLRQKPPVGEADPYRVKM | |
| | KLCILLHAFSTRVVTINRVMGYLSSA | |

TABLE 5
CD40 Agonist Payload Amino Acid Sequences.

| Description | Sequence | SEQ ID NO |
|---|---|---|
| [[C4LW84]] | NPQIAAHVISEASSKTTSVLQWAEK | 20 |
| Human CD40L ectodomain sequence | GYYTMSNNLVTLENGKQLTVKRQGL | |
| in human CD40 agonist payload. | YYIYAQVTFCSNREASSQAPFIASL | |
| | CLKSPGRFERILLRAANTHSSAKPC | |
| | GQQSIHLGGVFELQPGASVFVNVTD | |
| | PSQVSHGTGFTSFGLLKL | |
| [[C4LW84 & C4LW141]] | GYIPEAPRDGQAYVRKDGEWVLLST | 21 |
| Trimerization motif from the fibritin | FL | |
| protein of T4 phage in human and | | |
| mouse CD40 agonist payloads. | | |
| [[C4LW84 & C4LW141]] | GGGSGGGSGGGS | 22 |
| Glycine/serine linker between CD40L | | |
| ectodomains in human and mouse | | |
| CD40 agonist payloads. | | |
| [[C4LW84]] | GGGGS | 23 |
| Glycine/serine linker between | | |
| trimerization motif and CD40L | | |
| ectodomains in human CD40 agonist | | |
| payload. | | |
| [[C4LW84]] | MAWVWTLLFLMAAAQSIQA | 24 |
| Signal peptide sequence in human and | | |
| mouse CD40 agonist payloads. | | |
| [[C4LW84]] | MAWVWTLLFLMAAAQSIQAGYIPEA | 25 |
| Human CD40 agonist payload. | PRDGQAYVRKDGEWVLLSTFLGGGG | |
| | SNPQIAAHVISEASSKTTSVLQWAE | |
| | KGYYTMSNNLVTLENGKQLTVKRQG | |
| | LYYIYAQVTFCSNREASSQAPFIAS | |
| | LCLKSPGRFERILLRAANTHSSAKP | |
| | CGQQSIHLGGVFELQPGASVFVNVT | |
| | DPSQVSHGTGFTSFGLLKLGGGSGG | |
| | GSGGGSNPQIAAHVISEASSKTTSV | |
| | LQWAEKGYYTMSNNLVTLENGKQLT | |
| | VKRQGLYYIYAQVTFCSNREASSQA | |
| | PFIASLCLKSPGRFERILLRAANTH | |
| | SSAKPCGQQSIHLGGVFELQPGASV | |
| | FVNVTDPSQVSHGTGFTSFGLLKLG | |
| | GGSGGGSGGGSNPQIAAHVISEASS | |
| | KTTSVLQWAEKGYYTMSNNLVTLEN | |
| | GKQLTVKRQGLYYIYAQVTFCSNRE | |
| | ASSQAPFIASLCLKSPGRFERILLR | |
| | AANTHSSAKPCGQQSIHLGGVFELQ | |
| | PGASVFVNVTDPSQVSHGTGFTSFG | |
| | LLKL | |
| [[C4LW141]] | DPQIAAHVVSEANSNAASVLQWAKK | 26 |
| Mouse CD40L ectodomain in mouse | GYYTMKSNLVMLENGKQLTVKREGL | |
| CD40 agonist payload. | YYVYTQVTFCSNREPSSQRPFIVGL | |
| | WLKPSSGSERILLKAANTHSSSQLC | |
| | EQQSVHLGGVFELQAGASVFVNVTE | |
| | ASQVIHRVGFSSFGLLKL | |
| [[C4LW141]] | GGGGSGGGGS | 27 |
| Glycine/serine linker between fibritin | | |
| protein of T4 phage trimerization | | |
| motif and CD40L ectodomains in | | |
| mouse CD40 agonist payload. | | |
| [[C4LW141]] | MAWVWTLLFLMAAAQSIQAGYIPEA | 28 |
| Mouse CD40 agonist payload | PRDGQAYVRKDGEWVLLSTFLGGGG | |
| Trimerization motif from the fibritin | SGGGGSDPQIAAHVVSEANSNAASV | |
| protein of T4 phage is bolded and | LQWAKKGYYTMKSNLVMLENGKQLT | |
| underlined | VKREGLYYVYTQVTFCSNREPSSQR | |
| Mouse CD40L ectodomain sequences | PFIVGLWLKPSSGSERILLKAANTH | |
| are bolded. | SSSQLCEQQSVHLGGVFELQAGASV | |
| | FVNVTEASQVIHRVGFSSFGLLKLG | |
| | GGSGGGSGGGSDPQIAAHVVSEANS | |
| | NAASVLQWAKKGYYTMKSNLVMLEN | |
| | GKQLTVKREGLYYVYTQVTFCSNRE | |
| | PSSQRPFIVGLWLKPSSGSERILLK | |
| | AANTHSSSQLCEQQSVHLGGVFELQ | |
| | AGASVFVNVTEASQVIHRVGFSSFG | |
| | LLKLGGGSGGGSGGGSDPQIAAHVV | |
| | SEANSNAASVLQWAKKGYYTMKSNL | |
| | VMLENGKQLTVKREGLYYVYTQVTF | |
| | CSNREPSSQRPFIVGLWLKPSSGSE | |
| | RILLKAANTHSSSQLCEQQSVHLGG | |
| | VFELQAGASVFVNVTEASQVIHRVG | |
| | FSSFGLLKL | |
| [[C4LW141]] | GYIPEAPRDGQAYVRKDGEWVLLST | 29 |
| Mouse CD40 agonist payload without | FLGGGGSGGGGSDPQIAAHVVSEAN | |
| signal peptide. | SNAASVLQWAKKGYYTMKSNLVMLE | |
| | NGKQLTVKREGLYYVYTQVTFCSNR | |
| | EPSSQRPFIVGLWLKPSSGSERILL | |
| | KAANTHSSSQLCEQQSVHLGGVFEL | |
| | QAGASVFVNVTEASQVIHRVGFSSF | |
| | GLLKLGGGSGGGSGGGSDPQIAAHV | |
| | VSEANSNAASVLQWAKKGYYTMKSN | |
| | LVMLENGKQLTVKREGLYYVYTQVT | |
| | FCSNREPSSQRPFIVGLWLKPSSGS | |
| | ERILLKAANTHSSSQLCEQQSVHLG | |
| | GVFELQAGASVFVNVTEASQVIHRV | |
| | GFSSFGLLKLGGGSGGGSGGGSDPQ | |
| | IAAHVVSEANSNAASVLQWAKKGYY | |
| | TMKSNLVMLENGKQLTVKREGLYYV | |
| | YTQVTFCSNREPSSQRPFIVGLWLK | |
| | PSSGSERILLKAANTHSSSQLCEQQ | |
| | SVHLGGVFELQAGASVFVNVTEASQ | |
| | VIHRVGFSSFGLLKL | |
| [[C4LW84]] | GYIPEAPRDGQAYVRKDGEWVLLST | 30 |
| Human CD40 agonist payload without | FLGGGGSNPQIAAHVISEASSKTTS | |
| signal peptide. | VLQWAEKGYYTMSNNLVTLENGKQL | |
| | TVKRQGLYYIYAQVTFCSNREASSQ | |
| | APFIASLCLKSPGRFERILLRAANT | |
| | HSSAKPCGQQSIHLGGVFELQPGAS | |
| | VFVNVTDPSQVSHGTGFTSFGLLKL | |
| | GGGSGGGSGGGSNPQIAAHVISEAS | |
| | SKTTSVLQWAEKGYYTMSNNLVTLE | |
| | NGKQLTVKRQGLYYIYAQVTFCSNR | |
| | EASSQAPFIASLCLKSPGRFERILL | |
| | RAANTHSSAKPCGQQSIHLGGVFEL | |
| | QPGASVFVNVTDPSQVSHGTGFTSF | |
| | GLLKLGGGSGGGSGGGSNPQIAAHV | |
| | ISEASSKTTSVLQWAEKGYYTMSNN | |
| | LVTLENGKQLTVKRQGLYYIYAQVT | |
| | FCSNREASSQAPFIASLCLKSPGRF | |
| | ERILLRAANTHSSAKPCGQQSIHLG | |
| | GVFELQPGASVFVNVTDPSQVSHGT | |
| | GFTSFGLLKL | |

TABLE 6
CTLA-4 Antagonist Payload Amino Acid Sequences.

| Description | Sequence | SEQ ID NO |
|---|---|---|
| [[OVTSB29]] | GFTFSSYTMH | 40 |
| Human CTLA-4 antagonist payload | | |
| CDR-H1 | | |
| [[OVTSB29]] | FISYDGNNKY | 41 |
| Human CTLA-4 antagonist payload | | |
| CDR-H2 | | |
| [[OVTSB29]] | TGWLGPFDY | 42 |
| Human CTLA-4 antagonist payload | | |
| CDR-H3 | | |
| [[OVTSB29]] | RASQSVGSSYLA | 43 |
| Human CTLA-4 antagonist payload | | |
| CDR-L1 | | |
| [[OVTSB29]] | GAFSRAT | 44 |
| Human CTLA-4 antagonist payload | | |
| CDR-L2 | | |
| [[OVTSB29]] | QQYGSSPWT | 45 |
| Human CTLA-4 antagonist payload | | |
| CDR-L3 | | |
| [[OVTSB29]] | QVQLVESGGGVVQPGRSLRLSCAAS | 46 |
| Human CTLA-4 antagonist payload | GFTFSSYTMHWVRQAPGKGLEWVTF | |
| heavy chain variable domain | ISYDGNNKYYADSVKGRFTISRDNS | |
| sequence. | KNTLYLQMNSLRAEDTAIYYCARTG | |
| | WLGPFDYWGQGTLVTVSS | |
| [[OVTSB29]] | EIVLTQSPGTLSLSPGERATLSCRA | 47 |
| Human CTLA-4 antagonist payload | SQSVGSSYLAWYQQKPGQAPRLLIY | |
| light chain variable domain sequence. | GAFSRATGIPDRFSGSGSGTDFTLT | |
| | ISRLEPEDFAVYYCQQYGSSPWTFG | |
| | QGTKVEIK | |
| [[OVTSB29]] | EPKSSDKTHTCPPCPAPELLGGPSV | 48 |
| Human CTLA-4 antagonist payload | FLFPPKPKDTLMISRTPEVTCVVVD | |
| heavy chain of human IgG1_G1m(17). | VSHEDPEVKFNWYVDGVEVHNAKTK | |
| | PREEQYNSTYRVVSVLTVLHQDWLN | |
| | GKEYKCKVSNKALPAPIEKTISKAK | |
| | GQPREPQVYTLPPSRDELTKNQVSL | |
| | TCLVKGFYPSDIAVEWESNGQPENN | |
| | YKTTPPVLDSDGSFFLYSKLTVDKS | |
| | RWQQGNVFSCSVMHEALHNHYTQKS | |
| | LSLSPGK | |
| [[OVTSB29]] | MAWVWTLLFLMAAAQSIQA | 49 |
| Signal peptide sequence in human | | |
| CTLA-4 antagonist payload. | | |
| [[OVTSB29]] | MAWVWTLLFLMAAAQSIQAEIVLTQ | 50 |
| Human CTLA-4 antagonist payload | SPGTLSLSPGERATLSCRASQSVGS | |
| | SYLAWYQQKPGQAPRLLIYGAFSRA | |
| | TGIPDRFSGSGSGTDFTLTISRLEP | |
| | EDFAVYYCQQYGSSPWTFGQGTKVE | |
| | IKGGSEGKSSGSGSESKSTGGSQVQ | |
| | LVESGGGVVQPGRSLRLSCAASGFT | |
| | FSSYTMHWVRQAPGKGLEWVTFISY | |
| | DGNNKYYADSVKGRFTISRDNSKNT | |
| | LYLQMNSLRAEDTAIYYCARTGWLG | |
| | PFDYWGQGTLVTVSSEPKSSDKTHT | |
| | CPPCPAPELLGGPSVFLFPPKPKDT | |
| | LMISRTPEVTCVVVDVSHEDPEVKF | |
| | NWYVDGVEVHNAKTKPREEQYNSTY | |
| | RVVSVLTVLHQDWLNGKEYKCKVSN | |
| | KALPAPIEKTISKAKGQPREPQVYT | |
| | LPPSRDELTKNQVSLTCLVKGFYPS | |
| | DIAVEWESNGQPENNYKTTPPVLDS | |
| | DGSFFLYSKLTVDKSRWQQGNVFSC | |
| | SVMHEALHNHYTQKSLSLSPGK | |
| [[OVTSB24]] | GSTISSVAVG | 51 |
| Mouse CTLA-4 antagonist payload | | |
| CDR-H1 | | |
| [[OVTSB24]] | TSSTSSTTAT | 52 |
| Mouse CTLA-4 antagonist payload | | |
| CDR-H2 | | |
| [[OVTSB24]] | GLTN | 53 |
| Mouse CTLA-4 antagonist payload | | |
| CDR-H3 | | |
| [[OVTSB24]] | QVQLVESGGGLAQPGGSLRLSCAAS | 54 |
| Mouse CTLA-4 antagonist payload | GSTISSVAVGWYRQTPGNQREWVAT | |
| VHH sequence. | SSTSSTTATYADSVKGRFTISRDNA | |
| | KNTIYLQMNSLKPEDTAVYYCKTGL | |
| | TNWGRGTQVTVSS | |
| [[OVTSB24]] | MAWVWTLLFLMAAAQSIQA | 55 |
| Signal peptide sequence in mouse | | |
| CTLA-4 antagonist payload | | |
| [[OVTSB24]] | MAWVWTLLFLMAAAQSIQAQVQLVE | 56 |
| Mouse CTLA-4 antagonist payload | SGGGLAQPGGSLRLSCAASGSTISS | |
| (including C-terminal lysine). | VAVGWYRQTPGNQREWVATSSTSST | |
| | TATYADSVKGRFTISRDNAKNTIYL | |
| | QMNSLKPEDTAVYYCKTGLTNWGRG | |
| | TQVTVSSEPRGPTIKPCPPCKCPAP | |
| | NLLGGPSVFIFPPKIKDVLMISLSP | |
| | IVTCVVVDVSEDDPDVQISWFVNNV | |
| | EVHTAQTQTHREDYNSTLRVVSALP | |
| | IQHQDWMSGKEFKCKVNNKDLPAPI | |
| | ERTISKPKGSVRAPQVYVLPPPEEE | |
| | MTKKQVTLTCMVTDFMPEDIYVEWT | |
| | NNGKTELNYKNTEPVLDSDGSYFMY | |
| | SKLRVEKKNWVERNSYSCSVVHEGL | |
| | HNHHTTKSFSRTPGK | |
| [[OVTSB24]] | MAWVWTLLFLMAAAQSIQAQVQLVE | 57 |
| Mouse CTLA-4 antagonist payload | SGGGLAQPGGSLRLSCAASGSTISS | |
| (without C-terminal lysine). | VAVGWYRQTPGNQREWVATSSTSST | |
| | TATYADSVKGRFTISRDNAKNTIYL | |
| | QMNSLKPEDTAVYYCKTGLTNWGRG | |
| | TQVTVSSEPRGPTIKPCPPCKCPAP | |
| | NLLGGPSVFIFPPKIKDVLMISLSP | |
| | IVTCVVVDVSEDDPDVQISWFVNNV | |
| | EVHTAQTQTHREDYNSTLRVVSALP | |
| | IQHQDWMSGKEFKCKVNNKDLPAPI | |
| | ERTISKPKGSVRAPQVYVLPPPEEE | |
| | MTKKQVTLTCMVTDFMPEDIYVEWT | |
| | NNGKTELNYKNTEPVLDSDGSYFMY | |
| | SKLRVEKKNWVERNSYSCSVVHEGL | |
| | HNHHTTKSFSRTPG | |
| [[OVTSB24]] | QVQLVESGGGLAQPGGSLRLSCAAS | 58 |
| Mouse CTLA-4 antagonist payload | GSTISSVAVGWYRQTPGNQREWVAT | |
| without signal peptide. (including C- | SSTSSTTATYADSVKGRFTISRDNA | |
| terminal lysine). | KNTIYLQMNSLKPEDTAVYYCKTGL | |
| | TNWGRGTQVTVSSEPRGPTIKPCPP | |
| | CKCPAPNLLGGPSVFIFPPKIKDVL | |
| | MISLSPIVTCVVVDVSEDDPDVQIS | |
| | WFVNNVEVHTAQTQTHREDYNSTLR | |
| | VVSALPIQHQDWMSGKEFKCKVNNK | |
| | DLPAPIERTISKPKGSVRAPQVYVL | |
| | PPPEEEMTKKQVTLTCMVTDFMPED | |
| | IYVEWTNNGKTELNYKNTEPVLDSD | |
| | GSYFMYSKLRVEKKNWVERNSYSCS | |
| | VVHEGLHNHHTTKSFSRTPGK | |
| [[OVTSB24]] | QVQLVESGGGLAQPGGSLRLSCAAS | 59 |
| Mouse CTLA-4 antagonist payload | GSTISSVAVGWYRQTPGNQREWVAT | |
| without signal peptide (without C- | SSTSSTTATYADSVKGRFTISRDNA | |
| terminal lysine). | KNTIYLQMNSLKPEDTAVYYCKTGL | |
| | TNWGRGTQVTVSSEPRGPTIKPCPP | |
| | CKCPAPNLLGGPSVFIFPPKIKDVL | |
| | MISLSPIVTCVVVDVSEDDPDVQIS | |
| | WFVNNVEVHTAQTQTHREDYNSTLR | |
| | VVSALPIQHQDWMSGKEFKCKVNNK | |
| | DLPAPIERTISKPKGSVRAPQVYVL | |
| | PPPEEEMTKKQVTLTCMVTDFMPED | |
| | IYVEWTNNGKTELNYKNTEPVLDSD | |
| | GSYFMYSKLRVEKKNWVERNSYSCS | |
| | VVHEGLHNHHTTKSFSRTPG | |
| [[OVTSB29]] | EIVLTQSPGTLSLSPGERATLSCRA | 60 |
| Human CTLA-4 antagonist payload | SQSVGSSYLAWYQQKPGQAPRLLIY | |
| without signal peptide. | GAFSRATGIPDRFSGSGSGTDFTLT | |
| | ISRLEPEDFAVYYCQQYGSSPWTFG | |
| | QGTKVEIKGGSEGKSSGSGSESKST | |
| | GGSQVQLVESGGGVVQPGRSLRLSC | |
| | AASGFTFSSYTMHWVRQAPGKGLEW | |
| | VTFISYDGNNKYYADSVKGRFTISR | |
| | DNSKNTLYLQMNSLRAEDTAIYYCA | |
| | RTGWLGPFDYWGQGTLVTVSSEPKS | |
| | SDKTHTCPPCPAPELLGGPSVFLFP | |
| | PKPKDTLMISRTPEVTCVVVDVSHE | |
| | DPEVKFNWYVDGVEVHNAKTKPREE | |
| | QYNSTYRVVSVLTVLHQDWLNGKEY | |
| | KCKVSNKALPAPIEKTISKAKGQPR | |
| | EPQVYTLPPSRDELTKNQVSLTCLV | |
| | KGFYPSDIAVEWESNGQPENNYKTT | |
| | PPVLDSDGSFFLYSKLTVDKSRWQQ | |
| | GNVFSCSVMHEALHNHYTQKSLSLS | |
| | PGK | |
| [[OVTSB29]] | GGSEGKSSGSGSESKSTGGS | 61 |
| Human CTLA-4 antagonist payload | | |
| linker sequence | | |

TABLE 7
Human FLT3L Payload Amino Acid Sequences.

| | | SEQ |
|---|---|---|
| | | ID |
| Description | Sequence | NO |
| [[OVTSW33]] | MTVLAPAWSPTTYLL | 70 |
| Signal | LLLLLSSGLSG | |
| peptide | | |
| sequence | | |
| in hFLT3L | | |
| payload. | | |
| [OVTSW33] | MTVLAPAWSPTTYLL | 71 |
| hFLT3L | LLLLLSSGLSGTQDC | |
| payload | SFQHSPISSDFAVKI | |
| | RELSDYLLQDYPVTV | |
| | ASNLQDEELCGGLWR | |
| | LVLAQRWMERLKTVA | |
| | GSKMQGLLERVNTEI | |
| | HFVTKCAFQPPPSCL | |
| | RFVQTNISRLLQETS | |
| | EQLVALKPWITRQNF | |
| | SRCLELQCQPDSSTL | |
| | PPPWSPRPLEATAPT | |
| | APQPPLLLLLLLPVG | |
| | LLLLAAAWCLHWQRT | |
| | RRRTPRPGEQVPPVP | |
| | SPQDLLLVEH | |
| [[OVTSW33]] | TQDCSFQHSPISSDF | 72 |
| hFLT3L | AVKIRELSDYLLQDY | |
| payload | PVTVASNLQDEELCG | |
| truncated | GLWRLVLAQRWMERL | |
| sequence | KTVAGSKMQGLLERV | |
| without | NTEIHFVTKCAFQPP | |
| signal | PSCLRFVQTNISRLL | |
| peptide. | QETSEQLVALKPWIT | |
| | RQNFSRCLELQCQPD | |
| | SSTLPPPWSPRPLEA | |
| | TAPTAPQPPLLLLLL | |
| | LPVGLLLLAAAWCLH | |
| | WQRTRRRTPRPGEQV | |
| | PPVPSPQDLLLVEH | |

TABLE 8
Immune Stealth Protein Amino Acid Sequences.

| Description | Sequence | SEQ ID NO |
|---|---|---|
| US11 | MSQTQPPAPVGPGDPDVYLKG | 80 |
| protein | VPSAGMHPRGVHAPRGHPRMI | |
| | SGPPQRGDNDQAAGQCGDSGL | |
| | LRVGADTTISKPSEAVRPPTI | |
| | PRTPRVPREPRVPRPPREPRE | |
| | PRVPRDPRDPRVPRDPRDPRQ | |
| | PRSPREPRSPREPRTPRTPRE | |
| | PRTARGSV | |
| Signal | PRSPLIVAVVAAALFAIV | 81 |
| peptide | | |
| in | | |
| UL49.5 | | |
| protein | | |
| UL49.5 | PRSPLIVAVVAAALFAIVRGR | 82 |
| protein | DPLLDAMRREGAMDFWSAGCY | |
| | ARGVPLSEPPQALVVFYVALT | |
| | AVMVAVALYAYGLCFRLMGAS | |
| | GPNKKESRGRG | |
| UL49.5 | RGRDPLLDAMRREGAMDFWSA | 83 |
| protein | GCYARGVPLSEPPQALVVFYV | |
| without | ALTAVMVAVALYAYGLCFRLM | |
| signal | GASGPNKKESRGRG | |
| peptide. | | |

TABLE 9
Additional amino acid sequences.

| | | SEQ |
|---|---|---|
| | | ID |
| Description | Sequence | NO |
| US 10 | MIKRRGNVEIRVYYESVRTLRSRSHLK | 90 |
| protein | PSDRQQSPGHRVFPGSPGFRDHPENLG | |
| | NPEYREIPETPGYRVTPGIHDNPGLPG | |
| | SPGLPGSPGPHAPPANHVRLAGLYSPG | |
| | KYAPLASPDPFSPQDGAYARARVGIHT | |
| | AVRVPPTGSSTHTHLRQDPGDEPTSDD | |
| | SGLYPLDARALAHLVMLPADHRAFFRT | |
| | VVEVSRMCAANVRDPPPPATGAMLGRH | |
| | ARLVHTQWLRANQETSPLWPWRTAAIN | |
| | FITTMAPRVQTHRHMHDLLMACAFWCC | |
| | LTHASTCSYAGLYSTHCLHLFGAFGCG | |
| | DPALTPPLC | |
| P2A | GSGATNFSLLKQAGDVEENPGP | 91 |
| sequence | | |
| within | | |
| hFLT3L- | | |
| P2A-UL49.5 | | |
| polypeptide. | | |
| hFLT3L- | MTVLAPAWSPTTYLLLLLLLSSGLSGT | 92 |
| P2A- | QDCSFQHSPISSDFAVKIRELSDYLLQ | |
| UL49.5 | DYPVTVASNLQDEELCGGLWRLVLAQR | |
| polypeptide. | WMERLKTVAGSKMQGLLERVNTEIHFV | |
| | TKCAFQPPPSCLRFVQTNISRLLQETS | |
| | EQLVALKPWITRQNFSRCLELQCQPDS | |
| | STLPPPWSPRPLEATAPTAPQPPLLLL | |
| | LLLPVGLLLLAAAWCLHWQRTRRRTPR | |
| | PGEQVPPVPSPQDLLLVEHGSGATNFS | |
| | LLKQAGDVEENPGPRSPLIVAVVAAAL | |
| | FAIVRGRDPLLDAMRREGAMDFWSAGC | |
| | YARGVPLSEPPQALVVFYVALTAVMVA | |
| | VALYAYGLCFRLMGASGPNKKESRGRG | |
| hFLT3L-P2A- | TQDCSFQHSPISSDFAVKIRELSDYLL | 93 |
| UL49.5 | QDYPVTVASNLQDEELCGGLWRLVLAQ | |
| polypeptide | RWMERLKTVAGSKMQGLLERVNTEIHF | |
| without | VTKCAFQPPPSCLRFVQTNISRLLQET | |
| signal | SEQLVALKPWITRQNFSRCLELQCQPD | |
| peptide | SSTLPPPWSPRPLEATAPTAPQPPLLL | |
| sequence. | LLLLPVGLLLLAAAWCLHWQRTRRRTP | |
| | RPGEQVPPVPSPQDLLLVEHGSGATNF | |
| | SLLKQAGDVEENPGPRSPLIVAVVAAA | |
| | LFAIVRGRDPLLDAMRREGAMDFWSAG | |
| | CYARGVPLSEPPQALVVFYVALTAVMV | |
| | AVALYAYGLCFRLMGASGPNKKESRGR | |
| | G | |
| Soluble | TQDCSFQHSPISSDFAVKIRELSDYLL | 318 |
| FLT3L | QDYPVTVASNLQDEELCGGLWRLVLAQ | |
| | RWMERLKTVAGSKMQGLLERVNTEIHF | |
| | VTKCAFQPPPSCLRFVQTNISRLLQET | |
| | SEQLVALKPWITRQNFSRCLELQCQPD | |
| | SSTLPPPWSPRPLEATAPTAPQPP | |

TABLE 10
γ34.5 Locus Cassette Sequences.

| | | SEQ |
|---|---|---|
| | | ID |
| Description | Sequence | NO |
| γ34.5 locus | GCTATTGTCTTCCCAATCCTCCCCCTTGCTGTCCTGCCCCACCCCAC | 100 |
| cassette | CCCCCAGAATAGAATGACACCTACTCAGACAATGCGATGCAATTTCC | |
| (BGHpA- | TCATTTTATTAGGAAAGGACAGTGGGAGTGGCACCTTCCAGGGTCAA | |
| UL49.5- | GGAAGGCACGGGGGAGGGGCAAACAACAGATGGCTGGCAACTAGAAG | |
| P2A- | GCACAGTCGAGGCTGATCAGCGAGCTCTAGCATTTAGGTGACACTAT | |
| hFLT3L- | AGAATAGGGCCCTGCATGCTCGAGCTGGCCTGGGCTTCCTCAGCCCC | |
| CMV) | GCCCCCGCGACTCCTTTTTATTGGGCCCGCTGGCGCCCATGAGCCTA | |
| 3′ > 5′ | AAGCAAAGCCCGTACGCGTACAGGGCCACGGCGACCATTACCGCGGT | |
| orientation | CAGGGCCACGTAAAAAACAACCAGGGCCTGCGGTGGCTCCGAGAGCG | |
| | GCACCCCGCGCGCGTAGCAGCCTGCGCTCCAAAAGTCCATTGCCCCC | |
| | TCGCGCCGCATCGCGTCTAGCAGGGGGTCGCGGCCGCGCACGATGGC | |
| | AAACAGCGCGGCGGCCACAACCGCAACGATGAGCGGCGACCGAGGAC | |
| | CGGGGTTTTCTTCCACGTCTCCTGCTTGCTTTAACAGAGAGAAGTTC | |
| | GTGGCTCCAGAGCCGTGCTCCACAAGCAGCAGGTCCTGGGGACTGGG | |
| | GACGGGGGGCACCTGCTCCCCAGGGCGGGGTGTCCTCCGCCGCGTCC | |
| | TCTGCCAGTGCAGGCACCAGGCAGCGGCCAGCAGCAGGAGGCCCACG | |
| | GGCAGCAGCAGTAGGAGGAGCAGAGGGGGCTGCGGGGCTGTCGGGGC | |
| | TGTGGCCTCCAGGGGCCGGGGACTCCATGGGGGTGGCAGGGTTGAGG | |
| | AGTCGGGCTGACACTGCAGCTCCAGGCACCGGGAGAAGTTCTGGCGA | |
| | GTGATCCAGGGCTTCAGCGCCACCAGCTGCTCGGAGGTCTCCTGCAG | |
| | GAGGCGGGAGATGTTGGTCTGGACGAAGCGAAGACAGCTGGGGGGGG | |
| | GCTGAAAGGCACATTTGGTGACAAAGTGTATCTCCGTGTTCACGCGC | |
| | TCCAGCAAGCCTTGCATCTTGGACCCAGCGACAGTCTTGAGCCGCTC | |
| | CATCCAGCGCTGTGCCAGGACCAGCCGCCAGAGGCCCCCGCAGAGCT | |
| | CCTCGTCCTGCAGGTTGGAGGCCACGGTGACTGGGTAATCTTGAAGC | |
| | AGGTAGTCAGACAGCTCACGGATTTTGACAGCGAAGTCGGAGGAGAT | |
| | GGGGCTGTGTTGGAAGGAGCAGTCCTGGGTCCCACTGAGTCCCGAGC | |
| | TCAGCAGCAGCAGCAGGAGGAGATAGGTTGTTGGGCTCCAGGCTGGC | |
| | GCCAGCACTGTCATGGTGGCGACCGGTAGCGCTAGCGGATCTGACGG | |
| | TTCACTAAACGAGCTCTGCTTATATAGACCTCCCACCGTACACGCCT | |
| | ACCGCCCATTTGCGTCAATGGGGCGGAGTTGTTACGACATTTTGGAA | |
| | AGTCCCGTTGATTTTGGTGCCAAAACAAACTCCCATTGACGTCAATG | |
| | GGGTGGAGACTTGGAAATCCCCGTGAGTCAAACCGCTATCCACGCCC | |
| | ATTGATGTACTGCCAAAACCGCATCACCATGGTAATAGCGATGACTA | |
| | ATACGTAGATGTACTGCCAAGTAGGAAAGTCCCATAAGGTCATGTAC | |
| | TGGGCATAATGCCAGGCGGGCCATTTACCGTCATTGACGTCAATAGG | |
| | GGGCGTACTTGGCATATGATACACTTGATGTACTGCCAAGTGGGCAG | |
| | TTTACCGTAAATAGTCCACCCATTGACGTCAATGGAAAGTCCCTATT | |
| | GGCGTTACTATGGGAACATACGTCATTATTGACGTCAATGGGGGGGG | |
| | GTCGTTGGGCGGTCAGCCAGGCGGGCCATTTACCGTAAGTTATGTAA | |
| | CGCGGAACTCCATATATGGGCTATGAACTAATGACCCCGTAATTGAT | |
| | TACTATTAATAACTAGTCAATAATCAATGTCAAC | |
| γ34.5 locus | TGGAGGCGCTGGCCGAGGGCTTCGACGGCGACCTGGCGGCCGTCCCG | 101 |
| cassette | GGGCTGGCCGGGGCCCGGCCCGCCAGCCCCCCGCGGCCGGAGGGACC | |
| including left | CGCGGGCCCCGCTTCCCCGCCGCCGCCGCACGCCGACGCGCCCCGCC | |
| and right | TGCGCGCGTGGCTGCGCGAGCTGCGGTTCGTGCGCGACGCGCTGGTG | |
| homology | CTCATGCGCCTGCGCGGGGACCTGCGCGTGGCCGGCGGCAGCGAGGC | |
| arms | CGCCGTGGCCGCCGTGCGCGCCGTGAGCCTGGTCGCCGGGGCCCTGG | |
| 3′ > 5′ | GCCCCGCGCTGCCGCGGGACCCGCGCCTGCCGAGCTCCGCGGCCGCC | |
| orientation | GCCGCCGCGGACCTGCTGTTTGACAACCAGAGCCTGCGCCCCCTGCT | |
| | GGCGGCGGCGGCCAGCGCACCGGACGCCGCCGACGCGCTGGCGGCCG | |
| | CCGCCGCCTCCGCCGCGCCGCGGGAGGGGCGCAAGCGCAAGAGTCCC | |
| | GGCCCGGCCCGGCCGCCCGGAGGCGGCGGCCCGCGACCCCCGAAGAC | |
| | GAAGAAGAGCGGCGCGGACGCCCCCGGCTCGGACGCCCGCGCCCCCC | |
| | TCCCCGCGCCCGCGCCCCCCTCCACGCCCCCGGGGCCCGAGCCCGCC | |
| | CCCGCCCAGCCCGCGGCGCCCCGGGCCGCCGCGGCGCAGGCCCGCCC | |
| | GCGCCCCGTGGCCGTGTCGCGCCGGCCCGCCGAGGGCCCCGACCCCC | |
| | TGGGCGGCTGGCGGCGGCAGCCCCCGGGGCCCAGCCACACGGCGGCG | |
| | CCCGCGGCCGCCGCCCTGGAGGCCTACTGCTCCCCGCGCGCCGTGGC | |
| | CGAGCTCACGGACCACCCGCTGTTCCCCGTCCCCTGGCGACCGGCCC | |
| | TCATGTTTGACCCGCGGGCCCTGGCCTCGATCGCCGCGCGGTGCGCC | |
| | GGGCCCGCCCCCGCCGCCCAGGCCGCGTGCGGCGGCGGCGACGACGA | |
| | CGATAACCCCCACCCCCACGGGGCCGCCGGGGGCCGCCTCTTTGGCC | |
| | CCCTGCGCGCCTCGGGCCCGCTGCGCCGCATGGCGGCCTGGATGCGC | |
| | CAGATCCCCGACCCCGAGGACGTGCGCGTGGTGGTGCTGTACTCGCC | |
| | GCTGCCGGGCGAGGACCTGGCCGGCGGCGGGGCCTCGGGGGGGCCGC | |
| | CGGAGTGGTCCGCCGAGCGCGGCGGGCTGTCCTGCCTGCTGGCGGCC | |
| | CTGGCCAACCGGCTGTGCGGGCCGGACACGGCCGCCTGGGCGGGCAA | |
| | TTGGACCGGCGCCCCCGACGTGTCGGCGCTGGGCGCACAGGGCGTGC | |
| | TGCTGCTGTCCACGCGGGACCTGGCCTTCGCCGGGGCCGTGGAGTTT | |
| | CTGGGGCTGCTCGCCAGCGCCGGCGACCGGCGGCTCATCGTGGTCAA | |
| | CACCGTGCGCGCCTGCGACTGGCCCGCCGACGGGCCCGCGGTGTCGC | |
| | GGCAGCACGCCTACCTGGCGTGCGAGCTGCTGCCCGCCGTGCAGTGC | |
| | GCCGTGCGCTGGCCGGCGGCGCGGGACCTGCGCCGCACGGTGCTGGC | |
| | CTCGGGCCGCGTGTTCGGCCCGGGGGTCTTCGCGCGCGTGGAGGCCG | |
| | CGCACGCGCGCCTGTACCCCGACGCGCCGCCGCTGCGCCTGTGCCGC | |
| | GGCGGCAACGTGCGCTACCGCGTGCGCACGCGCTTCGGCCCGGACAC | |
| | GCCGGTGCCCATGTCCCCGCGCGAGTACCGCCGGGCCGTGCTGCCGG | |
| | CGCTGGACGGCCGGGCGGCGGCCTCGGGGACCACCGACGCCATGGCG | |
| | CCCGGCGCGCCGGACTTCTGCGAGGAGGAGGCCCACTCGCACGCCGC | |
| | CTGCGCGCGCTGGGGCCTGGGCGCGCCGCTGCGGCCCGTGTACGTGG | |
| | CGCTGGGGCGCGAGGCGGTGCGCGCCGGCCCGGCCCGGTGGCGCGGG | |
| | CCGCGGAGGGACTTTTGCGCCCGCGCCCTGCTGGAGCCCGACGACGA | |
| | CGCCCCCCCGCTGGTGCTGCGCGGCGACGACGACGGCCCGGGGGCCC | |
| | TGCCGCCGGCGCCGCCCGGGATTCGCTGGGCCTCGGCCACGGGCCGC | |
| | AGCGGCACCGTGCTGGCGGCGGCGGGGGCCGTGGAGGTGCTGGGGGC | |
| | GGAGGCGGGCTTGGCCACGCCCCCGCGGCGGGAAGTTGTGGACTGGG | |
| | AAGGCGCCTGGGACGAAGACGACGGCGGCGCGTTCGAGGGGGACGGG | |
| | GTGCTGTAACGGGCCGGGACGGGGCGGGGCGCTTGTGAGACCCGAAG | |
| | ACGCAATAAACGGCAACAACCTGATTAAGTTTTGCAGTAGCGTTGTT | |
| | TATTCGAGGGGGGGGAGGGGGCGAGGGGGGGGAGGGGGCGAGGGGCG | |
| | GGAGGGGGCGAGGGGGGGGAGGGGGCGAGGGGGGGGAGGGGGCGAGG | |
| | GGCGGGAGGGGGCGAGGGGGGGGAGGGGGCGAGGGGGGGGAGGGGGC | |
| | GAGGGGGGGGAGGGGGCGAGGGGGGGGAGGGGGCGAGGGGGGGGAGG | |
| | GGGCGAGGGGGGGGAGGGGGCGAGGGGGGGGAGGGGGCGAGGGGCGG | |
| | GAGGGGGCGAGGGGGGGGAGGGGGCGAGGGGGGGGAGGGGGCGAGGG | |
| | GCGGGAGGGGGCGAGGGGGGGGAGGGGGCGAGGGGGGGGAGGGGGCG | |
| | AGGGGCGGTGGTGGTGCGCGGGCGCCCCCGGAGGGTTTGGATCTCTG | |
| | ACCTGAGATTGGCGGCACTGAGGTAGAGATGCCCGAACCCCCCCGAG | |
| | GGAGCGCGGGACGCGCCGGGGAGGGCTGGGGCCGGGGAGGGCTGGGG | |
| | CCGGGGAGGGCTGGGGCCGGGGAGGGCTGGGGCCGGGGAGGGCTGGG | |
| | GCCGGGGAGGGCTGGGGCCGGGGAGGGCTGGGGCTGGGGAGGGCTGG | |
| | GGCTGGGGAGGGGGCGGTGGTGTGTAGCAGGAGCGGTGTGTTGCGCC | |
| | GGGGTACGTCTGGAGGAGCGGGAGGTGCGCGGTGACGTGTGGATGAG | |
| | GAACAGGAGTTGTTGCGCGGTGAGTTGTCGCTGTGAGTTGTGTTGTT | |
| | GGGCAGGTGTGGTGGATGACGTGACGTGTGACGTGCGGAGTGCGCCG | |
| | TGCTCTGTTGGTTTCACCTGTGGCAGCCCGGGCCCCCCGCGGGCGCG | |
| | CGCGCGCGCAAAAAAGGCGGGCGGCGGTCCGGGCGGCGTGCGCGCGC | |
| | GCGGCGGGCGTTGGGGGCGGGGCCGCGGGAGCGGGGGGAGGAGCGGG | |
| | GGGAGGAGCGGGGGGAGGAGCGGGGGGAGGAGCGGGGGGAGGAGCGG | |
| | GGGGAGGAGCGGGGGGAGGAGCGGCCAGACCCCGAAAACGGGCCCCC | |
| | CCAAAACACACCCCCCGGGGGTCGCGCGCGGCCCTTTAAAGCGCGGC | |
| | GGCGGGCAGCCCGGGCCCCCCGCGGCCGAGACTAGCGAGTTAGACAG | |
| | GCAAGCACTACTCGCCTCTGCACGCACATGCTTGCCTGTCAAACTCT | |
| | ACCACCCCGGCACGCTCTCTGTCTCCATGGCCCGCCGCCGCCGCCAT | |
| | CGCGGCCCCCGCCGCCCCCGGCCGCCCGGGCCCACGGGCGCGGTCCC | |
| | AACCGCACAGTCCCAGGTAACCTCCACGCCCAACTCGGAACCCGTGG | |
| | TCAGGAGCGCGCCCGCGGCCGCTATTGTCTTCCCAATCCTCCCCCTT | |
| | GCTGTCCTGCCCCACCCCACCCCCCAGAATAGAATGACACCTACTCA | |
| | GACAATGCGATGCAATTTCCTCATTTTATTAGGAAAGGACAGTGGGA | |
| | GTGGCACCTTCCAGGGTCAAGGAAGGCACGGGGGAGGGGCAAACAAC | |
| | AGATGGCTGGCAACTAGAAGGCACAGTCGAGGCTGATCAGCGAGCTC | |
| | TAGCATTTAGGTGACACTATAGAATAGGGCCCTGCATGCTCGAGCTG | |
| | GCCTGGGCTTCCTCAGCCCCGCCCCCGCGACTCCTTTTTATTGGGCC | |
| | CGCTGGCGCCCATGAGCCTAAAGCAAAGCCCGTACGCGTACAGGGCC | |
| | ACGGCGACCATTACCGCGGTCAGGGCCACGTAAAAAACAACCAGGGC | |
| | CTGCGGTGGCTCCGAGAGCGGCACCCCGCGCGCGTAGCAGCCTGCGC | |
| | TCCAAAAGTCCATTGCCCCCTCGCGCCGCATCGCGTCTAGCAGGGGG | |
| | TCGCGGCCGCGCACGATGGCAAACAGCGCGGCGGCCACAACCGCAAC | |
| | GATGAGCGGCGACCGAGGACCGGGGTTTTCTTCCACGTCTCCTGCTT | |
| | GCTTTAACAGAGAGAAGTTCGTGGCTCCAGAGCCGTGCTCCACAAGC | |
| | AGCAGGTCCTGGGGACTGGGGACGGGGGGCACCTGCTCCCCAGGGCG | |
| | GGGTGTCCTCCGCCGCGTCCTCTGCCAGTGCAGGCACCAGGCAGCGG | |
| | CCAGCAGCAGGAGGCCCACGGGCAGCAGCAGTAGGAGGAGCAGAGGG | |
| | GGCTGCGGGGCTGTCGGGGCTGTGGCCTCCAGGGGCCGGGGACTCCA | |
| | TGGGGGTGGCAGGGTTGAGGAGTCGGGCTGACACTGCAGCTCCAGGC | |
| | ACCGGGAGAAGTTCTGGCGAGTGATCCAGGGCTTCAGCGCCACCAGC | |
| | TGCTCGGAGGTCTCCTGCAGGAGGCGGGAGATGTTGGTCTGGACGAA | |
| | GCGAAGACAGCTGGGGGGGGGCTGAAAGGCACATTTGGTGACAAAGT | |
| | GTATCTCCGTGTTCACGCGCTCCAGCAAGCCTTGCATCTTGGACCCA | |
| | GCGACAGTCTTGAGCCGCTCCATCCAGCGCTGTGCCAGGACCAGCCG | |
| | CCAGAGGCCCCCGCAGAGCTCCTCGTCCTGCAGGTTGGAGGCCACGG | |
| | TGACTGGGTAATCTTGAAGCAGGTAGTCAGACAGCTCACGGATTTTG | |
| | ACAGCGAAGTCGGAGGAGATGGGGCTGTGTTGGAAGGAGCAGTCCTG | |
| | GGTCCCACTGAGTCCCGAGCTCAGCAGCAGCAGCAGGAGGAGATAGG | |
| | TTGTTGGGCTCCAGGCTGGCGCCAGCACTGTCATGGTGGCGACCGGT | |
| | AGCGCTAGCGGATCTGACGGTTCACTAAACGAGCTCTGCTTATATAG | |
| | ACCTCCCACCGTACACGCCTACCGCCCATTTGCGTCAATGGGGCGGA | |
| | GTTGTTACGACATTTTGGAAAGTCCCGTTGATTTTGGTGCCAAAACA | |
| | AACTCCCATTGACGTCAATGGGGTGGAGACTTGGAAATCCCCGTGAG | |
| | TCAAACCGCTATCCACGCCCATTGATGTACTGCCAAAACCGCATCAC | |
| | CATGGTAATAGCGATGACTAATACGTAGATGTACTGCCAAGTAGGAA | |
| | AGTCCCATAAGGTCATGTACTGGGCATAATGCCAGGCGGGCCATTTA | |
| | CCGTCATTGACGTCAATAGGGGGCGTACTTGGCATATGATACACTTG | |
| | ATGTACTGCCAAGTGGGCAGTTTACCGTAAATAGTCCACCCATTGAC | |
| | GTCAATGGAAAGTCCCTATTGGCGTTACTATGGGAACATACGTCATT | |
| | ATTGACGTCAATGGGGGGGGGTCGTTGGGCGGTCAGCCAGGCGGGCC | |
| | ATTTACCGTAAGTTATGTAACGCGGAACTCCATATATGGGCTATGAA | |
| | CTAATGACCCCGTAATTGATTACTATTAATAACTAGTCAATAATCAA | |
| | TGTCAACTCTAGATCTGCTGAGGTCTAGAGCGGCCGCCAGCGCGGCG | |
| | GGGCCCGGCCAACCAGCGTCCGCCGAGTCGTCGGGGCCCGGCCCACT | |
| | GGGCGGGAGTTACCGCCCAGTGGGCCGGGCCGCCCACTTCCCGGTAT | |
| | GGTAATTAAAAACTTGCAGAGGCCTTGTTCCGCTTCCCGGTATGGTA | |
| | ATTAGAAACTCATTAATGGGCGGCCCCGGCCGCCCTTCCCGCTTCCG | |
| | GCAATTCCCGCGGCCCTTAATGGGCAACCCCGGTATTCCCCGCCTCC | |
| | CGCGCCGCGCGTAACCAATCCCCTGGGGTTCCGGGTTATGCTAATTG | |
| | CTTTTTTGGCGGAACACACGGCCCCTCGCGCATTGGCCCGCGGGTCG | |
| | CTCAATGAACCCGCATTGGTCCCCTGGGGTTCCGGGTATGGTAATGA | |
| | GTTTCTTCGGGAAGGCGGGAAGCCCCGGGGCACCGACGCAGGCCAAG | |
| | CCCCTGTTGCGTCGGCGGGAGGGGCATGCTAATGGGGTTCTTTGGGG | |
| | GACACCGGGTTGGGCCCCCAAATCGGGGGCCGGGCCGTGCATGCTAA | |
| | TGATATTCTTTGGGGGCGCCGGGTTGGTCCCCGGGGACGGGGCCGCC | |
| | CCGCGGTGGGCCTGCCTCCCCTGGGACGCGCGGCCATTGGGGGAATC | |
| | GTCACTGCCGCCCCTTTGGGGAGGGGAAAGGCGTGGGGTATAAGTTA | |
| | GCCCTGGCCCGACAGTCTGGTCGCATTTGCACCTCGGCACTCGGAGC | |
| | GAGACGCAGCAGCCAGGCAGACTCGGGCCGCCCCCTCTCCGCATCAC | |
| | CACAGAAGCCCCGCCTACGTTGCGACCCCCAGGGACCCTCCGTCCGC | |
| | GACCCTCCAGCCGCATACGACCCCCATGGAGCCCCGCCCCGGAGCGA | |
| | GTACCCGCCGGCCTGAGGGCCGCCCCCAGCGCGAGGTGAGGGGCCGG | |
| | GCGCCATGTCTGGGGCGCCATATTGGGGGGCGCCATATTGGGGGGCG | |
| | CCATGTTGGGGGACCCCCGACCCTTACACTGGAACCGGCCGCCATGT | |
| | TGGGGGACCCCCACTCATACACGGGAGCCGGGCGCCATGTTGGGGCG | |
| | CCATGTTAGGGGGCGTGGAACCCCGTGACACTATATATACAGGGACC | |
| | GGGGGCGCCATGTTAGGGGGTGCGGAACCCCCTGACCCTATATATAC | |
| | AGGGACCGGGGTCGCCCTGTTGGGGGTCGCCATGTGACCCCCTGACT | |
| | TTATATATACAGACCCCCAACACATACACATGGCCCCTTTGACTCAG | |
| | ACGCAGGGCCCGGGGTCGCCGTGGGACCCCCTGACTCATACACAGAG | |
| | ACACGCCCCCACAACAAACACACAAGGACCGGGGTCGCCGTGTTGGG | |
| | GGCGTGGTCCCCACTGACTCATACGCAGGCCCCCCTTACTCACACGC | |
| | ATCTAGGGGGGTGGGGAGGAGCCGCCCGCCATATTTGGGGGACGCCG | |
| | TGGGACCCCCGACTCCGGTGCGTCTGGAGGGCGGGAGAAGAGGGAAG | |
| | AAGAGGGGTCGG | |
| BGHpA | GCTATTGTCTTCCCAATCCTCCCCCTTGCTGTCCTGCCCCACCCCAC | 102 |
| 3′ > 5′ | CCCCCAGAATAGAATGACACCTACTCAGACAATGCGATGCAATTTCC | |
| orientation | TCATTTTATTAGGAAAGGACAGTGGGAGTGGCACCTTCCAGGGTCAA | |
| | GGAAGGCACGGGGGAGGGGCAAACAACAGATGGCTGGCAACTAGAAG | |
| | GCACAG | |
| UL49.5 | TCAGCCCCGCCCCCGCGACTCCTTTTTATTGGGCCCGCTGGCGCCCA | 103 |
| 3′ > 5′ | TGAGCCTAAAGCAAAGCCCGTACGCGTACAGGGCCACGGCGACCATT | |
| orientation | ACCGCGGTCAGGGCCACGTAAAAAACAACCAGGGCCTGCGGTGGCTC | |
| | CGAGAGCGGCACCCCGCGCGCGTAGCAGCCTGCGCTCCAAAAGTCCA | |
| | TTGCCCCCTCGCGCCGCATCGCGTCTAGCAGGGGGTCGCGGCCGCGC | |
| | ACGATGGCAAACAGCGCGGCGGCCACAACCGCAACGATGAGCGGCGA | |
| | CCGAGG | |
| P2A self- | AGGACCGGGGTTTTCTTCCACGTCTCCTGCTTGCTTTAACAGAGAGA | 104 |
| cleaving | AGTTCGTGGCTCCAGAGCC | |
| peptide | | |
| 3′ > 5′ | | |
| orientation | | |
| hFLT3L | GTGCTCCACAAGCAGCAGGTCCTGGGGACTGGGGACGGGGGGCACCT | 105 |
| 3′ > 5′ | GCTCCCCAGGGCGGGGTGTCCTCCGCCGCGTCCTCTGCCAGTGCAGG | |
| orientation | CACCAGGCAGCGGCCAGCAGCAGGAGGCCCACGGGCAGCAGCAGTAG | |
| | GAGGAGCAGAGGGGGCTGCGGGGCTGTCGGGGCTGTGGCCTCCAGGG | |
| | GCCGGGGACTCCATGGGGGTGGCAGGGTTGAGGAGTCGGGCTGACAC | |
| | TGCAGCTCCAGGCACCGGGAGAAGTTCTGGCGAGTGATCCAGGGCTT | |
| | CAGCGCCACCAGCTGCTCGGAGGTCTCCTGCAGGAGGCGGGAGATGT | |
| | TGGTCTGGACGAAGCGAAGACAGCTGGGGGGGGGCTGAAAGGCACAT | |
| | TTGGTGACAAAGTGTATCTCCGTGTTCACGCGCTCCAGCAAGCCTTG | |
| | CATCTTGGACCCAGCGACAGTCTTGAGCCGCTCCATCCAGCGCTGTG | |
| | CCAGGACCAGCCGCCAGAGGCCCCCGCAGAGCTCCTCGTCCTGCAGG | |
| | TTGGAGGCCACGGTGACTGGGTAATCTTGAAGCAGGTAGTCAGACAG | |
| | CTCACGGATTTTGACAGCGAAGTCGGAGGAGATGGGGCTGTGTTGGA | |
| | AGGAGCAGTCCTGGGTCCCACTGAGTCCCGAGCTCAGCAGCAGCAGC | |
| | AGGAGGAGATAGGTTGTTGGGCTCCAGGCTGGCGCCAGCACTGTCAT | |
| hFLT3L- | TCAGCCCCGCCCCCGCGACTCCTTTTTATTGGGCCCGCTGGCGCCCA | 106 |
| P2A-UL49.5 | TGAGCCTAAAGCAAAGCCCGTACGCGTACAGGGCCACGGCGACCATT | |
| 3′ > 5′ | ACCGCGGTCAGGGCCACGTAAAAAACAACCAGGGCCTGCGGTGGCTC | |
| orientation | CGAGAGCGGCACCCCGCGCGCGTAGCAGCCTGCGCTCCAAAAGTCCA | |
| | TTGCCCCCTCGCGCCGCATCGCGTCTAGCAGGGGGTCGCGGCCGCGC | |
| | ACGATGGCAAACAGCGCGGCGGCCACAACCGCAACGATGAGCGGCGA | |
| | CCGAGGACCGGGGTTTTCTTCCACGTCTCCTGCTTGCTTTAACAGAG | |
| | AGAAGTTCGTGGCTCCAGAGCCGTGCTCCACAAGCAGCAGGTCCTGG | |
| | GGACTGGGGACGGGGGGCACCTGCTCCCCAGGGCGGGGTGTCCTCCG | |
| | CCGCGTCCTCTGCCAGTGCAGGCACCAGGCAGCGGCCAGCAGCAGGA | |
| | GGCCCACGGGCAGCAGCAGTAGGAGGAGCAGAGGGGGCTGCGGGGCT | |
| | GTCGGGGCTGTGGCCTCCAGGGGCCGGGGACTCCATGGGGGTGGCAG | |
| | GGTTGAGGAGTCGGGCTGACACTGCAGCTCCAGGCACCGGGAGAAGT | |
| | TCTGGCGAGTGATCCAGGGCTTCAGCGCCACCAGCTGCTCGGAGGTC | |
| | TCCTGCAGGAGGCGGGAGATGTTGGTCTGGACGAAGCGAAGACAGCT | |
| | GGGGGGGGGCTGAAAGGCACATTTGGTGACAAAGTGTATCTCCGTGT | |
| | TCACGCGCTCCAGCAAGCCTTGCATCTTGGACCCAGCGACAGTCTTG | |
| | AGCCGCTCCATCCAGCGCTGTGCCAGGACCAGCCGCCAGAGGCCCCC | |
| | GCAGAGCTCCTCGTCCTGCAGGTTGGAGGCCACGGTGACTGGGTAAT | |
| | CTTGAAGCAGGTAGTCAGACAGCTCACGGATTTTGACAGCGAAGTCG | |
| | GAGGAGATGGGGCTGTGTTGGAAGGAGCAGTCCTGGGTCCCACTGAG | |
| | TCCCGAGCTCAGCAGCAGCAGCAGGAGGAGATAGGTTGTTGGGCTCC | |
| | AGGCTGGCGCCAGCACTGTCAT | |
| hCMV | GATCTGACGGTTCACTAAACGAGCTCTGCTTATATAGACCTCCCACC | 107 |
| promoter | GTACACGCCTACCGCCCATTTGCGTCAATGGGGCGGAGTTGTTACGA | |
| | CATTTTGGAAAGTCCCGTTGATTTTGGTGCCAAAACAAACTCCCATT | |
| | GACGTCAATGGGGTGGAGACTTGGAAATCCCCGTGAGTCAAACCGCT | |
| | ATCCACGCCCATTGATGTACTGCCAAAACCGCATCACCATGGTAATA | |
| | GCGATGACTAATACGTAGATGTACTGCCAAGTAGGAAAGTCCCATAA | |
| | GGTCATGTACTGGGCATAATGCCAGGCGGGCCATTTACCGTCATTGA | |
| | CGTCAATAGGGGGCGTACTTGGCATATGATACACTTGATGTACTGCC | |
| | AAGTGGGCAGTTTACCGTAAATAGTCCACCCATTGACGTCAATGGAA | |
| | AGTCCCTATTGGCGTTACTATGGGAACATACGTCATTATTGACGTCA | |
| | ATGGGCGGGGGTCGTTGGGCGGTCAGCCAGGCGGGCCATTTACCGTA | |
| | AGTTATGTAACGCGGAACTCCATATATGGGCTATGAACTAATGACCC | |
| | CGTAATTGATTACTATTAATAACTAGTCAATAATCAATGTCAAC | |

TABLE 11
US10-12 Locus Cassette Sequences.

| | | |
|---|---|---|
| | | SEQ |
| | | ID |
| Description | Sequence | NO |

Human payload expression cassette sequences

| | | |
|---|---|---|
| 37E | TTAATTAAGGGTGGCATCCCTGTGACCCCTCCCCAGTGCCTCTCCTG | 200 |
| expression | GCCCTGGAAGTTGCCACTCCAGTGCCCACCAGCCTTGTCCTAATAAA | |
| cassette | ATTAAGTTGCATCATTTTGTCTGACTAGGTGTCCTTCTATAATATTA | |
| encoding | TGGGGTGGAGGGGGGTGGTATGGAGCAAGGGGCAAGTTGGGAAGACA | |
| human | ACCTGTAGGGCCTGCGGGGTCTATTGGGAACCAAGCTGGAGTGCAGT | |
| CTLA-4 | GGCACAATCTTGGCTCACTGCAATCTCCGCCTCCTGGGTTCAAGCGA | |
| antagonist, | TTCTCCTGCCTCAGCCTCCCGAGTTGTTGGGATTCCAGGCATGCATG | |
| human | ACCAGGCTCAGCTAATTTTTGTTTTTTTGGTAGAGACGGGGTTTCAC | |
| CD40 | CATATTGGCCAGGCTGGTCTCCAACTCCTAATCTCAGGTGATCTACC | |
| agonist, | CACCTTGGCCTCCCAAATTGCTGGGATTACAGGCGTGAACCACTGCT | |
| and | CCCTTCCCTGTCCTTTGGCACGTATACTGAGTCATTAGGGACTTTCC | |
| human | AATGGGTTTTGCCCAGTACATAAGGTCAATAGGGGTGAATCAACAGG | |
| scIL-12 | AAAGTCCCATTGGAGCCAAGTACACTGAGTCAATAGGGACTTTCCAT | |
| payloads. | TGGGTTTTGCCCAGTACAAAAGGTCAATAGGGGGTGAGTCAATGGGT | |
| 5′ > 3′ | TTTTCCCATTATTGGCACGTACATAAGGTCAATAGGGGTGAGTCATT | |
| direction | GGGTTTTTCCAGCCAATTTAATTTAAACGCCATGTACTTTCCCACCA | |
| | TTGACGTCAATGGGCTATTGAAACTAATGCAACGTGACCTTTAAACG | |
| | GTACTTTCCCATAGCTGATTAATGGGAAAGTACCGTTCTCGAGCCAA | |
| | TACACGTCAATGGGAAGTGAAAGGGCAGCCAAAACGTAACACCGCCC | |
| | CGGTTTTCCCCTGGAAATTCCATATTGGCACGCATTCTATTGGCTGA | |
| | GCTGCGTTCTACGTGGGTATAAGAGGCGCGACCAGCGTCGGTACCGT | |
| | CGCAGTCTTCGGTCTGACCACCGTAGAACGCAGAGCCACCATGGCTT | |
| | GGGTTTGGACCCTGCTGTTCCTGATGGCCGCTGCTCAGTCTATCCAG | |
| | GCTGAGATTGTGCTGACACAGAGCCCTGGCACACTGAGCCTTAGTCC | |
| | TGGCGAAAGAGCCACACTGTCCTGCAGAGCCTCTCAGTCTGTGGGCA | |
| | GCTCTTACCTGGCCTGGTATCAGCAGAAGCCTGGACAGGCTCCCCGG | |
| | CTGTTGATCTACGGCGCCTTTTCTAGAGCCACAGGCATCCCCGATAG | |
| | ATTCAGCGGCTCTGGCAGCGGCACCGATTTCACCCTGACAATCAGCA | |
| | GACTGGAACCCGAGGACTTCGCCGTGTACTACTGTCAGCAGTACGGC | |
| | AGCAGCCCTTGGACATTTGGCCAGGGCACCAAGGTGGAAATCAAAGG | |
| | CGGCAGCGAGGGCAAGTCTAGCGGATCTGGATCTGAGAGCAAGAGCA | |
| | CAGGCGGATCTCAGGTGCAGCTGGTGGAATCTGGTGGCGGAGTTGTG | |
| | CAGCCTGGCAGATCCCTGAGACTGTCTTGTGCCGCCAGCGGCTTCAC | |
| | CTTCAGCAGCTACACAATGCACTGGGTCCGACAGGCCCCTGGCAAAG | |
| | GACTGGAATGGGTCACCTTTATCAGCTACGACGGCAACAACAAGTAC | |
| | TACGCCGACAGCGTGAAGGGCAGATTCACCATCTCCAGAGACAACAG | |
| | CAAGAACACCCTGTACCTGCAGATGAACAGCCTGAGAGCCGAGGACA | |
| | CCGCCATCTACTACTGCGCCAGAACAGGATGGCTGGGCCCCTTTGAT | |
| | TATTGGGGCCAGGGAACCCTGGTCACCGTGTCATCTGAGCCCAAGTC | |
| | TTCCGACAAGACCCACACATGCCCACCTTGTCCAGCACCAGAGCTGC | |
| | TGGGAGGACCATCCGTGTTCCTGTTTCCACCCAAGCCTAAGGATACC | |
| | CTGATGATCTCCAGGACCCCTGAGGTGACATGCGTGGTGGTGGACGT | |
| | GTCTCACGAGGATCCAGAGGTGAAGTTTAACTGGTACGTGGACGGCG | |
| | TGGAGGTGCACAATGCCAAGACAAAGCCCCGGGAGGAGCAGTACAAC | |
| | AGCACCTATAGAGTGGTGTCCGTGCTGACAGTGCTGCACCAGGATTG | |
| | GCTGAACGGCAAGGAGTATAAGTGCAAGGTGAGCAATAAGGCCCTGC | |
| | CTGCCCCAATCGAGAAGACCATCTCCAAGGCAAAGGGACAGCCAAGG | |
| | GAGCCACAGGTGTACACACTGCCTCCAAGCAGAGACGAGCTGACCAA | |
| | GAACCAGGTGTCCCTGACATGTCTGGTGAAGGGCTTCTATCCATCCG | |
| | ATATCGCCGTGGAGTGGGAGTCTAATGGCCAGCCCGAGAACAATTAC | |
| | AAGACCACACCCCCTGTGCTGGACAGCGATGGCTCCTTCTTTCTGTA | |
| | TTCTAAGCTGACCGTGGACAAGAGCCGGTGGCAGCAGGGCAACGTGT | |
| | TCAGCTGCTCTGTGATGCACGAGGCACTGCACAACCACTACACCCAG | |
| | AAATCCCTGTCACTGTCCCCCGGAAAGTAGGACCCCTGGACCACCAG | |
| | CCCCAGCAAGAGCACAAGAGGAAGAGAGAGACCCTCACTGCTGGGGA | |
| | GTCCCTGCCACACTCAGTCCCCCACCACACTGAATCTCCCCTCCTCA | |
| | CAGTTGCCATGTAGACCCCTTGAAGAGGGGAGGGGCCTAGGGAGCCG | |
| | CACCTTGTCATGTACCATCAATAAAATATCTTTATTTTCATTACATC | |
| | TGTGTGTTGGTTTTTTGTGTGTTTCGATAGTACTAACATACGCTCTC | |
| | CATCAAAACAAAACGAAACAAAACAAACTAGCAAAATAGGCTGTCCC | |
| | CAGTGCAAGTGCAGGTGCCAGAACATTTCTCTTCAGTGCCTCTATCT | |
| | GGAGGCCAGGTAGGGCTGGCCTTGGGGGAGGGGGAGGCCAGAATGAC | |
| | TCCAAGAGCTACAGGAAGGCAGGTCAGAGACCCCACTGGACAAACAG | |
| | TGGCTGGACTCTGCACCATAACACACAATCAACAGGGGAGTGAGCTG | |
| | GATCCCTCAAAAATAAAATAAAATAAAAATTAAAAAAAAAGGTTACT | |
| | CTGGCTATGAACCCTACCATATATTATCCTGTAAAATACAGCATAGC | |
| | AAAACTTTAACCTCCAAATCAAGCCTCTACTTGAATCCTTTTCTGAG | |
| | GGATGAATAAGGCATAGGCATCAGGGGCTGTTGCCAATGTGCATTAG | |
| | CTGTTTGCAGCCTCACCTTCTTTCATGGAGTTTAAGATATAGTGTAT | |
| | TTTCCCAAGGTTTGAACTAGCTCTTCATTTCTTTATGTTTTAAATGC | |
| | ACTGACCTCCCACATTCCCTTTTTAGTAAAATATTCAGAAATAATTT | |
| | AAATACATCATTGCAATGAAAATAAATGTTTTTTATTAGGCAGAATC | |
| | CAGATGCTCAAGGCCCTTCATAATATCCCCCAGTTTAGTAGTTGGAC | |
| | TTAGGGAACAAAGGAACCTTTAATAGAAATTGGACAGCAAGAAAGCG | |
| | AGCTCACAGTTTCAGCAGCCCAAAGCTAGTGAACCCAGTGCCATGGG | |
| | AAACCTGAGATGGATCTGTCACGTTGACGAAAACAGAGGCGCCAGGT | |
| | TGGAGTTCGAACACCCCTCCAAGGTGGATGGATTGCTGTCCACAGGG | |
| | CTTAGCGGAAGAGTGGGTATTTGCGGCCCTGAGCAGGATCCTTTCGA | |
| | AGCGTCCCGGACTCTTGAGACACAGGCTTGCTATGAATGGTGCCTGG | |
| | CTGCTGGCTTCCCGATTGGAACAAAATGTGACTTGTGCGTAGATATA | |
| | GTAAAGTCCCTGCCGTTTCACAGTGAGTTGCTTTCCGTTCTCGAGCG | |
| | TAACCAAGTTGTTAGACATTGTGTAATATCCTTTCTCTGCCCATTGC | |
| | AGGACGGAGGTGGTTTTGGAGCTGGCTTCGCTTATGACGTGGGCTGC | |
| | GATCTGTGGGTTAGAGCCTCCGCCGGAACCGCCGCCGGAGCCACCGC | |
| | CGAGCTTGAGCAGTCCGAAGCTGGTGAAGCCTGTTCCGTGAGACACT | |
| | TGAGAAGGATCGGTGACGTTCACAAACACGGAGGCCCCTGGCTGCAG | |
| | TTCGAAAACGCCTCCGAGATGAATGCTCTGCTGGCCGCATGGCTTAG | |
| | CAGAGCTGTGGGTGTTAGCAGCCCGCAGCAGGATTCTCTCAAATCTG | |
| | CCGGGGCTTTTCAGACACAGGGAAGCAATGAAAGGAGCCTGGCTAGA | |
| | AGCCTCGCGGTTGGAGCAGAACGTGACTTGAGCATAGATATAATAGA | |
| | GCCCCTGCCGCTTGACTGTCAGCTGTTTCCCATTCTCGAGGGTCACG | |
| | AGATTGTTGGACATCGTATAATACCCCTTTTCAGCCCACTGGAGGAC | |
| | AGATGTGGTCTTGCTAGAGGCCTCGGAGATCACATGGGCAGCAATCT | |
| | GTGGGTTACTTCCTCCGCCAGAGCCGCCTCCAGAGCCGCCGCCCAGC | |
| | TTCAGCAGGCCGAAAGATGTAAAGCCGGTGCCGTGGGACACCTGGCT | |
| | AGGGTCTGTCACATTCACGAACACGCTTGCGCCAGGCTGCAGCTCAA | |
| | ACACTCCGCCGAGGTGAATAGACTGCTGGCCACAAGGTTTGGCGCTG | |
| | CTGTGTGTGTTGGCGGCTCTCAGCAGAATCCGCTCGAATCTGCCAGG | |
| | GGACTTCAGGCACAGGCTGGCGATAAAAGGGGCCTGAGAGCTGGCCT | |
| | CTCTGTTGCTGCAGAAGGTCACTTGGGCGTAGATGTAGTACAGGCCC | |
| | TGTCTCTTCACGGTCAGCTGCTTGCCGTTTTCCAGGGTGACCAGGTT | |
| | GTTGCTCATGGTGTAGTAGCCCTTCTCGGCCCACTGCAGCACGCTTG | |
| | TTGTCTTGCTGCTGGCCTCGCTGATCACGTGAGCGGCAATCTGTGGG | |
| | TTAGATCCACCACCACCAAGGAATGTTGACAGAAGCACCCACTCTCC | |
| | ATCTTTGCGAACGTAGGCTTGGCCGTCCCTTGGTGCTTCTGGGATAT | |
| | AACCGGCCTGTATACTTTGGGCAGCTGCCATCAGGAATAGCAAGGTC | |
| | CACACCCAAGCCATGGTGGCCCAGTGAAGGGTCCCCGGCTAGGCTAG | |
| | GCCGCTCGGTCCCGGTGGCTGGTCCGGAGAACGTGGGTCCCTCACGC | |
| | TGTCTTGAAGGCGAATGGCGGTCTGCTAACCGGGCTCTGCTTATATA | |
| | CCCGTAGGACACCCATGGTCCGCCCACGAACCGCCCAGTAACGCCCA | |
| | TAACTCCGCCCGGGAACGCCCATGGTCACTCCCCGTTACGCCCCTTT | |
| | CCACTGACGTCAATGGAAAGTCCCCGCGGTTTTGGTGCCAAGAACAT | |
| | TGACTAATATGGAATTTCCCCACCCACCATTGTCGGTAATGGTGGGA | |
| | AAGGGGAACTGGCCCCGTTCCCATTGACGTCACTGGCTATTGCCAAG | |
| | GACATTGACTAATAATAGAAATCCCCCTTTTTGGTGCCAAATGAGGC | |
| | GGTGGGCTTATGGAAAGTCCCCATTTAACCCCTATTCTGGTGCCAGG | |
| | ACGGATGTATATGCTTGCCAAGTCATTGATTTATAATTAAGTAACGC | |
| | CATTTTGCAAGGCATGGAAAAATACATAACTGAGAATAGAGAAGTTC | |
| | AGATCAAGGTCAGGAACAGATGGAACAGCTGAATATGGGCCAAACAG | |
| | GATATCTGTGGTAAGCAGTTCCTGCCCCGGCTCAGGGCCAAGAACAG | |
| | ATGGAACAGCTGAATATGGGCCAAACAGGATATCTGTGGTAAGCAGT | |
| | TCCTGCCCCGGCTCAGGGCCAAGAACAGATGGTCCCCAGATGCGGTC | |
| | CAGCCCTCAGCAGTTTCTAGAGAACCATCAGATGTTTCCAGGGTGCC | |
| | CCAAGGACCTGAAATGACCCTGTGCCTTATTTGAACTAACCAATCAG | |
| | TTCGCTTCTCGCTTCTGTTCGCGCGCTTCTGCTCCCCGAGCTCAATA | |
| | AAAGAGCCCACAACCCCTCACTCGGGGCGCCAGTCCTCCGATTGACT | |
| | GAGTCGCCCGCTTAAGGCCACCATGTGCCATCAGCAGCTGGTCATTA | |
| | GTTGGTTTAGCCTGGTCTTTCTGGCATCACCTCTGGTCGCAATCTGG | |
| | GAACTGAAAAAGGACGTGTACGTGGTGGAGCTGGACTGGTATCCTGA | |
| | TGCCCCAGGCGAGATGGTGGTGCTGACCTGCGACACACCTGAGGAGG | |
| | ATGGCATCACCTGGACACTGGATCAGAGCTCCGAGGTGCTGGGAAGC | |
| | GGCAAGACCCTGACAATCCAGGTGAAGGAGTTCGGCGACGCCGGCCA | |
| | GTACACCTGTCACAAGGGAGGAGAGGTGCTGAGCCACTCCCTGCTGC | |
| | TGCTGCACAAGAAGGAGGATGGCATCTGGAGCACAGACATCCTGAAG | |
| | GATCAGAAGGAGCCCAAGAACAAGACCTTCCTGAGGTGCGAGGCCAA | |
| | GAATTATTCCGGCCGCTTTACCTGTTGGTGGCTGACCACAATCAGCA | |
| | CCGATCTGACATTTTCCGTGAAGTCTAGCAGGGGCTCCTCTGACCCT | |
| | CAGGGAGTGACATGCGGAGCAGCCACCCTGTCTGCCGAGCGGGTGAG | |
| | AGGCGATAACAAGGAGTACGAGTATTCCGTGGAGTGCCAGGAGGACT | |
| | CTGCCTGTCCAGCAGCAGAGGAGTCCCTGCCAATCGAAGTGATGGTG | |
| | GATGCCGTGCACAAGCTGAAGTACGAGAATTATACAAGCTCCTTCTT | |
| | TATCCGGGACATCATCAAGCCCGATCCCCCTAAGAACCTGCAGCTGA | |
| | AGCCTCTGAAGAATTCTAGACAGGTGGAGGTGAGCTGGGAGTACCCC | |
| | GACACCTGGAGCACACCTCACTCCTATTTCTCTCTGACCTTTTGCGT | |
| | GCAGGTGCAGGGCAAGTCTAAGCGGGAGAAGAAGGACAGAGTGTTCA | |
| | CCGATAAGACAAGCGCCACCGTGATCTGTAGGAAGAACGCCTCTATC | |
| | AGCGTGAGGGCCCAGGATCGCTACTATTCTAGCTCCTGGAGCGAGTG | |
| | GGCCTCCGTGCCTTGCTCTGGAGGAGGAGGAGGAGGCTCCCGCAATC | |
| | TGCCAGTGGCAACCCCAGACCCAGGAATGTTCCCATGTCTGCACCAC | |
| | AGCCAGAACCTGCTGCGGGCCGTGTCCAATATGCTGCAGAAGGCCAG | |
| | ACAGACACTGGAGTTTTACCCTTGCACCTCCGAGGAGATCGACCACG | |
| | AGGATATCACAAAGGATAAGACCTCTACAGTGGAGGCCTGCCTGCCA | |
| | CTGGAGCTGACCAAGAACGAGAGCTGTCTGAATTCCAGGGAGACATC | |
| | TTTCATCACCAACGGCAGCTGCCTGGCCTCCCGCAAGACATCTTTTA | |
| | TGATGGCCCTGTGCCTGTCTAGCATCTACGAGGACCTGAAGATGTAT | |
| | CAGGTGGAGTTCAAGACCATGAACGCCAAGCTGCTGATGGACCCCAA | |
| | GAGGCAGATTTTCCTGGATCAGAATATGCTGGCCGTGATCGACGAGC | |
| | TGATGCAGGCCCTGAACTTCAATAGCGAGACAGTGCCTCAGAAGTCC | |
| | TCTCTGGAGGAGCCAGACTTTTACAAGACCAAGATCAAGCTGTGCAT | |
| | CCTGCTGCACGCTTTTCGGATTAGAGCTGTGACCATTGATAGAGTGA | |
| | TGTCCTACCTGAATGCTTCCTGAGGCGCGCCGCAATTTGTACCCTTA | |
| | ATAAATTTCACAAACAGATTTTATCGCATCGTGTCTTATTGCGGGGG | |
| | AGAAAACCGATGTCGGCATAGAAAACCGCCATGATTCTAAGACGTCC | |
| | GAACGCGAGTGGGTGGGGAACAACCCATACCGGACAGATGCCGATGA | |
| | GCCACCCGCACCCTTGGGTGCGGGAGGTACGGGGTGGTTTGTTCATC | |
| | CTATGGTTCCGACCCCACAAACAGCCCCCAGAGTCGGTTTGGGTATG | |
| | GTTACATTTTCTGTCTGGTGGTCGGGCTTGTTTCTTCCTTG | |
| US10-12 | TTCGCACTTCGTCCCAATATATATATATTATTAGGGCGAAGTGCGAG | 201 |
| expression | CACTGGCGCCGTGCCCGACTCCGCGCCGGCCCCGGGGGGGGCCCGG | |
| cassette | GCGGCGGGGGGCGGGTCTCTCCGGCGCACATAAAGGCCCGGCGCGAC | |
| encoding | CGACGCCCGC | |
| human | AGACGGCGCCGGCCACGAACGACGGGAGCGGCTGCGGAGCACGCGGA | |
| CTLA-4 | CCGGGAGCGGGAGTCGCAGAGGGCCGTCGGAGCGGACGGCGTCGGCA | |
| antagonist, | TCGCGACGCCCCGGCTCGGGATCGGGATCGCATCGGAAAGGGACACG | |
| human | CGGACGCGGGGGGGAAAGACCCGCCCACCCCACCCACGAAACACAGG | |
| CD40 | GGACGCACCCCGGGGGCCTCCGACGACAGAAACCCACCGGTCCGCCT | |
| agonist, and | TTTTTGCACGGGTAAGCACCTTGGGTGGGCGGAGGAGGGGGGGACGC | |
| human | GGGGGCGGAGGAGGGGGGGACGCGGGGGCGGAGGAGGGGGGGACGCG | |
| scIL-12 | GGGGCGGAGGAGGGGGGGACGCGGGGGGGGAGGAGGGGGCTCACCCG | |
| payloads, | CGTTCGTGCCTTCCCGCAGGAGGAACGCCCTCGTCGAGGCGACCGGC | |
| and US10 | GGCGACCGTTGCGTGGACCGCTTCCTGCTCGTCGGGGGGGAGCTAGC | |
| and US11 | GCTACCGGTCGCCACCATGTCTCAGACACAACCTCCAGCTCCAGTCG | |
| proteins. | GCCCTGGCGATCCTGATGTGTATCTGAAGGGCGTGCCAAGCGCCGGC | |
| 5′ > 3′ | ATGCATCCTAGGGGTGTTCATGCCCCTAGAGGACACCCCAGAATGAT | |
| orientation | CTCTGGCCCTCCTCAGAGAGGCGACAACGATCAGGCTGCTGGACAGT | |
| | GTGGCGATAGCGGACTGCTGAGAGTGGGCGCCGATACCACAATCAGC | |
| | AAGCCATCTGAGGCTGTGCGGCCTCCTACAATCCCCAGAACCCCTAG | |
| | AGTGCCCCGCGAGCCAAGAGTGCCTAGACCTCCTAGAGAGCCCAGAG | |
| | AACCCCGGGTGCCAAGAGATCCCAGGGATCCTAGAGTCCCTCGGGAC | |
| | CCCAGAGATCCAAGGCAGCCTAGATCACCCAGAGAGCCTCGGAGCCC | |
| | AAGAGAGCCAAGAACACCACGGACACCCCGGGAACCTAGAACAGCCA | |
| | GAGGAAGCGTTTAGGGAAGCCCAGGCCAGACGCGTATGTCGTGGGCC | |
| | CTGGAAATGGCGGACACCTTCCTGGACACCATGCGGGTTGGGCCCAG | |
| | GACGTACGCCGACGTACGCGATGAGATCAATAAAAGGGGGCGTGAGG | |
| | ACCGGGAGGCGGCCAGAACCGCCGTGCACGACCCGGAGCGTCCCCTG | |
| | CTGCGCTCTCCCGGGCTGCTGCCCGAAATCGCCCCCAACGCATCCTT | |
| | GGGTGTGGCACATCGAAGAACCGGCGGGACCGTGACCGACAGTCCCC | |
| | GTAATCCGGTAACCCGTTGAGTCCCGGGTACGACCATCACCCGAGTC | |
| | TCTGGGCGGAGGGTGGTTCCCCCCCGTGGCTCTCGAGATGAGCCAGA | |
| | CCCAACCCCCGGCCCCAGTTGGGCCGGGCGACCCAGATGTTTACTTA | |
| | AAAGGCGTGCCGTCCGCCGGCATGCACCCCAGAGGTGTTCACGCACC | |
| | TCGAGGACACCCGCGCATGATCTCCGGACCCCCGCAACGGGGTGATA | |
| | ATGATCAAGCGGCGGGGCAATGTGGAGATTCGGGTCTACTACGAGTC | |
| | GGTGCGGACACTACGATCTCGAAGCCATCTGAAGCCGTCCGACCGCC | |
| | AACAATCCCCAGGACACCGCGTGTTCCCCGGGAGCCCCGGGTTCCGC | |
| | GACCACCCCGAGAACCTAGGGAACCCAGAGTACCGCGAGATCCCAGA | |
| | GACCCCAGGGTACCGCGTGACCCCAGGGATCCACGACAACCCCGGTC | |
| | TCCCAGGGAGCCCCGGTCTCCCCGGGAGCCCCGGACCCCACGCACCC | |
| | CCCGCGAACCACGTACGGCTCGCGGGTCTGTATAGCCCGGGCAAGTA | |
| | TGCCCCCCTGGCGAGCCCAGACCCCTTCTCCCCACAAGATGGAGCAT | |
| | ACGCTCGGGCCCGCGTCGGGATCCACACCGCGGTTCGCGTCCCGCCC | |
| | ACCGGAAGCTCAACCCACACGCACTTGCGGCAAGACCCGGGCGATGA | |
| | GCCAACCTCGGATGACTCAGGGCTCTACCCTCTGGACGCCCGGGCGC | |
| | TTGCGCACCTGGTGATGTTGCCCGCGGACCACCGGGCCTTCTTTCGA | |
| | ACCGTGGTCGAGGTGTCTCGCATGTGCGCTGCAAACGTGCGCGATCC | |
| | CCCGCCCCCGGCTACAGGGGCCATGTTGGGCCGCCACGCGCGGCTGG | |
| | TCCACACCCAGTGGCTCCGGGCCAACCAAGAGACGTCGCCCCTGTGG | |
| | CCCTGGCGGACGGCGGCCATTAACTTTATCACCACCATGGCCCCCCG | |
| | CGTCCAAACCCACCGACACATGCACGACCTGTTGATGGCCTGTGCTT | |
| | TCTGGTGCTGTCTGACACACGCATCGACGTGTTCGTACGCGGGGCTG | |
| | TACTCGACCCACTGCCTGCATCTGTTTGGTGCGTTTGGGTGTGGGGA | |
| | CCCGGCCCTAACCCCACCCCTGTGCTAGGTCTAGATTAATTAAGGGT | |
| | GGCATCCCTGTGACCCCTCCCCAGTGCCTCTCCTGGCCCTGGAAGTT | |
| | GCCACTCCAGTGCCCACCAGCCTTGTCCTAATAAAATTAAGTTGCAT | |
| | CATTTTGTCTGACTAGGTGTCCTTCTATAATATTATGGGGTGGAGGG | |
| | GGGTGGTATGGAGCAAGGGGCAAGTTGGGAAGACAACCTGTAGGGCC | |
| | TGCGGGGTCTATTGGGAACCAAGCTGGAGTGCAGTGGCACAATCTTG | |
| | GCTCACTGCAATCTCCGCCTCCTGGGTTCAAGCGATTCTCCTGCCTC | |
| | AGCCTCCCGAGTTGTTGGGATTCCAGGCATGCATGACCAGGCTCAGC | |
| | TAATTTTTGTTTTTTTGGTAGAGACGGGGTTTCACCATATTGGCCAG | |
| | GCTGGTCTCCAACTCCTAATCTCAGGTGATCTACCCACCTTGGCCTC | |
| | CCAAATTGCTGGGATTACAGGCGTGAACCACTGCTCCCTTCCCTGTC | |
| | CTTTGGCACGTATACTGAGTCATTAGGGACTTTCCAATGGGTTTTGC | |
| | CCAGTACATAAGGTCAATAGGGGTGAATCAACAGGAAAGTCCCATTG | |
| | GAGCCAAGTACACTGAGTCAATAGGGACTTTCCATTGGGTTTTGCCC | |
| | AGTACAAAAGGTCAATAGGGGGTGAGTCAATGGGTTTTTCCCATTAT | |
| | TGGCACGTACATAAGGTCAATAGGGGTGAGTCATTGGGTTTTTCCAG | |
| | CCAATTTAATTTAAACGCCATGTACTTTCCCACCATTGACGTCAATG | |
| | GGCTATTGAAACTAATGCAACGTGACCTTTAAACGGTACTTTCCCAT | |
| | AGCTGATTAATGGGAAAGTACCGTTCTCGAGCCAATACACGTCAATG | |
| | GGAAGTGAAAGGGCAGCCAAAACGTAACACCGCCCCGGTTTTCCCCT | |
| | GGAAATTCCATATTGGCACGCATTCTATTGGCTGAGCTGCGTTCTAC | |
| | GTGGGTATAAGAGGCGCGACCAGCGTCGGTACCGTCGCAGTCTTCGG | |
| | TCTGACCACCGTAGAACGCAGAGCCACCATGGCTTGGGTTTGGACCC | |
| | TGCTGTTCCTGATGGCCGCTGCTCAGTCTATCCAGGCTGAGATTGTG | |
| | CTGACACAGAGCCCTGGCACACTGAGCCTTAGTCCTGGCGAAAGAGC | |
| | CACACTGTCCTGCAGAGCCTCTCAGTCTGTGGGCAGCTCTTACCTGG | |
| | CCTGGTATCAGCAGAAGCCTGGACAGGCTCCCCGGCTGTTGATCTAC | |
| | GGCGCCTTTTCTAGAGCCACAGGCATCCCCGATAGATTCAGCGGCTC | |
| | TGGCAGCGGCACCGATTTCACCCTGACAATCAGCAGACTGGAACCCG | |
| | AGGACTTCGCCGTGTACTACTGTCAGCAGTACGGCAGCAGCCCTTGG | |
| | ACATTTGGCCAGGGCACCAAGGTGGAAATCAAAGGCGGCAGCGAGGG | |
| | CAAGTCTAGCGGATCTGGATCTGAGAGCAAGAGCACAGGCGGATCTC | |
| | AGGTGCAGCTGGTGGAATCTGGTGGCGGAGTTGTGCAGCCTGGCAGA | |
| | TCCCTGAGACTGTCTTGTGCCGCCAGCGGCTTCACCTTCAGCAGCTA | |
| | CACAATGCACTGGGTCCGACAGGCCCCTGGCAAAGGACTGGAATGGG | |
| | TCACCTTTATCAGCTACGACGGCAACAACAAGTACTACGCCGACAGC | |
| | GTGAAGGGCAGATTCACCATCTCCAGAGACAACAGCAAGAACACCCT | |
| | GTACCTGCAGATGAACAGCCTGAGAGCCGAGGACACCGCCATCTACT | |
| | ACTGCGCCAGAACAGGATGGCTGGGCCCCTTTGATTATTGGGGCCAG | |
| | GGAACCCTGGTCACCGTGTCATCTGAGCCCAAGTCTTCCGACAAGAC | |
| | CCACACATGCCCACCTTGTCCAGCACCAGAGCTGCTGGGAGGACCAT | |
| | CCGTGTTCCTGTTTCCACCCAAGCCTAAGGATACCCTGATGATCTCC | |
| | AGGACCCCTGAGGTGACATGCGTGGTGGTGGACGTGTCTCACGAGGA | |
| | TCCAGAGGTGAAGTTTAACTGGTACGTGGACGGCGTGGAGGTGCACA | |
| | ATGCCAAGACAAAGCCCCGGGAGGAGCAGTACAACAGCACCTATAGA | |
| | GTGGTGTCCGTGCTGACAGTGCTGCACCAGGATTGGCTGAACGGCAA | |
| | GGAGTATAAGTGCAAGGTGAGCAATAAGGCCCTGCCTGCCCCAATCG | |
| | AGAAGACCATCTCCAAGGCAAAGGGACAGCCAAGGGAGCCACAGGTG | |
| | TACACACTGCCTCCAAGCAGAGACGAGCTGACCAAGAACCAGGTGTC | |
| | CCTGACATGTCTGGTGAAGGGCTTCTATCCATCCGATATCGCCGTGG | |
| | AGTGGGAGTCTAATGGCCAGCCCGAGAACAATTACAAGACCACACCC | |
| | CCTGTGCTGGACAGCGATGGCTCCTTCTTTCTGTATTCTAAGCTGAC | |
| | CGTGGACAAGAGCCGGTGGCAGCAGGGCAACGTGTTCAGCTGCTCTG | |
| | TGATGCACGAGGCACTGCACAACCACTACACCCAGAAATCCCTGTCA | |
| | CTGTCCCCCGGAAAGTAGGACCCCTGGACCACCAGCCCCAGCAAGAG | |
| | CACAAGAGGAAGAGAGAGACCCTCACTGCTGGGGAGTCCCTGCCACA | |
| | CTCAGTCCCCCACCACACTGAATCTCCCCTCCTCACAGTTGCCATGT | |
| | AGACCCCTTGAAGAGGGGAGGGGCCTAGGGAGCCGCACCTTGTCATG | |
| | TACCATCAATAAAATATCTTTATTTTCATTACATCTGTGTGTTGGTT | |
| | TTTTGTGTGTTTCGATAGTACTAACATACGCTCTCCATCAAAACAAA | |
| | ACGAAACAAAACAAACTAGCAAAATAGGCTGTCCCCAGTGCAAGTGC | |
| | AGGTGCCAGAACATTTCTCTTCAGTGCCTCTATCTGGAGGCCAGGTA | |
| | GGGCTGGCCTTGGGGGAGGGGGAGGCCAGAATGACTCCAAGAGCTAC | |
| | AGGAAGGCAGGTCAGAGACCCCACTGGACAAACAGTGGCTGGACTCT | |
| | GCACCATAACACACAATCAACAGGGGAGTGAGCTGGATCCCTCAAAA | |
| | ATAAAATAAAATAAAAATTAAAAAAAAAGGTTACTCTGGCTATGAAC | |
| | CCTACCATATATTATCCTGTAAAATACAGCATAGCAAAACTTTAACC | |
| | TCCAAATCAAGCCTCTACTTGAATCCTTTTCTGAGGGATGAATAAGG | |
| | CATAGGCATCAGGGGCTGTTGCCAATGTGCATTAGCTGTTTGCAGCC | |
| | TCACCTTCTTTCATGGAGTTTAAGATATAGTGTATTTTCCCAAGGTT | |
| | TGAACTAGCTCTTCATTTCTTTATGTTTTAAATGCACTGACCTCCCA | |
| | CATTCCCTTTTTAGTAAAATATTCAGAAATAATTTAAATACATCATT | |
| | GCAATGAAAATAAATGTTTTTTATTAGGCAGAATCCAGATGCTCAAG | |
| | GCCCTTCATAATATCCCCCAGTTTAGTAGTTGGACTTAGGGAACAAA | |
| | GGAACCTTTAATAGAAATTGGACAGCAAGAAAGCGAGCTCACAGTTT | |
| | CAGCAGCCCAAAGCTAGTGAACCCAGTGCCATGGGAAACCTGAGATG | |
| | GATCTGTCACGTTGACGAAAACAGAGGCGCCAGGTTGGAGTTCGAAC | |
| | ACCCCTCCAAGGTGGATGGATTGCTGTCCACAGGGCTTAGCGGAAGA | |
| | GTGGGTATTTGCGGCCCTGAGCAGGATCCTTTCGAAGCGTCCCGGAC | |
| | TCTTGAGACACAGGCTTGCTATGAATGGTGCCTGGCTGCTGGCTTCC | |
| | CGATTGGAACAAAATGTGACTTGTGCGTAGATATAGTAAAGTCCCTG | |
| | CCGTTTCACAGTGAGTTGCTTTCCGTTCTCGAGCGTAACCAAGTTGT | |
| | TAGACATTGTGTAATATCCTTTCTCTGCCCATTGCAGGACGGAGGTG | |
| | GTTTTGGAGCTGGCTTCGCTTATGACGTGGGCTGCGATCTGTGGGTT | |
| | AGAGCCTCCGCCGGAACCGCCGCCGGAGCCACCGCCGAGCTTGAGCA | |
| | GTCCGAAGCTGGTGAAGCCTGTTCCGTGAGACACTTGAGAAGGATCG | |
| | GTGACGTTCACAAACACGGAGGCCCCTGGCTGCAGTTCGAAAACGCC | |
| | TCCGAGATGAATGCTCTGCTGGCCGCATGGCTTAGCAGAGCTGTGGG | |
| | TGTTAGCAGCCCGCAGCAGGATTCTCTCAAATCTGCCGGGGCTTTTC | |
| | AGACACAGGGAAGCAATGAAAGGAGCCTGGCTAGAAGCCTCGCGGTT | |
| | GGAGCAGAACGTGACTTGAGCATAGATATAATAGAGCCCCTGCCGCT | |
| | TGACTGTCAGCTGTTTCCCATTCTCGAGGGTCACGAGATTGTTGGAC | |
| | ATCGTATAATACCCCTTTTCAGCCCACTGGAGGACAGATGTGGTCTT | |
| | GCTAGAGGCCTCGGAGATCACATGGGCAGCAATCTGTGGGTTACTTC | |
| | CTCCGCCAGAGCCGCCTCCAGAGCCGCCGCCCAGCTTCAGCAGGCCG | |
| | AAAGATGTAAAGCCGGTGCCGTGGGACACCTGGCTAGGGTCTGTCAC | |
| | ATTCACGAACACGCTTGCGCCAGGCTGCAGCTCAAACACTCCGCCGA | |
| | GGTGAATAGACTGCTGGCCACAAGGTTTGGCGCTGCTGTGTGTGTTG | |
| | GCGGCTCTCAGCAGAATCCGCTCGAATCTGCCAGGGGACTTCAGGCA | |
| | CAGGCTGGCGATAAAAGGGGCCTGAGAGCTGGCCTCTCTGTTGCTGC | |
| | AGAAGGTCACTTGGGCGTAGATGTAGTACAGGCCCTGTCTCTTCACG | |
| | GTCAGCTGCTTGCCGTTTTCCAGGGTGACCAGGTTGTTGCTCATGGT | |
| | GTAGTAGCCCTTCTCGGCCCACTGCAGCACGCTTGTTGTCTTGCTGC | |
| | TGGCCTCGCTGATCACGTGAGCGGCAATCTGTGGGTTAGATCCACCA | |
| | CCACCAAGGAATGTTGACAGAAGCACCCACTCTCCATCTTTGCGAAC | |
| | GTAGGCTTGGCCGTCCCTTGGTGCTTCTGGGATATAACCGGCCTGTA | |
| | TACTTTGGGCAGCTGCCATCAGGAATAGCAAGGTCCACACCCAAGCC | |
| | ATGGTGGCCCAGTGAAGGGTCCCCGGCTAGGCTAGGCCGCTCGGTCC | |
| | CGGTGGCTGGTCCGGAGAACGTGGGTCCCTCACGCTGTCTTGAAGGC | |
| | GAATGGCGGTCTGCTAACCGGGCTCTGCTTATATACCCGTAGGACAC | |
| | CCATGGTCCGCCCACGAACCGCCCAGTAACGCCCATAACTCCGCCCG | |
| | GGAACGCCCATGGTCACTCCCCGTTACGCCCCTTTCCACTGACGTCA | |
| | ATGGAAAGTCCCCGCGGTTTTGGTGCCAAGAACATTGACTAATATGG | |
| | AATTTCCCCACCCACCATTGTCGGTAATGGTGGGAAAGGGGAACTGG | |
| | CCCCGTTCCCATTGACGTCACTGGCTATTGCCAAGGACATTGACTAA | |
| | TAATAGAAATCCCCCTTTTTGGTGCCAAATGAGGCGGTGGGCTTATG | |
| | GAAAGTCCCCATTTAACCCCTATTCTGGTGCCAGGACGGATGTATAT | |
| | GCTTGCCAAGTCATTGATTTATAATTAAGTAACGCCATTTTGCAAGG | |
| | CATGGAAAAATACATAACTGAGAATAGAGAAGTTCAGATCAAGGTCA | |
| | GGAACAGATGGAACAGCTGAATATGGGCCAAACAGGATATCTGTGGT | |
| | AAGCAGTTCCTGCCCCGGCTCAGGGCCAAGAACAGATGGAACAGCTG | |
| | AATATGGGCCAAACAGGATATCTGTGGTAAGCAGTTCCTGCCCCGGC | |
| | TCAGGGCCAAGAACAGATGGTCCCCAGATGCGGTCCAGCCCTCAGCA | |
| | GTTTCTAGAGAACCATCAGATGTTTCCAGGGTGCCCCAAGGACCTGA | |
| | AATGACCCTGTGCCTTATTTGAACTAACCAATCAGTTCGCTTCTCGC | |
| | TTCTGTTCGCGCGCTTCTGCTCCCCGAGCTCAATAAAAGAGCCCACA | |
| | ACCCCTCACTCGGGGCGCCAGTCCTCCGATTGACTGAGTCGCCCGCT | |
| | TAAGGCCACCATGTGCCATCAGCAGCTGGTCATTAGTTGGTTTAGCC | |
| | TGGTCTTTCTGGCATCACCTCTGGTCGCAATCTGGGAACTGAAAAAG | |
| | GACGTGTACGTGGTGGAGCTGGACTGGTATCCTGATGCCCCAGGCGA | |
| | GATGGTGGTGCTGACCTGCGACACACCTGAGGAGGATGGCATCACCT | |
| | GGACACTGGATCAGAGCTCCGAGGTGCTGGGAAGCGGCAAGACCCTG | |
| | ACAATCCAGGTGAAGGAGTTCGGCGACGCCGGCCAGTACACCTGTCA | |
| | CAAGGGAGGAGAGGTGCTGAGCCACTCCCTGCTGCTGCTGCACAAGA | |
| | AGGAGGATGGCATCTGGAGCACAGACATCCTGAAGGATCAGAAGGAG | |
| | CCCAAGAACAAGACCTTCCTGAGGTGCGAGGCCAAGAATTATTCCGG | |
| | CCGCTTTACCTGTTGGTGGCTGACCACAATCAGCACCGATCTGACAT | |
| | TTTCCGTGAAGTCTAGCAGGGGCTCCTCTGACCCTCAGGGAGTGACA | |
| | TGCGGAGCAGCCACCCTGTCTGCCGAGCGGGTGAGAGGCGATAACAA | |
| | GGAGTACGAGTATTCCGTGGAGTGCCAGGAGGACTCTGCCTGTCCAG | |
| | CAGCAGAGGAGTCCCTGCCAATCGAAGTGATGGTGGATGCCGTGCAC | |
| | AAGCTGAAGTACGAGAATTATACAAGCTCCTTCTTTATCCGGGACAT | |
| | CATCAAGCCCGATCCCCCTAAGAACCTGCAGCTGAAGCCTCTGAAGA | |
| | ATTCTAGACAGGTGGAGGTGAGCTGGGAGTACCCCGACACCTGGAGC | |
| | ACACCTCACTCCTATTTCTCTCTGACCTTTTGCGTGCAGGTGCAGGG | |
| | CAAGTCTAAGCGGGAGAAGAAGGACAGAGTGTTCACCGATAAGACAA | |
| | GCGCCACCGTGATCTGTAGGAAGAACGCCTCTATCAGCGTGAGGGCC | |
| | CAGGATCGCTACTATTCTAGCTCCTGGAGCGAGTGGGCCTCCGTGCC | |
| | TTGCTCTGGAGGAGGAGGAGGAGGCTCCCGCAATCTGCCAGTGGCAA | |
| | CCCCAGACCCAGGAATGTTCCCATGTCTGCACCACAGCCAGAACCTG | |
| | CTGCGGGCCGTGTCCAATATGCTGCAGAAGGCCAGACAGACACTGGA | |
| | GTTTTACCCTTGCACCTCCGAGGAGATCGACCACGAGGATATCACAA | |
| | AGGATAAGACCTCTACAGTGGAGGCCTGCCTGCCACTGGAGCTGACC | |
| | AAGAACGAGAGCTGTCTGAATTCCAGGGAGACATCTTTCATCACCAA | |
| | CGGCAGCTGCCTGGCCTCCCGCAAGACATCTTTTATGATGGCCCTGT | |
| | GCCTGTCTAGCATCTACGAGGACCTGAAGATGTATCAGGTGGAGTTC | |
| | AAGACCATGAACGCCAAGCTGCTGATGGACCCCAAGAGGCAGATTTT | |
| | CCTGGATCAGAATATGCTGGCCGTGATCGACGAGCTGATGCAGGCCC | |
| | TGAACTTCAATAGCGAGACAGTGCCTCAGAAGTCCTCTCTGGAGGAG | |
| | CCAGACTTTTACAAGACCAAGATCAAGCTGTGCATCCTGCTGCACGC | |
| | TTTTCGGATTAGAGCTGTGACCATTGATAGAGTGATGTCCTACCTGA | |
| | ATGCTTCCTGAGGCGCGCC | |
| US10-12 | AAGCTGTCACCTACGGACTCTCGATGGGGGGAGGGGGCGAGACCCAC | 202 |
| expression | GGACCCCGACGACCCCCGCCGTCGACGCGGAACTAGCGCGGACCGGT | |
| cassette | CGATGCTTGGGTGGGAAAAAGGACAGGGACGGCCGATCCCCCTCCCG | |
| including | CGCTTCGTCCGCGTATCGGCGTCCCGGCGCGGCGAGCGTCTGACGGT | |
| left and | CTGTCTCTGGCGGTCCCGCGTCGGGTCGTGGATCCGTGTCGGCAGCC | |
| right | GCGCTCCGTGTGGACGATCGGGGCGTCCTCGGGCTCATATAGTCCCA | |
| homology | GGGGCCGGCGGGAAGGAGGAGCAGCGGAGGCCGCCGGCCCCCCGCCC | |
| arms. | CCCCGGCGGGCCCACCCCGAACGGAATTCCATTATGCACGACCCCGC | |
| 5′ > 3′ | CCCGACGCCGGCACGCCGGGGGCCCGTGGCCGCGGCCCGTTGGTCGA | |
| orientation | ACCCCCGGCCCCGCCCATCCGCGCCATCTGCCATGGGGGGGGCGCGA | |
| | GGGCGGGTGGGTCCGCGCCCCGCCCCGCATGGCATCTCATTACCGCC | |
| | CGATCCGGCGGTTTCCGCTTCCGTTCCGCATGCTAACGAGGAACGGG | |
| | CAGGGGGCGGGGCCCGGGCCCCGACTTCCCGGTTCGGCGGTAATGAG | |
| | ATACGAGCCCCGCGCGCCCGTTGGCCGTCCCCGGGCCCCCCGGTCCC | |
| | GCCCGCCGGACGCCGGGACCAACGGGACGGCGGGCGGCCCAAGGGCC | |
| | GCCCGCCTTGCCGCCCCCCCATTGGCCGGCGGGCGGGACCGCCCCAA | |
| | GGGGGCGGGGCCGCCGGGTAAAAGAAGTGAGAACGCGAAGCGTTCGC | |
| | ACTTCGTCCCAATATATATATATTATTAGGGCGAAGTGCGAGCACTG | |
| | GCGCCGTGCCCGACTCCGCGCCGGCCCCGGGGGCGGGCCCGGGCGGC | |
| | GGGGGGCGGGTCTCTCCGGCGCACATAAAGGCCCGGCGCGACCGACG | |
| | CCCGCAGACGGCGCCGGCCACGAACGACGGGAGCGGCTGCGGAGCAC | |
| | GCGGACCGGGAGCGGGAGTCGCAGAGGGCCGTCGGAGCGGACGGCGT | |
| | CGGCATCGCGACGCCCCGGCTCGGGATCGGGATCGCATCGGAAAGGG | |
| | ACACGCGGACGCGGGGGGGAAAGACCCGCCCACCCCACCCACGAAAC | |
| | ACAGGGGACGCACCCCGGGGGCCTCCGACGACAGAAACCCACCGGTC | |
| | CGCCTTTTTTGCACGGGTAAGCACCTTGGGTGGGCGGAGGAGGGGGG | |
| | GACGCGGGGGTGGAGGAGGGGGGACGCGGGGGGGGAGGAGGGGGGAC | |
| | GCGGGGGCGGAGGAGGGGGGACGCGGGGGGGGAGGAGGGGGGACGCG | |
| | GGGGCGGAGGAGGGGGGACGCGGGGGGGGAGGAGGGGGGACGCGGGG | |
| | GCGGAGGAGGGGGCTCACCCGCGTTCGTGCCTTCCCGCAGGAGGAAC | |
| | GTCCTCGTCGAGGCGACCGGCGGCGACCGTTGCGTGGACCGCTTCCT | |
| | GCTCGTCGGGGGGGAGCTAGCGCTACCGGTCGCCACCATGTCTCAGA | |
| | CACAACCTCCAGCTCCAGTCGGCCCTGGCGATCCTGATGTGTATCTG | |
| | AAGGGCGTGCCAAGCGCCGGCATGCATCCTAGGGGTGTTCATGCCCC | |
| | TAGAGGACACCCCAGAATGATCTCTGGCCCTCCTCAGAGAGGCGACA | |
| | ACGATCAGGCTGCTGGACAGTGTGGCGATAGCGGACTGCTGAGAGTG | |
| | GGCGCCGATACCACAATCAGCAAGCCATCTGAGGCTGTGCGGCCTCC | |
| | TACAATCCCCAGAACCCCTAGAGTGCCCCGCGAGCCAAGAGTGCCTA | |
| | GACCTCCTAGAGAGCCCAGAGAACCCCGGGTGCCAAGAGATCCCAGG | |
| | GATCCTAGAGTCCCTCGGGACCCCAGAGATCCAAGGCAGCCTAGATC | |
| | ACCCAGAGAGCCTCGGAGCCCAAGAGAGCCAAGAACACCACGGACAC | |
| | CCCGGGAACCTAGAACAGCCAGAGGAAGCGTTTAGGGAAGCCCAGGC | |
| | CAGACGCGTATGTCGTGGGCCCTGGAAATGGCGGACACCTTCCTGGA | |
| | CACCATGCGGGTTGGGCCCAGGACGTACGCCGACGTACGCGATGAGA | |
| | TCAATAAAAGGGGGCGTGAGGACCGGGAGGCGGCCAGAACCGCCGTG | |
| | CACGACCCGGAGCGTCCCCTGCTGCGCTCTCCCGGGCTGCTGCCCGA | |
| | AATCGCCCCCAACGCATCCTTGGGTGTGGCACATCGAAGAACCGGCG | |
| | GGACCGTGACCGACAGTCCCCGTAATCCGGTAACCCGTTGAGTCCCG | |
| | GGTACGACCATCACCCGAGTCTCTGGGCGGAGGGTGGTTCCCCCCCG | |
| | TGGCTCTCGAGATGAGCCAGACCCAACCCCCGGCCCCAGTTGGGCCG | |
| | GGCGACCCAGATGTTTACTTAAAAGGCGTGCCGTCCGCCGGCATGCA | |
| | CCCCAGAGGTGTTCACGCACCTCGAGGACACCCGCGCATGATCTCCG | |
| | GACCCCCGCAACGGGGTGATAATGATCAAGCGGCGGGGCAATGTGGA | |
| | GATTCGGGTCTACTACGAGTCGGTGCGGACACTACGATCTCGAAGCC | |
| | ATCTGAAGCCGTCCGACCGCCAACAATCCCCAGGACACCGCGTGTTC | |
| | CCCGGGAGCCCCGGGTTCCGCGACCACCCCGAGAACCTAGGGAACCC | |
| | AGAGTACCGCGAGATCCCAGAGACCCCAGGGTACCGCGTGACCCCAG | |
| | GGATCCACGACAACCCCGGTCTCCCAGGGAGCCCCGGTCTCCCCGGG | |
| | AGCCCCGGACCCCACGCACCCCCCGCGAACCACGTACGGCTCGCGGG | |
| | TCTGTATAGCCCGGGCAAGTATGCCCCCCTGGCGAGCCCAGACCCCT | |
| | TCTCCCCACAAGATGGAGCATACGCTCGGGCCCGCGTCGGGATCCAC | |
| | ACCGCGGTTCGCGTCCCGCCCACCGGAAGCTCAACCCACACGCACTT | |
| | GCGGCAAGACCCGGGCGATGAGCCAACCTCGGATGACTCAGGGCTCT | |
| | ACCCTCTGGACGCCCGGGCGCTTGCGCACCTGGTGATGTTGCCCGCG | |
| | GACCACCGGGCCTTCTTTCGAACCGTGGTCGAGGTGTCTCGCATGTG | |
| | CGCTGCAAACGTGCGCGATCCCCCGCCCCCGGCTACAGGGGCCATGT | |
| | TGGGCCGCCACGCGCGGCTGGTCCACACCCAGTGGCTCCGGGCCAAC | |
| | CAAGAGACGTCGCCCCTGTGGCCCTGGCGGACGGCGGCCATTAACTT | |
| | TATCACCACCATGGCCCCCCGCGTCCAAACCCACCGACACATGCACG | |
| | ACCTGTTGATGGCCTGTGCTTTCTGGTGCTGTCTGACACACGCATCG | |
| | ACGTGTTCGTACGCGGGGCTGTACTCGACCCACTGCCTGCATCTGTT | |
| | TGGTGCGTTTGGGTGTGGGGACCCGGCCCTAACCCCACCCCTGTGCT | |
| | AGGTCTAGATTAATTAAGGGTGGCATCCCTGTGACCCCTCCCCAGTG | |
| | CCTCTCCTGGCCCTGGAAGTTGCCACTCCAGTGCCCACCAGCCTTGT | |
| | CCTAATAAAATTAAGTTGCATCATTTTGTCTGACTAGGTGTCCTTCT | |
| | ATAATATTATGGGGTGGAGGGGGGTGGTATGGAGCAAGGGGCAAGTT | |
| | GGGAAGACAACCTGTAGGGCCTGCGGGGTCTATTGGGAACCAAGCTG | |
| | GAGTGCAGTGGCACAATCTTGGCTCACTGCAATCTCCGCCTCCTGGG | |
| | TTCAAGCGATTCTCCTGCCTCAGCCTCCCGAGTTGTTGGGATTCCAG | |
| | GCATGCATGACCAGGCTCAGCTAATTTTTGTTTTTTTGGTAGAGACG | |
| | GGGTTTCACCATATTGGCCAGGCTGGTCTCCAACTCCTAATCTCAGG | |
| | TGATCTACCCACCTTGGCCTCCCAAATTGCTGGGATTACAGGCGTGA | |
| | ACCACTGCTCCCTTCCCTGTCCTTTGGCACGTATACTGAGTCATTAG | |
| | GGACTTTCCAATGGGTTTTGCCCAGTACATAAGGTCAATAGGGGTGA | |
| | ATCAACAGGAAAGTCCCATTGGAGCCAAGTACACTGAGTCAATAGGG | |
| | ACTTTCCATTGGGTTTTGCCCAGTACAAAAGGTCAATAGGGGGTGAG | |
| | TCAATGGGTTTTTCCCATTATTGGCACGTACATAAGGTCAATAGGGG | |
| | TGAGTCATTGGGTTTTTCCAGCCAATTTAATTTAAACGCCATGTACT | |
| | TTCCCACCATTGACGTCAATGGGCTATTGAAACTAATGCAACGTGAC | |
| | CTTTAAACGGTACTTTCCCATAGCTGATTAATGGGAAAGTACCGTTC | |
| | TCGAGCCAATACACGTCAATGGGAAGTGAAAGGGCAGCCAAAACGTA | |
| | ACACCGCCCCGGTTTTCCCCTGGAAATTCCATATTGGCACGCATTCT | |
| | ATTGGCTGAGCTGCGTTCTACGTGGGTATAAGAGGCGCGACCAGCGT | |
| | CGGTACCGTCGCAGTCTTCGGTCTGACCACCGTAGAACGCAGAGCCA | |
| | CCATGGCTTGGGTTTGGACCCTGCTGTTCCTGATGGCCGCTGCTCAG | |
| | TCTATCCAGGCTGAGATTGTGCTGACACAGAGCCCTGGCACACTGAG | |
| | CCTTAGTCCTGGCGAAAGAGCCACACTGTCCTGCAGAGCCTCTCAGT | |
| | CTGTGGGCAGCTCTTACCTGGCCTGGTATCAGCAGAAGCCTGGACAG | |
| | GCTCCCCGGCTGTTGATCTACGGCGCCTTTTCTAGAGCCACAGGCAT | |
| | CCCCGATAGATTCAGCGGCTCTGGCAGCGGCACCGATTTCACCCTGA | |
| | CAATCAGCAGACTGGAACCCGAGGACTTCGCCGTGTACTACTGTCAG | |
| | CAGTACGGCAGCAGCCCTTGGACATTTGGCCAGGGCACCAAGGTGGA | |
| | AATCAAAGGCGGCAGCGAGGGCAAGTCTAGCGGATCTGGATCTGAGA | |
| | GCAAGAGCACAGGCGGATCTCAGGTGCAGCTGGTGGAATCTGGTGGC | |
| | GGAGTTGTGCAGCCTGGCAGATCCCTGAGACTGTCTTGTGCCGCCAG | |
| | CGGCTTCACCTTCAGCAGCTACACAATGCACTGGGTCCGACAGGCCC | |
| | CTGGCAAAGGACTGGAATGGGTCACCTTTATCAGCTACGACGGCAAC | |
| | AACAAGTACTACGCCGACAGCGTGAAGGGCAGATTCACCATCTCCAG | |
| | AGACAACAGCAAGAACACCCTGTACCTGCAGATGAACAGCCTGAGAG | |
| | CCGAGGACACCGCCATCTACTACTGCGCCAGAACAGGATGGCTGGGC | |
| | CCCTTTGATTATTGGGGCCAGGGAACCCTGGTCACCGTGTCATCTGA | |
| | GCCCAAGTCTTCCGACAAGACCCACACATGCCCACCTTGTCCAGCAC | |
| | CAGAGCTGCTGGGAGGACCATCCGTGTTCCTGTTTCCACCCAAGCCT | |
| | AAGGATACCCTGATGATCTCCAGGACCCCTGAGGTGACATGCGTGGT | |
| | GGTGGACGTGTCTCACGAGGATCCAGAGGTGAAGTTTAACTGGTACG | |
| | TGGACGGCGTGGAGGTGCACAATGCCAAGACAAAGCCCCGGGAGGAG | |
| | CAGTACAACAGCACCTATAGAGTGGTGTCCGTGCTGACAGTGCTGCA | |
| | CCAGGATTGGCTGAACGGCAAGGAGTATAAGTGCAAGGTGAGCAATA | |
| | AGGCCCTGCCTGCCCCAATCGAGAAGACCATCTCCAAGGCAAAGGGA | |
| | CAGCCAAGGGAGCCACAGGTGTACACACTGCCTCCAAGCAGAGACGA | |
| | GCTGACCAAGAACCAGGTGTCCCTGACATGTCTGGTGAAGGGCTTCT | |
| | ATCCATCCGATATCGCCGTGGAGTGGGAGTCTAATGGCCAGCCCGAG | |
| | AACAATTACAAGACCACACCCCCTGTGCTGGACAGCGATGGCTCCTT | |
| | CTTTCTGTATTCTAAGCTGACCGTGGACAAGAGCCGGTGGCAGCAGG | |
| | GCAACGTGTTCAGCTGCTCTGTGATGCACGAGGCACTGCACAACCAC | |
| | TACACCCAGAAATCCCTGTCACTGTCCCCCGGATAGGACCCCTGGAC | |
| | CACCAGCCCCAGCAAGAGCACAAGAGGAAGAGAGAGACCCTCACTGC | |
| | TGGGGAGTCCCTGCCACACTCAGTCCCCCACCACACTGAATCTCCCC | |
| | TCCTCACAGTTGCCATGTAGACCCCTTGAAGAGGGGAGGGGCCTAGG | |
| | GAGCCGCACCTTGTCATGTACCATCAATAAAATATCTTTATTTTCAT | |
| | TACATCTGTGTGTTGGTTTTTTGTGTGTTTCGATAGTACTAACATAC | |
| | GCTCTCCATCAAAACAAAACGAAACAAAACAAACTAGCAAAATAGGC | |
| | TGTCCCCAGTGCAAGTGCAGGTGCCAGAACATTTCTCTTCAGTGCCT | |
| | CTATCTGGAGGCCAGGTAGGGCTGGCCTTGGGGGAGGGGGAGGCCAG | |
| | AATGACTCCAAGAGCTACAGGAAGGCAGGTCAGAGACCCCACTGGAC | |
| | AAACAGTGGCTGGACTCTGCACCATAACACACAATCAACAGGGGAGT | |
| | GAGCTGGATCCCTCAAAAATAAAATAAAATAAAAATTAAAAAAAAAG | |
| | GTTACTCTGGCTATGAACCCTACCATATATTATCCTGTAAAATACAG | |
| | CATAGCAAAACTTTAACCTCCAAATCAAGCCTCTACTTGAATCCTTT | |
| | TCTGAGGGATGAATAAGGCATAGGCATCAGGGGCTGTTGCCAATGTG | |
| | CATTAGCTGTTTGCAGCCTCACCTTCTTTCATGGAGTTTAAGATATA | |
| | GTGTATTTTCCCAAGGTTTGAACTAGCTCTTCATTTCTTTATGTTTT | |
| | AAATGCACTGACCTCCCACATTCCCTTTTTAGTAAAATATTCAGAAA | |
| | TAATTTAAATACATCATTGCAATGAAAATAAATGTTTTTTATTAGGC | |
| | AGAATCCAGATGCTCAAGGCCCTTCATAATATCCCCCAGTTTAGTAG | |
| | TTGGACTTAGGGAACAAAGGAACCTTTAATAGAAATTGGACAGCAAG | |
| | AAAGCGAGCTCACAGTTTCAGCAGCCCAAAGCTAGTGAACCCAGTGC | |
| | CATGGGAAACCTGAGATGGATCTGTCACGTTGACGAAAACAGAGGCG | |
| | CCAGGTTGGAGTTCGAACACCCCTCCAAGGTGGATGGATTGCTGTCC | |
| | ACAGGGCTTAGCGGAAGAGTGGGTATTTGCGGCCCTGAGCAGGATCC | |
| | TTTCGAAGCGTCCCGGACTCTTGAGACACAGGCTTGCTATGAATGGT | |
| | GCCTGGCTGCTGGCTTCCCGATTGGAACAAAATGTGACTTGTGCGTA | |
| | GATATAGTAAAGTCCCTGCCGTTTCACAGTGAGTTGCTTTCCGTTCT | |
| | CGAGCGTAACCAAGTTGTTAGACATTGTGTAATATCCTTTCTCTGCC | |
| | CATTGCAGGACGGAGGTGGTTTTGGAGCTGGCTTCGCTTATGACGTG | |
| | GGCTGCGATCTGTGGGTTAGAGCCTCCGCCGGAACCGCCGCCGGAGC | |
| | CACCGCCGAGCTTGAGCAGTCCGAAGCTGGTGAAGCCTGTTCCGTGA | |
| | GACACTTGAGAAGGATCGGTGACGTTCACAAACACGGAGGCCCCTGG | |
| | CTGCAGTTCGAAAACGCCTCCGAGATGAATGCTCTGCTGGCCGCATG | |
| | GCTTAGCAGAGCTGTGGGTGTTAGCAGCCCGCAGCAGGATTCTCTCA | |
| | AATCTGCCGGGGCTTTTCAGACACAGGGAAGCAATGAAAGGAGCCTG | |
| | GCTAGAAGCCTCGCGGTTGGAGCAGAACGTGACTTGAGCATAGATAT | |
| | AATAGAGCCCCTGCCGCTTGACTGTCAGCTGTTTCCCATTCTCGAGG | |
| | GTCACGAGATTGTTGGACATCGTATAATACCCCTTTTCAGCCCACTG | |
| | GAGGACAGATGTGGTCTTGCTAGAGGCCTCGGAGATCACATGGGCAG | |
| | CAATCTGTGGGTTACTTCCTCCGCCAGAGCCGCCTCCAGAGCCGCCG | |
| | CCCAGCTTCAGCAGGCCGAAAGATGTAAAGCCGGTGCCGTGGGACAC | |
| | CTGGCTAGGGTCTGTCACATTCACGAACACGCTTGCGCCAGGCTGCA | |
| | GCTCAAACACTCCGCCGAGGTGAATAGACTGCTGGCCACAAGGTTTG | |
| | GCGCTGCTGTGTGTGTTGGCGGCTCTCAGCAGAATCCGCTCGAATCT | |
| | GCCAGGGGACTTCAGGCACAGGCTGGCGATAAAAGGGGCCTGAGAGC | |
| | TGGCCTCTCTGTTGCTGCAGAAGGTCACTTGGGCGTAGATGTAGTAC | |
| | AGGCCCTGTCTCTTCACGGTCAGCTGCTTGCCGTTTTCCAGGGTGAC | |
| | CAGGTTGTTGCTCATGGTGTAGTAGCCCTTCTCGGCCCACTGCAGCA | |
| | CGCTTGTTGTCTTGCTGCTGGCCTCGCTGATCACGTGAGCGGCAATC | |
| | TGTGGGTTAGATCCACCACCACCAAGGAATGTTGACAGAAGCACCCA | |
| | CTCTCCATCTTTGCGAACGTAGGCTTGGCCGTCCCTTGGTGCTTCTG | |
| | GGATATAACCGGCCTGTATACTTTGGGCAGCTGCCATCAGGAATAGC | |
| | AAGGTCCACACCCAAGCCATGGTGGCCCAGTGAAGGGTCCCCGGCTA | |
| | GGCTAGGCCGCTCGGTCCCGGTGGCTGGTCCGGAGAACGTGGGTCCC | |
| | TCACGCTGTCTTGAAGGCGAATGGCGGTCTGCTAACCGGGCTCTGCT | |
| | TATATACCCGTAGGACACCCATGGTCCGCCCACGAACCGCCCAGTAA | |
| | CGCCCATAACTCCGCCCGGGAACGCCCATGGTCACTCCCCGTTACGC | |
| | CCCTTTCCACTGACGTCAATGGAAAGTCCCCGCGGTTTTGGTGCCAA | |
| | GAACATTGACTAATATGGAATTTCCCCACCCACCATTGTCGGTAATG | |
| | GTGGGAAAGGGGAACTGGCCCCGTTCCCATTGACGTCACTGGCTATT | |
| | GCCAAGGACATTGACTAATAATAGAAATCCCCCTTTTTGGTGCCAAA | |
| | TGAGGCGGTGGGCTTATGGAAAGTCCCCATTTAACCCCTATTCTGGT | |
| | GCCAGGACGGATGTATATGCTTGCCAAGTCATTGATTTATAATTAAG | |
| | TAACGCCATTTTGCAAGGCATGGAAAAATACATAACTGAGAATAGAG | |
| | AAGTTCAGATCAAGGTCAGGAACAGATGGAACAGCTGAATATGGGCC | |
| | AAACAGGATATCTGTGGTAAGCAGTTCCTGCCCCGGCTCAGGGCCAA | |
| | GAACAGATGGAACAGCTGAATATGGGCCAAACAGGATATCTGTGGTA | |
| | AGCAGTTCCTGCCCCGGCTCAGGGCCAAGAACAGATGGTCCCCAGAT | |
| | GCGGTCCAGCCCTCAGCAGTTTCTAGAGAACCATCAGATGTTTCCAG | |
| | GGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGC | |
| | TCAATAAAAGAGCCCACAACCCCTCACTCGGGGCGCCAGTCCTCCGA | |
| | TTGACTGAGTCGCCCGCTTAAGGCCACCATGTGCCATCAGCAGCTGG | |
| | TCATTAGTTGGTTTAGCCTGGTCTTTCTGGCATCACCTCTGGTCGCA | |
| | ATCTGGGAACTGAAAAAGGACGTGTACGTGGTGGAGCTGGACTGGTA | |
| | TCCTGATGCCCCAGGCGAGATGGTGGTGCTGACCTGCGACACACCTG | |
| | AGGAGGATGGCATCACCTGGACACTGGATCAGAGCTCCGAGGTGCTG | |
| | GGAAGCGGCAAGACCCTGACAATCCAGGTGAAGGAGTTCGGCGACGC | |
| | CGGCCAGTACACCTGTCACAAGGGAGGAGAGGTGCTGAGCCACTCCC | |
| | TGCTGCTGCTGCACAAGAAGGAGGATGGCATCTGGAGCACAGACATC | |
| | CTGAAGGATCAGAAGGAGCCCAAGAACAAGACCTTCCTGAGGTGCGA | |
| | GGCCAAGAATTATTCCGGCCGCTTTACCTGTTGGTGGCTGACCACAA | |
| | TCAGCACCGATCTGACATTTTCCGTGAAGTCTAGCAGGGGCTCCTCT | |
| | GACCCTCAGGGAGTGACATGCGGAGCAGCCACCCTGTCTGCCGAGCG | |
| | GGTGAGAGGCGATAACAAGGAGTACGAGTATTCCGTGGAGTGCCAGG | |
| | AGGACTCTGCCTGTCCAGCAGCAGAGGAGTCCCTGCCAATCGAAGTG | |
| | ATGGTGGATGCCGTGCACAAGCTGAAGTACGAGAATTATACAAGCTC | |
| | CTTCTTTATCCGGGACATCATCAAGCCCGATCCCCCTAAGAACCTGC | |
| | AGCTGAAGCCTCTGAAGAATTCTAGACAGGTGGAGGTGAGCTGGGAG | |
| | TACCCCGACACCTGGAGCACACCTCACTCCTATTTCTCTCTGACCTT | |
| | TTGCGTGCAGGTGCAGGGCAAGTCTAAGCGGGAGAAGAAGGACAGAG | |
| | TGTTCACCGATAAGACAAGCGCCACCGTGATCTGTAGGAAGAACGCC | |
| | TCTATCAGCGTGAGGGCCCAGGATCGCTACTATTCTAGCTCCTGGAG | |
| | CGAGTGGGCCTCCGTGCCTTGCTCTGGAGGAGGAGGAGGAGGCTCCC | |
| | GCAATCTGCCAGTGGCAACCCCAGACCCAGGAATGTTCCCATGTCTG | |
| | CACCACAGCCAGAACCTGCTGCGGGCCGTGTCCAATATGCTGCAGAA | |
| | GGCCAGACAGACACTGGAGTTTTACCCTTGCACCTCCGAGGAGATCG | |
| | ACCACGAGGATATCACAAAGGATAAGACCTCTACAGTGGAGGCCTGC | |
| | CTGCCACTGGAGCTGACCAAGAACGAGAGCTGTCTGAATTCCAGGGA | |
| | GACATCTTTCATCACCAACGGCAGCTGCCTGGCCTCCCGCAAGACAT | |
| | CTTTTATGATGGCCCTGTGCCTGTCTAGCATCTACGAGGACCTGAAG | |
| | ATGTATCAGGTGGAGTTCAAGACCATGAACGCCAAGCTGCTGATGGA | |
| | CCCCAAGAGGCAGATTTTCCTGGATCAGAATATGCTGGCCGTGATCG | |
| | ACGAGCTGATGCAGGCCCTGAACTTCAATAGCGAGACAGTGCCTCAG | |
| | AAGTCCTCTCTGGAGGAGCCAGACTTTTACAAGACCAAGATCAAGCT | |
| | GTGCATCCTGCTGCACGCTTTTCGGATTAGAGCTGTGACCATTGATA | |
| | GAGTGATGTCCTACCTGAATGCTTCCTGAGGCGCGCCGCAATTTGTA | |
| | CCCTTAATAAATTTCACAAACAGATTTTATCGCATCGTGTCTTATTG | |
| | CGGGGGAGAAAACCGATGTCGGCATAGAAAACCGCCATGATTCTAAG | |
| | ACGTCCGAACGCGAGTGGGTGGGGAACAACCCATACCGGACAGATGC | |
| | CGATGAGCCACCCGCACCCTTGGGTGCGGGAGGTACGGGGTGGTTTG | |
| | TTCATCCTATGGTTCCGACCCCACAAACAGCCCCCAGAGTCGGTTTG | |
| | GGTATGGTTACATTTTCTGTCTGGTGGTCGGGCTTGTTTCTTCCTTG | |
| | CCACTCCCCACCCACCCACTCCCCACCCACCCACTCCCCACCCACCC | |
| | ACTCCCCACCCACCCACTCCCCACCCACCCACTCCCCACCCACCCAC | |
| | TCCCCACCCACCCACTCCCCACCCACCCACTCCCCACCCACCCACTC | |
| | CCCACCCACCCAAAAATCAACCGGGAGACAACATTGCCAATCGAACC | |
| | CAATTTAATGTAGTTAAAGGCTGGGTGCAAATTGCGGGGTGATGGGG | |
| | GGGAAGAGAGACGACAAGAAGGACGCGCGTGTCGATGCGGTCTTTTA | |
| | GCGGAGCAGCCACATCAGGAGCGCCCCAAATCCGCCCGACAGAACGG | |
| | CCACGAGGAGACAGGCGATCACCATGCCGACGCAGCGGGTGCGTCTG | |
| | CGTCGACGCCTTAATACCGACTGTTGGCGGCCCATGCGTACGAGGAA | |
| | GTCGTTGGCCGCCTCGTCTTCGCTTTCCGAGTAGTAGGCTTCGACCG | |
| | AAACTGGCGAGGCCGTGGGATAAAGCGGCACGGACATGTCGGATCGC | |
| | GCTGAGGAGTTGGGATCGGAGAGCCGGGACGTCATCGAGGCCGGAAG | |
| | AAAGCTCCGGGTGGGAAGTTGCGGTCGCTGTGACTCACGATTTTTAA | |
| | TTTGCTGCGGCTAGGCGGACCACCGGCCCTTTATGCGCCTCGGGCAA | |
| | TTGACGTCACATACCACGCAATCCCACACAGGACGGCCCCCAGGCCG | |
| | GAAGCCCCCCGGAGCCACCGAGCGGCCAGCCAGGCGACAAACAGGGA | |
| | GGGGGCGTCGACAGCCTGGAGGGCCATCGGGGAGACAACGGCCGTGT | |
| | AGCCCGGGGGTCGCGGGTGTGGCGAGGGCGCGGTCGACGTGGCGAGG | |
| | GGCGGGCGGTCATGTCGGGGGGGTCCGCGTTCGTCGGAAATCGCGAT | |
| | TAGCTCGTCTCCGACGTCCACCT | |
| Minimal IE | TTCGCACTTCGTCCCAATATATATATATTATTAGGGCGAAGTGCGAG | 203 |
| US12 | CACTGGCGCCGTGCCCGACTCCGCGCCGGCCCCGGGGGCGGGCCCGG | |
| promoter | GCGGCGGGGGGCGGGTCTCTCCGGCGCACATAAAGGCCCGGCGCGAC | |
| and | CGACGCCCGC | |
| transcription | | |
| start site | | |
| 5′ > 3′ | | |
| orientation | | |
| 5′ UTR | AGACGGCGCCGGCCACGAACGACGGGAGCGGCTGCGGAGCACGCGGA | 223 |
| 5′ > 3′ | CCGGGAGCGGGAGTCGCAGAGGGCCGTCGGAGCGGACGGCGTCGGCA | |
| orientation | TCGCGACGCCCCGGCTCGGGATCGGGATCGCATCGGAAAGGGACACG | |
| | CGGACGCGGGGGGGAAAGACCCGCCCACCCCACCCACGAAACACAGG | |
| | GGACGCACCCCGGGGGCCTCCGACGACAGAAACCCACCGGTCCGCCT | |
| | TTTTTGCACGGGTAAGCACCTTGGGTGGGCGGAGGAGGGGGGGACGC | |
| | GGGGGTGGAGGAGGGGGGACGCGGGGGGGGAGGAGGGGGGACGCGGG | |
| | GGCGGAGGAGGGGGGACGCGGGGGGGGAGGAGGGGGGACGCGGGGGC | |
| | GGAGGAGGGGGGACGCGGGGGGGGAGGAGGGGGGACGCGGGGGCGGA | |
| | GGAGGGGGCTCACCCGCGTTCGTGCCTTCCCGCAGGAGGAACGTCCT | |
| | CGTCGAGGCGACCGGCGGCGACCGTTGCGTGGACCGCTTCCTGCTCG | |
| | TCGGGGGGGAGCTAGCGCTACCGGTCGCCACC | |
| Human | ATGTCTCAGACACAACCTCCAGCTCCAGTCGGCCCTGGCGATCCTGA | 204 |
| Codon | TGTGTATCTGAAGGGCGTGCCAAGCGCCGGCATGCATCCTAGGGGTG | |
| Optimized | TTCATGCCCCTAGAGGACACCCCAGAATGATCTCTGGCCCTCCTCAG | |
| US11 | AGAGGCGACAACGATCAGGCTGCTGGACAGTGTGGCGATAGCGGACT | |
| 5′ > 3′ | GCTGAGAGTGGGCGCCGATACCACAATCAGCAAGCCATCTGAGGCTG | |
| orientation | TGCGGCCTCCTACAATCCCCAGAACCCCTAGAGTGCCCCGCGAGCCA | |
| | AGAGTGCCTAGACCTCCTAGAGAGCCCAGAGAACCCCGGGTGCCAAG | |
| | AGATCCCAGGGATCCTAGAGTCCCTCGGGACCCCAGAGATCCAAGGC | |
| | AGCCTAGATCACCCAGAGAGCCTCGGAGCCCAAGAGAGCCAAGAACA | |
| | CCACGGACACCCCGGGAACCTAGAACAGCCAGAGGAAGCGTTTAG | |
| Spacer | GGAAGCCCAGGCCAGACGCGT | 205 |
| sequence | | |
| and US12 5′ | | |
| UTR | | |
| 5′ > 3′ | | |
| orientation | | |
| US12 | ATGTCGTGGGCCCTGGAAATGGCGGACACCTTCCTGGACACCATGCG | 206 |
| 5′ > 3′ | GGTTGGGCCCAGGACGTACGCCGACGTACGCGATGAGATCAATAAAA | |
| orientation | GGGGGCGTGAGGACCGGGAGGCGGCCAGAACCGCCGTGCACGACCCG | |
| | GAGCGTCCCCTGCTGCGCTCTCCCGGGCTGCTGCCCGAAATCGCCCC | |
| | CAACGCATCCTTGGGTGTGGCACATCGAAGAACCGGCGGGACCGTGA | |
| | CCGACAGTCCCCGTAATCCGGTAACCCGTTGA | |
| Spacer | GTCCCGGGTACGACCATCACCCGAGTCTCTGGGCGGAGGGTGGTTCC | 207 |
| sequence | CCCCCGTGGCTCTCGAG | |
| and | | |
| endogenous | | |
| late US11 5′ | | |
| UTR | | |
| 5′ > 3′ | | |
| orientation | | |
| US11-US10 | ATGAGCCAGACCCAACCCCCGGCCCCAGTTGGGCCGGGCGACCCAGA | 208 |
| 5′ > 3′ | TGTTTACTTAAAAGGCGTGCCGTCCGCCGGCATGCACCCCAGAGGTG | |
| orientation | TTCACGCACCTCGAGGACACCCGCGCATGATCTCCGGACCCCCGCAA | |
| | CGGGGTGATAATGATCAAGCGGCGGGGCAATGTGGAGATTCGGGTCT | |
| | ACTACGAGTCGGTGCGGACACTACGATCTCGAAGCCATCTGAAGCCG | |
| | TCCGACCGCCAACAATCCCCAGGACACCGCGTGTTCCCCGGGAGCCC | |
| | CGGGTTCCGCGACCACCCCGAGAACCTAGGGAACCCAGAGTACCGCG | |
| | AGATCCCAGAGACCCCAGGGTACCGCGTGACCCCAGGGATCCACGAC | |
| | AACCCCGGTCTCCCAGGGAGCCCCGGTCTCCCCGGGAGCCCCGGACC | |
| | CCACGCACCCCCCGCGAACCACGTACGGCTCGCGGGTCTGTATAGCC | |
| | CGGGCAAGTATGCCCCCCTGGCGAGCCCAGACCCCTTCTCCCCACAA | |
| | GATGGAGCATACGCTCGGGCCCGCGTCGGGATCCACACCGCGGTTCG | |
| | CGTCCCGCCCACCGGAAGCTCAACCCACACGCACTTGCGGCAAGACC | |
| | CGGGCGATGAGCCAACCTCGGATGACTCAGGGCTCTACCCTCTGGAC | |
| | GCCCGGGCGCTTGCGCACCTGGTGATGTTGCCCGCGGACCACCGGGC | |
| | CTTCTTTCGAACCGTGGTCGAGGTGTCTCGCATGTGCGCTGCAAACG | |
| | TGCGCGATCCCCCGCCCCCGGCTACAGGGGCCATGTTGGGCCGCCAC | |
| | GCGCGGCTGGTCCACACCCAGTGGCTCCGGGCCAACCAAGAGACGTC | |
| | GCCCCTGTGGCCCTGGCGGACGGCGGCCATTAACTTTATCACCACCA | |
| | TGGCCCCCCGCGTCCAAACCCACCGACACATGCACGACCTGTTGATG | |
| | GCCTGTGCTTTCTGGTGCTGTCTGACACACGCATCGACGTGTTCGTA | |
| | CGCGGGGCTGTACTCGACCCACTGCCTGCATCTGTTTGGTGCGTTTG | |
| | GGTGTGGGGACCCGGCCCTAACCCCACCCCTGTGCTAG | |
| hGH polyA | GGGTGGCATCCCTGTGACCCCTCCCCAGTGCCTCTCCTGGCCCTGGA | 209 |
| 5′ > 3′ | AGTTGCCACTCCAGTGCCCACCAGCCTTGTCCTAATAAAATTAAGTT | |
| orientation | GCATCATTTTGTCTGACTAGGTGTCCTTCTATAATATTATGGGGTGG | |
| | AGGGGGGTGGTATGGAGCAAGGGGCAAGTTGGGAAGACAACCTGTAG | |
| | GGCCTGCGGGGTCTATTGGGAACCAAGCTGGAGTGCAGTGGCACAAT | |
| | CTTGGCTCACTGCAATCTCCGCCTCCTGGGTTCAAGCGATTCTCCTG | |
| | CCTCAGCCTCCCGAGTTGTTGGGATTCCAGGCATGCATGACCAGGCT | |
| | CAGCTAATTTTTGTTTTTTTGGTAGAGACGGGGTTTCACCATATTGG | |
| | CCAGGCTGGTCTCCAACTCCTAATCTCAGGTGATCTACCCACCTTGG | |
| | CCTCCCAAATTGCTGGGATTACAGGCGTGAACCACTGCTCCCTTCCC | |
| | TGTCCTT | |
| mCMV | TGGCACGTATACTGAGTCATTAGGGACTTTCCAATGGGTTTTGCCCA | 210 |
| promoter | GTACATAAGGTCAATAGGGGTGAATCAACAGGAAAGTCCCATTGGAG | |
| 5′ > 3′ | CCAAGTACACTGAGTCAATAGGGACTTTCCATTGGGTTTTGCCCAGT | |
| orientation | ACAAAAGGTCAATAGGGGGTGAGTCAATGGGTTTTTCCCATTATTGG | |
| | CACGTACATAAGGTCAATAGGGGTGAGTCATTGGGTTTTTCCAGCCA | |
| | ATTTAATTTAAACGCCATGTACTTTCCCACCATTGACGTCAATGGGC | |
| | TATTGAAACTAATGCAACGTGACCTTTAAACGGTACTTTCCCATAGC | |
| | TGATTAATGGGAAAGTACCGTTCTCGAGCCAATACACGTCAATGGGA | |
| | AGTGAAAGGGCAGCCAAAACGTAACACCGCCCCGGTTTTCCCCTGGA | |
| | AATTCCATATTGGCACGCATTCTATTGGCTGAGCTGCGTTCTACGTG | |
| | GGTATAAGAGGCGCGACCAGCGTCGGTACCGTCGCAGTCTTCGGTCT | |
| | GACCACCGTAGAACGCAGA | |
| Partial | GCCACC | |
| Kozak | | |
| sequence | | |
| for | | |
| expression | | |
| of CTLA-4 | | |
| antagonist | | |
| payload. | | |
| 5′ > 3′ | | |
| orientation | | |
| Human | ATGGCTTGGGTTTGGACCCTGCTGTTCCTGATGGCCGCTGCTCAGTC | 212 |
| CTLA-4 | TATCCAGGCTGAGATTGTGCTGACACAGAGCCCTGGCACACTGAGCC | |
| Antagonist | TTAGTCCTGGCGAAAGAGCCACACTGTCCTGCAGAGCCTCTCAGTCT | |
| Payload | GTGGGCAGCTCTTACCTGGCCTGGTATCAGCAGAAGCCTGGACAGGC | |
| 5′ > 3′ | TCCCCGGCTGTTGATCTACGGCGCCTTTTCTAGAGCCACAGGCATCC | |
| orientation | CCGATAGATTCAGCGGCTCTGGCAGCGGCACCGATTTCACCCTGACA | |
| | ATCAGCAGACTGGAACCCGAGGACTTCGCCGTGTACTACTGTCAGCA | |
| | GTACGGCAGCAGCCCTTGGACATTTGGCCAGGGCACCAAGGTGGAAA | |
| | TCAAAGGCGGCAGCGAGGGCAAGTCTAGCGGATCTGGATCTGAGAGC | |
| | AAGAGCACAGGCGGATCTCAGGTGCAGCTGGTGGAATCTGGTGGCGG | |
| | AGTTGTGCAGCCTGGCAGATCCCTGAGACTGTCTTGTGCCGCCAGCG | |
| | GCTTCACCTTCAGCAGCTACACAATGCACTGGGTCCGACAGGCCCCT | |
| | GGCAAAGGACTGGAATGGGTCACCTTTATCAGCTACGACGGCAACAA | |
| | CAAGTACTACGCCGACAGCGTGAAGGGCAGATTCACCATCTCCAGAG | |
| | ACAACAGCAAGAACACCCTGTACCTGCAGATGAACAGCCTGAGAGCC | |
| | GAGGACACCGCCATCTACTACTGCGCCAGAACAGGATGGCTGGGCCC | |
| | CTTTGATTATTGGGGCCAGGGAACCCTGGTCACCGTGTCATCTGAGC | |
| | CCAAGTCTTCCGACAAGACCCACACATGCCCACCTTGTCCAGCACCA | |
| | GAGCTGCTGGGAGGACCATCCGTGTTCCTGTTTCCACCCAAGCCTAA | |
| | GGATACCCTGATGATCTCCAGGACCCCTGAGGTGACATGCGTGGTGG | |
| | TGGACGTGTCTCACGAGGATCCAGAGGTGAAGTTTAACTGGTACGTG | |
| | GACGGCGTGGAGGTGCACAATGCCAAGACAAAGCCCCGGGAGGAGCA | |
| | GTACAACAGCACCTATAGAGTGGTGTCCGTGCTGACAGTGCTGCACC | |
| | AGGATTGGCTGAACGGCAAGGAGTATAAGTGCAAGGTGAGCAATAAG | |
| | GCCCTGCCTGCCCCAATCGAGAAGACCATCTCCAAGGCAAAGGGACA | |
| | GCCAAGGGAGCCACAGGTGTACACACTGCCTCCAAGCAGAGACGAGC | |
| | TGACCAAGAACCAGGTGTCCCTGACATGTCTGGTGAAGGGCTTCTAT | |
| | CCATCCGATATCGCCGTGGAGTGGGAGTCTAATGGCCAGCCCGAGAA | |
| | CAATTACAAGACCACACCCCCTGTGCTGGACAGCGATGGCTCCTTCT | |
| | TTCTGTATTCTAAGCTGACCGTGGACAAGAGCCGGTGGCAGCAGGGC | |
| | AACGTGTTCAGCTGCTCTGTGATGCACGAGGCACTGCACAACCACTA | |
| | CACCCAGAAATCCCTGTCACTGTCCCCCGGATAG | |
| GAPDH SP | GACCCCTGGACCACCAGCCCCAGCAAGAGCACAAGAGGAAGAGAGAG | 213 |
| A for | ACCCTCACTGCTGGGGAGTCCCTGCCACACTCAGTCCCCCACCACAC | |
| expression | TGAATCTCCCCTCCTCACAGTTGCCATGTAGACCCCTTGAAGAGGGG | |
| of human | AGGGGCCTAGGGAGCCGCACCTTGTCATGTACCATCAATAAAATATC | |
| CTLA-4 | TTTATTTTCATTACATCTGTGTGTTGGTTTTTTGTGTGTTTCGATAG | |
| antagonist | TACTAACATACGCTCTCCATCAAAACAAAACGAAACAAAACAAACTA | |
| payload. | GCAAAATAGGCTGTCCCCAGTGCAAGTGCAGGTGCCAGAACATTTCT | |
| 5′ > 3′ | CT | |
| orientation | | |
| C2 pause | TCAGTGCCTCTATCTGGAGGCCAGGTAGGGCTGGCCTTGGGGGAGGG | 214 |
| site for | GGAGGCCAGAATGACTCCAAGAGCTACAGGAAGGCAGGTCAGAGACC | |
| expression | CCACTGGACAAACAGTGGCTGGACTCTGCACCATAACACACAATCAA | |
| of human | CAGGGGAGTGAGCTGGATCC | |
| CTLA-4 | | |
| antagonist | | |
| payload. | | |
| 5′ > 3′ | | |
| orientation | | |
| hGT pause | CTCAAAAATAAAATAAAATAAAAATTAAAAAAAAAGGTTACTCTGGC | 215 |
| site for | TATGAACCCTACCATATATTATCCTG | |
| expression | | |
| of human | | |
| CD40 | | |
| agonist | | |
| payload. | | |
| 3′ > 5′ | | |
| orientation | | |
| Human Beta | TAAAATACAGCATAGCAAAACTTTAACCTCCAAATCAAGCCTCTACT | 216 |
| Globin | TGAATCCTTTTCTGAGGGATGAATAAGGCATAGGCATCAGGGGCTGT | |
| polyA | TGCCAATGTGCATTAGCTGTTTGCAGCCTCACCTTCTTTCATGGAGT | |
| (hBGpA) | TTAAGATATAGTGTATTTTCCCAAGGTTTGAACTAGCTCTTCATTTC | |
| for | TTTATGTTTTAAATGCACTGACCTCCCACATTCCCTTTTTAGTAAAA | |
| expression | TATTCAGAAATAATTTAAATACATCATTGCAATGAAAATAAATGTTT | |
| of human | TTTATTAGGCAGAATCCAGATGCTCAAGGCCCTTCATAATATCCCCC | |
| CD40 | AGTTTAGTAGTTGGACTTAGGGAACAAAGGAACCTTTAATAGAAATT | |
| agonist | GGACAGCAAGAAAGCGAGC | |
| payload. | | |
| 3′ > 5′ | | |
| orientation | | |
| Human | TCACAGTTTCAGCAGCCCAAAGCTAGTGAACCCAGTGCCATGGGAAA | 217 |
| CD40 | CCTGAGATGGATCTGTCACGTTGACGAAAACAGAGGCGCCAGGTTGG | |
| agonist | AGTTCGAACACCCCTCCAAGGTGGATGGATTGCTGTCCACAGGGCTT | |
| payload. | AGCGGAAGAGTGGGTATTTGCGGCCCTGAGCAGGATCCTTTCGAAGC | |
| 3′ > 5′ | GTCCCGGACTCTTGAGACACAGGCTTGCTATGAATGGTGCCTGGCTG | |
| orientation | CTGGCTTCCCGATTGGAACAAAATGTGACTTGTGCGTAGATATAGTA | |
| | AAGTCCCTGCCGTTTCACAGTGAGTTGCTTTCCGTTCTCGAGCGTAA | |
| | CCAAGTTGTTAGACATTGTGTAATATCCTTTCTCTGCCCATTGCAGG | |
| | ACGGAGGTGGTTTTGGAGCTGGCTTCGCTTATGACGTGGGCTGCGAT | |
| | CTGTGGGTTAGAGCCTCCGCCGGAACCGCCGCCGGAGCCACCGCCGA | |
| | GCTTGAGCAGTCCGAAGCTGGTGAAGCCTGTTCCGTGAGACACTTGA | |
| | GAAGGATCGGTGACGTTCACAAACACGGAGGCCCCTGGCTGCAGTTC | |
| | GAAAACGCCTCCGAGATGAATGCTCTGCTGGCCGCATGGCTTAGCAG | |
| | AGCTGTGGGTGTTAGCAGCCCGCAGCAGGATTCTCTCAAATCTGCCG | |
| | GGGCTTTTCAGACACAGGGAAGCAATGAAAGGAGCCTGGCTAGAAGC | |
| | CTCGCGGTTGGAGCAGAACGTGACTTGAGCATAGATATAATAGAGCC | |
| | CCTGCCGCTTGACTGTCAGCTGTTTCCCATTCTCGAGGGTCACGAGA | |
| | TTGTTGGACATCGTATAATACCCCTTTTCAGCCCACTGGAGGACAGA | |
| | TGTGGTCTTGCTAGAGGCCTCGGAGATCACATGGGCAGCAATCTGTG | |
| | GGTTACTTCCTCCGCCAGAGCCGCCTCCAGAGCCGCCGCCCAGCTTC | |
| | AGCAGGCCGAAAGATGTAAAGCCGGTGCCGTGGGACACCTGGCTAGG | |
| | GTCTGTCACATTCACGAACACGCTTGCGCCAGGCTGCAGCTCAAACA | |
| | CTCCGCCGAGGTGAATAGACTGCTGGCCACAAGGTTTGGCGCTGCTG | |
| | TGTGTGTTGGCGGCTCTCAGCAGAATCCGCTCGAATCTGCCAGGGGA | |
| | CTTCAGGCACAGGCTGGCGATAAAAGGGGCCTGAGAGCTGGCCTCTC | |
| | TGTTGCTGCAGAAGGTCACTTGGGCGTAGATGTAGTACAGGCCCTGT | |
| | CTCTTCACGGTCAGCTGCTTGCCGTTTTCCAGGGTGACCAGGTTGTT | |
| | GCTCATGGTGTAGTAGCCCTTCTCGGCCCACTGCAGCACGCTTGTTG | |
| | TCTTGCTGCTGGCCTCGCTGATCACGTGAGCGGCAATCTGTGGGTTA | |
| | GATCCACCACCACCAAGGAATGTTGACAGAAGCACCCACTCTCCATC | |
| | TTTGCGAACGTAGGCTTGGCCGTCCCTTGGTGCTTCTGGGATATAAC | |
| | CGGCCTGTATACTTTGGGCAGCTGCCATCAGGAATAGCAAGGTCCAC | |
| | ACCCAAGCCAT | |
| Partial | GGTGGC | |
| Kozak | | |
| sequence | | |
| for | | |
| expression | | |
| of the | | |
| human | | |
| CD40 | | |
| agonist | | |
| payload. | | |
| 3′ > 5′ | | |
| orientation | | |
| AoHV1 | CCAGTGAAGGGTCCCCGGCTAGGCTAGGCCGCTCGGTCCCGGTGGCT | 219 |
| promoter | GGTCCGGAGAACGTGGGTCCCTCACGCTGTCTTGAAGGCGAATGGCG | |
| for | GTCTGCTAACCGGGCTCTGCTTATATACCCGTAGGACACCCATGGTC | |
| expression | CGCCCACGAACCGCCCAGTAACGCCCATAACTCCGCCCGGGAACGCC | |
| of the | CATGGTCACTCCCCGTTACGCCCCTTTCCACTGACGTCAATGGAAAG | |
| human | TCCCCGCGGTTTTGGTGCCAAGAACATTGACTAATATGGAATTTCCC | |
| CD40 | CACCCACCATTGTCGGTAATGGTGGGAAAGGGGAACTGGCCCCGTTC | |
| agonist | CCATTGACGTCACTGGCTATTGCCAAGGACATTGACTAATAATAGAA | |
| payload. | ATCCCCCTTTTTGGTGCCAAATGAGGCGGTGGGCTTATGGAAAGTCC | |
| 3′ > 5′ | CCATTTAACCCCTATTCTGGTGCCAGGACGGATGTATATGCTTGCCA | |
| orientation | AGTCATTGATTT | |
| MMLV | ATAATTAAGTAACGCCATTTTGCAAGGCATGGAAAAATACATAACTG | 220 |
| promoter | AGAATAGAGAAGTTCAGATCAAGGTCAGGAACAGATGGAACAGCTGA | |
| for | ATATGGGCCAAACAGGATATCTGTGGTAAGCAGTTCCTGCCCCGGCT | |
| expression | CAGGGCCAAGAACAGATGGAACAGCTGAATATGGGCCAAACAGGATA | |
| of human | TCTGTGGTAAGCAGTTCCTGCCCCGGCTCAGGGCCAAGAACAGATGG | |
| single chain | TCCCCAGATGCGGTCCAGCCCTCAGCAGTTTCTAGAGAACCATCAGA | |
| IL-12 | TGTTTCCAGGGTGCCCCAAGGACCTGAAATGACCCTGTGCCTTATTT | |
| payload. | GAACTAACCAATCAGTTCGCTTCTCGCTTCTGTTCGCGCGCTTCTGC | |
| 5′ > 3′ | TCCCCGAGCTCAATAAAAGAGCCCACAACCCCTCACTCGGGGCGCCA | |
| orientation | GTCCTCCGATTGACTGAGTCGCCCGCTTAAG | |
| Partial | GCCACC | |
| Kozak | | |
| sequence | | |
| for | | |
| expression | | |
| of human | | |
| single chain | | |
| IL-12 | | |
| payload. | | |
| 5′ > 3′ | | |
| orientation | | |
| Human | ATGTGCCATCAGCAGCTGGTCATTAGTTGGTTTAGCCTGGTCTTTCT | 222 |
| single chain | GGCATCACCTCTGGTCGCAATCTGGGAACTGAAAAAGGACGTGTACG | |
| IL-12 | TGGTGGAGCTGGACTGGTATCCTGATGCCCCAGGCGAGATGGTGGTG | |
| payload. | CTGACCTGCGACACACCTGAGGAGGATGGCATCACCTGGACACTGGA | |
| 5′ > 3′ | TCAGAGCTCCGAGGTGCTGGGAAGCGGCAAGACCCTGACAATCCAGG | |
| orientation | TGAAGGAGTTCGGCGACGCCGGCCAGTACACCTGTCACAAGGGAGGA | |
| | GAGGTGCTGAGCCACTCCCTGCTGCTGCTGCACAAGAAGGAGGATGG | |
| | CATCTGGAGCACAGACATCCTGAAGGATCAGAAGGAGCCCAAGAACA | |
| | AGACCTTCCTGAGGTGCGAGGCCAAGAATTATTCCGGCCGCTTTACC | |
| | TGTTGGTGGCTGACCACAATCAGCACCGATCTGACATTTTCCGTGAA | |
| | GTCTAGCAGGGGCTCCTCTGACCCTCAGGGAGTGACATGCGGAGCAG | |
| | CCACCCTGTCTGCCGAGCGGGTGAGAGGCGATAACAAGGAGTACGAG | |
| | TATTCCGTGGAGTGCCAGGAGGACTCTGCCTGTCCAGCAGCAGAGGA | |
| | GTCCCTGCCAATCGAAGTGATGGTGGATGCCGTGCACAAGCTGAAGT | |
| | ACGAGAATTATACAAGCTCCTTCTTTATCCGGGACATCATCAAGCCC | |
| | GATCCCCCTAAGAACCTGCAGCTGAAGCCTCTGAAGAATTCTAGACA | |
| | GGTGGAGGTGAGCTGGGAGTACCCCGACACCTGGAGCACACCTCACT | |
| | CCTATTTCTCTCTGACCTTTTGCGTGCAGGTGCAGGGCAAGTCTAAG | |
| | CGGGAGAAGAAGGACAGAGTGTTCACCGATAAGACAAGCGCCACCGT | |
| | GATCTGTAGGAAGAACGCCTCTATCAGCGTGAGGGCCCAGGATCGCT | |
| | ACTATTCTAGCTCCTGGAGCGAGTGGGCCTCCGTGCCTTGCTCTGGA | |
| | GGAGGAGGAGGAGGCTCCCGCAATCTGCCAGTGGCAACCCCAGACCC | |
| | AGGAATGTTCCCATGTCTGCACCACAGCCAGAACCTGCTGCGGGCCG | |
| | TGTCCAATATGCTGCAGAAGGCCAGACAGACACTGGAGTTTTACCCT | |
| | TGCACCTCCGAGGAGATCGACCACGAGGATATCACAAAGGATAAGAC | |
| | CTCTACAGTGGAGGCCTGCCTGCCACTGGAGCTGACCAAGAACGAGA | |
| | GCTGTCTGAATTCCAGGGAGACATCTTTCATCACCAACGGCAGCTGC | |
| | CTGGCCTCCCGCAAGACATCTTTTATGATGGCCCTGTGCCTGTCTAG | |
| | CATCTACGAGGACCTGAAGATGTATCAGGTGGAGTTCAAGACCATGA | |
| | ACGCCAAGCTGCTGATGGACCCCAAGAGGCAGATTTTCCTGGATCAG | |
| | AATATGCTGGCCGTGATCGACGAGCTGATGCAGGCCCTGAACTTCAA | |
| | TAGCGAGACAGTGCCTCAGAAGTCCTCTCTGGAGGAGCCAGACTTTT | |
| | ACAAGACCAAGATCAAGCTGTGCATCCTGCTGCACGCTTTTCGGATT | |
| | AGAGCTGTGACCATTGATAGAGTGATGTCCTACCTGAATGCTTCCTG | |
| | A | |

Mouse payload expression cassettes sequences

| | | |
|---|---|---|
| m37E | TTAATTAAGGGTGGCATCCCTGTGACCCCTCCCCAGTGCCTCTCCTG | 300 |
| cassette | GCCCTGGAAGTTGCCACTCCAGTGCCCACCAGCCTTGTCCTAATAAA | |
| encoding | ATTAAGTTGCATCATTTTGTCTGACTAGGTGTCCTTCTATAATATTA | |
| mouse | TGGGGTGGAGGGGGGTGGTATGGAGCAAGGGGCAAGTTGGGAAGACA | |
| CTLA-4 | ACCTGTAGGGCCTGCGGGGTCTATTGGGAACCAAGCTGGAGTGCAGT | |
| antagonist, | GGCACAATCTTGGCTCACTGCAATCTCCGCCTCCTGGGTTCAAGCGA | |
| mouse | TTCTCCTGCCTCAGCCTCCCGAGTTGTTGGGATTCCAGGCATGCATG | |
| CD40 | ACCAGGCTCAGCTAATTTTTGTTTTTTTGGTAGAGACGGGGTTTCAC | |
| agonist, | CATATTGGCCAGGCTGGTCTCCAACTCCTAATCTCAGGTGATCTACC | |
| and | CACCTTGGCCTCCCAAATTGCTGGGATTACAGGCGTGAACCACTGCT | |
| mouse scIL- | CCCTTCCCTGTCCTTTGGCACGTATACTGAGTCATTAGGGACTTTCC | |
| 12 payloads. | AATGGGTTTTGCCCAGTACATAAGGTCAATAGGGGTGAATCAACAGG | |
| 5′ > 3′ | AAAGTCCCATTGGAGCCAAGTACACTGAGTCAATAGGGACTTTCCAT | |
| direction | TGGGTTTTGCCCAGTACAAAAGGTCAATAGGGGGTGAGTCAATGGGT | |
| | TTTTCCCATTATTGGCACGTACATAAGGTCAATAGGGGTGAGTCATT | |
| | GGGTTTTTCCAGCCAATTTAATTTAAACGCCATGTACTTTCCCACCA | |
| | TTGACGTCAATGGGCTATTGAAACTAATGCAACGTGACCTTTAAACG | |
| | GTACTTTCCCATAGCTGATTAATGGGAAAGTACCGTTCTCGAGCCAA | |
| | TACACGTCAATGGGAAGTGAAAGGGCAGCCAAAACGTAACACCGCCC | |
| | CGGTTTTCCCCTGGAAATTCCATATTGGCACGCATTCTATTGGCTGA | |
| | GCTGCGTTCTACGTGGGTATAAGAGGCGCGACCAGCGTCGGTACCGT | |
| | CGCAGTCTTCGGTCTGACCACCGTAGAACGCAGAGCCACCATGGCTT | |
| | GGGTTTGGACCCTGCTGTTCCTGATGGCCGCTGCTCAGTCTATCCAG | |
| | GCTCAGGTGCAGCTGGTGGAATCTGGCGGAGGACTTGCTCAACCTGG | |
| | CGGCTCTCTGAGACTGTCTTGTGCCGCTTCTGGCAGCACCATCAGCT | |
| | CTGTGGCTGTCGGCTGGTACAGACAGACACCTGGCAACCAGAGAGAG | |
| | TGGGTCGCCACCAGCAGCACAAGCTCTACCACAGCCACATACGCCGA | |
| | CAGCGTGAAGGGCAGATTCACCATCTCCAGAGACAACGCCAAGAACA | |
| | CCATCTACCTGCAGATGAACAGCCTGAAGCCTGAGGACACCGCCGTG | |
| | TACTACTGCAAGACCGGCCTGACAAACTGGGGCAGAGGCACCCAAGT | |
| | GACCGTGTCTAGCGAACCTAGAGGCCCCACCATCAAGCCCTGTCCTC | |
| | CATGCAAATGCCCCGCTCCTAATCTGCTCGGCGGACCCAGCGTGTTC | |
| | ATCTTCCCACCTAAGATCAAGGACGTGCTGATGATCTCTCTGAGCCC | |
| | CATCGTGACCTGCGTGGTGGTGGATGTGTCTGAGGACGACCCTGACG | |
| | TGCAGATTAGTTGGTTCGTGAACAACGTCGAGGTGCACACAGCCCAG | |
| | ACACAGACCCACAGAGAGGACTACAACAGCACCCTGAGAGTGGTGTC | |
| | TGCCCTGCCTATCCAGCACCAGGATTGGATGAGCGGCAAAGAATTCA | |
| | AGTGCAAAGTGAACAACAAGGACCTGCCTGCTCCTATCGAGAGAACC | |
| | ATCAGCAAGCCCAAGGGCTCTGTCAGGGCTCCTCAGGTGTACGTTCT | |
| | GCCACCTCCTGAGGAAGAGATGACCAAGAAACAAGTGACCCTGACCT | |
| | GTATGGTCACCGACTTCATGCCCGAGGACATCTACGTGGAATGGACC | |
| | AACAACGGCAAGACCGAGCTGAACTACAAGAACACAGAGCCCGTGCT | |
| | GGACAGCGACGGCAGCTACTTCATGTACAGCAAGCTGCGCGTCGAGA | |
| | AGAAGAACTGGGTCGAGAGAAACAGCTACAGCTGCAGCGTGGTGCAC | |
| | GAGGGACTGCACAACCACCACACCACCAAGAGCTTCAGCAGAACCCC | |
| | TGGCAAGTGATAAGACCCCTGGACCACCAGCCCCAGCAAGAGCACAA | |
| | GAGGAAGAGAGAGACCCTCACTGCTGGGGAGTCCCTGCCACACTCAG | |
| | TCCCCCACCACACTGAATCTCCCCTCCTCACAGTTGCCATGTAGACC | |
| | CCTTGAAGAGGGGAGGGGCCTAGGGAGCCGCACCTTGTCATGTACCA | |
| | TCAATAAAATATCTTTATTTTCATTACATCTGTGTGTTGGTTTTTTG | |
| | TGTGTTTCGATAGTACTAACATACGCTCTCCATCAAAACAAAACGAA | |
| | ACAAAACAAACTAGCAAAATAGGCTGTCCCCAGTGCAAGTGCAGGTG | |
| | CCAGAACATTTCTCTTCAGTGCCTCTATCTGGAGGCCAGGTAGGGCT | |
| | GGCCTTGGGGGAGGGGGAGGCCAGAATGACTCCAAGAGCTACAGGAA | |
| | GGCAGGTCAGAGACCCCACTGGACAAACAGTGGCTGGACTCTGCACC | |
| | ATAACACACAATCAACAGGGGAGTGAGCTGGATCCCTCAAAAATAAA | |
| | ATAAAATAAAAATTAAAAAAAAAGGTTACTCTGGCTATGAACCCTAC | |
| | CATATATTATCCTGTAAAATACAGCATAGCAAAACTTTAACCTCCAA | |
| | ATCAAGCCTCTACTTGAATCCTTTTCTGAGGGATGAATAAGGCATAG | |
| | GCATCAGGGGCTGTTGCCAATGTGCATTAGCTGTTTGCAGCCTCACC | |
| | TTCTTTCATGGAGTTTAAGATATAGTGTATTTTCCCAAGGTTTGAAC | |
| | TAGCTCTTCATTTCTTTATGTTTTAAATGCACTGACCTCCCACATTC | |
| | CCTTTTTAGTAAAATATTCAGAAATAATTTAAATACATCATTGCAAT | |
| | GAAAATAAATGTTTTTTATTAGGCAGAATCCAGATGCTCAAGGCCCT | |
| | TCATAATATCCCCCAGTTTAGTAGTTGGACTTAGGGAACAAAGGAAC | |
| | CTTTAATAGAAATTGGACAGCAAGAAAGCGAGCCTATCACAGCTTCA | |
| | GTAAACCGAAGGAGGAGAAGCCCACACGATGGATAACTTGGCTGGCC | |
| | TCGGTCACGTTCACGAACACGGAGGCGCCAGCTTGCAGCTCGAACAC | |
| | GCCTCCTAAATGGACGGACTGCTGCTCGCACAGCTGGGAGGAGGAGT | |
| | GGGTGTTGGCGGCCTTCAGTAAAATCCTCTCGGAGCCGGAGCTGGGC | |
| | TTCAGCCATAAACCCACGATGAAGGGCCTCTGGCTGGAGGGCTCACG | |
| | ATTGGAGCAGAAGGTAACTTGGGTGTACACGTAGTATAAACCCTCCC | |
| | TCTTCACGGTCAGCTGCTTGCCGTTCTCCAGCATCACTAAATTGCTC | |
| | TTCATGGTGTAGTAGCCCTTTTTGGCCCACTGCAGCACGGAGGCGGC | |
| | ATTGGAGTTGGCCTCGGACACCACGTGGGCGGCAATCTGAGGGTCGG | |
| | AGCCGCCGCCAGAGCCGCCGCCGGAGCCGCCACCCAGCTTCAGTAAA | |
| | CCGAAGGAGCTGAAGCCCACACGATGGATAACTTGGGAGGCCTCGGT | |
| | CACGTTCACGAACACGGAGGCGCCAGCTTGCAGCTCAAACACGCCTC | |
| | CTAAATGCACGGACTGCTGCTCGCACAGCTGGCTGGAGGAGTGGGTG | |
| | TTGGCGGCCTTCAGCAGAATCCTCTCGCTGCCGGAGGAGGGCTTCAG | |
| | CCACAGTCCCACGATGAAAGGCCTCTGGGAGGAGGGCTCACGATTGG | |
| | AGCAGAAGGTAACTTGGGTGTACACGTAGTATAAACCTTCCCTCTTC | |
| | ACGGTCAGCTGCTTGCCGTTCTCCAGCATCACTAAATTGCTCTTCAT | |
| | GGTGTAGTAGCCCTTCTTGGCCCACTGCAGCACGCTAGCGGCGTTGG | |
| | AGTTGGCCTCGGACACCACGTGAGCGGCGATCTGGGGGTCGGAGCCG | |
| | CCGCCAGAGCCGCCGCCGGAGCCGCCACCCAGCTTCAGTAAACCAAA | |
| | GGAGGAGAAGCCCACACGATGGATAACTTGGGAGGCCTCGGTCACGT | |
| | TCACGAACACGCTGGCGCCAGCTTGCAGCTCAAACACGCCTCCTAAA | |
| | TGCACGGACTGCTGCTCGCACAGCTGGGAGGAGGAGTGGGTGTTGGC | |
| | GGCCTTCAGTAAAATCCTCTCGGAGCCGCTGCTAGGCTTCAGCCATA | |
| | AACCCACGATGAAGGGCCTCTGGGAGGAAGGCTCCCGGTTGGAGCAG | |
| | AAGGTAACTTGGGTGTACACGTAGTATAAACCCTCCCTCTTCACTGT | |
| | CAGCTGCTTGCCGTTCTCCAGCATCACTAAATTGGACTTCATGGTGT | |
| | AGTAGCCCTTCTTGGCCCACTGCAGCACGGAAGCGGCGTTGGAGTTG | |
| | GCCTCGGACACCACGTGGGCAGCGATCTGGGGGTCAGATCCACCACC | |
| | ACCAGAACCGCCTCCGCCAAGGAATGTTGACAGAAGCACCCACTCTC | |
| | CATCTTTGCGAACGTAGGCTTGGCCGTCCCTTGGTGCTTCTGGGATA | |
| | TAACCGGCCTGTATACTTTGGGCAGCTGCCATCAGGAATAGCAAGGT | |
| | CCACACCCAAGCCATGGTGGCCCAGTGAAGGGTCCCCGGCTAGGCTA | |
| | GGCCGCTCGGTCCCGGTGGCTGGTCCGGAGAACGTGGGTCCCTCACG | |
| | CTGTCTTGAAGGCGAATGGCGGTCTGCTAACCGGGCTCTGCTTATAT | |
| | ACCCGTAGGACACCCATGGTCCGCCCACGAACCGCCCAGTAACGCCC | |
| | ATAACTCCGCCCGGGAACGCCCATGGTCACTCCCCGTTACGCCCCTT | |
| | TCCACTGACGTCAATGGAAAGTCCCCGCGGTTTTGGTGCCAAGAACA | |
| | TTGACTAATATGGAATTTCCCCACCCACCATTGTCGGTAATGGTGGG | |
| | AAAGGGGAACTGGCCCCGTTCCCATTGACGTCACTGGCTATTGCCAA | |
| | GGACATTGACTAATAATAGAAATCCCCCTTTTTGGTGCCAAATGAGG | |
| | CGGTGGGCTTATGGAAAGTCCCCATTTAACCCCTATTCTGGTGCCAG | |
| | GACGGATGTATATGCTTGCCAAGTCATTGATTTATAATTAAGTAACG | |
| | CCATTTTGCAAGGCATGGAAAAATACATAACTGAGAATAGAGAAGTT | |
| | CAGATCAAGGTCAGGAACAGATGGAACAGCTGAATATGGGCCAAACA | |
| | GGATATCTGTGGTAAGCAGTTCCTGCCCCGGCTCAGGGCCAAGAACA | |
| | GATGGAACAGCTGAATATGGGCCAAACAGGATATCTGTGGTAAGCAG | |
| | TTCCTGCCCCGGCTCAGGGCCAAGAACAGATGGTCCCCAGATGCGGT | |
| | CCAGCCCTCAGCAGTTTCTAGAGAACCATCAGATGTTTCCAGGGTGC | |
| | CCCAAGGACCTGAAATGACCCTGTGCCTTATTTGAACTAACCAATCA | |
| | GTTCGCTTCTCGCTTCTGTTCGCGCGCTTCTGCTCCCCGAGCTCAAT | |
| | AAAAGAGCCCACAACCCCTCACTCGGGGCGCCAGTCCTCCGATTGAC | |
| | TGAGTCGCCCGCTTAAGGCCACCATGTGCCCTCAGAAGCTGACCATC | |
| | AGTTGGTTCGCCATCGTGCTGCTGGTGTCCCCACTGATGGCTATGTG | |
| | GGAACTCGAGAAGGACGTGTACGTGGTGGAAGTGGACTGGACCCCTG | |
| | ATGCTCCTGGCGAGACAGTGAACCTGACCTGCGACACACCTGAAGAG | |
| | GACGACATCACCTGGACCAGCGATCAGAGACACGGCGTGATCGGCTC | |
| | TGGCAAGACCCTGACAATTACCGTGAAAGAGTTCCTGGACGCCGGCC | |
| | AGTACACCTGTCACAAAGGCGGAGAGACACTGAGCCACTCTCATCTG | |
| | CTGCTGCACAAGAAAGAGAACGGCATCTGGTCCACCGAGATCCTGAA | |
| | GAACTTCAAGAACAAGACCTTCCTGAAGTGCGAGGCCCCTAACTACA | |
| | GCGGCAGATTCACCTGTAGCTGGCTGGTGCAGAGAAACATGGACCTG | |
| | AAGTTCAACATCAAGTCCTCCAGCAGCAGCCCCGACAGCAGAGCTGT | |
| | GACATGTGGCATGGCTTCTCTGAGCGCCGAGAAAGTGACACTGGACC | |
| | AGAGAGACTACGAGAAGTACAGCGTGTCCTGCCAAGAGGACGTGACC | |
| | TGTCCTACCGCCGAGGAAACACTGCCTATCGAGCTGGCCCTGGAAGC | |
| | CAGACAGCAGAACAAATACGAGAACTACTCTACCAGCTTCTTCATCC | |
| | GGGACATCATCAAGCCCGATCCTCCAAAGAACCTGCAGATGAAGCCT | |
| | CTGAAGAACAGCCAGGTCGAGGTGTCCTGGGAGTACCCTGACAGCTG | |
| | GTCTACCCCTCACAGCTACTTCAGCCTGAAATTCTTCGTGCGGATCC | |
| | AGCGCAAGAAAGAAAAGATGAAGGAAACCGAGGAAGGCTGCAACCAG | |
| | AAAGGCGCTTTCCTGGTGGAAAAGACCAGCACCGAGGTGCAGTGCAA | |
| | AGGCGGCAATGTCTGTGTGCAGGCCCAGGACCGGTACTACAACAGCA | |
| | GCTGTAGCAAGTGGGCCTGCGTGCCATGCAGAGTTAGAAGCGGAGGC | |
| | GGAGGTTCTGGTGGCGGAGGAAGTGGCGGCGGAGGATCTAGAGTGAT | |
| | CCCTGTGTCTGGCCCTGCCAGATGTCTGAGCCAGAGCAGAAACCTGC | |
| | TGAAAACCACCGACGACATGGTCAAGACCGCCAGAGAGAAGCTGAAG | |
| | CACTACTCCTGCACAGCCGAGGACATCGACCACGAGGATATCACCAG | |
| | GGACCAGACAAGCACCCTGAAAACCTGCCTGCCTCTGGAACTGCATA | |
| | AGAACGAGAGCTGCCTGGCCACCAGAGAAACCAGCTCTACCACAAGA | |
| | GGCAGCTGTCTGCCTCCTCAGAAAACCAGCCTGATGATGACCCTGTG | |
| | CCTGGGCAGCATCTACGAGGATCTGAAGATGTACCAGACCGAGTTCC | |
| | AGGCCATCAACGCCGCTCTGCAGAACCACAACCACCAGCAGATCATC | |
| | CTGGACAAGGGCATGCTGGTGGCTATCGACGAGCTGATGCAGAGCCT | |
| | GAACCATAACGGCGAGACACTGCGGCAGAAGCCTCCAGTTGGAGAGG | |
| | CCGATCCTTACAGAGTGAAGATGAAGCTGTGCATCCTGCTGCACGCC | |
| | TTCAGCACCAGAGTGGTCACCATCAACAGAGTGATGGGCTACCTGTC | |
| | CAGCGCCTGAGGCGCGCCGCAATTTGTACCCTTAATAAATTTCACAA | |
| | ACAGATTTTATCGCATCGTGTCTTATTGCGGGGGAGAAAACCGATGT | |
| | CGGCATAGAAAACCGCCATGATTCTAAGACGTCCGAACGCGAGTGGG | |
| | TGGGGAACAACCCATACCGGACAGATGCCGATGAGCCACCCGCACCC | |
| | TTGGGTGCGGGAGGTACGGGGTGGTTTGTTCATCCTATGGTTCCGAC | |
| | CCCACAAACAGCCCCCAGAGTCGGTTTGGGTATGGTTACATTTTCTG | |
| | TCTGGTGGTCGGGCTTGTTTCTTCCTTG | |
| mCMV | TGGCACGTATACTGAGTCATTAGGGACTTTCCAATGGGTTTTGCCCA | 301 |
| promoter | GTACATAAGGTCAATAGGGGTGAATCAACAGGAAAGTCCCATTGGAG | |
| for | CCAAGTACACTGAGTCAATAGGGACTTTCCATTGGGTTTTGCCCAGT | |
| expression | ACAAAAGGTCAATAGGGGGTGAGTCAATGGGTTTTTCCCATTATTGG | |
| of the | CACGTACATAAGGTCAATAGGGGTGAGTCATTGGGTTTTTCCAGCCA | |
| mouse | ATTTAATTTAAACGCCATGTACTTTCCCACCATTGACGTCAATGGGC | |
| CTLA-4 | TATTGAAACTAATGCAACGTGACCTTTAAACGGTACTTTCCCATAGC | |
| antagonist | TGATTAATGGGAAAGTACCGTTCTCGAGCCAATACACGTCAATGGGA | |
| payload. | AGTGAAAGGGCAGCCAAAACGTAACACCGCCCCGGTTTTCCCCTGGA | |
| 5′ > 3′ | AATTCCATATTGGCACGCATTCTATTGGCTGAGCTGCGTTCTACGTG | |
| direction | GGTATAAGAGGCGCGACCAGCGTCGGTACCGTCGCAGTCTTCGGTCT | |
| | GACCACCGTAGAACGCAGA | |
| Partial | GCCACC | |
| Kozak | | |
| sequence | | |
| for | | |
| expression | | |
| of the | | |
| mouse | | |
| CTLA-4 | | |
| antagonist | | |
| payload. | | |
| 5′ > 3′ | | |
| direction | | |
| Mouse | ATGGCTTGGGTTTGGACCCTGCTGTTCCTGATGGCCGCTGCTCAGTC | 303 |
| CTLA-4 | TATCCAGGCTCAGGTGCAGCTGGTGGAATCTGGCGGAGGACTTGCTC | |
| antagonist | AACCTGGCGGCTCTCTGAGACTGTCTTGTGCCGCTTCTGGCAGCACC | |
| payload. | ATCAGCTCTGTGGCTGTCGGCTGGTACAGACAGACACCTGGCAACCA | |
| 5′ > 3′ | GAGAGAGTGGGTCGCCACCAGCAGCACAAGCTCTACCACAGCCACAT | |
| direction | ACGCCGACAGCGTGAAGGGCAGATTCACCATCTCCAGAGACAACGCC | |
| | AAGAACACCATCTACCTGCAGATGAACAGCCTGAAGCCTGAGGACAC | |
| | CGCCGTGTACTACTGCAAGACCGGCCTGACAAACTGGGGCAGAGGCA | |
| | CCCAAGTGACCGTGTCTAGCGAACCTAGAGGCCCCACCATCAAGCCC | |
| | TGTCCTCCATGCAAATGCCCCGCTCCTAATCTGCTCGGCGGACCCAG | |
| | CGTGTTCATCTTCCCACCTAAGATCAAGGACGTGCTGATGATCTCTC | |
| | TGAGCCCCATCGTGACCTGCGTGGTGGTGGATGTGTCTGAGGACGAC | |
| | CCTGACGTGCAGATTAGTTGGTTCGTGAACAACGTCGAGGTGCACAC | |
| | AGCCCAGACACAGACCCACAGAGAGGACTACAACAGCACCCTGAGAG | |
| | TGGTGTCTGCCCTGCCTATCCAGCACCAGGATTGGATGAGCGGCAAA | |
| | GAATTCAAGTGCAAAGTGAACAACAAGGACCTGCCTGCTCCTATCGA | |
| | GAGAACCATCAGCAAGCCCAAGGGCTCTGTCAGGGCTCCTCAGGTGT | |
| | ACGTTCTGCCACCTCCTGAGGAAGAGATGACCAAGAAACAAGTGACC | |
| | CTGACCTGTATGGTCACCGACTTCATGCCCGAGGACATCTACGTGGA | |
| | ATGGACCAACAACGGCAAGACCGAGCTGAACTACAAGAACACAGAGC | |
| | CCGTGCTGGACAGCGACGGCAGCTACTTCATGTACAGCAAGCTGCGC | |
| | GTCGAGAAGAAGAACTGGGTCGAGAGAAACAGCTACAGCTGCAGCGT | |
| | GGTGCACGAGGGACTGCACAACCACCACACCACCAAGAGCTTCAGCA | |
| | GAACCCCTGGCAAGTGATAA | |
| GAPDH_SP | GACCCCTGGACCACCAGCCCCAGCAAGAGCACAAGAGGAAGAGAGAG | 304 |
| A sequence | ACCCTCACTGCTGGGGAGTCCCTGCCACACTCAGTCCCCCACCACAC | |
| for | TGAATCTCCCCTCCTCACAGTTGCCATGTAGACCCCTTGAAGAGGGG | |
| expression | AGGGGCCTAGGGAGCCGCACCTTGTCATGTACCATCAATAAAATATC | |
| of the | TTTATTTTCATTACATCTGTGTGTTGGTTTTTTGTGTGTTTCGATAG | |
| mouse | TACTAACATACGCTCTCCATCAAAACAAAACGAAACAAAACAAACTA | |
| CTLA-4 | GCAAAATAGGCTGTCCCCAGTGCAAGTGCAGGTGCCAGAACATTTCT | |
| antagonist | CT | |
| payload. | | |
| 5′ > 3′ | | |
| direction | | |
| C2 pause | TCAGTGCCTCTATCTGGAGGCCAGGTAGGGCTGGCCTTGGGGGAGGG | 305 |
| site for | GGAGGCCAGAATGACTCCAAGAGCTACAGGAAGGCAGGTCAGAGACC | |
| expression | CCACTGGACAAACAGTGGCTGGACTCTGCACCATAACACACAATCAA | |
| of the | CAGGGGAGTGAGCTGGATCC | |
| mouse | | |
| CTLA-4 | | |
| antagonist | | |
| payload. | | |
| 5′ > 3′ | | |
| direction | | |
| hGT pause | CTCAAAAATAAAATAAAATAAAAATTAAAAAAAAAGGTTACTCTGGC | 306 |
| site for | TATGAACCCTACCATATATTATCCTG | |
| expression | | |
| of mouse | | |
| CD40 | | |
| agonist | | |
| payload | | |
| 3′ > 5′ | | |
| direction | | |
| hBG poly A | TAAAATACAGCATAGCAAAACTTTAACCTCCAAATCAAGCCTCTACT | 307 |
| for | TGAATCCTTTTCTGAGGGATGAATAAGGCATAGGCATCAGGGGCTGT | |
| expression | TGCCAATGTGCATTAGCTGTTTGCAGCCTCACCTTCTTTCATGGAGT | |
| of mouse | TTAAGATATAGTGTATTTTCCCAAGGTTTGAACTAGCTCTTCATTTC | |
| CD40 | TTTATGTTTTAAATGCACTGACCTCCCACATTCCCTTTTTAGTAAAA | |
| agonist | TATTCAGAAATAATTTAAATACATCATTGCAATGAAAATAAATGTTT | |
| payload. | TTTATTAGGCAGAATCCAGATGCTCAAGGCCCTTCATAATATCCCCC | |
| 3′ > 5′ | AGTTTAGTAGTTGGACTTAGGGAACAAAGGAACCTTTAATAGAAATT | |
| direction | GGACAGCAAGAAAGCGAGC | |
| Mouse | CTATCACAGCTTCAGTAAACCGAAGGAGGAGAAGCCCACACGATGGA | 308 |
| CD40 | TAACTTGGCTGGCCTCGGTCACGTTCACGAACACGGAGGCGCCAGCT | |
| agonist | TGCAGCTCGAACACGCCTCCTAAATGGACGGACTGCTGCTCGCACAG | |
| payload. | CTGGGAGGAGGAGTGGGTGTTGGCGGCCTTCAGTAAAATCCTCTCGG | |
| 3′ > 5′ | AGCCGGAGCTGGGCTTCAGCCATAAACCCACGATGAAGGGCCTCTGG | |
| direction | CTGGAGGGCTCACGATTGGAGCAGAAGGTAACTTGGGTGTACACGTA | |
| | GTATAAACCCTCCCTCTTCACGGTCAGCTGCTTGCCGTTCTCCAGCA | |
| | TCACTAAATTGCTCTTCATGGTGTAGTAGCCCTTTTTGGCCCACTGC | |
| | AGCACGGAGGCGGCATTGGAGTTGGCCTCGGACACCACGTGGGCGGC | |
| | AATCTGAGGGTCGGAGCCGCCGCCAGAGCCGCCGCCGGAGCCGCCAC | |
| | CCAGCTTCAGTAAACCGAAGGAGCTGAAGCCCACACGATGGATAACT | |
| | TGGGAGGCCTCGGTCACGTTCACGAACACGGAGGCGCCAGCTTGCAG | |
| | CTCAAACACGCCTCCTAAATGCACGGACTGCTGCTCGCACAGCTGGC | |
| | TGGAGGAGTGGGTGTTGGCGGCCTTCAGCAGAATCCTCTCGCTGCCG | |
| | GAGGAGGGCTTCAGCCACAGTCCCACGATGAAAGGCCTCTGGGAGGA | |
| | GGGCTCACGATTGGAGCAGAAGGTAACTTGGGTGTACACGTAGTATA | |
| | AACCTTCCCTCTTCACGGTCAGCTGCTTGCCGTTCTCCAGCATCACT | |
| | AAATTGCTCTTCATGGTGTAGTAGCCCTTCTTGGCCCACTGCAGCAC | |
| | GCTAGCGGCGTTGGAGTTGGCCTCGGACACCACGTGAGCGGCGATCT | |
| | GGGGGTCGGAGCCGCCGCCAGAGCCGCCGCCGGAGCCGCCACCCAGC | |
| | TTCAGTAAACCAAAGGAGGAGAAGCCCACACGATGGATAACTTGGGA | |
| | GGCCTCGGTCACGTTCACGAACACGCTGGCGCCAGCTTGCAGCTCAA | |
| | ACACGCCTCCTAAATGCACGGACTGCTGCTCGCACAGCTGGGAGGAG | |
| | GAGTGGGTGTTGGCGGCCTTCAGTAAAATCCTCTCGGAGCCGCTGCT | |
| | AGGCTTCAGCCATAAACCCACGATGAAGGGCCTCTGGGAGGAAGGCT | |
| | CCCGGTTGGAGCAGAAGGTAACTTGGGTGTACACGTAGTATAAACCC | |
| | TCCCTCTTCACTGTCAGCTGCTTGCCGTTCTCCAGCATCACTAAATT | |
| | GGACTTCATGGTGTAGTAGCCCTTCTTGGCCCACTGCAGCACGGAAG | |
| | CGGCGTTGGAGTTGGCCTCGGACACCACGTGGGCAGCGATCTGGGGG | |
| | TCAGATCCACCACCACCAGAACCGCCTCCGCCAAGGAATGTTGACAG | |
| | AAGCACCCACTCTCCATCTTTGCGAACGTAGGCTTGGCCGTCCCTTG | |
| | GTGCTTCTGGGATATAACCGGCCTGTATACTTTGGGCAGCTGCCATC | |
| | AGGAATAGCAAGGTCCACACCCAAGCCAT | |
| Partial | GGTGGC | |
| Kozak | | |
| sequence | | |
| for | | |
| expression | | |
| of mouse | | |
| CD40 | | |
| agonist | | |
| payload. | | |
| 3′ > 5′ | | |
| direction | | |
| AoHV1 | CCAGTGAAGGGTCCCCGGCTAGGCTAGGCCGCTCGGTCCCGGTGGCT | 310 |
| promoter | GGTCCGGAGAACGTGGGTCCCTCACGCTGTCTTGAAGGCGAATGGCG | |
| for | GTCTGCTAACCGGGCTCTGCTTATATACCCGTAGGACACCCATGGTC | |
| expression | CGCCCACGAACCGCCCAGTAACGCCCATAACTCCGCCCGGGAACGCC | |
| of mouse | CATGGTCACTCCCCGTTACGCCCCTTTCCACTGACGTCAATGGAAAG | |
| CD40 | TCCCCGCGGTTTTGGTGCCAAGAACATTGACTAATATGGAATTTCCC | |
| agonist | CACCCACCATTGTCGGTAATGGTGGGAAAGGGGAACTGGCCCCGTTC | |
| payload. | CCATTGACGTCACTGGCTATTGCCAAGGACATTGACTAATAATAGAA | |
| 3′ > 5′ | ATCCCCCTTTTTGGTGCCAAATGAGGCGGTGGGCTTATGGAAAGTCC | |
| direction | CCATTTAACCCCTATTCTGGTGCCAGGACGGATGTATATGCTTGCCA | |
| | AGTCATTGATTT | |
| MMLV | ATAATTAAGTAACGCCATTTTGCAAGGCATGGAAAAATACATAACTG | 311 |
| promoter | AGAATAGAGAAGTTCAGATCAAGGTCAGGAACAGATGGAACAGCTGA | |
| for | ATATGGGCCAAACAGGATATCTGTGGTAAGCAGTTCCTGCCCCGGCT | |
| expression | CAGGGCCAAGAACAGATGGAACAGCTGAATATGGGCCAAACAGGATA | |
| of mouse | TCTGTGGTAAGCAGTTCCTGCCCCGGCTCAGGGCCAAGAACAGATGG | |
| scIL-12 | TCCCCAGATGCGGTCCAGCCCTCAGCAGTTTCTAGAGAACCATCAGA | |
| payload. | TGTTTCCAGGGTGCCCCAAGGACCTGAAATGACCCTGTGCCTTATTT | |
| 5′ > 3′ | GAACTAACCAATCAGTTCGCTTCTCGCTTCTGTTCGCGCGCTTCTGC | |
| direction. | TCCCCGAGCTCAATAAAAGAGCCCACAACCCCTCACTCGGGGCGCCA | |
| | GTCCTCCGATTGACTGAGTCGCCCGCTTAAG | |
| Partial | GCCACC | |
| Kozak | | |
| sequence | | |
| for | | |
| expression | | |
| of mouse | | |
| scIL-12 | | |
| payload. | | |
| 5′ > 3′ | | |
| direction. | | |
| Mouse | ATGTGCCCTCAGAAGCTGACCATCAGTTGGTTCGCCATCGTGCTGCT | 313 |
| scIL-12 | GGTGTCCCCACTGATGGCTATGTGGGAACTCGAGAAGGACGTGTACG | |
| payload | TGGTGGAAGTGGACTGGACCCCTGATGCTCCTGGCGAGACAGTGAAC | |
| 5′ > 3′ | CTGACCTGCGACACACCTGAAGAGGACGACATCACCTGGACCAGCGA | |
| direction. | TCAGAGACACGGCGTGATCGGCTCTGGCAAGACCCTGACAATTACCG | |
| | TGAAAGAGTTCCTGGACGCCGGCCAGTACACCTGTCACAAAGGCGGA | |
| | GAGACACTGAGCCACTCTCATCTGCTGCTGCACAAGAAAGAGAACGG | |
| | CATCTGGTCCACCGAGATCCTGAAGAACTTCAAGAACAAGACCTTCC | |
| | TGAAGTGCGAGGCCCCTAACTACAGCGGCAGATTCACCTGTAGCTGG | |
| | CTGGTGCAGAGAAACATGGACCTGAAGTTCAACATCAAGTCCTCCAG | |
| | CAGCAGCCCCGACAGCAGAGCTGTGACATGTGGCATGGCTTCTCTGA | |
| | GCGCCGAGAAAGTGACACTGGACCAGAGAGACTACGAGAAGTACAGC | |
| | GTGTCCTGCCAAGAGGACGTGACCTGTCCTACCGCCGAGGAAACACT | |
| | GCCTATCGAGCTGGCCCTGGAAGCCAGACAGCAGAACAAATACGAGA | |
| | ACTACTCTACCAGCTTCTTCATCCGGGACATCATCAAGCCCGATCCT | |
| | CCAAAGAACCTGCAGATGAAGCCTCTGAAGAACAGCCAGGTCGAGGT | |
| | GTCCTGGGAGTACCCTGACAGCTGGTCTACCCCTCACAGCTACTTCA | |
| | GCCTGAAATTCTTCGTGCGGATCCAGCGCAAGAAAGAAAAGATGAAG | |
| | GAAACCGAGGAAGGCTGCAACCAGAAAGGCGCTTTCCTGGTGGAAAA | |
| | GACCAGCACCGAGGTGCAGTGCAAAGGCGGCAATGTCTGTGTGCAGG | |
| | CCCAGGACCGGTACTACAACAGCAGCTGTAGCAAGTGGGCCTGCGTG | |
| | CCATGCAGAGTTAGAAGCGGAGGCGGAGGTTCTGGTGGCGGAGGAAG | |
| | TGGCGGCGGAGGATCTAGAGTGATCCCTGTGTCTGGCCCTGCCAGAT | |
| | GTCTGAGCCAGAGCAGAAACCTGCTGAAAACCACCGACGACATGGTC | |
| | AAGACCGCCAGAGAGAAGCTGAAGCACTACTCCTGCACAGCCGAGGA | |
| | CATCGACCACGAGGATATCACCAGGGACCAGACAAGCACCCTGAAAA | |
| | CCTGCCTGCCTCTGGAACTGCATAAGAACGAGAGCTGCCTGGCCACC | |
| | AGAGAAACCAGCTCTACCACAAGAGGCAGCTGTCTGCCTCCTCAGAA | |
| | AACCAGCCTGATGATGACCCTGTGCCTGGGCAGCATCTACGAGGATC | |
| | TGAAGATGTACCAGACCGAGTTCCAGGCCATCAACGCCGCTCTGCAG | |
| | AACCACAACCACCAGCAGATCATCCTGGACAAGGGCATGCTGGTGGC | |
| | TATCGACGAGCTGATGCAGAGCCTGAACCATAACGGCGAGACACTGC | |
| | GGCAGAAGCCTCCAGTTGGAGAGGCCGATCCTTACAGAGTGAAGATG | |
| | AAGCTGTGCATCCTGCTGCACGCCTTCAGCACCAGAGTGGTCACCAT | |
| | CAACAGAGTGATGGGCTACCTGTCCAGCGCCTGA | |

TABLE 12
Additional Nucleotide Sequences.

| | | SEQ |
|---|---|---|
| Descrip- | | ID |
| tion | Sequence | NO |
| US9-10 | GGCAATTTGTACCCTTAATAAATTTCACAAACAG | 314 |
| polyA | ATTTTATCGCATCGTGTCTTATTGCGGGGGAGAA | |
| 3′>5′ | AACCGATGTCGGCATAGAAAACCGCCATGATTCT | |
| direc- | AAGACGTCCGAACGCGAGTGGGTGGGGAACAACC | |
| tion | CATACCGGACAGATGCCGATGAGCCACCCGCACC | |
| | CTTGGGTGCGGGAGGTACGGGGTGGTTTGTTCAT | |
| | CCTATGGTTCCGACCCCACAAACAGCCCCCAGAG | |
| | TCGGTTTGGGTATGGTTACATTTTCTGTCTGGTG | |
| | GTCGGGCTTGTTTCTTCCTTG | |
| Pbidir3 | GTTAACGTAGGTAGGGTCGTATCGAGCCGCGGTC | 315 |
| promo- | CGTCCTGCTCTGGGCTCGAATGGCATGGGGGACA | |
| ter | GCAATTATATGGTTAACTCCGCCCGTTTTATGAC | |
| | TAGAACCAATAGTTTTTAATGCCAAATGCACTGA | |
| | AATCCCCTAATTTGCAAAGCCAAACGCCCCCTAT | |
| | GTGAGTAATACGGGGACTTTTTACCCAATTTCCC | |
| | ACGCGGAAAGCCCCCTAATACACTCATATGGCAT | |
| | ATGAATCAGCACGGTCATGCACTCTAATGGCGGC | |
| | CCATAGGGACTTTCCACATAGGGGGCGTTCACCA | |
| | TTTCCCAGCATAGGGGTGGTGACTCAATGGCCTT | |
| | TACCCAAGTACATTGGGTCAATGGGAGGTAAGCC | |
| | AATGGGTTTTTCCCATTACTGGCAAGCACACTGA | |
| | GTCAAATGGGACTTTCCACTGGGTTTTGCCCAAG | |
| | TACATTGGGTCAATGGGAGGTGAGCCAATGGGAA | |
| | AAACCCATTGCTGCCAAGTACACTGACTCAATAG | |
| | GGACTTTCCAATGGGTTTTTCCATTGTTGGCAAG | |
| | CATATAAGGTCAATGTGGGTGAGTCAATAGGGAC | |
| | TTTCCATTGTATTCTGCCCAGTACATAAGGTCAA | |
| | TAGGGGGTGAATCAACAGGAAAGTCCCATTGGAG | |
| | CCAAGTACACTGCGTCAATAGGGACTTTCCATTG | |
| | GGTTTTGCCCAGTACATAAGGTCAATAGGGGATG | |
| | AGTCAATGGGAAAAACCCATTGGAGCCAAGTACA | |
| | CTGACTCAATAGGGACTTTCCATTGGGTTTTGCC | |
| | CAGTACATAAGGTCAATAGGGGGTGAGTCAACAG | |
| | GAAAGTTCCATTGGAGCCAAGTACATTGAGTCAA | |
| | TAGGGACTTTCCAATGGGTTTTGCCCAGTACATA | |
| | AGGTCAATGGGAGGTAAGCCAATGGGTTTTTCCC | |
| | ATTACTGGCACGTATACTGAGTCATTAGGGACTT | |
| | TCCAATGGGTTTTGCCCAGTACATAAGGTCAATA | |
| | GGGGTGAATCAACAGGAAAGTCCCATTGGAGCCA | |
| | AGTACACTGAGTCAATAGGGACTTTCCATTGGGT | |
| | TTTGCCCAGTACAAAAGGTCAATAGGGGGTGAGT | |
| | CAATGGGTTTTTCCCATTATTGGCACGTACATAA | |
| | GGTCAATAGGGGTGAGTCATTGGGTTTTTCCAGC | |
| | CAATTTAATTTAAACGCCATGTACTTTCCCACCA | |
| | TTGACGTCAATGGGCTATTGAAACTAATGCAACG | |
| | TGACCTTTAAACGGTACTTTCCCATAGCTGATTA | |
| | ATGGGAAAGTACCGTTCTCGAGCCAATACACGTC | |
| | AATGGGAAGTGAAAGGGCAGCCAAAACGTAACAC | |
| | CGCCCCGGTTTTCCCCTGGAAATTCCATATTGGC | |
| | ACGCATTCTATTGGCTGAGCTGCGTTCTACGTGG | |
| | GTATAAGAGGCGCGACCAGCGTCGGTACCGTCGC | |
| | AGTCTTCGGTCTGACCACCGTAGAACGCAGA | |
| SV40pA | GACATGATAAGATACATTGATGAGTTTGGACAAA | 316 |
| | CCACAACTAGAATGCAGTGAAAAAAATGCTTTAT | |
| | TTGTGAAATTTGTGATGCTATTGCTTTATTTGTA | |
| | ACCATTATAAGCTGCAATAAACAAGTTAACAACA | |
| | ACAATTGCATTCATTTTATGTTTCAGGTTCAGGG | |
| | GGAGGTGTGGGAGGTTTTTTAAAGCAAGTAAAAC | |
| | CTCTACAAATGTGGTA | |
| rBG_pA | AAATGATTTGCCCTCCCATATGTCCTTCCGAGTG | 317 |
| | AGAGACACAAAAAATTCCAACACACTATTGCAAT | |
| | GAAAATAAATTTCCTTTATTAGCCAGAGGTC | |

What is claimed is:

1. An oncolytic herpes simplex type 1 virus (HSV-1) comprising
a. a cassette integrated in one or both of the γ34.5 loci comprising in order, from upstream to downstream, a CMV promoter, a polynucleotide encoding hFLT3L, a P2A cleavage sequence, a polynucleotide encoding UL49.5, and a polyadenylation signal; and
b. another cassette integrated in the US10-12 locus comprising in order, from upstream to downstream, a polynucleotide comprising a variant US11 gene encoding native US11 protein, an additional polynucleotide encoding native US11 protein, a polynucleotide encoding US10 protein, a polyadenylation signal that is operably linked to the polynucleotide encoding the US10 protein, a CMV promoter, a polynucleotide encoding a CTLA-4 binding protein, a polyadenylation signal that is operably linked to the polynucleotide encoding the CTLA-4 binding protein, a polyadenylation signal that is operably linked to a polynucleotide encoding a CD40 agonist, a polynucleotide encoding a CD40 agonist, an AoHV1 promoter that controls expression of the CD40 agonist, an MMLV promoter that controls expression of an IL-12, a polynucleotide encoding an IL-12, and a polyadenylation signal that is operably linked to the polynucleotide encoding the IL-12, wherein
the polynucleotide for hFLT3L encodes the amino acid sequence set forth in SEQ ID NO:71, the polynucleotide for UL49.5 encodes the amino acid sequence set forth in SEQ ID NO: 82, the polynucleotide for IL-12 encodes the amino acid sequence set forth in SEQ ID NO: 4, the polynucleotide for CD40 agonist encodes the amino acid sequence set forth in SEQ ID NO: 25, and the polynucleotide for CTLA-4 binding protein encodes the amino acid sequence set forth in SEQ ID NO: 50, the polynucleotide for variant US11 gene comprises the polynucleotide sequence set forth in SEQ ID NO: 204, the additional polynucleotide encoding for US11 encodes the amino acid sequence set forth in SEQ ID NO:80, and the polynucleotide for US10 encodes the amino acid sequence set forth in SEQ ID NO:90.

2. An oncolytic herpes simplex type 1 virus (HSV-1) comprising
a. a cassette integrated in one or both of the γ34.5 loci comprising in order, from upstream to downstream, a CMV promoter, a polynucleotide encoding hFLT3 protein, a P2A cleavage sequence, a polynucleotide encoding UL49.5, and a polyadenylation signal; and
b. another cassette integrated in the US10-12 locus comprising in order, from upstream to downstream, a polynucleotide comprising a variant US11 gene encoding native US11 protein, an additional polynucleotide encoding native US11 protein, a polynucleotide encoding US10 protein, a polyadenylation signal that is operably linked to the polynucleotide encoding the US10 protein, a CMV promoter, a polynucleotide encoding a CTLA-4 binding protein, a polyadenylation signal that is operably linked to the polynucleotide encoding the CTLA-4 binding protein, a polyadenylation signal that is operably linked to a polynucleotide encoding a CD40 agonist, a polynucleotide encoding a CD40 agonist, an AoHV1 promoter that controls expression of the CD40 agonist, an MMLV promoter that controls expression of an IL-12, a polynucleotide encoding an IL-12, and a polyadenylation signal that is operably linked to the polynucleotide encoding the IL-12, wherein
the polynucleotide for hFLT3L encodes the amino acid sequence set forth in SEQ ID NO:71, the polynucleotide for UL49.5 encodes the amino acid sequence set forth in SEQ ID NO: 82, the polynucleotide for IL-12 encodes the amino acid sequence set forth in SEQ ID NO: 8, the polynucleotide for CD40 agonist encodes the amino acid sequence set forth in SEQ ID NO: 28, and the polynucleotide for CTLA-4 binding protein encodes the amino acid sequence set forth in SEQ ID NO: 56, the polynucleotide for variant US11 gene comprises the polynucleotide sequence set forth in SEQ ID NO: 204, the additional polynucleotide for US11 encodes the amino acid sequence set forth in SEQ ID NO:80, and the polynucleotide for US10 encodes the amino acid sequence set forth in SEQ ID NO:90.

3. An oncolytic virus, comprising one or more expression cassettes comprising a polynucleotide encoding IL-12, a polynucleotide encoding a CD40 agonist, and a polynucleotide encoding a CTLA-4 binding protein.

4. The oncolytic virus of claim 3, wherein one or both native γ34.5 genes are inactivated by deletion, substitution, or insertion in the backbone nucleic acid.

5. The oncolytic virus of any one of claim 3, wherein a native US12 gene of the virus is inactivated by deletion, substitution, or insertion in the backbone nucleic acid.

6. The oncolytic virus of claim 3, wherein the one or more expression cassettes comprise a polynucleotide encoding a FLT3 ligand (FLT3L).

7. The oncolytic virus of claim 3, wherein the one or more one or more expression cassettes further comprises a polynucleotide comprising a variant US11 gene.

8. The oncolytic virus of claim 7, wherein the variant US11 gene is operably associated with an immediate-early promoter.

9. The oncolytic virus of claim 3, comprising a native late US11 gene.

10. The oncolytic virus of claim 3, further comprising a transporter associated with antigen processing (TAP) inhibitor.

11. The oncolytic virus of claim 3, wherein one of the expression cassettes is inserted at the native US10-US12 locus.

12. A pharmaceutical composition comprising the oncolytic virus of claim 1 or claim 4 and a pharmaceutically acceptable excipient.

13. An expression cassette comprising
a. a polynucleotide encoding a TAP inhibitor, and
b. a polynucleotide encoding a FLT3 ligand.

14. A modified HSV genome comprising the expression cassette of claim 13.

15. The modified HSV genome of claim 15, wherein one or both γ34.5 loci of the modified HSV genome is replaced with the cassette.

16. An oncolytic virus comprising the expression cassette of claim 13.

17. The oncolytic virus of claim 16, wherein one or both γ34.5 loci of the oncolytic virus are replaced with the expression cassette.

18. An expression cassette comprising:
a. a polynucleotide encoding an IL-12,
b. a polynucleotide encoding a CD40 agonist, and
c. a polynucleotide encoding a CTLA-4 binding protein.

19. The expression cassette of claim 18, further comprising a polynucleotide encoding a US10 protein.

20. The expression cassette of claim 18, wherein the expression cassette comprises a native late US11 gene.

21. The expression cassette of claim 18, wherein the expression cassette comprises a variant polynucleotide encoding a US11 protein.

22. A modified HSV genome, comprising the expression cassette of claim 18.

23. The modified HSV genome of claim 22, wherein the expression cassette is integrated in the US10-12 locus of the modified HSV genome.

24. An oncolytic virus comprising the expression cassette of claim 13 or claim 18.

25. The oncolytic virus of claim 24, wherein the virus is a herpes simplex virus (HSV).

26. A method of treating cancer in an individual comprising administering to the individual an effective amount of the oncolytic virus of claim 1 or claim 3.

27. A method of killing tumor cells in an individual comprising administering the oncolytic virus of claim 1 or claim 3.

28. A method of producing an oncolytic comprising culturing a cell comprising the oncolytic virus of claim 1 or claim 3, lysing the cell to produce a cell lysate, and purifying the oncolytic virus from the cell lysate.

29. A CD40 agonist protein comprising three single-chain trimeric CD40 ligand ectodomains, each fused to a trimerization motif, wherein the CD40 agonist protein is a trimer of trimers.

30. The CD40 agonist protein of claim 29, wherein the trimerization motif is a T4 fibritin trimerization motif.

31. A polynucleotide encoding the CD40 agonist protein of claim 29.

32. A vector comprising the polynucleotide of claim 31.

33. A host cell comprising the vector of claim 32.

34. A method of inhibiting tumor growth in an individual comprising administering an effective amount of the CD40 agonist protein of claim 29.


SEQUENCE LISTING (321 sequences)

SEQ ID NO: 1 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 7 aa, synthetic construct
GGGGGGS

SEQ ID NO: 4 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 193 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - protein, 15 aa, synthetic construct
GGGGSGGGGSGGGGS

SEQ ID NO: 8 - protein, 521 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 9 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 510 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 521 aa, synthetic construct; identical to SEQ ID NO: 8

SEQ ID NO: 13-19 - skipped (7 entries)

SEQ ID NO: 20 - protein, 143 aa, synthetic construct
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 21 - protein, 27 aa, synthetic construct
GYIPEAPRDGQAYVRKDGEWVLLSTFL

SEQ ID NO: 22 - protein, 12 aa, synthetic construct
GGGSGGGSGGGS

SEQ ID NO: 23 - protein, 5 aa, synthetic construct
GGGGS

SEQ ID NO: 24 - protein, 19 aa, synthetic construct
MAWVWTLLFLMAAAQSIQA

SEQ ID NO: 25 - protein, 504 aa, synthetic construct
MAWVWTLLFLMAAAQSIQAGYIPEAPRDGQAYVRKDGEWVLLSTFLGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 26 - protein, 143 aa, synthetic construct
DPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 27 - protein, 10 aa, synthetic construct
GGGGSGGGGS

SEQ ID NO: 28 - protein, 509 aa, synthetic construct
MAWVWTLLFLMAAAQSIQAGYIPEAPRDGQAYVRKDGEWVLLSTFLGGGGSGGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGSGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGSGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 29 - protein, 490 aa, synthetic construct
GYIPEAPRDGQAYVRKDGEWVLLSTFLGGGGSGGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGSGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGSGGGSDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 30 - protein, 485 aa, synthetic construct
GYIPEAPRDGQAYVRKDGEWVLLSTFLGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 31-39 - skipped (9 entries)

SEQ ID NO: 40 - protein, 10 aa, synthetic construct
GFTFSSYTMH

SEQ ID NO: 41 - protein, 10 aa, synthetic construct
FISYDGNNKY

SEQ ID NO: 42 - protein, 9 aa, synthetic construct
TGWLGPFDY

SEQ ID NO: 43 - protein, 12 aa, synthetic construct
RASQSVGSSYLA

SEQ ID NO: 44 - protein, 7 aa, synthetic construct
GAFSRAT

SEQ ID NO: 45 - protein, 9 aa, synthetic construct
QQYGSSPWT

SEQ ID NO: 46 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 47 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 48 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 19 aa, synthetic construct
MAWVWTLLFLMAAAQSIQA

SEQ ID NO: 50 - protein, 497 aa, synthetic construct
MAWVWTLLFLMAAAQSIQAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSEGKSSGSGSESKSTGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 10 aa, synthetic construct
GSTISSVAVG

SEQ ID NO: 52 - protein, 10 aa, synthetic construct
TSSTSSTTAT

SEQ ID NO: 53 - protein, 4 aa, synthetic construct
GLTN

SEQ ID NO: 54 - protein, 113 aa, synthetic construct
QVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSS

SEQ ID NO: 55 - protein, 19 aa, synthetic construct
MAWVWTLLFLMAAAQSIQA

SEQ ID NO: 56 - protein, 365 aa, synthetic construct
MAWVWTLLFLMAAAQSIQAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - protein, 364 aa, synthetic construct
MAWVWTLLFLMAAAQSIQAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 58 - protein, 346 aa, synthetic construct
QVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 59 - protein, 345 aa, synthetic construct
QVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 60 - protein, 478 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSEGKSSGSGSESKSTGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 20 aa, synthetic construct
GGSEGKSSGSGSESKSTGGS

SEQ ID NO: 62-69 - skipped (8 entries)

SEQ ID NO: 70 - protein, 26 aa, synthetic construct
MTVLAPAWSPTTYLLLLLLLSSGLSG

SEQ ID NO: 71 - protein, 235 aa, synthetic construct
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 72 - protein, 209 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 73-79 - skipped (7 entries)

SEQ ID NO: 80 - protein, 155 aa, synthetic construct
MSQTQPPAPVGPGDPDVYLKGVPSAGMHPRGVHAPRGHPRMISGPPQRGDNDQAAGQCGDSGLLRVGADTTISKPSEAVRPPTIPRTPRVPREPRVPRPPREPREPRVPRDPRDPRVPRDPRDPRQPRSPREPRSPREPRTPRTPREPRTARGSV

SEQ ID NO: 81 - protein, 18 aa, synthetic construct
PRSPLIVAVVAAALFAIV

SEQ ID NO: 82 - protein, 95 aa, synthetic construct
PRSPLIVAVVAAALFAIVRGRDPLLDAMRREGAMDFWSAGCYARGVPLSEPPQALVVFYVALTAVMVAVALYAYGLCFRLMGASGPNKKESRGRG

SEQ ID NO: 83 - protein, 77 aa, synthetic construct
RGRDPLLDAMRREGAMDFWSAGCYARGVPLSEPPQALVVFYVALTAVMVAVALYAYGLCFRLMGASGPNKKESRGRG

SEQ ID NO: 84-89 - skipped (6 entries)

SEQ ID NO: 90 - protein, 306 aa, synthetic construct
MIKRRGNVEIRVYYESVRTLRSRSHLKPSDRQQSPGHRVFPGSPGFRDHPENLGNPEYREIPETPGYRVTPGIHDNPGLPGSPGLPGSPGPHAPPANHVRLAGLYSPGKYAPLASPDPFSPQDGAYARARVGIHTAVRVPPTGSSTHTHLRQDPGDEPTSDDSGLYPLDARALAHLVMLPADHRAFFRTVVEVSRMCAANVRDPPPPATGAMLGRHARLVHTQWLRANQETSPLWPWRTAAINFITTMAPRVQTHRHMHDLLMACAFWCCLTHASTCSYAGLYSTHCLHLFGAFGCGDPALTPPLC

SEQ ID NO: 91 - protein, 22 aa, synthetic construct
GSGATNFSLLKQAGDVEENPGP

SEQ ID NO: 92 - protein, 351 aa, synthetic construct
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEHGSGATNFSLLKQAGDVEENPGPRSPLIVAVVAAALFAIVRGRDPLLDAMRREGAMDFWSAGCYARGVPLSEPPQALVVFYVALTAVMVAVALYAYGLCFRLMGASGPNKKESRGRG

SEQ ID NO: 93 - protein, 325 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEHGSGATNFSLLKQAGDVEENPGPRSPLIVAVVAAALFAIVRGRDPLLDAMRREGAMDFWSAGCYARGVPLSEPPQALVVFYVALTAVMVAVALYAYGLCFRLMGASGPNKKESRGRG

SEQ ID NO: 94-99 - skipped (6 entries)

SEQ ID NO: 100 - DNA, 1961 bp, synthetic construct
  source 1..1961: mol_type = other DNA
caactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctgataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctaggcgatcgcgatggccagcggtggtactgtcacgaccgcggtcggacctcgggttgttggatagaggaggacgacgacgacgactcgagccctgagtcaccctgggtcctgacgaggaaggttgtgtcggggtagaggaggctgaagcgacagttttaggcactcgacagactgatggacgaagttctaatgggtcagtggcaccggaggttggacgtcctgctcctcgagacgcccccggagaccgccgaccaggaccgtgtcgcgacctacctcgccgagttctgacagcgacccaggttctacgttccgaacgacctcgcgcacttgtgcctctatgtgaaacagtggtttacacggaaagtcgggggggggtcgacagaagcgaagcaggtctggttgtagagggcggaggacgtcctctggaggctcgtcgaccaccgcgacttcgggacctagtgagcggtcttgaagagggccacggacctcgacgtcacagtcgggctgaggagttgggacggtgggggtacctcaggggccggggacctccggtgtcggggctgtcggggcgtcgggggagacgaggaggatgacgacgacgggcacccggaggacgacgaccggcgacggaccacggacgtgaccgtctcctgcgccgcctcctgtggggcgggacccctcgtccacggggggcaggggtcaggggtcctggacgacgaacacctcgtgccgagacctcggtgcttgaagagagacaatttcgttcgtcctctgcaccttcttttggggccaggagccagcggcgagtagcaacgccaacaccggcggcgcgacaaacggtagcacgcgccggcgctgggggacgatctgcgctacgccgcgctcccccgttacctgaaaacctcgcgtccgacgatgcgcgcgccccacggcgagagcctcggtggcgtccgggaccaacaaaaaatgcaccgggactggcgccattaccagcggcaccgggacatgcgcatgcccgaaacgaaatccgagtacccgcggtcgcccgggttatttttcctcagcgcccccgccccgactccttcgggtccggtcgagctcgtacgtcccgggataagatatcacagtggatttacgatctcgagcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcg

SEQ ID NO: 101 - DNA, 7015 bp, synthetic construct
  source 1..7015: mol_type = other DNA
ggctggggagaagaagggagaagagggcgggaggtctgcgtggcctcagcccccagggtgccgcagggggtttataccgcccgccgaggaggggtggggggatctacgcacactcattccccccggacgcatactcagtcacccctggtgcgggggttgtgccgctggggccaggaacacacaaacaacacccccgcacagagacacatactcagtcccccagggtgccgctggggcccgggacgcagactcagtttccccggtacacatacacaacccccagacatatatatttcagtcccccagtgtaccgctgggggttgtcccgctggggccagggacatatatatcccagtcccccaaggcgtgggggattgtaccgcgggggccagggacatatatatcacagtgccccaaggtgcgggggattgtaccgcggggttgtaccgcgggccgagggcacatactcacccccagggggttgtaccgccggccaaggtcacattcccagcccccagggggttgtaccgcggggggttataccgcggggggttataccgcggggtctgtaccgcgggccggggagtggagcgcgacccccgccgggagtccggccgcccatgagcgaggccccgccccgaggtacccccagcatacgccgacctcccagcgcctgcctcccagggacccccagcgttgcatccgccccgaagacaccactacgcctctcccccgccgggctcagacggaccgacgacgcagagcgaggctcacggctccacgtttacgctggtctgacagcccggtcccgattgaatatggggtgcggaaaggggaggggtttccccgccgtcactgctaagggggttaccggcgcgcagggtcccctccgtccgggtggcgccccgccggggcaggggcccctggttgggccgcgggggtttcttatagtaatcgtacgtgccgggccgggggctaaacccccgggttgggccacagggggtttcttggggtaatcgtacggggagggcggctgcgttgtccccgaaccggacgcagccacggggccccgaagggcggaagggcttctttgagtaatggtatgggccttggggtcccctggttacgcccaagtaactcgctgggcgcccggttacgcgctccccggcacacaaggcggttttttcgttaatcgtattgggccttggggtcccctaaccaatgcgcgccgcgccctccgccccttatggccccaacgggtaattcccggcgcccttaacggccttcgcccttcccgccggccccggcgggtaattactcaaagattaatggtatggcccttcgccttgttccggagacgttcaaaaattaatggtatggcccttcacccgccgggccgggtgacccgccattgagggcgggtcacccggcccggggctgctgagccgcctgcgaccaaccggcccggggcggcgcgaccgccggcgagatctggagtcgtctagatctcaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctgataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctaggcgatcgcgatggccagcggtggtactgtcacgaccgcggtcggacctcgggttgttggatagaggaggacgacgacgacgactcgagccctgagtcaccctgggtcctgacgaggaaggttgtgtcggggtagaggaggctgaagcgacagttttaggcactcgacagactgatggacgaagttctaatgggtcagtggcaccggaggttggacgtcctgctcctcgagacgcccccggagaccgccgaccaggaccgtgtcgcgacctacctcgccgagttctgacagcgacccaggttctacgttccgaacgacctcgcgcacttgtgcctctatgtgaaacagtggtttacacggaaagtcgggggggggtcgacagaagcgaagcaggtctggttgtagagggcggaggacgtcctctggaggctcgtcgaccaccgcgacttcgggacctagtgagcggtcttgaagagggccacggacctcgacgtcacagtcgggctgaggagttgggacggtgggggtacctcaggggccggggacctccggtgtcggggctgtcggggcgtcgggggagacgaggaggatgacgacgacgggcacccggaggacgacgaccggcgacggaccacggacgtgaccgtctcctgcgccgcctcctgtggggcgggacccctcgtccacggggggcaggggtcaggggtcctggacgacgaacacctcgtgccgagacctcggtgcttgaagagagacaatttcgttcgtcctctgcaccttcttttggggccaggagccagcggcgagtagcaacgccaacaccggcggcgcgacaaacggtagcacgcgccggcgctgggggacgatctgcgctacgccgcgctcccccgttacctgaaaacctcgcgtccgacgatgcgcgcgccccacggcgagagcctcggtggcgtccgggaccaacaaaaaatgcaccgggactggcgccattaccagcggcaccgggacatgcgcatgcccgaaacgaaatccgagtacccgcggtcgcccgggttatttttcctcagcgcccccgccccgactccttcgggtccggtcgagctcgtacgtcccgggataagatatcacagtggatttacgatctcgagcgactagtcggagctgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgccggcgcccgcgcgaggactggtgcccaaggctcaacccgcacctccaatggaccctgacacgccaaccctggcgcgggcacccgggcccgccggcccccgccgcccccggcgctaccgccgccgccgcccggtacctctgtctctcgcacggccccaccatctcaaactgtccgttcgtacacgcacgtctccgctcatcacgaacggacagattgagcgatcagagccggcgccccccgggcccgacgggcggcggcgcgaaatttcccggcgcgcgctgggggccccccacacaaaacccccccgggcaaaagccccagaccggcgaggaggggggcgaggaggggggcgaggaggggggcgaggaggggggcgaggaggggggcgaggaggggggcgaggaggggggcgagggcgccggggcgggggttgcgggcggcgcgcgcgcgtgcggcgggcctggcggcgggcggaaaaaacgcgcgcgcgcgcgggcgccccccgggcccgacggtgtccactttggttgtctcgtgccgcgtgaggcgtgcagtgtgcagtgcagtaggtggtgtggacgggttgttgtgttgagtgtcgctgttgagtggcgcgttgttgaggacaaggagtaggtgtgcagtggcgcgtggagggcgaggaggtctgcatggggccgcgttgtgtggcgaggacgatgtgtggtggcgggggaggggtcggggtcgggaggggtcggggtcgggaggggccggggtcgggaggggccggggtcgggaggggccggggtcgggaggggccggggtcgggaggggccggggtcgggaggggccggggtcgggaggggccgcgcagggcgcgagggagcccccccaagcccgtagagatggagtcacggcggttagagtccagtctctaggtttgggaggcccccgcgggcgcgtggtggtggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcgggggagggcggggagcttatttgttgcgatgacgttttgaattagtccaacaacggcaaataacgcagaagcccagagtgttcgcggggcggggcagggccgggcaatgtcgtggggcagggggagcttgcgcggcggcagcagaagcagggtccgcggaagggtcaggtgttgaagggcggcgcccccgcaccggttcgggcggaggcgggggtcgtggaggtgccgggggcggcggcggtcgtgccacggcgacgccgggcaccggctccgggtcgcttagggcccgccgcggccgccgtcccgggggcccggcagcagcagcggcgcgtcgtggtcgcccccccgcagcagcagcccgaggtcgtcccgcgcccgcgttttcagggaggcgccgggcgcggtggcccggcccggccgcgcgtggcggagcgcggggtcgcggtgcatgtgcccggcgtcgccgcgcgggtccggggtcgcgcgcgtccgccgcacgctcacccggaggaggagcgtcttcaggccgcgcggcccgcggtaccgcagccaccaggggctccggcggcgggccggcaggtcgcggccgtcgtgccgggccgccatgagcgcgcccctgtacccgtggccgcacaggcccggcttcgcgcacgcgtgcgccatcgcgtgcaacggcggcgccgtgtccgcgtcgccgccgcgcagccccatgtccgcgcgcacgcgccggaggtgcgcgcgcttctgggggcccggcttgtgcgccgggctccggtcgtggcacgccgcgtccagggcgcggcggccggtcgcgtgccgcgtgacgtgccgcccgtcgtcgagcgtgcggtccatccgcacgacggcgctgtggcgcccgggcagccgcccggtcagcgtccgcgcgtgccacaactggtgctactcggcggccagcggccgcgaccgctcgtcggggtctttgaggtgccggggccgcttccggtccagggcgcacctgtcgtcgtcgtgcgggacacgcgggtcgcggctgtgcagcccccgcggccaggttaacgggcgggtccgccggcacaggccgggcgtgtcggccaaccggtcccggcggtcgtccgtcctgtcgggcggcgcgagccgcctggtgaggccgccgggggggctccggggcggcggccggtccaggagcgggccgtcgccgctcatgtcgtggtggtgcgcgtgcaggagccccagcccctagaccgcgtaggtccggcggtacgccgcgtcgcccgggctccgcgcgtcccccggtttctccgccgggggccgccggggcacccccacccccaatagcagcagcagcggcggcggcgtgcgccggacccgccgcccccgcccgggccgcgtggcgcgccgctagctccggtcccgggcgcccagtttgtactcccggccagcggtcccctgccccttgtcgcccaccaggcactcgagccggtgccgcgcgcccctcgtcatccggaggtcccgccgccggcgcccgcggcggcacaccgacccggggcccccgacggcggcggtcggcgggtcccccagccccgggagccgcccggccgcgctgtgccggtgccccgcgcccgcccggacgcggcgccgccgggccccgcggcgcccgacccgcccccgcccgagcccggggcccccgcacctccccccgcgcccgcgcccctccccccgcgcccgcaggctcggcccccgcaggcgcggcgagaagaagcagaagcccccagcgcccggcggcggaggcccgccggcccggcccggccctgagaacgcgaacgcggggagggcgccgcgccgcctccgccgccgccggcggtcgcgcagccgccgcaggccacgcgaccggcggcggcggtcgtcccccgcgtccgagaccaacagtttgtcgtccaggcgccgccgccgccggcgcctcgagccgtccgcgcccagggcgccgtcgcgccccgggtcccggggccgctggtccgagtgccgcgcgtgccgccggtgccgccggagcgacggcggccggtgcgcgtccaggggcgcgtccgcgtactcgtggtcgcgcagcgcgtgcttggcgtcgagcgcgtcggtgcgcgcgtccgccccgcgcagccgcacgccgccgccgccccttcgccccgggcgcccagggaggccggcgccccccgaccgcccggcccggggccggtcggggccctgccggcggtccagcggcagcttcgggagccggtcgcggaggt

SEQ ID NO: 102 - DNA, 194 bp, synthetic construct
  source 1..194: mol_type = other DNA
gacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcg

SEQ ID NO: 103 - DNA, 288 bp, synthetic construct
  source 1..288: mol_type = other DNA
ggagccagcggcgagtagcaacgccaacaccggcggcgcgacaaacggtagcacgcgccggcgctgggggacgatctgcgctacgccgcgctcccccgttacctgaaaacctcgcgtccgacgatgcgcgcgccccacggcgagagcctcggtggcgtccgggaccaacaaaaaatgcaccgggactggcgccattaccagcggcaccgggacatgcgcatgcccgaaacgaaatccgagtacccgcggtcgcccgggttatttttcctcagcgcccccgccccgact

SEQ ID NO: 104 - DNA, 66 bp, synthetic construct
  source 1..66: mol_type = other DNA
ccgagacctcggtgcttgaagagagacaatttcgttcgtcctctgcaccttcttttggggccagga

SEQ ID NO: 105 - DNA, 705 bp, synthetic construct
  source 1..705: mol_type = other DNA
tactgtcacgaccgcggtcggacctcgggttgttggatagaggaggacgacgacgacgactcgagccctgagtcaccctgggtcctgacgaggaaggttgtgtcggggtagaggaggctgaagcgacagttttaggcactcgacagactgatggacgaagttctaatgggtcagtggcaccggaggttggacgtcctgctcctcgagacgcccccggagaccgccgaccaggaccgtgtcgcgacctacctcgccgagttctgacagcgacccaggttctacgttccgaacgacctcgcgcacttgtgcctctatgtgaaacagtggtttacacggaaagtcgggggggggtcgacagaagcgaagcaggtctggttgtagagggcggaggacgtcctctggaggctcgtcgaccaccgcgacttcgggacctagtgagcggtcttgaagagggccacggacctcgacgtcacagtcgggctgaggagttgggacggtgggggtacctcaggggccggggacctccggtgtcggggctgtcggggcgtcgggggagacgaggaggatgacgacgacgggcacccggaggacgacgaccggcgacggaccacggacgtgaccgtctcctgcgccgcctcctgtggggcgggacccctcgtccacggggggcaggggtcaggggtcctggacgacgaacacctcgtg

SEQ ID NO: 106 - DNA, 1056 bp, synthetic construct
  source 1..1056: mol_type = other DNA
tactgtcacgaccgcggtcggacctcgggttgttggatagaggaggacgacgacgacgactcgagccctgagtcaccctgggtcctgacgaggaaggttgtgtcggggtagaggaggctgaagcgacagttttaggcactcgacagactgatggacgaagttctaatgggtcagtggcaccggaggttggacgtcctgctcctcgagacgcccccggagaccgccgaccaggaccgtgtcgcgacctacctcgccgagttctgacagcgacccaggttctacgttccgaacgacctcgcgcacttgtgcctctatgtgaaacagtggtttacacggaaagtcgggggggggtcgacagaagcgaagcaggtctggttgtagagggcggaggacgtcctctggaggctcgtcgaccaccgcgacttcgggacctagtgagcggtcttgaagagggccacggacctcgacgtcacagtcgggctgaggagttgggacggtgggggtacctcaggggccggggacctccggtgtcggggctgtcggggcgtcgggggagacgaggaggatgacgacgacgggcacccggaggacgacgaccggcgacggaccacggacgtgaccgtctcctgcgccgcctcctgtggggcgggacccctcgtccacggggggcaggggtcaggggtcctggacgacgaacacctcgtgccgagacctcggtgcttgaagagagacaatttcgttcgtcctctgcaccttcttttggggccaggagccagcggcgagtagcaacgccaacaccggcggcgcgacaaacggtagcacgcgccggcgctgggggacgatctgcgctacgccgcgctcccccgttacctgaaaacctcgcgtccgacgatgcgcgcgccccacggcgagagcctcggtggcgtccgggaccaacaaaaaatgcaccgggactggcgccattaccagcggcaccgggacatgcgcatgcccgaaacgaaatccgagtacccgcggtcgcccgggttatttttcctcagcgcccccgccccgact

SEQ ID NO: 107 - DNA, 608 bp, synthetic construct
  source 1..608: mol_type = other DNA
gatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaac

SEQ ID NO: 108-199 - skipped (92 entries)

SEQ ID NO: 200 - DNA, 7843 bp, synthetic construct
  source 1..7843: mol_type = other DNA
ttaattaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagccaccatggcttgggtttggaccctgctgttcctgatggccgctgctcagtctatccaggctgagattgtgctgacacagagccctggcacactgagccttagtcctggcgaaagagccacactgtcctgcagagcctctcagtctgtgggcagctcttacctggcctggtatcagcagaagcctggacaggctccccggctgttgatctacggcgccttttctagagccacaggcatccccgatagattcagcggctctggcagcggcaccgatttcaccctgacaatcagcagactggaacccgaggacttcgccgtgtactactgtcagcagtacggcagcagcccttggacatttggccagggcaccaaggtggaaatcaaaggcggcagcgagggcaagtctagcggatctggatctgagagcaagagcacaggcggatctcaggtgcagctggtggaatctggtggcggagttgtgcagcctggcagatccctgagactgtcttgtgccgccagcggcttcaccttcagcagctacacaatgcactgggtccgacaggcccctggcaaaggactggaatgggtcacctttatcagctacgacggcaacaacaagtactacgccgacagcgtgaagggcagattcaccatctccagagacaacagcaagaacaccctgtacctgcagatgaacagcctgagagccgaggacaccgccatctactactgcgccagaacaggatggctgggcccctttgattattggggccagggaaccctggtcaccgtgtcatctgagcccaagtcttccgacaagacccacacatgcccaccttgtccagcaccagagctgctgggaggaccatccgtgttcctgtttccacccaagcctaaggataccctgatgatctccaggacccctgaggtgacatgcgtggtggtggacgtgtctcacgaggatccagaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtataagtgcaaggtgagcaataaggccctgcctgccccaatcgagaagaccatctccaaggcaaagggacagccaagggagccacaggtgtacacactgcctccaagcagagacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattctaagctgaccgtggacaagagccggtggcagcagggcaacgtgttcagctgctctgtgatgcacgaggcactgcacaaccactacacccagaaatccctgtcactgtcccccggaaagtaggacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaaatatctttattttcattacatctgtgtgttggttttttgtgtgtttcgatagtactaacatacgctctccatcaaaacaaaacgaaacaaaacaaactagcaaaataggctgtccccagtgcaagtgcaggtgccagaacatttctcttcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccctcaaaaataaaataaaataaaaattaaaaaaaaaggttactctggctatgaaccctaccatatattatcctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcacagtttcagcagcccaaagctagtgaacccagtgccatgggaaacctgagatggatctgtcacgttgacgaaaacagaggcgccaggttggagttcgaacacccctccaaggtggatggattgctgtccacagggcttagcggaagagtgggtatttgcggccctgagcaggatcctttcgaagcgtcccggactcttgagacacaggcttgctatgaatggtgcctggctgctggcttcccgattggaacaaaatgtgacttgtgcgtagatatagtaaagtccctgccgtttcacagtgagttgctttccgttctcgagcgtaaccaagttgttagacattgtgtaatatcctttctctgcccattgcaggacggaggtggttttggagctggcttcgcttatgacgtgggctgcgatctgtgggttagagcctccgccggaaccgccgccggagccaccgccgagcttgagcagtccgaagctggtgaagcctgttccgtgagacacttgagaaggatcggtgacgttcacaaacacggaggcccctggctgcagttcgaaaacgcctccgagatgaatgctctgctggccgcatggcttagcagagctgtgggtgttagcagcccgcagcaggattctctcaaatctgccggggcttttcagacacagggaagcaatgaaaggagcctggctagaagcctcgcggttggagcagaacgtgacttgagcatagatataatagagcccctgccgcttgactgtcagctgtttcccattctcgagggtcacgagattgttggacatcgtataataccccttttcagcccactggaggacagatgtggtcttgctagaggcctcggagatcacatgggcagcaatctgtgggttacttcctccgccagagccgcctccagagccgccgcccagcttcagcaggccgaaagatgtaaagccggtgccgtgggacacctggctagggtctgtcacattcacgaacacgcttgcgccaggctgcagctcaaacactccgccgaggtgaatagactgctggccacaaggtttggcgctgctgtgtgtgttggcggctctcagcagaatccgctcgaatctgccaggggacttcaggcacaggctggcgataaaaggggcctgagagctggcctctctgttgctgcagaaggtcacttgggcgtagatgtagtacaggccctgtctcttcacggtcagctgcttgccgttttccagggtgaccaggttgttgctcatggtgtagtagcccttctcggcccactgcagcacgcttgttgtcttgctgctggcctcgctgatcacgtgagcggcaatctgtgggttagatccaccaccaccaaggaatgttgacagaagcacccactctccatctttgcgaacgtaggcttggccgtcccttggtgcttctgggatataaccggcctgtatactttgggcagctgccatcaggaatagcaaggtccacacccaagccatggtggcccagtgaagggtccccggctaggctaggccgctcggtcccggtggctggtccggagaacgtgggtccctcacgctgtcttgaaggcgaatggcggtctgctaaccgggctctgcttatatacccgtaggacacccatggtccgcccacgaaccgcccagtaacgcccataactccgcccgggaacgcccatggtcactccccgttacgcccctttccactgacgtcaatggaaagtccccgcggttttggtgccaagaacattgactaatatggaatttccccacccaccattgtcggtaatggtgggaaaggggaactggccccgttcccattgacgtcactggctattgccaaggacattgactaataatagaaatccccctttttggtgccaaatgaggcggtgggcttatggaaagtccccatttaacccctattctggtgccaggacggatgtatatgcttgccaagtcattgatttataattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaaggccaccatgtgccatcagcagctggtcattagttggtttagcctggtctttctggcatcacctctggtcgcaatctgggaactgaaaaaggacgtgtacgtggtggagctggactggtatcctgatgccccaggcgagatggtggtgctgacctgcgacacacctgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggagcacagacatcctgaaggatcagaaggagcccaagaacaagaccttcctgaggtgcgaggccaagaattattccggccgctttacctgttggtggctgaccacaatcagcaccgatctgacattttccgtgaagtctagcaggggctcctctgaccctcagggagtgacatgcggagcagccaccctgtctgccgagcgggtgagaggcgataacaaggagtacgagtattccgtggagtgccaggaggactctgcctgtccagcagcagaggagtccctgccaatcgaagtgatggtggatgccgtgcacaagctgaagtacgagaattatacaagctccttctttatccgggacatcatcaagcccgatccccctaagaacctgcagctgaagcctctgaagaattctagacaggtggaggtgagctgggagtaccccgacacctggagcacacctcactcctatttctctctgaccttttgcgtgcaggtgcagggcaagtctaagcgggagaagaaggacagagtgttcaccgataagacaagcgccaccgtgatctgtaggaagaacgcctctatcagcgtgagggcccaggatcgctactattctagctcctggagcgagtgggcctccgtgccttgctctggaggaggaggaggaggctcccgcaatctgccagtggcaaccccagacccaggaatgttcccatgtctgcaccacagccagaacctgctgcgggccgtgtccaatatgctgcagaaggccagacagacactggagttttacccttgcacctccgaggagatcgaccacgaggatatcacaaaggataagacctctacagtggaggcctgcctgccactggagctgaccaagaacgagagctgtctgaattccagggagacatctttcatcaccaacggcagctgcctggcctcccgcaagacatcttttatgatggccctgtgcctgtctagcatctacgaggacctgaagatgtatcaggtggagttcaagaccatgaacgccaagctgctgatggaccccaagaggcagattttcctggatcagaatatgctggccgtgatcgacgagctgatgcaggccctgaacttcaatagcgagacagtgcctcagaagtcctctctggaggagccagacttttacaagaccaagatcaagctgtgcatcctgctgcacgcttttcggattagagctgtgaccattgatagagtgatgtcctacctgaatgcttcctgaggcgcgccgcaatttgtacccttaataaatttcacaaacagattttatcgcatcgtgtcttattgcgggggagaaaaccgatgtcggcatagaaaaccgccatgattctaagacgtccgaacgcgagtgggtggggaacaacccataccggacagatgccgatgagccacccgcacccttgggtgcgggaggtacggggtggtttgttcatcctatggttccgaccccacaaacagcccccagagtcggtttgggtatggttacattttctgtctggtggtcgggcttgtttcttccttg

SEQ ID NO: 201 - DNA, 10087 bp, synthetic construct
  source 1..10087: mol_type = other DNA
ttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggagggggggacgcgggggcggaggagggggggacgcgggggcggaggagggggggacgcgggggcggaggagggggctcacccgcgttcgtgccttcccgcaggaggaacgccctcgtcgaggcgaccggcggcgaccgttgcgtggaccgcttcctgctcgtcgggggggagctagcgctaccggtcgccaccatgtctcagacacaacctccagctccagtcggccctggcgatcctgatgtgtatctgaagggcgtgccaagcgccggcatgcatcctaggggtgttcatgcccctagaggacaccccagaatgatctctggccctcctcagagaggcgacaacgatcaggctgctggacagtgtggcgatagcggactgctgagagtgggcgccgataccacaatcagcaagccatctgaggctgtgcggcctcctacaatccccagaacccctagagtgccccgcgagccaagagtgcctagacctcctagagagcccagagaaccccgggtgccaagagatcccagggatcctagagtccctcgggaccccagagatccaaggcagcctagatcacccagagagcctcggagcccaagagagccaagaacaccacggacaccccgggaacctagaacagccagaggaagcgtttagggaagcccaggccagacgcgtatgtcgtgggccctggaaatggcggacaccttcctggacaccatgcgggttgggcccaggacgtacgccgacgtacgcgatgagatcaataaaagggggcgtgaggaccgggaggcggccagaaccgccgtgcacgacccggagcgtcccctgctgcgctctcccgggctgctgcccgaaatcgcccccaacgcatccttgggtgtggcacatcgaagaaccggcgggaccgtgaccgacagtccccgtaatccggtaacccgttgagtcccgggtacgaccatcacccgagtctctgggcggagggtggttcccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagatcccagagaccccagggtaccgcgtgaccccagggatccacgacaaccccggtctcccagggagccccggtctccccgggagccccggaccccacgcaccccccgcgaaccacgtacggctcgcgggtctgtatagcccgggcaagtatgcccccctggcgagcccagaccccttctccccacaagatggagcatacgctcgggcccgcgtcgggatccacaccgcggttcgcgtcccgcccaccggaagctcaacccacacgcacttgcggcaagacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctaggtctagattaattaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagccaccatggcttgggtttggaccctgctgttcctgatggccgctgctcagtctatccaggctgagattgtgctgacacagagccctggcacactgagccttagtcctggcgaaagagccacactgtcctgcagagcctctcagtctgtgggcagctcttacctggcctggtatcagcagaagcctggacaggctccccggctgttgatctacggcgccttttctagagccacaggcatccccgatagattcagcggctctggcagcggcaccgatttcaccctgacaatcagcagactggaacccgaggacttcgccgtgtactactgtcagcagtacggcagcagcccttggacatttggccagggcaccaaggtggaaatcaaaggcggcagcgagggcaagtctagcggatctggatctgagagcaagagcacaggcggatctcaggtgcagctggtggaatctggtggcggagttgtgcagcctggcagatccctgagactgtcttgtgccgccagcggcttcaccttcagcagctacacaatgcactgggtccgacaggcccctggcaaaggactggaatgggtcacctttatcagctacgacggcaacaacaagtactacgccgacagcgtgaagggcagattcaccatctccagagacaacagcaagaacaccctgtacctgcagatgaacagcctgagagccgaggacaccgccatctactactgcgccagaacaggatggctgggcccctttgattattggggccagggaaccctggtcaccgtgtcatctgagcccaagtcttccgacaagacccacacatgcccaccttgtccagcaccagagctgctgggaggaccatccgtgttcctgtttccacccaagcctaaggataccctgatgatctccaggacccctgaggtgacatgcgtggtggtggacgtgtctcacgaggatccagaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtataagtgcaaggtgagcaataaggccctgcctgccccaatcgagaagaccatctccaaggcaaagggacagccaagggagccacaggtgtacacactgcctccaagcagagacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattctaagctgaccgtggacaagagccggtggcagcagggcaacgtgttcagctgctctgtgatgcacgaggcactgcacaaccactacacccagaaatccctgtcactgtcccccggaaagtaggacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaaatatctttattttcattacatctgtgtgttggttttttgtgtgtttcgatagtactaacatacgctctccatcaaaacaaaacgaaacaaaacaaactagcaaaataggctgtccccagtgcaagtgcaggtgccagaacatttctcttcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccctcaaaaataaaataaaataaaaattaaaaaaaaaggttactctggctatgaaccctaccatatattatcctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcacagtttcagcagcccaaagctagtgaacccagtgccatgggaaacctgagatggatctgtcacgttgacgaaaacagaggcgccaggttggagttcgaacacccctccaaggtggatggattgctgtccacagggcttagcggaagagtgggtatttgcggccctgagcaggatcctttcgaagcgtcccggactcttgagacacaggcttgctatgaatggtgcctggctgctggcttcccgattggaacaaaatgtgacttgtgcgtagatatagtaaagtccctgccgtttcacagtgagttgctttccgttctcgagcgtaaccaagttgttagacattgtgtaatatcctttctctgcccattgcaggacggaggtggttttggagctggcttcgcttatgacgtgggctgcgatctgtgggttagagcctccgccggaaccgccgccggagccaccgccgagcttgagcagtccgaagctggtgaagcctgttccgtgagacacttgagaaggatcggtgacgttcacaaacacggaggcccctggctgcagttcgaaaacgcctccgagatgaatgctctgctggccgcatggcttagcagagctgtgggtgttagcagcccgcagcaggattctctcaaatctgccggggcttttcagacacagggaagcaatgaaaggagcctggctagaagcctcgcggttggagcagaacgtgacttgagcatagatataatagagcccctgccgcttgactgtcagctgtttcccattctcgagggtcacgagattgttggacatcgtataataccccttttcagcccactggaggacagatgtggtcttgctagaggcctcggagatcacatgggcagcaatctgtgggttacttcctccgccagagccgcctccagagccgccgcccagcttcagcaggccgaaagatgtaaagccggtgccgtgggacacctggctagggtctgtcacattcacgaacacgcttgcgccaggctgcagctcaaacactccgccgaggtgaatagactgctggccacaaggtttggcgctgctgtgtgtgttggcggctctcagcagaatccgctcgaatctgccaggggacttcaggcacaggctggcgataaaaggggcctgagagctggcctctctgttgctgcagaaggtcacttgggcgtagatgtagtacaggccctgtctcttcacggtcagctgcttgccgttttccagggtgaccaggttgttgctcatggtgtagtagcccttctcggcccactgcagcacgcttgttgtcttgctgctggcctcgctgatcacgtgagcggcaatctgtgggttagatccaccaccaccaaggaatgttgacagaagcacccactctccatctttgcgaacgtaggcttggccgtcccttggtgcttctgggatataaccggcctgtatactttgggcagctgccatcaggaatagcaaggtccacacccaagccatggtggcccagtgaagggtccccggctaggctaggccgctcggtcccggtggctggtccggagaacgtgggtccctcacgctgtcttgaaggcgaatggcggtctgctaaccgggctctgcttatatacccgtaggacacccatggtccgcccacgaaccgcccagtaacgcccataactccgcccgggaacgcccatggtcactccccgttacgcccctttccactgacgtcaatggaaagtccccgcggttttggtgccaagaacattgactaatatggaatttccccacccaccattgtcggtaatggtgggaaaggggaactggccccgttcccattgacgtcactggctattgccaaggacattgactaataatagaaatccccctttttggtgccaaatgaggcggtgggcttatggaaagtccccatttaacccctattctggtgccaggacggatgtatatgcttgccaagtcattgatttataattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaaggccaccatgtgccatcagcagctggtcattagttggtttagcctggtctttctggcatcacctctggtcgcaatctgggaactgaaaaaggacgtgtacgtggtggagctggactggtatcctgatgccccaggcgagatggtggtgctgacctgcgacacacctgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggagcacagacatcctgaaggatcagaaggagcccaagaacaagaccttcctgaggtgcgaggccaagaattattccggccgctttacctgttggtggctgaccacaatcagcaccgatctgacattttccgtgaagtctagcaggggctcctctgaccctcagggagtgacatgcggagcagccaccctgtctgccgagcgggtgagaggcgataacaaggagtacgagtattccgtggagtgccaggaggactctgcctgtccagcagcagaggagtccctgccaatcgaagtgatggtggatgccgtgcacaagctgaagtacgagaattatacaagctccttctttatccgggacatcatcaagcccgatccccctaagaacctgcagctgaagcctctgaagaattctagacaggtggaggtgagctgggagtaccccgacacctggagcacacctcactcctatttctctctgaccttttgcgtgcaggtgcagggcaagtctaagcgggagaagaaggacagagtgttcaccgataagacaagcgccaccgtgatctgtaggaagaacgcctctatcagcgtgagggcccaggatcgctactattctagctcctggagcgagtgggcctccgtgccttgctctggaggaggaggaggaggctcccgcaatctgccagtggcaaccccagacccaggaatgttcccatgtctgcaccacagccagaacctgctgcgggccgtgtccaatatgctgcagaaggccagacagacactggagttttacccttgcacctccgaggagatcgaccacgaggatatcacaaaggataagacctctacagtggaggcctgcctgccactggagctgaccaagaacgagagctgtctgaattccagggagacatctttcatcaccaacggcagctgcctggcctcccgcaagacatcttttatgatggccctgtgcctgtctagcatctacgaggacctgaagatgtatcaggtggagttcaagaccatgaacgccaagctgctgatggaccccaagaggcagattttcctggatcagaatatgctggccgtgatcgacgagctgatgcaggccctgaacttcaatagcgagacagtgcctcagaagtcctctctggaggagccagacttttacaagaccaagatcaagctgtgcatcctgctgcacgcttttcggattagagctgtgaccattgatagagtgatgtcctacctgaatgcttcctgaggcgcgcc

SEQ ID NO: 202 - DNA, 12149 bp, synthetic construct
  source 1..12149: mol_type = other DNA
aagctgtcacctacggactctcgatggggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgcccccccggcgggcccaccccgaacggaattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggtggaggaggggggacgcgggggcggaggaggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggggggagctagcgctaccggtcgccaccatgtctcagacacaacctccagctccagtcggccctggcgatcctgatgtgtatctgaagggcgtgccaagcgccggcatgcatcctaggggtgttcatgcccctagaggacaccccagaatgatctctggccctcctcagagaggcgacaacgatcaggctgctggacagtgtggcgatagcggactgctgagagtgggcgccgataccacaatcagcaagccatctgaggctgtgcggcctcctacaatccccagaacccctagagtgccccgcgagccaagagtgcctagacctcctagagagcccagagaaccccgggtgccaagagatcccagggatcctagagtccctcgggaccccagagatccaaggcagcctagatcacccagagagcctcggagcccaagagagccaagaacaccacggacaccccgggaacctagaacagccagaggaagcgtttagggaagcccaggccagacgcgtatgtcgtgggccctggaaatggcggacaccttcctggacaccatgcgggttgggcccaggacgtacgccgacgtacgcgatgagatcaataaaagggggcgtgaggaccgggaggcggccagaaccgccgtgcacgacccggagcgtcccctgctgcgctctcccgggctgctgcccgaaatcgcccccaacgcatccttgggtgtggcacatcgaagaaccggcgggaccgtgaccgacagtccccgtaatccggtaacccgttgagtcccgggtacgaccatcacccgagtctctgggcggagggtggttcccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagatcccagagaccccagggtaccgcgtgaccccagggatccacgacaaccccggtctcccagggagccccggtctccccgggagccccggaccccacgcaccccccgcgaaccacgtacggctcgcgggtctgtatagcccgggcaagtatgcccccctggcgagcccagaccccttctccccacaagatggagcatacgctcgggcccgcgtcgggatccacaccgcggttcgcgtcccgcccaccggaagctcaacccacacgcacttgcggcaagacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctaggtctagattaattaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagccaccatggcttgggtttggaccctgctgttcctgatggccgctgctcagtctatccaggctgagattgtgctgacacagagccctggcacactgagccttagtcctggcgaaagagccacactgtcctgcagagcctctcagtctgtgggcagctcttacctggcctggtatcagcagaagcctggacaggctccccggctgttgatctacggcgccttttctagagccacaggcatccccgatagattcagcggctctggcagcggcaccgatttcaccctgacaatcagcagactggaacccgaggacttcgccgtgtactactgtcagcagtacggcagcagcccttggacatttggccagggcaccaaggtggaaatcaaaggcggcagcgagggcaagtctagcggatctggatctgagagcaagagcacaggcggatctcaggtgcagctggtggaatctggtggcggagttgtgcagcctggcagatccctgagactgtcttgtgccgccagcggcttcaccttcagcagctacacaatgcactgggtccgacaggcccctggcaaaggactggaatgggtcacctttatcagctacgacggcaacaacaagtactacgccgacagcgtgaagggcagattcaccatctccagagacaacagcaagaacaccctgtacctgcagatgaacagcctgagagccgaggacaccgccatctactactgcgccagaacaggatggctgggcccctttgattattggggccagggaaccctggtcaccgtgtcatctgagcccaagtcttccgacaagacccacacatgcccaccttgtccagcaccagagctgctgggaggaccatccgtgttcctgtttccacccaagcctaaggataccctgatgatctccaggacccctgaggtgacatgcgtggtggtggacgtgtctcacgaggatccagaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtataagtgcaaggtgagcaataaggccctgcctgccccaatcgagaagaccatctccaaggcaaagggacagccaagggagccacaggtgtacacactgcctccaagcagagacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattctaagctgaccgtggacaagagccggtggcagcagggcaacgtgttcagctgctctgtgatgcacgaggcactgcacaaccactacacccagaaatccctgtcactgtcccccggataggacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaaatatctttattttcattacatctgtgtgttggttttttgtgtgtttcgatagtactaacatacgctctccatcaaaacaaaacgaaacaaaacaaactagcaaaataggctgtccccagtgcaagtgcaggtgccagaacatttctcttcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccctcaaaaataaaataaaataaaaattaaaaaaaaaggttactctggctatgaaccctaccatatattatcctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagctcacagtttcagcagcccaaagctagtgaacccagtgccatgggaaacctgagatggatctgtcacgttgacgaaaacagaggcgccaggttggagttcgaacacccctccaaggtggatggattgctgtccacagggcttagcggaagagtgggtatttgcggccctgagcaggatcctttcgaagcgtcccggactcttgagacacaggcttgctatgaatggtgcctggctgctggcttcccgattggaacaaaatgtgacttgtgcgtagatatagtaaagtccctgccgtttcacagtgagttgctttccgttctcgagcgtaaccaagttgttagacattgtgtaatatcctttctctgcccattgcaggacggaggtggttttggagctggcttcgcttatgacgtgggctgcgatctgtgggttagagcctccgccggaaccgccgccggagccaccgccgagcttgagcagtccgaagctggtgaagcctgttccgtgagacacttgagaaggatcggtgacgttcacaaacacggaggcccctggctgcagttcgaaaacgcctccgagatgaatgctctgctggccgcatggcttagcagagctgtgggtgttagcagcccgcagcaggattctctcaaatctgccggggcttttcagacacagggaagcaatgaaaggagcctggctagaagcctcgcggttggagcagaacgtgacttgagcatagatataatagagcccctgccgcttgactgtcagctgtttcccattctcgagggtcacgagattgttggacatcgtataataccccttttcagcccactggaggacagatgtggtcttgctagaggcctcggagatcacatgggcagcaatctgtgggttacttcctccgccagagccgcctccagagccgccgcccagcttcagcaggccgaaagatgtaaagccggtgccgtgggacacctggctagggtctgtcacattcacgaacacgcttgcgccaggctgcagctcaaacactccgccgaggtgaatagactgctggccacaaggtttggcgctgctgtgtgtgttggcggctctcagcagaatccgctcgaatctgccaggggacttcaggcacaggctggcgataaaaggggcctgagagctggcctctctgttgctgcagaaggtcacttgggcgtagatgtagtacaggccctgtctcttcacggtcagctgcttgccgttttccagggtgaccaggttgttgctcatggtgtagtagcccttctcggcccactgcagcacgcttgttgtcttgctgctggcctcgctgatcacgtgagcggcaatctgtgggttagatccaccaccaccaaggaatgttgacagaagcacccactctccatctttgcgaacgtaggcttggccgtcccttggtgcttctgggatataaccggcctgtatactttgggcagctgccatcaggaatagcaaggtccacacccaagccatggtggcccagtgaagggtccccggctaggctaggccgctcggtcccggtggctggtccggagaacgtgggtccctcacgctgtcttgaaggcgaatggcggtctgctaaccgggctctgcttatatacccgtaggacacccatggtccgcccacgaaccgcccagtaacgcccataactccgcccgggaacgcccatggtcactccccgttacgcccctttccactgacgtcaatggaaagtccccgcggttttggtgccaagaacattgactaatatggaatttccccacccaccattgtcggtaatggtgggaaaggggaactggccccgttcccattgacgtcactggctattgccaaggacattgactaataatagaaatccccctttttggtgccaaatgaggcggtgggcttatggaaagtccccatttaacccctattctggtgccaggacggatgtatatgcttgccaagtcattgatttataattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaaggccaccatgtgccatcagcagctggtcattagttggtttagcctggtctttctggcatcacctctggtcgcaatctgggaactgaaaaaggacgtgtacgtggtggagctggactggtatcctgatgccccaggcgagatggtggtgctgacctgcgacacacctgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggagcacagacatcctgaaggatcagaaggagcccaagaacaagaccttcctgaggtgcgaggccaagaattattccggccgctttacctgttggtggctgaccacaatcagcaccgatctgacattttccgtgaagtctagcaggggctcctctgaccctcagggagtgacatgcggagcagccaccctgtctgccgagcgggtgagaggcgataacaaggagtacgagtattccgtggagtgccaggaggactctgcctgtccagcagcagaggagtccctgccaatcgaagtgatggtggatgccgtgcacaagctgaagtacgagaattatacaagctccttctttatccgggacatcatcaagcccgatccccctaagaacctgcagctgaagcctctgaagaattctagacaggtggaggtgagctgggagtaccccgacacctggagcacacctcactcctatttctctctgaccttttgcgtgcaggtgcagggcaagtctaagcgggagaagaaggacagagtgttcaccgataagacaagcgccaccgtgatctgtaggaagaacgcctctatcagcgtgagggcccaggatcgctactattctagctcctggagcgagtgggcctccgtgccttgctctggaggaggaggaggaggctcccgcaatctgccagtggcaaccccagacccaggaatgttcccatgtctgcaccacagccagaacctgctgcgggccgtgtccaatatgctgcagaaggccagacagacactggagttttacccttgcacctccgaggagatcgaccacgaggatatcacaaaggataagacctctacagtggaggcctgcctgccactggagctgaccaagaacgagagctgtctgaattccagggagacatctttcatcaccaacggcagctgcctggcctcccgcaagacatcttttatgatggccctgtgcctgtctagcatctacgaggacctgaagatgtatcaggtggagttcaagaccatgaacgccaagctgctgatggaccccaagaggcagattttcctggatcagaatatgctggccgtgatcgacgagctgatgcaggccctgaacttcaatagcgagacagtgcctcagaagtcctctctggaggagccagacttttacaagaccaagatcaagctgtgcatcctgctgcacgcttttcggattagagctgtgaccattgatagagtgatgtcctacctgaatgcttcctgaggcgcgccgcaatttgtacccttaataaatttcacaaacagattttatcgcatcgtgtcttattgcgggggagaaaaccgatgtcggcatagaaaaccgccatgattctaagacgtccgaacgcgagtgggtggggaacaacccataccggacagatgccgatgagccacccgcacccttgggtgcgggaggtacggggtggtttgttcatcctatggttccgaccccacaaacagcccccagagtcggtttgggtatggttacattttctgtctggtggtcgggcttgtttcttccttgccactccccacccacccactccccacccacccactccccacccacccactccccacccacccactccccacccacccactccccacccacccactccccacccacccactccccacccacccactccccacccacccactccccacccacccaaaaatcaaccgggagacaacattgccaatcgaacccaatttaatgtagttaaaggctgggtgcaaattgcggggtgatgggggggaagagagacgacaagaaggacgcgcgtgtcgatgcggtcttttagcggagcagccacatcaggagcgccccaaatccgcccgacagaacggccacgaggagacaggcgatcaccatgccgacgcagcgggtgcgtctgcgtcgacgccttaataccgactgttggcggcccatgcgtacgaggaagtcgttggccgcctcgtcttcgctttccgagtagtaggcttcgaccgaaactggcgaggccgtgggataaagcggcacggacatgtcggatcgcgctgaggagttgggatcggagagccgggacgtcatcgaggccggaagaaagctccgggtgggaagttgcggtcgctgtgactcacgatttttaatttgctgcggctaggcggaccaccggccctttatgcgcctcgggcaattgacgtcacataccacgcaatcccacacaggacggcccccaggccggaagccccccggagccaccgagcggccagccaggcgacaaacagggagggggcgtcgacagcctggagggccatcggggagacaacggccgtgtagcccgggggtcgcgggtgtggcgagggcgcggtcgacgtggcgaggggcgggcggtcatgtcgggggggtccgcgttcgtcggaaatcgcgattagctcgtctccgacgtccacct

SEQ ID NO: 203 - DNA, 151 bp, synthetic construct
  source 1..151: mol_type = other DNA
ttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgc

SEQ ID NO: 204 - DNA, 468 bp, synthetic construct
  source 1..468: mol_type = other DNA
atgtctcagacacaacctccagctccagtcggccctggcgatcctgatgtgtatctgaagggcgtgccaagcgccggcatgcatcctaggggtgttcatgcccctagaggacaccccagaatgatctctggccctcctcagagaggcgacaacgatcaggctgctggacagtgtggcgatagcggactgctgagagtgggcgccgataccacaatcagcaagccatctgaggctgtgcggcctcctacaatccccagaacccctagagtgccccgcgagccaagagtgcctagacctcctagagagcccagagaaccccgggtgccaagagatcccagggatcctagagtccctcgggaccccagagatccaaggcagcctagatcacccagagagcctcggagcccaagagagccaagaacaccacggacaccccgggaacctagaacagccagaggaagcgtttag

SEQ ID NO: 205 - DNA, 21 bp, synthetic construct
  source 1..21: mol_type = other DNA
ggaagcccaggccagacgcgt

SEQ ID NO: 206 - DNA, 267 bp, synthetic construct
  source 1..267: mol_type = other DNA
atgtcgtgggccctggaaatggcggacaccttcctggacaccatgcgggttgggcccaggacgtacgccgacgtacgcgatgagatcaataaaagggggcgtgaggaccgggaggcggccagaaccgccgtgcacgacccggagcgtcccctgctgcgctctcccgggctgctgcccgaaatcgcccccaacgcatccttgggtgtggcacatcgaagaaccggcgggaccgtgaccgacagtccccgtaatccggtaacccgttga

SEQ ID NO: 207 - DNA, 64 bp, synthetic construct
  source 1..64: mol_type = other DNA
gtcccgggtacgaccatcacccgagtctctgggcggagggtggttcccccccgtggctctcgag

SEQ ID NO: 208 - DNA, 1072 bp, synthetic construct
  source 1..1072: mol_type = other DNA
atgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagatcccagagaccccagggtaccgcgtgaccccagggatccacgacaaccccggtctcccagggagccccggtctccccgggagccccggaccccacgcaccccccgcgaaccacgtacggctcgcgggtctgtatagcccgggcaagtatgcccccctggcgagcccagaccccttctccccacaagatggagcatacgctcgggcccgcgtcgggatccacaccgcggttcgcgtcccgcccaccggaagctcaacccacacgcacttgcggcaagacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctag

SEQ ID NO: 209 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 210 - DNA, 536 bp, synthetic construct
  source 1..536: mol_type = other DNA
tggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcaga

SEQ ID NO: 211 - skipped

SEQ ID NO: 212 - DNA, 1491 bp, synthetic construct
  source 1..1491: mol_type = other DNA
atggcttgggtttggaccctgctgttcctgatggccgctgctcagtctatccaggctgagattgtgctgacacagagccctggcacactgagccttagtcctggcgaaagagccacactgtcctgcagagcctctcagtctgtgggcagctcttacctggcctggtatcagcagaagcctggacaggctccccggctgttgatctacggcgccttttctagagccacaggcatccccgatagattcagcggctctggcagcggcaccgatttcaccctgacaatcagcagactggaacccgaggacttcgccgtgtactactgtcagcagtacggcagcagcccttggacatttggccagggcaccaaggtggaaatcaaaggcggcagcgagggcaagtctagcggatctggatctgagagcaagagcacaggcggatctcaggtgcagctggtggaatctggtggcggagttgtgcagcctggcagatccctgagactgtcttgtgccgccagcggcttcaccttcagcagctacacaatgcactgggtccgacaggcccctggcaaaggactggaatgggtcacctttatcagctacgacggcaacaacaagtactacgccgacagcgtgaagggcagattcaccatctccagagacaacagcaagaacaccctgtacctgcagatgaacagcctgagagccgaggacaccgccatctactactgcgccagaacaggatggctgggcccctttgattattggggccagggaaccctggtcaccgtgtcatctgagcccaagtcttccgacaagacccacacatgcccaccttgtccagcaccagagctgctgggaggaccatccgtgttcctgtttccacccaagcctaaggataccctgatgatctccaggacccctgaggtgacatgcgtggtggtggacgtgtctcacgaggatccagaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtataagtgcaaggtgagcaataaggccctgcctgccccaatcgagaagaccatctccaaggcaaagggacagccaagggagccacaggtgtacacactgcctccaagcagagacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattctaagctgaccgtggacaagagccggtggcagcagggcaacgtgttcagctgctctgtgatgcacgaggcactgcacaaccactacacccagaaatccctgtcactgtcccccggatag

SEQ ID NO: 213 - DNA, 331 bp, synthetic construct
  source 1..331: mol_type = other DNA
gacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaaatatctttattttcattacatctgtgtgttggttttttgtgtgtttcgatagtactaacatacgctctccatcaaaacaaaacgaaacaaaacaaactagcaaaataggctgtccccagtgcaagtgcaggtgccagaacatttctct

SEQ ID NO: 214 - DNA, 161 bp, synthetic construct
  source 1..161: mol_type = other DNA
tcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatcc

SEQ ID NO: 215 - DNA, 73 bp, synthetic construct
  source 1..73: mol_type = other DNA
gtcctattatataccatcccaagtatcggtctcattggaaaaaaaaattaaaaataaaataaaataaaaactc

SEQ ID NO: 216 - DNA, 395 bp, synthetic construct
  source 1..395: mol_type = other DNA
cgagcgaaagaacgacaggttaaagataatttccaaggaaacaagggattcaggttgatgatttgaccccctataatacttcccggaactcgtagacctaagacggattattttttgtaaataaaagtaacgttactacataaatttaataaagacttataaaatgatttttcccttacaccctccagtcacgtaaattttgtatttctttacttctcgatcaagtttggaacccttttatgtgatatagaatttgaggtactttcttccactccgacgtttgtcgattacgtgtaaccgttgtcggggactacggatacggaataagtagggagtcttttcctaagttcatctccgaactaaacctccaatttcaaaacgatacgacataaaat

SEQ ID NO: 217 - DNA, 1515 bp, synthetic construct
  source 1..1515: mol_type = other DNA
taccgaacccacacctggaacgataaggactaccgtcgacgggtttcatatgtccggccaatatagggtcttcgtggttccctgccggttcggatgcaagcgtttctacctctcacccacgaagacagttgtaaggaaccaccaccacctagattgggtgtctaacggcgagtgcactagtcgctccggtcgtcgttctgttgttcgcacgacgtcacccggctcttcccgatgatgtggtactcgttgttggaccagtgggaccttttgccgttcgtcgactggcacttctctgtcccggacatgatgtagatgcgggttcactggaagacgtcgttgtctctccggtcgagagtccggggaaaatagcggtcggacacggacttcaggggaccgtctaagctcgcctaagacgactctcggcggttgtgtgtgtcgtcgcggtttggaacaccggtcgtcagataagtggagccgcctcacaaactcgacgtcggaccgcgttcgcacaagcacttacactgtctgggatcggtccacagggtgccgtggccgaaatgtagaaagccggacgacttcgacccgccgccgagacctccgccgagaccgcctccttcattgggtgtctaacgacgggtacactagaggctccggagatcgttctggtgtagacaggaggtcacccgacttttccccataatatgctacaggttgttagagcactgggagctcttaccctttgtcgactgtcagttcgccgtccccgagataatatagatacgagttcagtgcaagacgaggttggcgctccgaagatcggtccgaggaaagtaacgaagggacacagacttttcggggccgtctaaactctcttaggacgacgcccgacgattgtgggtgtcgagacgattcggtacgccggtcgtctcgtaagtagagcctccgcaaaagcttgacgtcggtccccggaggcacaaacacttgcagtggctaggaagagttcacagagtgccttgtccgaagtggtcgaagcctgacgagttcgagccgccaccgaggccgccgccaaggccgcctccgagattgggtgtctagcgtcgggtgcagtattcgcttcggtcgaggttttggtggaggcaggacgttacccgtctctttcctataatgtgttacagattgttgaaccaatgcgagctcttgcctttcgttgagtgacactttgccgtccctgaaatgatatagatgcgtgttcagtgtaaaacaaggttagcccttcggtcgtcggtccgtggtaagtatcgttcggacacagagttctcaggccctgcgaagctttcctaggacgagtcccggcgtttatgggtgagaaggcgattcgggacacctgtcgttaggtaggtggaacctccccacaagcttgaggttggaccgcggagacaaaagcagttgcactgtctaggtagagtccaaagggtaccgtgacccaagtgatcgaaacccgacgactttgacact

SEQ ID NO: 218 - skipped

SEQ ID NO: 219 - DNA, 482 bp, synthetic construct
  source 1..482: mol_type = other DNA
tttagttactgaaccgttcgtatatgtaggcaggaccgtggtcttatccccaatttacccctgaaaggtattcgggtggcggagtaaaccgtggtttttccccctaaagataataatcagttacaggaaccgttatcggtcactgcagttacccttgccccggtcaaggggaaagggtggtaatggctgttaccacccacccctttaaggtataatcagttacaagaaccgtggttttggcgcccctgaaaggtaactgcagtcacctttccccgcattgcccctcactggtacccgcaagggcccgcctcaatacccgcaatgacccgccaagcacccgcctggtacccacaggatgcccatatattcgtctcgggccaatcgtctggcggtaagcggaagttctgtcgcactccctgggtgcaagaggcctggtcggtggccctggctcgccggatcggatcggcccctgggaagtgacc

SEQ ID NO: 220 - DNA, 454 bp, synthetic construct
  source 1..454: mol_type = other DNA
ataattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaag

SEQ ID NO: 221 - skipped

SEQ ID NO: 222 - DNA, 1599 bp, synthetic construct
  source 1..1599: mol_type = other DNA
atgtgccatcagcagctggtcattagttggtttagcctggtctttctggcatcacctctggtcgcaatctgggaactgaaaaaggacgtgtacgtggtggagctggactggtatcctgatgccccaggcgagatggtggtgctgacctgcgacacacctgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggagcacagacatcctgaaggatcagaaggagcccaagaacaagaccttcctgaggtgcgaggccaagaattattccggccgctttacctgttggtggctgaccacaatcagcaccgatctgacattttccgtgaagtctagcaggggctcctctgaccctcagggagtgacatgcggagcagccaccctgtctgccgagcgggtgagaggcgataacaaggagtacgagtattccgtggagtgccaggaggactctgcctgtccagcagcagaggagtccctgccaatcgaagtgatggtggatgccgtgcacaagctgaagtacgagaattatacaagctccttctttatccgggacatcatcaagcccgatccccctaagaacctgcagctgaagcctctgaagaattctagacaggtggaggtgagctgggagtaccccgacacctggagcacacctcactcctatttctctctgaccttttgcgtgcaggtgcagggcaagtctaagcgggagaagaaggacagagtgttcaccgataagacaagcgccaccgtgatctgtaggaagaacgcctctatcagcgtgagggcccaggatcgctactattctagctcctggagcgagtgggcctccgtgccttgctctggaggaggaggaggaggctcccgcaatctgccagtggcaaccccagacccaggaatgttcccatgtctgcaccacagccagaacctgctgcgggccgtgtccaatatgctgcagaaggccagacagacactggagttttacccttgcacctccgaggagatcgaccacgaggatatcacaaaggataagacctctacagtggaggcctgcctgccactggagctgaccaagaacgagagctgtctgaattccagggagacatctttcatcaccaacggcagctgcctggcctcccgcaagacatcttttatgatggccctgtgcctgtctagcatctacgaggacctgaagatgtatcaggtggagttcaagaccatgaacgccaagctgctgatggaccccaagaggcagattttcctggatcagaatatgctggccgtgatcgacgagctgatgcaggccctgaacttcaatagcgagacagtgcctcagaagtcctctctggaggagccagacttttacaagaccaagatcaagctgtgcatcctgctgcacgcttttcggattagagctgtgaccattgatagagtgatgtcctacctgaatgcttcctga

SEQ ID NO: 223 - DNA, 549 bp, synthetic construct
  source 1..549: mol_type = other DNA
agacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggtggaggaggggggacgcgggggcggaggaggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggggggagctagcgctaccggtcgccacc

SEQ ID NO: 224-299 - skipped (76 entries)

SEQ ID NO: 300 - DNA, 7501 bp, synthetic construct
  source 1..7501: mol_type = other DNA
ttaattaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagccaccatggcttgggtttggaccctgctgttcctgatggccgctgctcagtctatccaggctcaggtgcagctggtggaatctggcggaggacttgctcaacctggcggctctctgagactgtcttgtgccgcttctggcagcaccatcagctctgtggctgtcggctggtacagacagacacctggcaaccagagagagtgggtcgccaccagcagcacaagctctaccacagccacatacgccgacagcgtgaagggcagattcaccatctccagagacaacgccaagaacaccatctacctgcagatgaacagcctgaagcctgaggacaccgccgtgtactactgcaagaccggcctgacaaactggggcagaggcacccaagtgaccgtgtctagcgaacctagaggccccaccatcaagccctgtcctccatgcaaatgccccgctcctaatctgctcggcggacccagcgtgttcatcttcccacctaagatcaaggacgtgctgatgatctctctgagccccatcgtgacctgcgtggtggtggatgtgtctgaggacgaccctgacgtgcagattagttggttcgtgaacaacgtcgaggtgcacacagcccagacacagacccacagagaggactacaacagcaccctgagagtggtgtctgccctgcctatccagcaccaggattggatgagcggcaaagaattcaagtgcaaagtgaacaacaaggacctgcctgctcctatcgagagaaccatcagcaagcccaagggctctgtcagggctcctcaggtgtacgttctgccacctcctgaggaagagatgaccaagaaacaagtgaccctgacctgtatggtcaccgacttcatgcccgaggacatctacgtggaatggaccaacaacggcaagaccgagctgaactacaagaacacagagcccgtgctggacagcgacggcagctacttcatgtacagcaagctgcgcgtcgagaagaagaactgggtcgagagaaacagctacagctgcagcgtggtgcacgagggactgcacaaccaccacaccaccaagagcttcagcagaacccctggcaagtgataagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaaatatctttattttcattacatctgtgtgttggttttttgtgtgtttcgatagtactaacatacgctctccatcaaaacaaaacgaaacaaaacaaactagcaaaataggctgtccccagtgcaagtgcaggtgccagaacatttctcttcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccctcaaaaataaaataaaataaaaattaaaaaaaaaggttactctggctatgaaccctaccatatattatcctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcctatcacagcttcagtaaaccgaaggaggagaagcccacacgatggataacttggctggcctcggtcacgttcacgaacacggaggcgccagcttgcagctcgaacacgcctcctaaatggacggactgctgctcgcacagctgggaggaggagtgggtgttggcggccttcagtaaaatcctctcggagccggagctgggcttcagccataaacccacgatgaagggcctctggctggagggctcacgattggagcagaaggtaacttgggtgtacacgtagtataaaccctccctcttcacggtcagctgcttgccgttctccagcatcactaaattgctcttcatggtgtagtagccctttttggcccactgcagcacggaggcggcattggagttggcctcggacaccacgtgggcggcaatctgagggtcggagccgccgccagagccgccgccggagccgccacccagcttcagtaaaccgaaggagctgaagcccacacgatggataacttgggaggcctcggtcacgttcacgaacacggaggcgccagcttgcagctcaaacacgcctcctaaatgcacggactgctgctcgcacagctggctggaggagtgggtgttggcggccttcagcagaatcctctcgctgccggaggagggcttcagccacagtcccacgatgaaaggcctctgggaggagggctcacgattggagcagaaggtaacttgggtgtacacgtagtataaaccttccctcttcacggtcagctgcttgccgttctccagcatcactaaattgctcttcatggtgtagtagcccttcttggcccactgcagcacgctagcggcgttggagttggcctcggacaccacgtgagcggcgatctgggggtcggagccgccgccagagccgccgccggagccgccacccagcttcagtaaaccaaaggaggagaagcccacacgatggataacttgggaggcctcggtcacgttcacgaacacgctggcgccagcttgcagctcaaacacgcctcctaaatgcacggactgctgctcgcacagctgggaggaggagtgggtgttggcggccttcagtaaaatcctctcggagccgctgctaggcttcagccataaacccacgatgaagggcctctgggaggaaggctcccggttggagcagaaggtaacttgggtgtacacgtagtataaaccctccctcttcactgtcagctgcttgccgttctccagcatcactaaattggacttcatggtgtagtagcccttcttggcccactgcagcacggaagcggcgttggagttggcctcggacaccacgtgggcagcgatctgggggtcagatccaccaccaccagaaccgcctccgccaaggaatgttgacagaagcacccactctccatctttgcgaacgtaggcttggccgtcccttggtgcttctgggatataaccggcctgtatactttgggcagctgccatcaggaatagcaaggtccacacccaagccatggtggcccagtgaagggtccccggctaggctaggccgctcggtcccggtggctggtccggagaacgtgggtccctcacgctgtcttgaaggcgaatggcggtctgctaaccgggctctgcttatatacccgtaggacacccatggtccgcccacgaaccgcccagtaacgcccataactccgcccgggaacgcccatggtcactccccgttacgcccctttccactgacgtcaatggaaagtccccgcggttttggtgccaagaacattgactaatatggaatttccccacccaccattgtcggtaatggtgggaaaggggaactggccccgttcccattgacgtcactggctattgccaaggacattgactaataatagaaatccccctttttggtgccaaatgaggcggtgggcttatggaaagtccccatttaacccctattctggtgccaggacggatgtatatgcttgccaagtcattgatttataattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaaggccaccatgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggcttctctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagttagaagcggaggcggaggttctggtggcggaggaagtggcggcggaggatctagagtgatccctgtgtctggccctgccagatgtctgagccagagcagaaacctgctgaaaaccaccgacgacatggtcaagaccgccagagagaagctgaagcactactcctgcacagccgaggacatcgaccacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgtccagcgcctgaggcgcgccgcaatttgtacccttaataaatttcacaaacagattttatcgcatcgtgtcttattgcgggggagaaaaccgatgtcggcatagaaaaccgccatgattctaagacgtccgaacgcgagtgggtggggaacaacccataccggacagatgccgatgagccacccgcacccttgggtgcgggaggtacggggtggtttgttcatcctatggttccgaccccacaaacagcccccagagtcggtttgggtatggttacattttctgtctggtggtcgggcttgtttcttccttg

SEQ ID NO: 301 - DNA, 536 bp, synthetic construct; identical to SEQ ID NO: 210
  source 1..536: mol_type = other DNA

SEQ ID NO: 302 - skipped

SEQ ID NO: 303 - DNA, 1101 bp, synthetic construct
  source 1..1101: mol_type = other DNA
atggcttgggtttggaccctgctgttcctgatggccgctgctcagtctatccaggctcaggtgcagctggtggaatctggcggaggacttgctcaacctggcggctctctgagactgtcttgtgccgcttctggcagcaccatcagctctgtggctgtcggctggtacagacagacacctggcaaccagagagagtgggtcgccaccagcagcacaagctctaccacagccacatacgccgacagcgtgaagggcagattcaccatctccagagacaacgccaagaacaccatctacctgcagatgaacagcctgaagcctgaggacaccgccgtgtactactgcaagaccggcctgacaaactggggcagaggcacccaagtgaccgtgtctagcgaacctagaggccccaccatcaagccctgtcctccatgcaaatgccccgctcctaatctgctcggcggacccagcgtgttcatcttcccacctaagatcaaggacgtgctgatgatctctctgagccccatcgtgacctgcgtggtggtggatgtgtctgaggacgaccctgacgtgcagattagttggttcgtgaacaacgtcgaggtgcacacagcccagacacagacccacagagaggactacaacagcaccctgagagtggtgtctgccctgcctatccagcaccaggattggatgagcggcaaagaattcaagtgcaaagtgaacaacaaggacctgcctgctcctatcgagagaaccatcagcaagcccaagggctctgtcagggctcctcaggtgtacgttctgccacctcctgaggaagagatgaccaagaaacaagtgaccctgacctgtatggtcaccgacttcatgcccgaggacatctacgtggaatggaccaacaacggcaagaccgagctgaactacaagaacacagagcccgtgctggacagcgacggcagctacttcatgtacagcaagctgcgcgtcgagaagaagaactgggtcgagagaaacagctacagctgcagcgtggtgcacgagggactgcacaaccaccacaccaccaagagcttcagcagaacccctggcaagtgataa

SEQ ID NO: 304 - DNA, 331 bp, synthetic construct; identical to SEQ ID NO: 213
  source 1..331: mol_type = other DNA

SEQ ID NO: 305 - DNA, 161 bp, synthetic construct; identical to SEQ ID NO: 214
  source 1..161: mol_type = other DNA

SEQ ID NO: 306 - DNA, 73 bp, synthetic construct; identical to SEQ ID NO: 215
  source 1..73: mol_type = other DNA

SEQ ID NO: 307 - DNA, 395 bp, synthetic construct; identical to SEQ ID NO: 216
  source 1..395: mol_type = other DNA

SEQ ID NO: 308 - DNA, 1533 bp, synthetic construct
  source 1..1533: mol_type = other DNA
taccgaacccacacctggaacgataaggactaccgtcgacgggtttcatatgtccggccaatatagggtcttcgtggttccctgccggttcggatgcaagcgtttctacctctcacccacgaagacagttgtaaggaaccgcctccgccaagaccaccaccacctagactgggggtctagcgacgggtgcaccacaggctccggttgaggttgcggcgaaggcacgacgtcacccggttcttcccgatgatgtggtacttcaggttaaatcactacgacctcttgccgttcgtcgactgtcacttctccctcccaaatatgatgcacatgtgggttcaatggaagacgaggttggccctcggaaggagggtctccgggaagtagcacccaaataccgacttcggatcgtcgccgaggctctcctaaaatgacttccggcggttgtgggtgaggaggagggtcgacacgctcgtcgtcaggcacgtaaatcctccgcacaaactcgacgttcgaccgcggtcgcacaagcacttgcactggctccggagggttcaataggtagcacacccgaagaggaggaaaccaaatgacttcgacccaccgccgaggccgccgccgagaccgccgccgaggctgggggtctagcggcgagtgcaccacaggctccggttgaggttgcggcgatcgcacgacgtcacccggttcttcccgatgatgtggtacttctcgttaaatcactacgacctcttgccgttcgtcgactggcacttctcccttccaaatatgatgcacatgtgggttcaatggaagacgaggttagcactcgggaggagggtctccggaaagtagcaccctgacaccgacttcgggaggaggccgtcgctctcctaagacgacttccggcggttgtgggtgaggaggtcggtcgacacgctcgtcgtcaggcacgtaaatcctccgcacaaactcgacgttcgaccgcggaggcacaagcacttgcactggctccggagggttcaataggtagcacacccgaagtcgaggaagccaaatgacttcgacccaccgccgaggccgccgccgagaccgccgccgaggctgggagtctaacggcgggtgcaccacaggctccggttgaggttacggcggaggcacgacgtcacccggtttttcccgatgatgtggtacttctcgttaaatcactacgacctcttgccgttcgtcgactggcacttctccctcccaaatatgatgcacatgtgggttcaatggaagacgaggttagcactcgggaggtcggtctccgggaagtagcacccaaataccgacttcgggtcgaggccgaggctctcctaaaatgacttccggcggttgtgggtgaggaggagggtcgacacgctcgtcgtcaggcaggtaaatcctccgcacaagctcgacgttcgaccgcggaggcacaagcacttgcactggctccggtcggttcaataggtagcacacccgaagaggaggaagccaaatgacttcgacactatc

SEQ ID NO: 309 - skipped

SEQ ID NO: 310 - DNA, 482 bp, synthetic construct; identical to SEQ ID NO: 219
  source 1..482: mol_type = other DNA

SEQ ID NO: 311 - DNA, 454 bp, synthetic construct; identical to SEQ ID NO: 220
  source 1..454: mol_type = other DNA

SEQ ID NO: 312 - skipped

SEQ ID NO: 313 - DNA, 1632 bp, synthetic construct
  source 1..1632: mol_type = other DNA
atgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggcttctctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagttagaagcggaggcggaggttctggtggcggaggaagtggcggcggaggatctagagtgatccctgtgtctggccctgccagatgtctgagccagagcagaaacctgctgaaaaccaccgacgacatggtcaagaccgccagagagaagctgaagcactactcctgcacagccgaggacatcgaccacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgtccagcgcctga

SEQ ID NO: 314 - DNA, 293 bp, synthetic construct
  source 1..293: mol_type = other DNA
gttccttctttgttcgggctggtggtctgtcttttacattggtatgggtttggctgagacccccgacaaacaccccagccttggtatcctacttgtttggtggggcatggagggcgtgggttcccacgcccaccgagtagccgtagacaggccatacccaacaaggggtgggtgagcgcaagcctgcagaatcttagtaccgccaaaagatacggctgtagccaaaagagggggcgttattctgtgctacgctattttagacaaacactttaaataattcccatgtttaacgg

SEQ ID NO: 315 - DNA, 1459 bp, synthetic construct
  source 1..1459: mol_type = other DNA
gttaacgtaggtagggtcgtatcgagccgcggtccgtcctgctctgggctcgaatggcatgggggacagcaattatatggttaactccgcccgttttatgactagaaccaatagtttttaatgccaaatgcactgaaatcccctaatttgcaaagccaaacgccccctatgtgagtaatacggggactttttacccaatttcccacgcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaatttaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcaga

SEQ ID NO: 316 - DNA, 220 bp, synthetic construct
  source 1..220: mol_type = other DNA
gacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 317 - DNA, 99 bp, synthetic construct
  source 1..99: mol_type = other DNA
aaatgatttgccctcccatatgtccttccgagtgagagacacaaaaaattccaacacactattgcaatgaaaataaatttcctttattagccagaggtc

SEQ ID NO: 318 - protein, 159 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPP

SEQ ID NO: 319 - protein, 8 aa, synthetic construct
SIINFEKL

SEQ ID NO: 320 - protein, 6 aa, synthetic construct
HHHHHH

SEQ ID NO: 321 - DNA, 13 bp, synthetic construct
  source 1..13: mol_type = other DNA
ctggggctggggt